(19) United States
(12) Patent Application Publication
(10) Pub. No.: US 2024/0140952 A1
(43) Pub. Date: May 2, 2024

(54) INHIBITORS AND DEGRADERS OF JANUS KINASE 2

(71) Applicant: H. LEE MOFFITT CANCER CENTER AND RESEARCH INSTITUTE, INC., Tampa, FL (US)

(72) Inventors: Nicholas LAWRENCE, Lutz, FL (US); Harshani LAWRENCE, Lutz, FL (US); Ernst SCHÖNBRUNN, Tampa, FL (US); Gary REUTHER, Temple Terrace, FL (US)

(21) Appl. No.: 18/272,497

(22) PCT Filed: Jan. 18, 2022
(86) PCT No.: PCT/US2022/012772

§ 371 Date: Jul. 14, 2023

Related U.S. Application Data
(60) Provisional application No. 63/138,196, filed on Jan. 15, 2021.
(60) Provisional application No. 63/178,363, filed on Apr. 22, 2021.

(51) Int. Cl. C07D 487/04; C07D 401/14

(52) U.S. Cl. CPC C07D 487/04; C07D 401/14

(57) ABSTRACT

The present disclosure provides inhibitors of Janus Kinase 2 (JAK2) which may be used in the treatment of medical disorders such as cancer.

### CROSS-REFERENCE TO RELATED APPLICATIONS

This application claims the benefit of priority to U.S. Provisional Application No. 63/138,196, filed Jan. 15, 2021, and U.S. Provisional Application No. 63/178,363, filed Apr. 22, 2021, the disclosures of which are incorporated herein by reference in their entireties.

### STATEMENT REGARDING FEDERALLY SPONSORED RESEARCH OR DEVELOPMENT

This invention was made with government support under Grant No. R50CA211447 PI awarded by the National Institutes of Health and the National Cancer Institute. The Government has certain rights in the invention.

### TECHNICAL FIELD

This disclosure relates to compounds for the treatment of medical disorders, and more particularly to inhibitors and degraders of Janus Kinase 2 (JAK2).

### BACKGROUND

Protein kinases have become one of the most desired classes of drug targets given their crucial roles in the regulation of cellular proliferation, survival, signaling, metabolism, and homeostasis. As a mediator of cytokine receptor activation, Janus kinase 2 (JAK2) is a cytosolic tyrosine kinase that phosphorylates signal transducer and activator of transcription proteins (STATs) resulting in SH2-domain mediated dimerization and STAT activation. STATs govern many processes including cell proliferation, differentiation, and immunological responses vital for cell survival. (see Vogelstein, B.; Papadopoulos, N.; Velculescu, V. E.; Zhou, S.; Diaz, L. A., Jr.; Kinzler, K. W. Cancer genome landscapes. Science 2013, 339, 1546-1558) The JAK/STAT pathway has been listed as one of the twelve core cancer pathways demonstrating the importance of proper JAK2 regulation to maintain normal cell function. JAK2 is a multi-domain protein which undergoes trans-autophosphorylation on the activation loop of the kinase domain (KD) involving residues Tyr1007 and Tyr1008. The purpose of this phosphorylation is not fully known but is thought to aid in the recruitment and phosphorylation of STATs. (see Hubbard, S. R. Mechanistic insights into regulation of JAK2 tyrosine kinase. Front Endocrinol (Lausanne) 2017, 8, 361.) Type 1 inhibitors have been shown to bind to the ATP binding site of the active conformation of JAK2, but inhibition paradoxically leads to accumulation of phosphorylated residues on the activation loop with increasing inhibitor concentration. (see Kesarwani, M.; Huber, E.; Kincaid, Z.; Evelyn, C. R.; Biesiada, J.; Rance, M.; Thapa, M. B.; Shah, N. P.; Meller, J.; Zheng, Y.; Azam, M. Targeting substrate-site in Jak2 kinase prevents emergence of genetic resistance. Sci Rep 2015, 5, 14538; and Koppikar, P.; Bhagwat, N.; Kilpivaara, O.; Manshouri, T.; Adli, M.; Hricik, T.; Liu, F.; Saunders, L. M.; Mullally, A.; Abdel-Wahab, O.; Leung, L.; Weinstein, A.; Marubayashi, S.; Goel, A.; Gonen, M.; Estrov, Z.; Ebert, B. L.; Chiosis, G.; Nimer, S. D.; Bernstein, B. E.; Verstovsek, S.; Levine, R. L. Heterodimeric JAK-STAT activation as a mechanism of persistence to JAK2 inhibitor therapy. Nature 2012, 489, 155-159.) This phenomenon has been attributed to the protection of activation loop phosphotyrosines from phosphatases. (see Gorantla, S. P.; Babu, K. S.; Illert, A. L.; von Bubnoff, N.; Peschel, C.; Duyster, J. Ruxolitinib mediated paradox JAK2 hyperphosphorylation is due to the protection of activation loop phosphotyrosines from phosphatases. Blood 2013, 122, 2847; and Tvorogov, D.; Thomas, D.; Liau, N. P. D.; Dottore, M.; Barry, E. F.; Lathi, M.; Kan, W. L.; Hercus, T. R.; Stomski, F.; Hughes, T. P.; Tergaonkar, V.; Parker, M. W.; Ross, D. M.; Majeti, R.; Babon, J. J.; Lopez, A. F. Accumulation of JAK activation loop phosphorylation is linked to type I JAK inhibitor withdrawal syndrome in myelofibrosis. Sci Adv 2018, 4, eaat3834.) Higher levels of phosphorylated Tyr1007/1008, and therefore higher levels of constitutively active JAK2, have also been shown to be caused by a single hyperactivating point mutation, V617F, found in the pseudokinase domain (PKD) of JAK2. (see Bandaranayake, R. M.; Ungureanu, D.; Shan, Y.; Shaw, D. E.; Silvennoinen, O.; Hubbard, S. R. Crystal structures of the JAK2 pseudokinase domain and the pathogenic mutant V617F. Nat Struct Mol Biol 2012, 19, 754-759.)

Constitutively active JAK2 has been identified in several types of cancers including breast cancer, lymphomas, and myeloid malignancies. (see Meyer, S. C. Mechanisms of resistance to JAK2 inhibitors in myeloproliferative neoplasms. Hematol Oncol Clin North Am 2017, 31, 627-642; Bousoik, E.; Montazeri Aliabadi, H. “Do we know jack” about JAK? A closer look at JAK/STAT signaling pathway. Front Oncol 2018, 8, 287; and Karantanos, T.; Moliterno, A. R. The roles of JAK2 in DNA damage and repair in the myeloproliferative neoplasms: Opportunities for targeted therapy. Blood Rev 2018, 32, 426-432.) The V617F mutation is the most commonly identified mutation found in myeloproliferative neoplasms (MPNs), in more than 95% of polycythemia vera patients, and in more than 50% of all thrombocythemia and primary myelofibrosis cases. (see Schieber, M.; Crispino, J. D.; Stein, B. Myelofibrosis in 2019: moving beyond JAK2 inhibition. Blood Cancer J 2019, 9, 74.) The FDA approved the pyrrolopyrimidine JAK2 inhibitor ruxolitinib in 2011 and the dianilinopyrimidine inhibitor fedratinib in 2019 for treating myelofibrosis. (see Tefferi, A. JAK inhibitors for myeloproliferative neoplasms: clarifying facts from myths. Blood 2012, 119, 2721-2730; Quintas-Cardama, A.; Vaddi, K.; Liu, P.; Manshouri, T.; Li, J.; Scherle, P. A.; Caulder, E.; Wen, X.; Li, Y.; Waeltz, P.; Rupar, M.; Bum, T.; Lo, Y.; Kelley, J.; Covington, M.; Shepard, S.; Rodgers, J. D.; Haley, P.; Kantarjian, H.; Fridman, J. S.; Verstovsek, S. Preclinical characterization of the selective JAK1/2 inhibitor INCB018424: therapeutic implications for the treatment of myeloproliferative neoplasms. Blood 2010, 115, 3109-3117; and Caruso, C. Fedratinib becomes new option in myelofibrosis. Cancer Discov 2019, 9, 1332.) Baricitinib, an achiral analogue of ruxolitinib was approved for rheumatoid arthritis in 2018. (see Mullard, A. FDA approves Eli Lilly's baricitinib. Nat Rev Drug Discov 2018, 17, 460.) JAK2 inhibitors for MPN provide quality of life improvements but provide little efficacy at antagonizing the natural course of disease.

There is clear need for additional therapies which affect JAK2 which may be useful for the treatment of the above medical disorders.

### SUMMARY

The present disclosure is directed to compounds which find use as inhibitors and/or degraders of Janus Kinase 2 (JAK2), as well as the use of such compounds in the treatment of medical disorders such as cancer.

In one aspect, a compound of Formula I or Formula II is provided

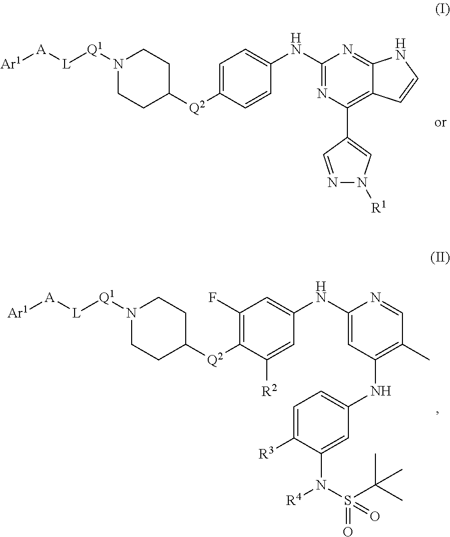

or a pharmaceutically acceptable salt thereof, wherein all variables are as defined further herein.

In another aspect, a compound is provided of Formula III or Formula IV:

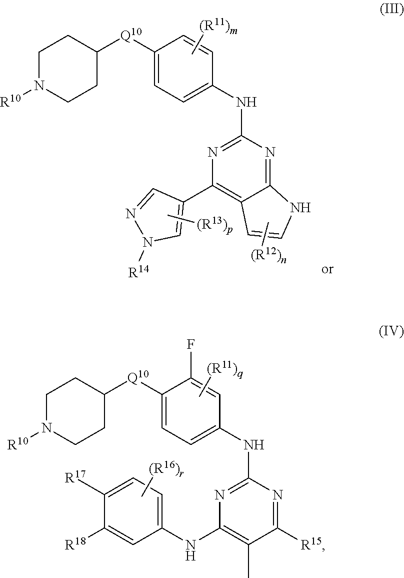

or a pharmaceutically acceptable salt thereof, wherein all variables are as defined further herein.

In yet another aspect, a compound is provided selected from:

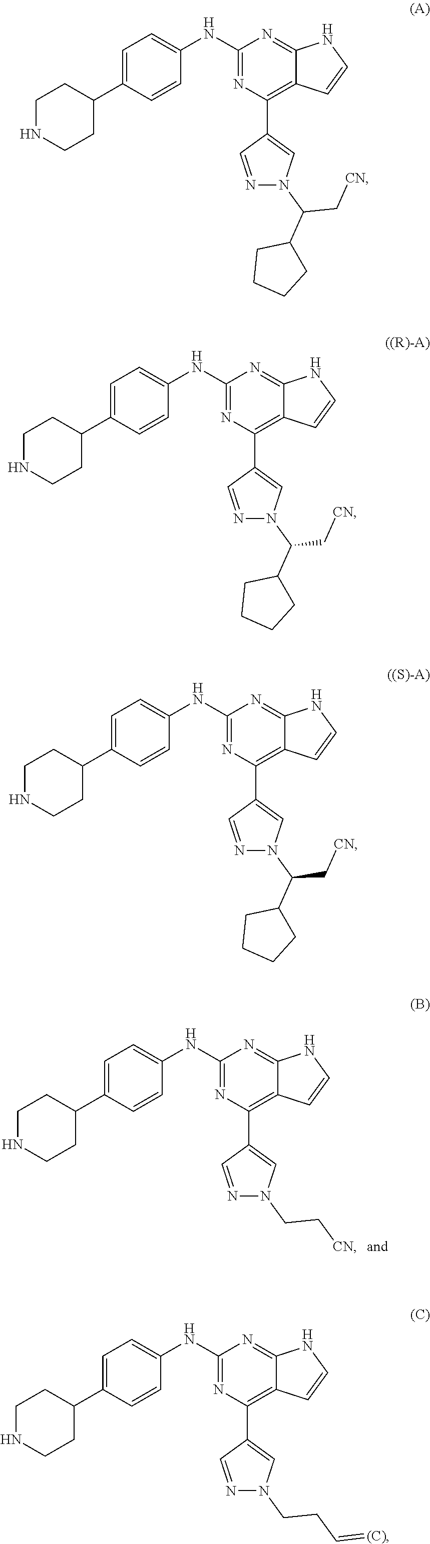

or a pharmaceutically acceptable salt thereof.

In yet another aspect, a compound is provided selected from:

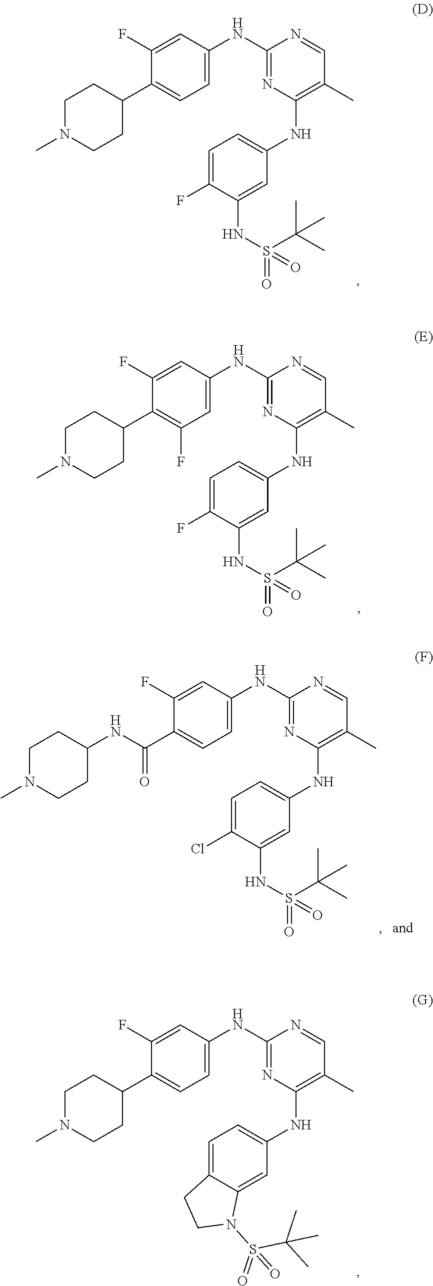

or a pharmaceutically acceptable salt thereof.

Pharmaceutical compositions are also provided comprising a compound described herein, or a pharmaceutically acceptable salt thereof, and a pharmaceutically acceptable carrier or excipient.

In a further aspect, methods are provided for treating cancer in a subject in need thereof comprising administering to the subject a therapeutically effective amount of a compound or composition described herein.

In yet another aspect, methods are provided for treating a JAK2-associated disorder in a subject in need thereof comprising administering to the subject a therapeutically effective amount of a compound or composition described herein.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIGS. 1A-1F show that ruxolitinib enhances the expression and purification of crystallization-grade JAK2 KD. FIG. 1A: Expi293F cells were transiently transfected with wildtype JAK2 KD. Cells were treated with increasing concentrations of ruxolitinib during the 24-hour transfection. Cell lysates were subjected to immunoblotting to detect phosphorylated and unphosphorylated JAK2 KD along with the loading control GAPDH. Densitometry of the Western blot is depicted as a bar graph. FIG. 1B: Same as FIG. 1A except 2 mM and 4 mM butyric acid (BA) or valproic acid (VPA) were added to cells with 1 μM ruxolitinib. FIG. 1C: Expi293F cells were transiently transfected with JAK2 KD+/−1 μM ruxolitinib and analyzed by cellular thermal shift assay (CETSA). Cells were incubated at the indicated temperatures for three minutes and lysates were probed for JAK2 KD and loading control actin. The graph shows the densitometry values relative to loading control as a function of temperature. Data were fit to a four parameter Hill equation, yielding EC₅₀ values of 45 and 47° C. for JAK2 KD expressed in the absence and presence of ruxolitinib, respectively. FIG. 1D: Cells were grown as in FIG. 1C for isothermal dose response (ITDR) against ruxolitinib at 47° C. for three minutes, and cell lysates were probed for JAK2 and GAPDH. The corresponding densitometry values were fit to a four parameter Hill equation yielding EC₅₀=3.8 μM. FIG. 1E: SDS-PAGE of a typical purification of JAK2 KD from a 1 L Expi293F cell culture grown in the presence of 1 μM ruxolitinib and 4 mM butyric acid for 24 hours. Lane 1 GenScript Broad Range Ladder, Lane 2 soluble lysate, Lane 3 GE HisTrap flow-through, Lane 4 GE HisTrap elution peak, Lane 5 GE S75 elution peak. FIG. 1F: Photograph of X-ray grade crystals grown from thus purified JAK2 KD.

FIGS. 2A-2H show that purified JAK2 KD from Expi293 cells is suitable for SAR studies by DSF. Three series of JAK2 inhibitors were subjected to SAR studies: FIG. 2A: Series A of Ruxolitinib stereoisomers and FDA approved derivatives, FIG. 2B: Series B of Piperidine-phenylamine analogues of ruxolitinib, FIG. 2C: Series C of Fedratinib and derived dual JAK2-BRD4 inhibitors. FIG. 2D: DSF derivative plots of JAK2 KD in the absence (DMSO) and presence of 100 μM inhibitor. FIG. 2E: Viability of UKE-1 cells in response to increasing inhibitor concentration. FIG. 2F: DSF and biochemical enzyme inhibition data correlate significantly. FIG. 2G: Analysis of DSF and UKE-1 cell growth inhibition data shows high correlation for series A, but not for series B and C. Series B likely suffers from poor cell permeability. FIG. 2H: For series C, cell inhibition data correlate significantly with binding potential for BRD4-1, suggesting predominant activity through inhibition of BRD4.

FIGS. 3A-3F show the structural basis of ruxolitinib interaction with JAK2. Co-crystal structure of JAK2 KD liganded with ruxolitinib determined at 1.9 Å resolution (PDB 6VGL). FIG. 3A: Electrostatic surface potential of the JAK2-ruxolitinib complex. The inhibitor is shown as spheres. FIG. 3B: Positioning of ruxolitinib in the ATP site FIG. 3C: Binding interactions of ruxolitinib in the ATP site. Potential H-bonding and hydrophobic VDW interactions are indicated as dotted lines. FIG. 3D: 2Fo-Fc electron density map (1) of bound ruxolitinib at 1.9 Å resolution. FIG. 3E: Binding pose of ruxolitinib in c-SRC (PDB 4U5J) and upon superposition with ruxolitinib in JAK2. FIG. 3F: 1.9 Å resolution co-crystal structure of JAK2 with baricitinib (PDB 6VN8) and superposition with ruxolitinib.

FIGS. 4A-4I show that JAK2 discriminates between the R and S stereoisomers of ruxolitinib and derivatives thereof. Distinct stereoisomers and the enantiomeric mixture of ruxolitinib and derivatives were subjected to crystallographic studies with JAK2 KD. FIG. 4A: Co-crystal structure of (S)-ruxolitinib (PDB 6VSN). FIG. 4B: Superposition of the R- and S-isomers of ruxolitinib reveals that (S)-ruxolitinib adopts shape complementarity with the ATP site through ˜1800 rotation about the stereocenter. FIG. 4C: Co-crystal structure obtained with the racemic mixture of ruxolitinib, (rac)-ruxolitinib, PDB 6VNK), reveals only the R-isomer bound. The inset shows the superposition of (rac)-ruxolitinib with ruxolitinib. FIG. 4D: Co-crystal structure showing the H-bonding interactions of (R)-1 (PDB 6VNC) with the hinge region. FIG. 4E: Same as FIG. 4D for the (S)-1 (PDB 6VNB). FIG. 4F: Superposition of (R)-1 and (S)-1 reveals the same adaptation as for ruxolitinib (B). FIG. 4G: Co-crystal structure obtained with (rac)-1 (PDB 6VS3) shows only the R-isomer bound. The inset is the superposition of (rac)-1 with (R)-1. FIG. 4H: Co-crystal structure with derivative 2 devoid of a stereocenter (PDB 6VNJ) reveals the same binding pose and inhibitor conformation as (R)-1. FIG. 4I: Same as FIG. 4H for derivative 3 (PDB 6VNM).

FIGS. 5A-5E show that diaminopyrimidine inhibitors mimic the binding pose of ruxolitinib aniline derivatives. FIG. 5A: Co-crystal structure of JAK2 with fedratinib (PDB 6VNE). The inset shows the superposition of fedratinib with (R)-1. FIG. 5B: Co-crystal structure with 4 (PDB 6VNG). The inset shows the superposition of 4 with fedratinib. FIG. 5C: Co-crystal structure with 5 (PDB 6VNH). FIG. 5D: Co-crystal structure with 6 (PDB 6VNL). FIG. 5E: Co-crystal structure with 7 (PDB 6VNF).

Like reference symbols in the various drawings indicate like elements.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose.

The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound or composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a cancer. The effect can be therapeutic in terms of a partial or complete cure of a disease, condition, symptom or adverse effect attributed to the disease, disorder, or condition. The term “treatment” as used herein can include any treatment of a disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

### Chemical Definitions

Some compounds disclosed herein contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“—”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

“Alkyl” is a straight chain or branched saturated aliphatic hydrocarbon group. In certain embodiments, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl)C₀-C₄alkyl, or —C₀-C₄(C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one embodiments, the alkyl group is optionally substituted as described herein.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In one embodiment, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one embodiment, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one embodiment, the alkynyl group is optionally substituted as described herein.

“Alkoxy” is an alkyl group as defined above covalently bound through an oxygen bridge (—O—). Examples of alkoxy include, but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—). In one embodiment, the alkoxy group is optionally substituted as described herein.

“Alkanoyl” is an alkyl group as defined above covalently bound through a carbonyl (C═O) bridge. The carbonyl carbon is included in the number of carbons, for example C₂alkanoyl is a CH₃(C═O)— group. In one embodiment, the alkanoyl group is optionally substituted as described herein.

“Halo” or “halogen” indicates, independently, any of fluoro, chloro, bromo or iodo.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. In one embodiment, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms (e.g., pyrrolidinyl, imidazolidinyl, piperidinyl, pyrrolinyl, azetidinyl, piperazinyl, and pyrazolidinyl); saturated 4- to 6-membered monocyclic groups containing 1 to 2 oxygen atoms and 1 to 3 nitrogen atoms (e.g., morpholinyl); and saturated 3- to 6-membered heteromonocyclic groups containing 1 to 2 sulfur atoms and 1 to 3 nitrogen atoms (e.g., thiazolidinyl_. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring which contains from 1 to 4, or in some embodiments 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 4, or in some embodiments from 1 to 3 or from 1 to 2, heteroatoms selected from N, O, S, B, or P, with remaining ring atoms being carbon. In one embodiments, the only heteroatom is nitrogen. In one embodiment, the only heteroatom is oxygen. In one embodiment, the only heteroatom is sulfur. Monocyclic heteroaryl groups typically have from 5 to 6 ring atoms. In some embodiments, bicyclic heteroaryl groups are 8- to 10-membered heteroaryl groups, that is groups containing 8 or 10 ring atoms in which one 5-, 6-, or 7-membered aromatic ring is fused to a second aromatic or non-aromatic ring, wherein the point of attachment is the aromatic ring. When the total number of S and O atoms in the heteroaryl group excess 1, these heteroatoms are not adjacent to one another. In one embodiment, the total number of S and O atoms in the heteroaryl group is not more than 2. In another embodiment, the total number of S and O atoms in the heteroaryl group is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produced the same counterion. Lists of additional suitable salts may be found, e.g., in Remington's Pharmaceutical Sciences, 17^{th} ed., Mack Publishing Company, Easton, PA., p. 1418 (1985).

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

### Compounds

In one aspect a compound of Formula I or Formula II is provided

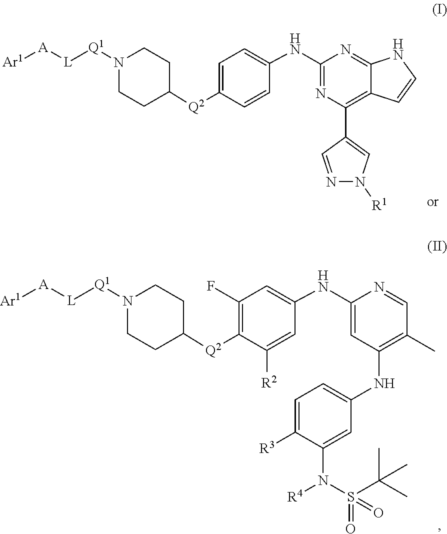

or a pharmaceutically acceptable salt thereof;
wherein:
Ar¹ is selected from

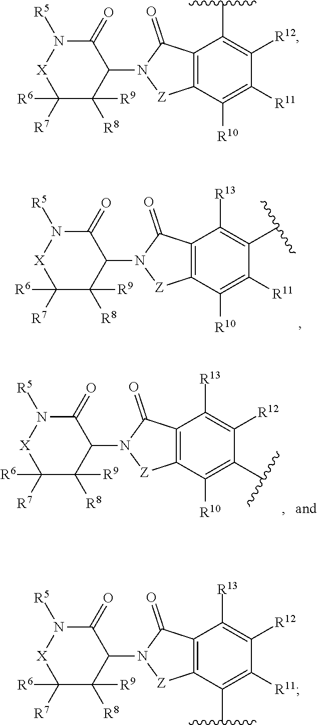

A is selected from —O—, —S—, —NH—, —CH₂—, —O—(C₁-C₄ alkyl)-C(O)NH—, and

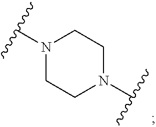 [SMILES: *N1CCN(*)CC1]

L is selected from C₂-C₁₅ alkyl and —(CH₂CH₂O)n(C₁-C₄ alkyl)-, wherein n is selected from 1, 2, 3, 4, 5, 6, 7, and 8;
Q¹ is a bond or —C(═O)—;
Q² is a bond, —NHC═O—, or —C(═O)NH—;
R¹ is selected from

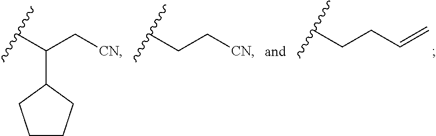

R² is selected from hydrogen or F;
R³ is selected from Cl or F;
R⁴ is hydrogen; or
R³ and R⁴ are brought together with the atoms to which they are attached to form a pyrrolidine ring;
X is selected from —C(═O)— and —CH₂;
Z is selected from —CH₂— and —C(═O)—;
R⁵ is selected from hydrogen and C₁-C₅ alkyl;
R⁶, R⁷, R⁸, and R⁹ are each independently selected from hydrogen, halogen, —NH₂, —OH, —NO₂, —CN, C₁-C₄ alkyl, C₂-C₄ alkenyl, C₁-C₄ haloalkyl, C₁-C₄ cyanoalkyl, C₁-C₄ hydroxyalkyl, C₁-C₄ haloalkoxy, C₁-C₄ alkoxy, C₁-C₄ alkylamino, (independently C₁-C₄ dialkylamino, and C₁-C₄ aminoalkyl; and
R¹⁰, R¹¹, R¹², and R¹³ are each independently selected from hydrogen, halogen, —NH₂, —OH, —NO₂, —CN, C₁-C₄ alkyl, C₂-C₄ alkenyl, C₁-C₄ haloalkyl, C₁-C₄ cyanoalkyl, C₁-C₄ hydroxyalkyl, C₁-C₄ haloalkoxy, C₁-C₄ alkoxy, C₁-C₄ alkylamino, independently C₁-C₄ dialkylamino, and C₁-C₄ aminoalkyl.

In some embodiments of Formula I or Formula II, A is —O—. In some embodiments of Formula I or Formula II, A is —S—. In some embodiments of Formula I or Formula II, A is —NH—. In some embodiments of Formula I or Formula II, A is —CH₂—. In some embodiments of Formula I or Formula II, A is —O—(C₁-C₄ alkyl)-C(O)NH—. In some embodiments of Formula I or Formula II, A is

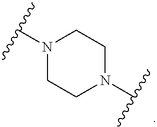 [SMILES: *N1CCN(*)CC1]

In some embodiments of Formula I or Formula II, L is C₂-C₁₅ alkyl. In some embodiments of Formula I or Formula II, L is C₂-C₁₂ alkyl. In some embodiments of Formula I or Formula II, L is C₂-C₈ alkyl. In some embodiments of Formula I or Formula II, L is C₂-C₆ alkyl. In some embodiments of Formula I or Formula II, L is C₂-C₄ alkyl. In some embodiments of Formula I or Formula II, L is selected from ethyl, n-propyl, and isopropyl. In some embodiments of Formula I or Formula II, L is ethyl. In some embodiments of Formula I or Formula II, L is C₅ alkyl. In some embodiments of Formula I or Formula II, L is selected from n-pentyl and neopentyl. In some embodiments of Formula I or Formula II, L is n-pentyl.

In some embodiments of Formula I or Formula II, L is —(CH₂CH₂O)n(C₁-C₄ alkyl)-, wherein n is selected from 1, 2, 3, 4, 5, 6, 7, and 8. In some embodiments of Formula I or Formula II, L is —(CH₂CH₂O)ₙ(C₁-C₄ alkyl)-, wherein n is 1. In some embodiments of Formula I or Formula II, L is —(CH₂CH₂O)ₙ(C₁-C₄ alkyl)-, wherein n is 2. In some embodiments of Formula I or Formula II, L is —(CH₂CH₂O)ₙ(C₁-C₄ alkyl)-, wherein n is 3. In some embodiments of Formula I or Formula II, L is —(CH₂CH₂O)ₙ(C₁-C₄ alkyl)-, wherein n is 4. In some embodiments of Formula I or Formula II, L is —(CH₂CH₂O)ₙ(C₁-C₄ alkyl)-, wherein n is 5. In some embodiments of Formula I or Formula II, L is —(CH₂CH₂O)ₙ(C₁-C₄ alkyl)-, wherein n is 6. In some embodiments of Formula I or Formula II, L is —(CH₂CH₂O)ₙ(C₁-C₄ alkyl)-, wherein n is 7. In some embodiments of Formula I or Formula II, L is —(CH₂CH₂O)ₙ(C₁-C₄ alkyl)-, wherein n is 8. In some embodiments of Formula I or Formula II, L is —(CH₂CH₂O)ₙ(C₁ alkyl)-, wherein n is selected from 1, 2, 3, 4, 5, 6, 7, and 8. In some embodiments of Formula I or Formula II, L is —(CH₂CH₂O)ₙ(C₂ alkyl)-, wherein n is selected from 1, 2, 3, 4, 5, 6, 7, and 8. In some embodiments of Formula I or Formula II, L is —(CH₂CH₂O)ₙ(C₃ alkyl)-, wherein n is selected from 1, 2, 3, 4, 5, 6, 7, and 8. In some embodiments of Formula I or Formula II, L is —(CH₂CH₂O)ₙ(C₄ alkyl)-, wherein n is selected from 1, 2, 3, 4, 5, 6, 7, and 8. In some embodiments of Formula I or Formula II, L is —CH₂CH₂OCH₂CH₂—.

In some embodiments of Formula I or Formula II, L is —(CH₂CH₂O)₁₋₆(C₁-C₄ alkyl)-. In some embodiments of Formula I or Formula II, L is —(CH₂CH₂O)₁₋₄(C₁-C₄ alkyl)-. In some embodiments of Formula I or Formula II, L is —(CH₂CH₂O)₁₋₂(C₁-C₄ alkyl)-. In some embodiments of Formula I or Formula II, L is —(CH₂CH₂O)(C₁-C₄ alkyl)-.

In some embodiments of Formula I or Formula II, L is selected from —(CH₂CH₂O)ₙ(CH₂)—, —(CH₂CH₂O)ₙ(CH₂CH₂)—, —(CH₂CH₂O)ₙ(CH₂CH₂CH₂)—, and —(CH₂CH₂O)ₙ(CH(CH₃)CH₂)—. In some embodiments of Formula I or Formula II, L is —(CH₂CH₂O)ₙ(CH₂CH₂)—. In some embodiments of Formula I or Formula II, L is selected from —(CH₂CH₂O)₃(CH₂CH₂)—, —(CH₂CH₂O)₄(CH₂CH₂)—, —(CH₂CH₂O)₅(CH₂CH₂)—, and —(CH₂CH₂O)₆(CH₂CH₂)—. In some embodiments of Formula II, L is —CH₂CH₂OCH₂CH₂—.

In some embodiments of Formula I or Formula II, Q¹ is a bond. In some embodiments of Formula I or Formula II, Q¹ is —C(═O)—.

In some embodiments of Formula I or Formula II, Q₂ is a bond. In some embodiments of Formula I or Formula II, Q² is —NH(C═O)—. In some embodiments of Formula I or Formula II, Q² is —C(═O)NH—.

In some embodiments of Formula I or Formula II, Ar¹ is

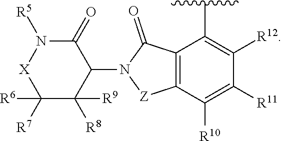

In some embodiments of Formula I or Formula II, Ar¹ is

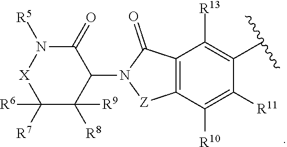

In some embodiments of Formula I or Formula II, Ar¹ is

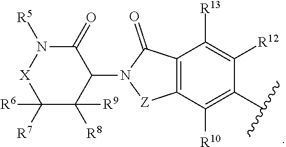

In some embodiments of Formula I or Formula II, Ar¹ is

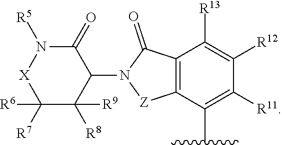

In some embodiments of Formula I or Formula II, Ar¹ is selected from

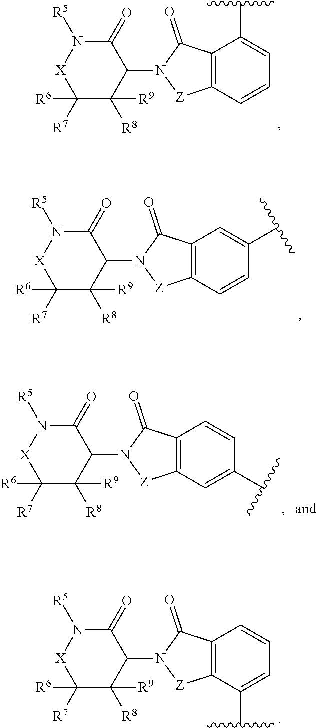

In some embodiments of Formula I or Formula II, Ar¹ is selected from

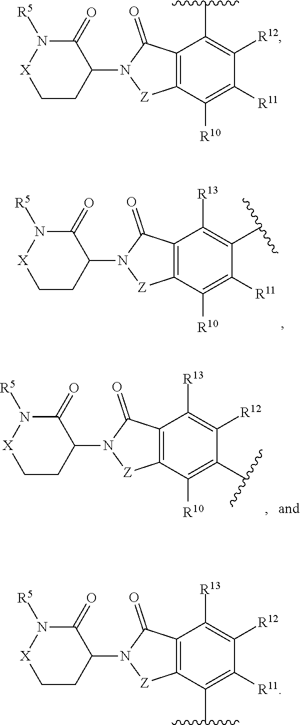

In some embodiments of Formula I or Formula II, Ar¹ is selected from

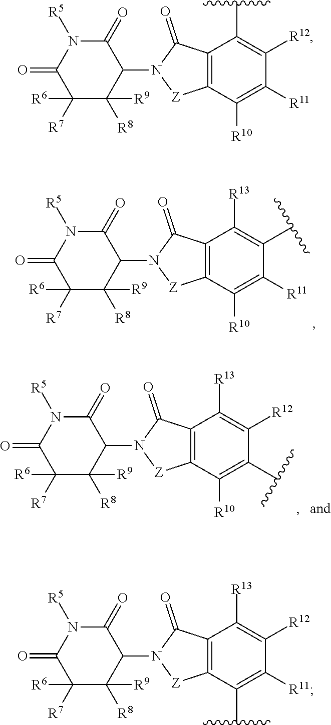

In some embodiments of Formula I or Formula II, Ar¹ selected from

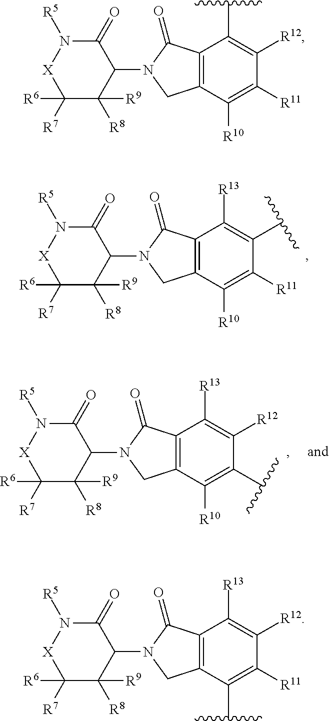

In some embodiments of Formula I or Formula II, Ar¹ is selected from

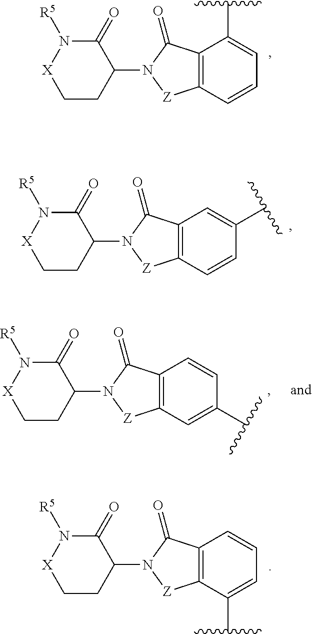

In some embodiments of Formula I or Formula II, Ar¹ is selected from

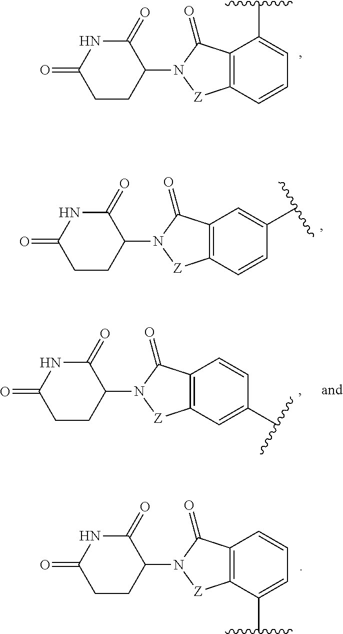

In some embodiments of Formula I or Formula II, Ar¹ is selected from

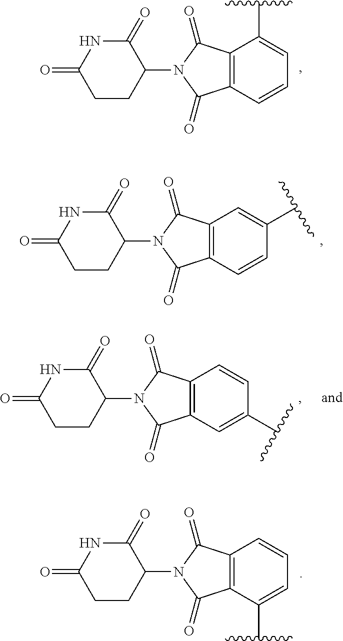

In some embodiments of Formula I or Formula II, Ar¹ is selected from

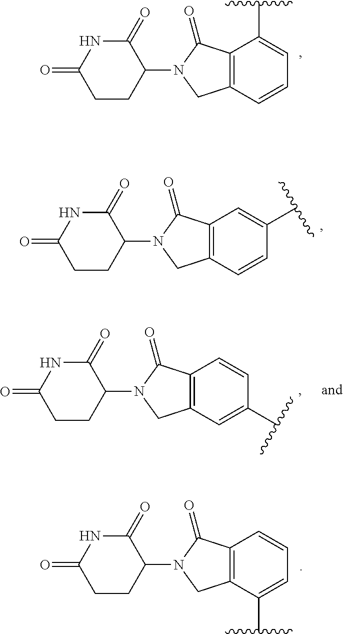 [SMILES: *c1cccc2c1C(=O)N(C1CCC(=O)NC1=O)C2 ; *c1ccc2c(c1)C(=O)N(C1CCC(=O)NC1=O)C2 ; *c1ccc2c(c1)CN(C1CCC(=O)NC1=O)C2=O ; *c1cccc2c1CN(C1CCC(=O)NC1=O)C2=O]

In some embodiments of Formula I or Formula II, Ar¹ is

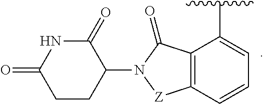

In some embodiments of Formula I or Formula II, Ar¹ is

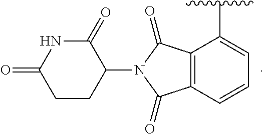 [SMILES: *c1cccc2c1C(=O)N(C1CCC(=O)NC1=O)C2=O]

In some embodiments of Formula I or Formula II, Ar¹ is

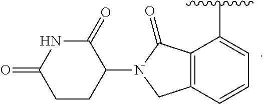 [SMILES: *c1cccc2c1C(=O)N(C1CCC(=O)NC1=O)C2]

In some embodiments of Formula I or Formula II, Ar¹ is

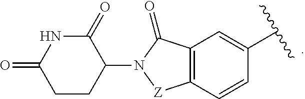

In some embodiments of Formula I or Formula II, Ar¹ is

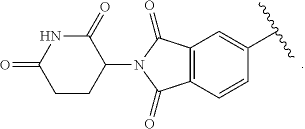 [SMILES: *c1ccc2c(c1)C(=O)N(C1CCC(=O)NC1=O)C2=O]

In some embodiments of Formula I or Formula II, Ar¹ is

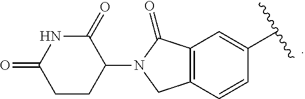 [SMILES: *c1ccc2c(c1)C(=O)N(C1CCC(=O)NC1=O)C2]

In some embodiments of Formula I or Formula II, R⁵ is hydrogen. In some embodiments of Formula I or Formula II, R⁵ is C₁-C₈ alkyl. In some embodiments of Formula I or Formula II, R⁵ is C₁-C₄ alkyl. In some embodiments of Formula I or Formula II, R⁵ is selected from methyl, ethyl, n-propyl, and isopropyl. In some embodiments of Formula I or Formula II, R⁵ is ethyl. In some embodiments of Formula I or Formula II, R⁵ is methyl.

In some embodiments of Formula I or Formula II, each of R⁶, R⁷, R⁸, and R⁹ is independently selected from hydrogen, halogen, —NH₂, —OH, —NO₂, —CN, C₁-C₄ alkyl, C₂-C₄ alkenyl, C₁-C₄ haloalkyl, C₁-C₄ cyanoalkyl, C₁-C₄ hydroxyalkyl, C₁-C₄ haloalkoxy, C₁-C₄ alkoxy, C₁-C₄ alkylamino, independently C₁-C₄ dialkylamino, and C₁-C₄ aminoalkyl. In some embodiments of Formula I or Formula II, each of R⁶, R⁷, R⁸, and R⁹ is independently selected from hydrogen, —F, —Cl, —NH₂, —OH, —NO₂, —CN, methyl, ethyl, n-propyl, isopropyl, ethenyl, n-propenyl, isopropenyl, —CH₂F, —CHF₂, —CF₃, —CH₂CH₂F, —CH₂CH₂CH₂F, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₂F, —OCHF₂, —OCF₃, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NCH₃, —NHCH₂CH₃, —NHCH₂CH₂CH₃, —NHCH(CH₃)₂, —N(CH₃)₂, —N(CH₃)CH₂CH₃, —N(CH₂CH₃)₂, —N(CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂.

In some embodiments of Formula I or Formula II, R⁶ is selected from hydrogen, —F, —Cl, —NH₂, —OH, —NO₂, —CN, methyl, ethyl, n-propyl, isopropyl, ethenyl, n-propenyl, isopropenyl, —CH₂F, —CHF₂, —CF₃, —CH₂CH₂F, —CH₂CH₂CH₂F, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₂F, —OCHF₂, —OCF₃, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NCH₃, —NHCH₂CH₃, —NHCH₂CH₂CH₃, —NHCH(CH₃)₂, —N(CH₃)₂, —N(CH₃)CH₂CH₃, —N(CH₂CH₃)₂, —N(CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂.

In some embodiments of Formula I or Formula II, R⁷ is selected from hydrogen, —F, —Cl, —NH₂, —OH, —NO₂, —CN, methyl, ethyl, n-propyl, isopropyl, ethenyl, n-propenyl, isopropenyl, —CH₂F, —CHF₂, —CF₃, —CH₂CH₂F, —CH₂CH₂CH₂F, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₂F, —OCHF₂, —OCF₃, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NCH₃, —NHCH₂CH₃, —NHCH₂CH₂CH₃, —NHCH(CH₃)₂, —N(CH₃)₂, —N(CH₃)CH₂CH₃, —N(CH₂CH₃)₂, —N(CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂.

In some embodiments of Formula I or Formula II, R⁸ is selected from hydrogen, —F, —Cl, —NH₂, —OH, —NO₂, —CN, methyl, ethyl, n-propyl, isopropyl, ethenyl, n-propenyl, isopropenyl, —CH₂F, —CHF₂, —CF₃, —CH₂CH₂F, —CH₂CH₂CH₂F, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₂F, —OCHF₂, —OCF₃, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NCH₃, —NHCH₂CH₃, —NHCH₂CH₂CH₃, —NHCH(CH₃)₂, —N(CH₃)₂, —N(CH₃)CH₂CH₃, —N(CH₂CH₃)₂, —N(CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂.

In some embodiments of Formula I or Formula II, R⁹ is selected from hydrogen, —F, —Cl, —NH₂, —OH, —NO₂, —CN, methyl, ethyl, n-propyl, isopropyl, ethenyl, n-propenyl, isopropenyl, —CH₂F, —CHF₂, —CF₃, —CH₂CH₂F, —CH₂CH₂CH₂F, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₂F, —OCHF₂, —OCF₃, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NCH₃, —NHCH₂CH₃, —NHCH₂CH₂CH₃, —NHCH(CH₃)₂, —N(CH₃)₂, —N(CH₃)CH₂CH₃, —N(CH₂CH₃)₂, —N(CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂.

In some embodiments of Formula I or Formula II, R⁶, R⁷, R⁸, and R⁹ are each hydrogen.

In some embodiments of Formula I or Formula II, each of R¹⁰, R¹¹, R¹², and R¹³ is independently selected from hydrogen, halogen, —NH₂, —OH, —NO₂, —CN, C₁-C₄ alkyl, C₂-C₄ alkenyl, C₁-C₄ haloalkyl, C₁-C₄ cyanoalkyl, C₁-C₄ hydroxyalkyl, C₁-C₄ haloalkoxy, C₁-C₄ alkoxy, C₁-C₄ alkylamino, independently C₁-C₄ dialkylamino, and C₁-C₄ aminoalkyl. In some embodiments of Formula I or Formula II, each of R¹⁰, R¹¹, R¹², and R¹³ is independently selected from hydrogen, —F, —Cl, —NH₂, —OH, —NO₂, —CN, methyl, ethyl, n-propyl, isopropyl, ethenyl, n-propenyl, isopropenyl, —CH₂F, —CHF₂, —CF₃, —CH₂CH₂F, —CH₂CH₂CH₂F, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₂F, —OCHF₂, —OCF₃, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NCH₃, —NHCH₂CH₃, —NHCH₂CH₂CH₃, —NHCH(CH₃)₂, —N(CH₃)₂, —N(CH₃)CH₂CH₃, —N(CH₂CH₃)₂, —N(CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂.

In some embodiments of Formula I or Formula II, R¹⁰ is selected from hydrogen, —F, —Cl, —NH₂, —OH, —NO₂, —CN, methyl, ethyl, n-propyl, isopropyl, ethenyl, n-propenyl, isopropenyl, —CH₂F, —CHF₂, —CF₃, —CH₂CH₂F, —CH₂CH₂CH₂F, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₂F, —OCHF₂, —OCF₃, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NCH₃, —NHCH₂CH₃, —NHCH₂CH₂CH₃, —NHCH(CH₃)₂, —N(CH₃)₂, —N(CH₃)CH₂CH₃, —N(CH₂CH₃)₂, —N(CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂.

In some embodiments of Formula I or Formula II, R¹¹ is selected from hydrogen, —F, —Cl, —NH₂, —OH, —NO₂, —CN, methyl, ethyl, n-propyl, isopropyl, ethenyl, n-propenyl, isopropenyl, —CH₂F, —CHF₂, —CF₃, —CH₂CH₂F, —CH₂CH₂CH₂F, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₂F, —OCHF₂, —OCF₃, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NCH₃, —NHCH₂CH₃, —NHCH₂CH₂CH₃, —NHCH(CH₃)₂, —N(CH₃)₂, —N(CH₃)CH₂CH₃, —N(CH₂CH₃)₂, —N(CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂.

In some embodiments of Formula I or Formula II, R¹² is selected from hydrogen, —F, —Cl, —NH₂, —OH, —NO₂, —CN, methyl, ethyl, n-propyl, isopropyl, ethenyl, n-propenyl, isopropenyl, —CH₂F, —CHF₂, —CF₃, —CH₂CH₂F, —CH₂CH₂CH₂F, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₂F, —OCHF₂, —OCF₃, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NCH₃, —NHCH₂CH₃, —NHCH₂CH₂CH₃, —NHCH(CH₃)₂, —N(CH₃)₂, —N(CH₃)CH₂CH₃, —N(CH₂CH₃)₂, —N(CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂.

In some embodiments of Formula I or Formula II, R¹³ is selected from hydrogen, —F, —Cl, —NH₂, —OH, —NO₂, —CN, methyl, ethyl, n-propyl, isopropyl, ethenyl, n-propenyl, isopropenyl, —CH₂F, —CHF₂, —CF₃, —CH₂CH₂F, —CH₂CH₂CH₂F, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₂F, —OCHF₂, —OCF₃, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NCH₃, —NHCH₂CH₃, —NHCH₂CH₂CH₃, —NHCH(CH₃)₂, —N(CH₃)₂, —N(CH₃)CH₂CH₃, —N(CH₂CH₃)₂, —N(CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂.

In some embodiments of Formula I or Formula II, R¹⁰, R¹¹, R¹², and R¹³ are each hydrogen.

In some embodiments of Formula I or Formula II,

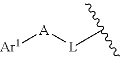

is selected from:

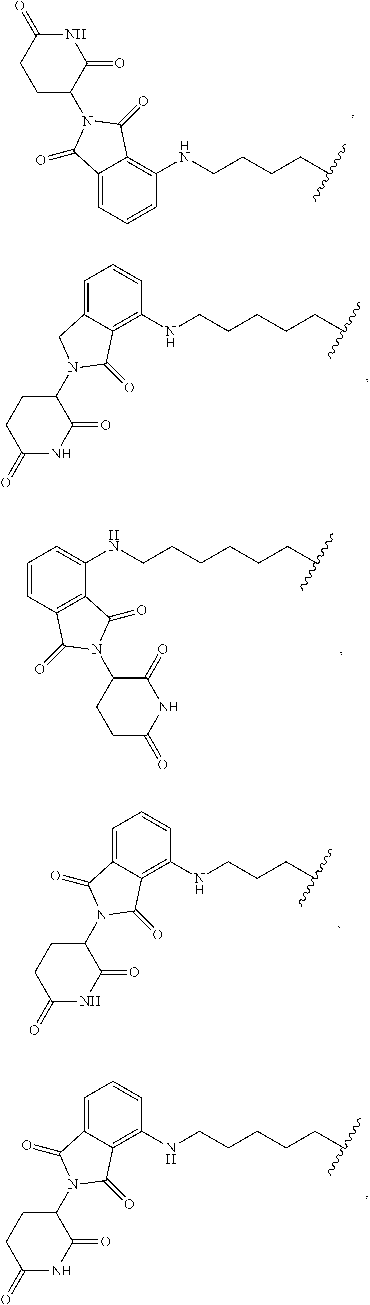

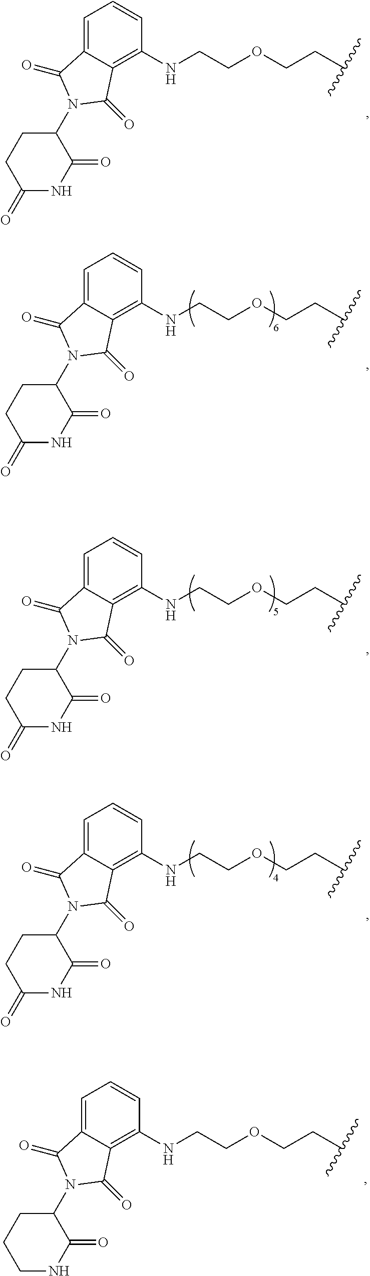

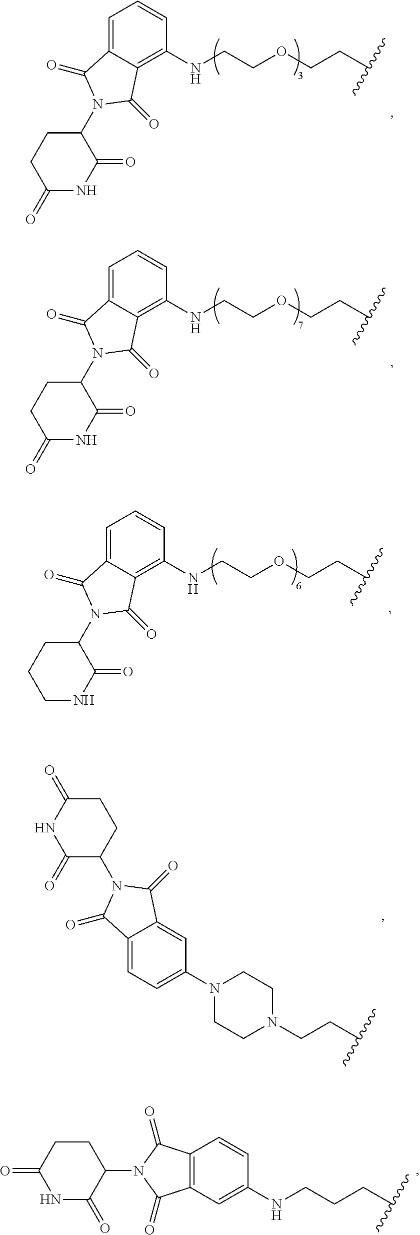

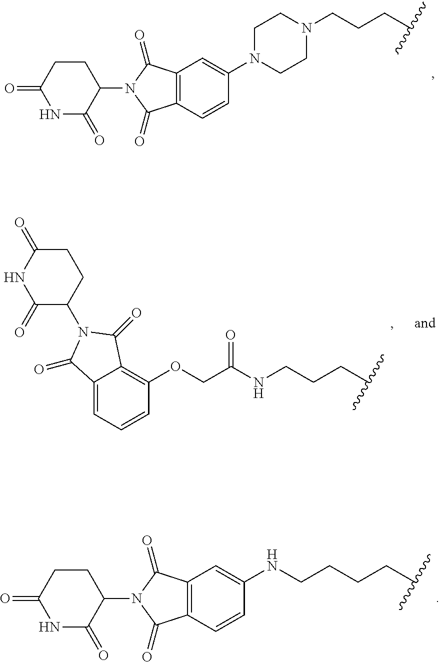 [SMILES: *CCCN1CCN(c2ccc3c(c2)C(=O)N(C2CCC(=O)NC2=O)C3=O)CC1 ; *CCCNC(=O)COc1cccc2c1C(=O)N(C1CCC(=O)NC1=O)C2=O ; *CCCCNc1ccc2c(c1)C(=O)N(C1CCC(=O)NC1=O)C2=O]

In some embodiments of Formula I, R¹ is

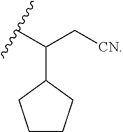 [SMILES: *C(CC#N)C1CCCC1]

In some embodiments of Formula I, R¹ is

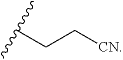 [SMILES: *CCC#N]

In some embodiments of Formula I, R¹ is

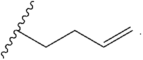 [SMILES: *CCC=C]

In some embodiments of Formula II, R² is hydrogen. In some embodiments of Formula II, R² is F.

In some embodiments of Formula II, R³ is Cl. In some embodiments of Formula II, R³ is F.

In some embodiments of Formula II, R⁴ is hydrogen.

In some embodiments of Formula II, R³ and R⁴ are brought together with the atoms to which they are attached to form a pyrrolidine ring.

Representative examples of compounds of Formula I include, but are not limited to, the compound found in Table A below:

TABLE A
Exemplary Compounds of Formula I

| Compound # | Structure |
|---|---|
| A-1 | 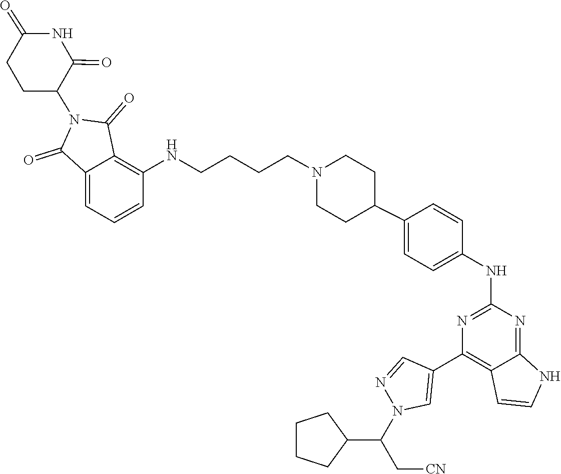 [SMILES: N#CCC(C1CCCC1)n1cc(-c2nc(Nc3ccc(C4CCN(CCCCNc5cccc6c5C(=O)N(C5CCC(=O)NC5=O)C6=O)CC4)cc3)nc3[nH]ccc23)cn1] |
| A-2 | 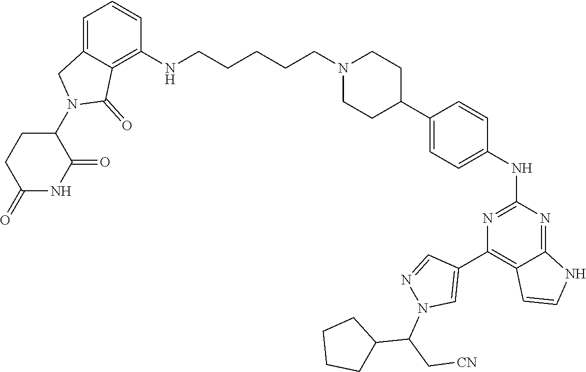 |
| A-3 | 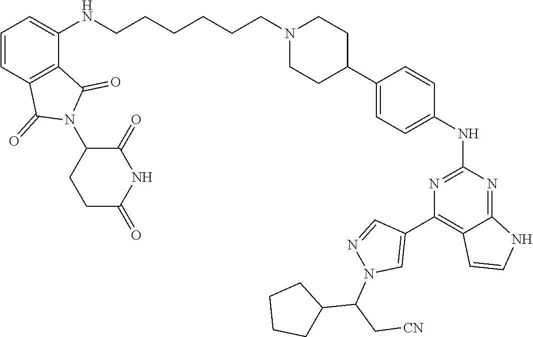 [SMILES: N#CCC(C1CCCC1)n1cc(-c2nc(Nc3ccc(C4CCN(CCCCCCNc5cccc6c5C(=O)N(C5CCC(=O)NC5=O)C6=O)CC4)cc3)nc3[nH]ccc23)cn1] |
| A-4 | 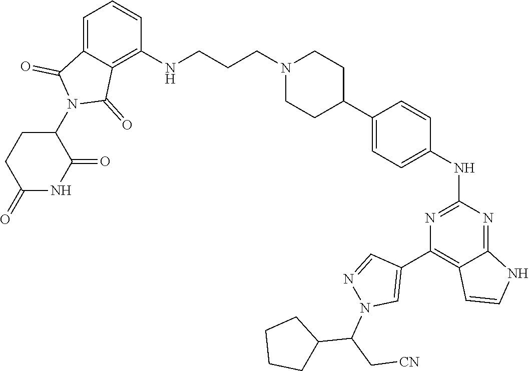 [SMILES: N#CCC(C1CCCC1)n1cc(-c2nc(Nc3ccc(C4CCN(CCCNc5cccc6c5C(=O)N(C5CCC(=O)NC5=O)C6=O)CC4)cc3)nc3[nH]ccc23)cn1] |
| A-5 | 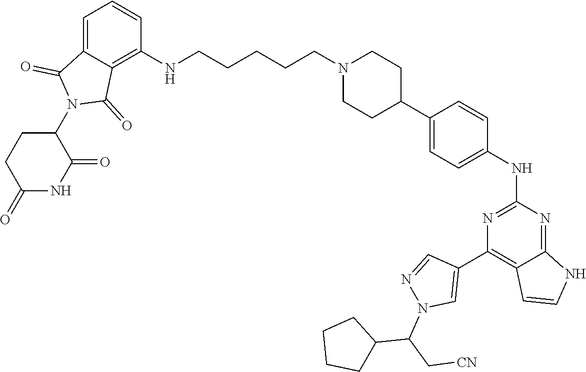 |
| A-6 | 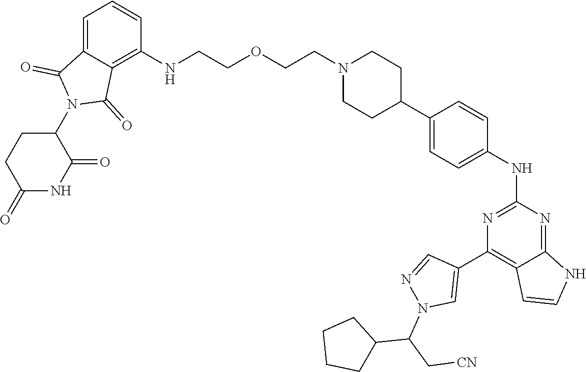 [SMILES: N#CCC(C1CCCC1)n1cc(-c2nc(Nc3ccc(C4CCN(CCOCCNc5cccc6c5C(=O)N(C5CCC(=O)NC5=O)C6=O)CC4)cc3)nc3[nH]ccc23)cn1] |
| A-7 | 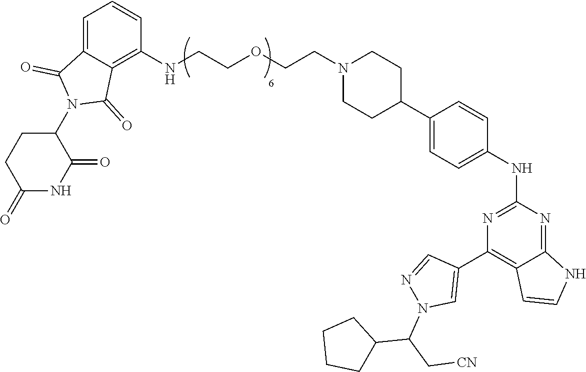 |
| A-8 | 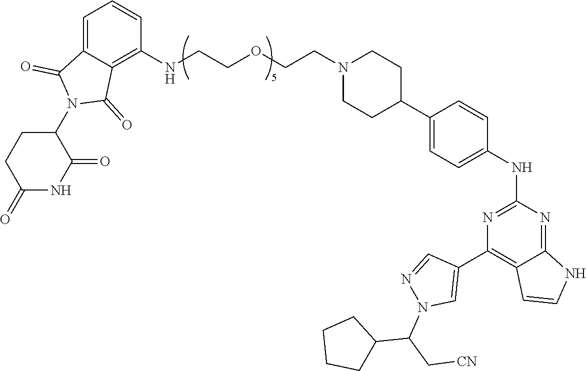 |
| A-9 | 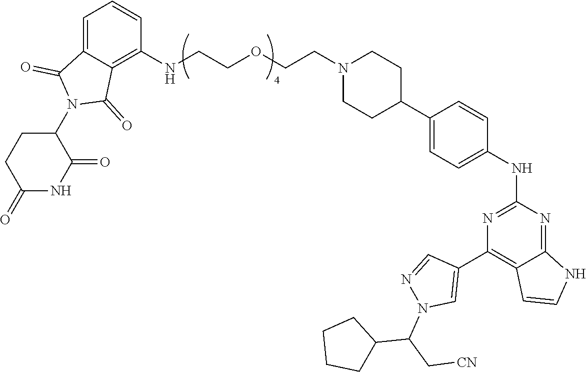 |
| A-10 | 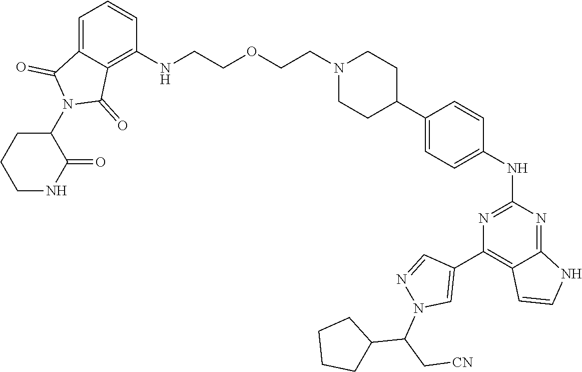 |
| A-11 | 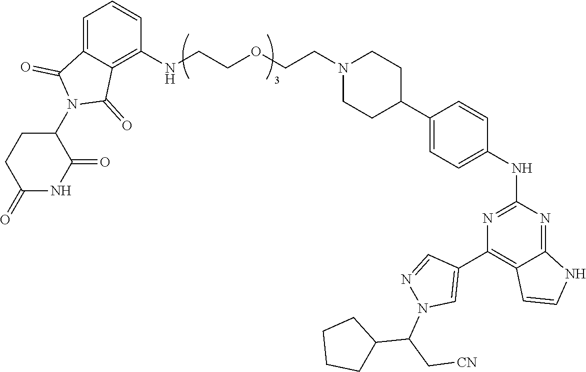 |
| A-12 | 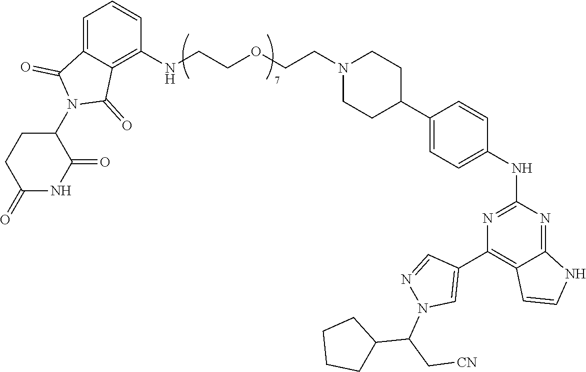 |
| A-13 | 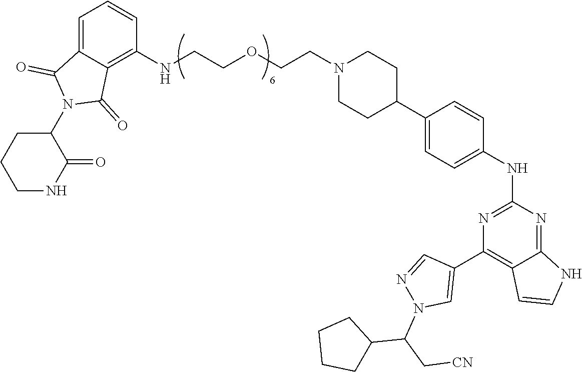 |
| A-14 | 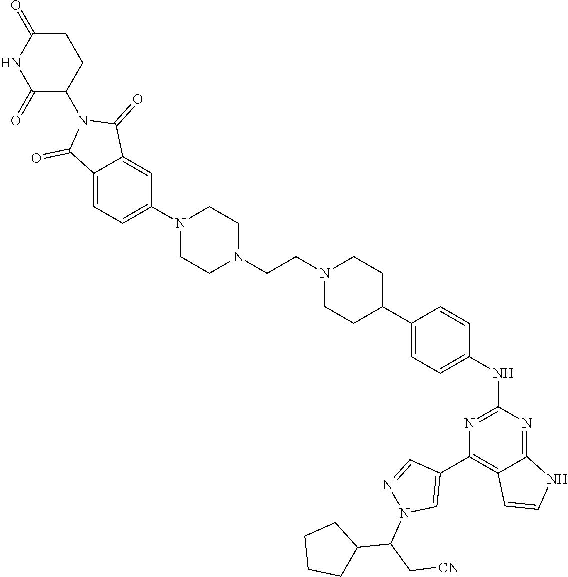 |
| A-15 | 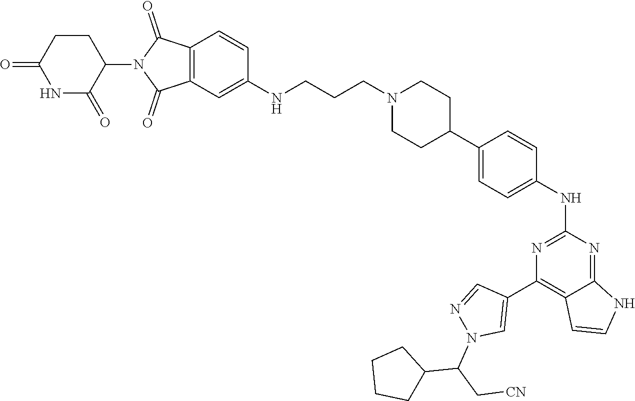 [SMILES: N#CCC(C1CCCC1)n1cc(-c2nc(Nc3ccc(C4CCN(CCCNc5ccc6c(c5)C(=O)N(C5CCC(=O)NC5=O)C6=O)CC4)cc3)nc3[nH]ccc23)cn1] |
| A-16 | 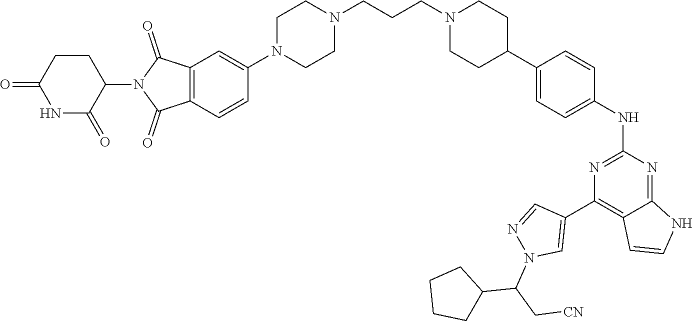 [SMILES: N#CCC(C1CCCC1)n1cc(-c2nc(Nc3ccc(C4CCN(CCCN5CCN(c6ccc7c(c6)C(=O)N(C6CCC(=O)NC6=O)C7=O)CC5)CC4)cc3)nc3[nH]ccc23)cn1] |
| A-17 | 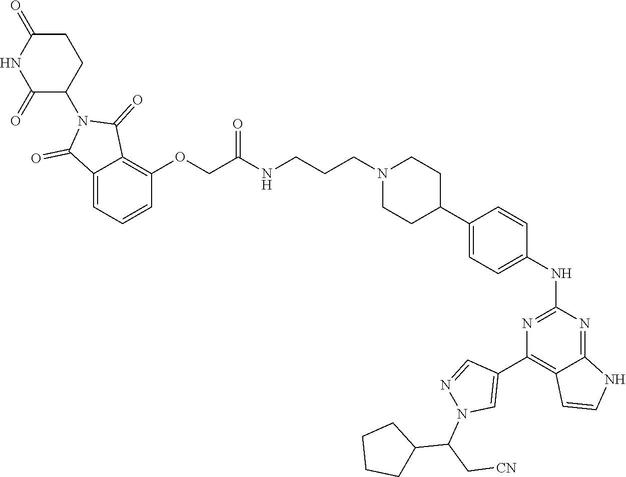 |
| A-18 | 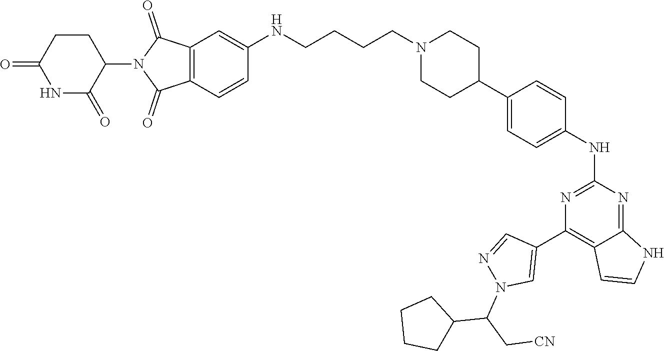 |

Further representative examples of compounds of Formula I include, but are not limited to, the compounds provided in Table B below:

TABLE B
Further Exemplary Compounds of Formula I

| Compound # | Structure |
|---|---|
| B-1 | 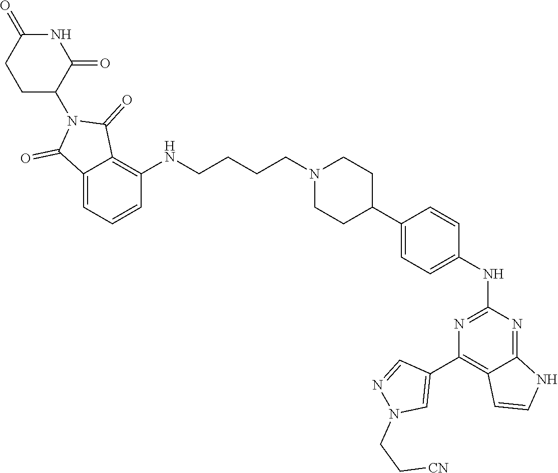 |
| B-2 | 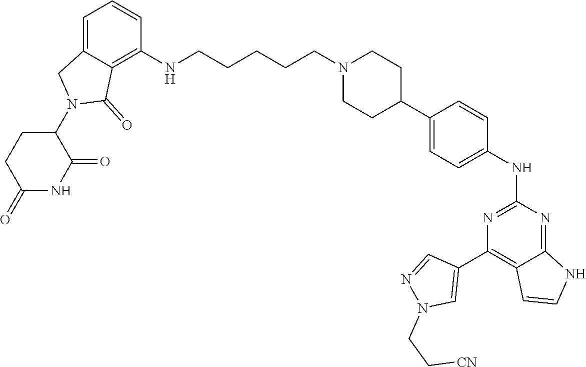 |
| B-3 | 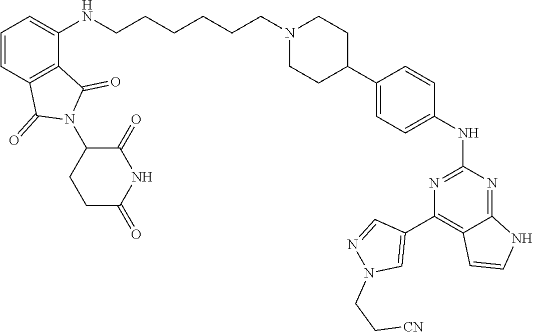 |
| B-4 | 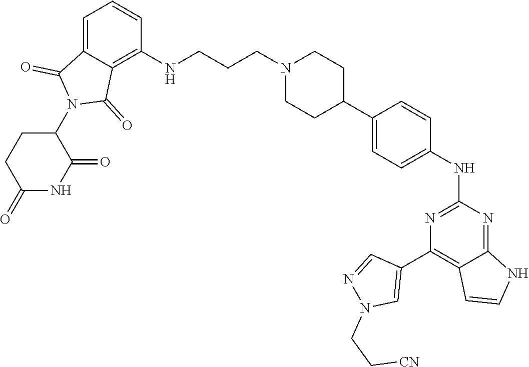 [SMILES: N#CCCn1cc(-c2nc(Nc3ccc(C4CCN(CCCNc5cccc6c5C(=O)N(C5CCC(=O)NC5=O)C6=O)CC4)cc3)nc3[nH]ccc23)cn1] |
| B-5 | 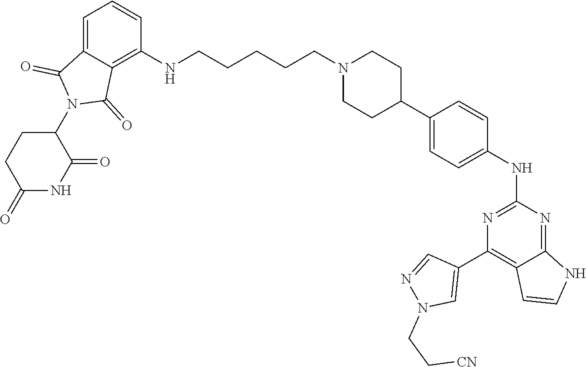 |
| B-6 | 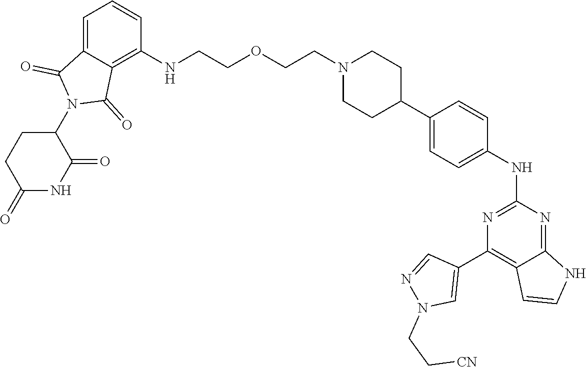 |
| B-7 | 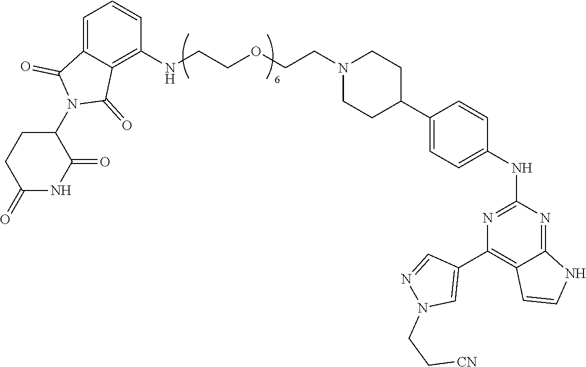 |
| B-8 | 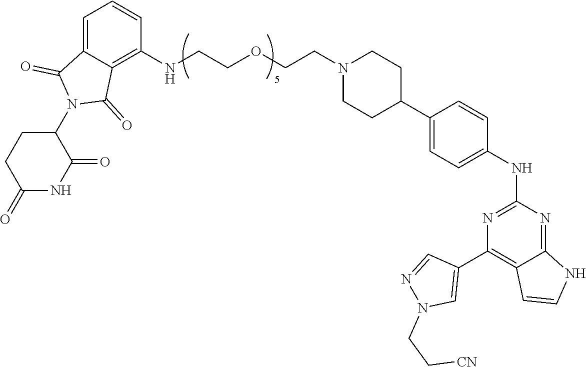 |
| B-9 | 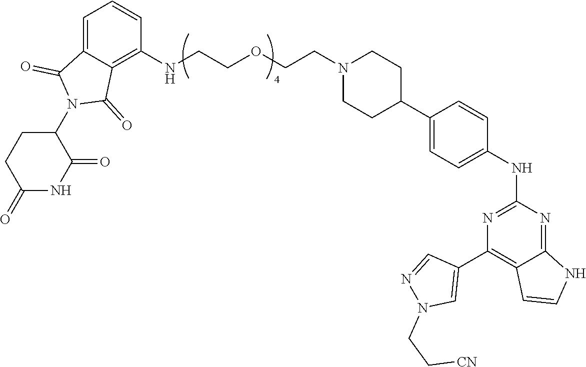 |
| B-10 | 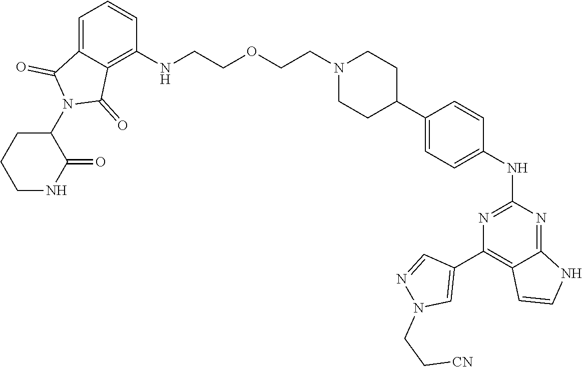 |
| B-11 | 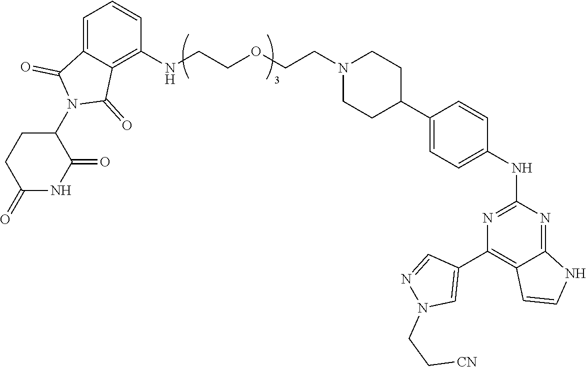 |
| B-12 | 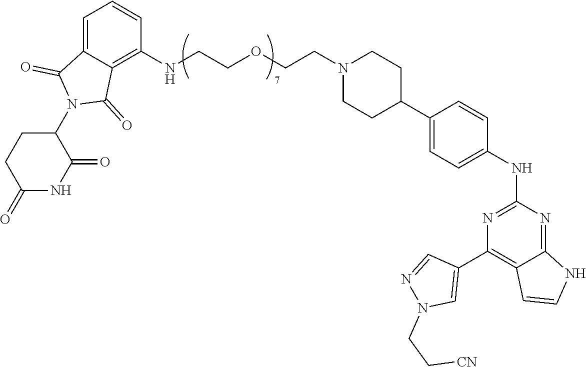 |
| B-13 | 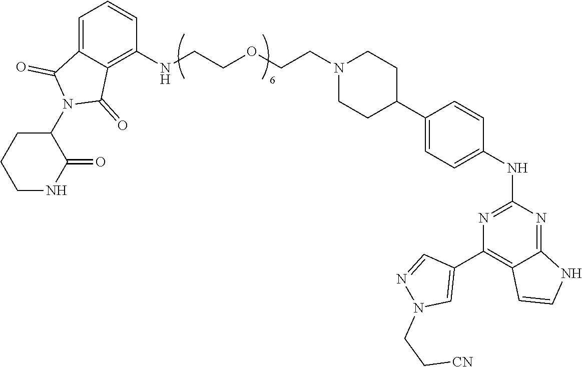 |
| B-14 | 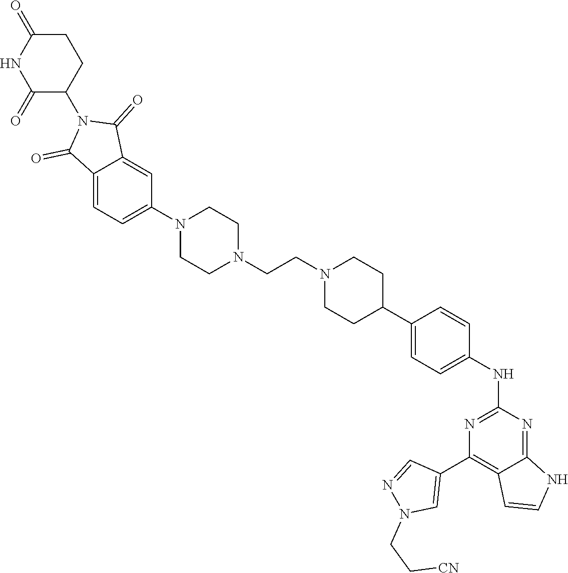 [SMILES: N#CCCn1cc(-c2nc(Nc3ccc(C4CCN(CCN5CCN(c6ccc7c(c6)C(=O)N(C6CCC(=O)NC6=O)C7=O)CC5)CC4)cc3)nc3[nH]ccc23)cn1] |
| B-15 | 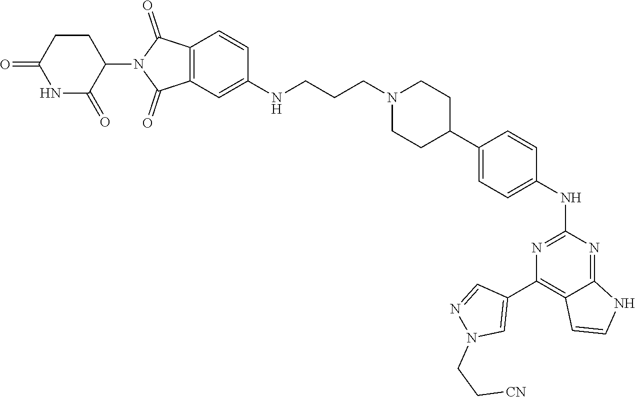 |
| B-16 | 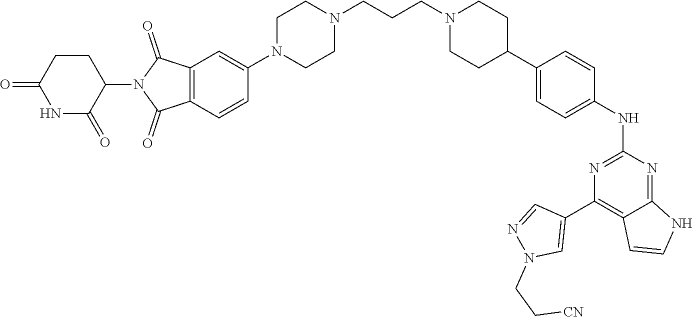 |
| B-17 | 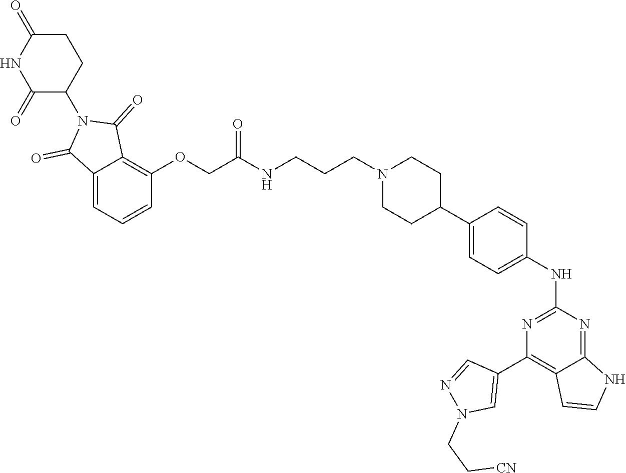 |
| B-18 | 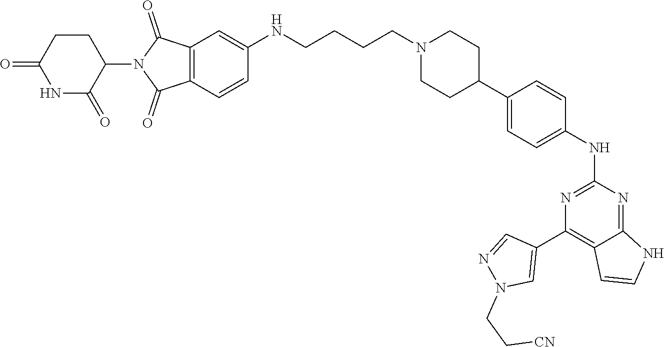 |

Further representative examples of compounds of Formula I include, but are not limited to, the compounds found in Table C below:

TABLE C
Further Exemplary Compounds of Formula I

| Compound # | Structure |
|---|---|
| C-1 | 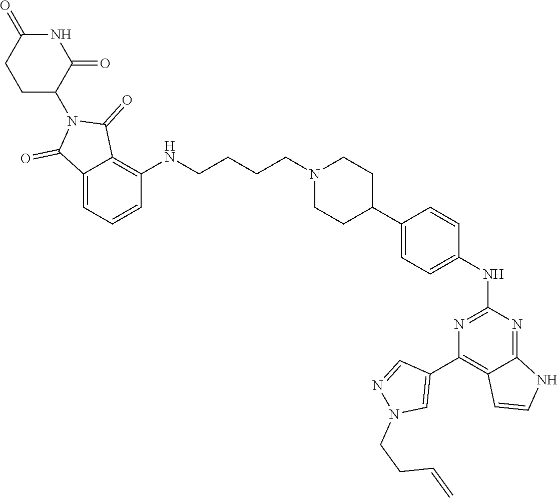 |
| C-2 | 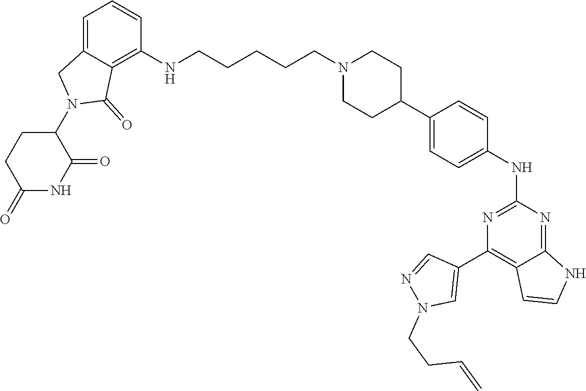 |
| C-3 | 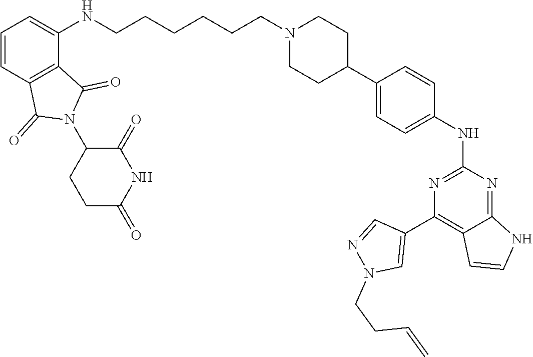 [SMILES: C=CCCn1cc(-c2nc(Nc3ccc(C4CCN(CCCCCCNc5cccc6c5C(=O)N(C5CCC(=O)NC5=O)C6=O)CC4)cc3)nc3[nH]ccc23)cn1] |
| C-4 | 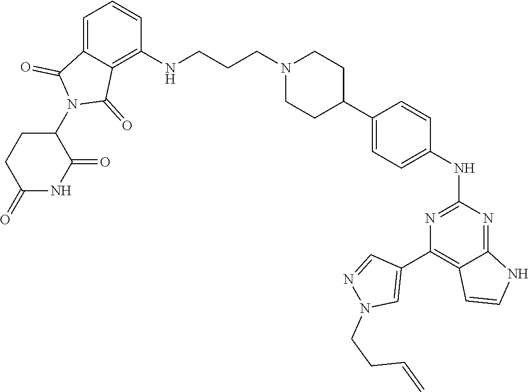 [SMILES: C=CCCn1cc(-c2nc(Nc3ccc(C4CCN(CCCNc5cccc6c5C(=O)N(C5CCC(=O)NC5=O)C6=O)CC4)cc3)nc3[nH]ccc23)cn1] |
| C-5 | 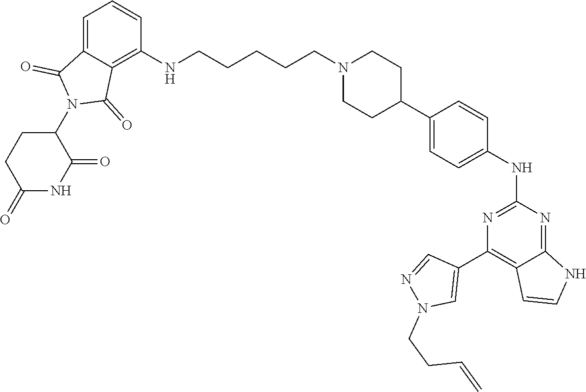 |
| C-6 | 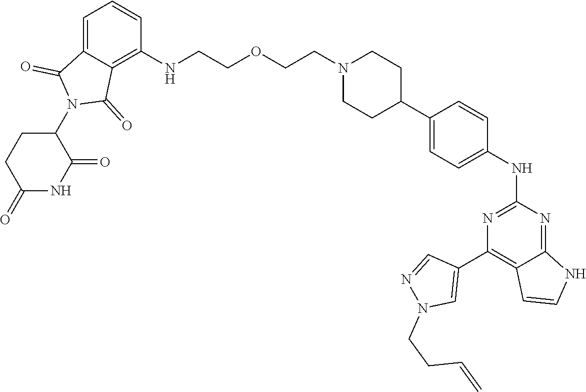 |
| C-7 | 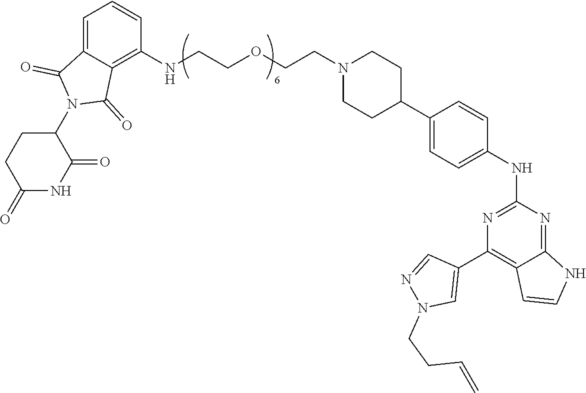 |
| C-8 | 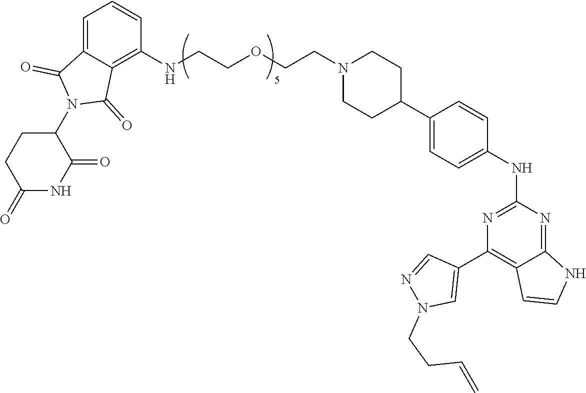 |
| C-9 | 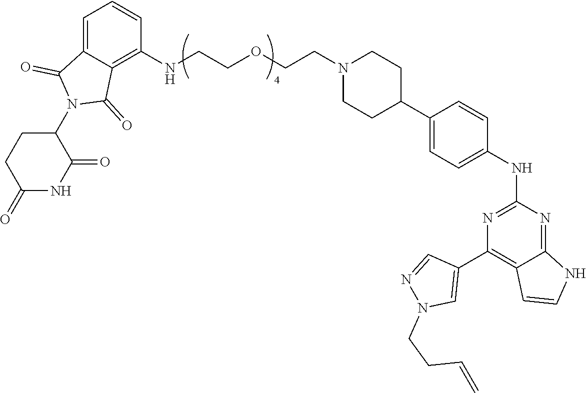 |
| C-10 | 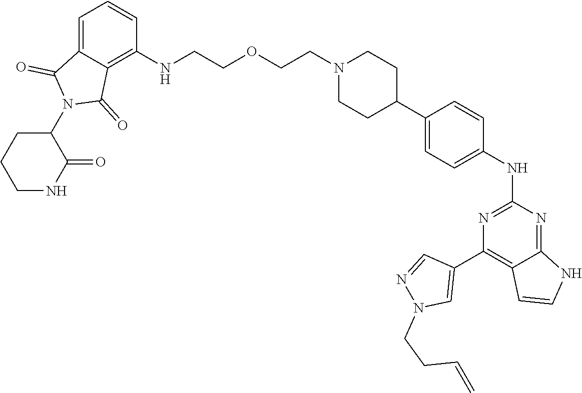 [SMILES: C=CCCn1cc(-c2nc(Nc3ccc(C4CCN(CCOCCNc5cccc6c5C(=O)N(C5CCCNC5=O)C6=O)CC4)cc3)nc3[nH]ccc23)cn1] |
| C-11 | 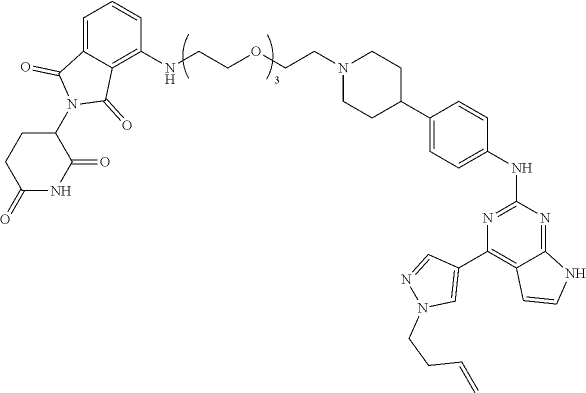 |
| C-12 | 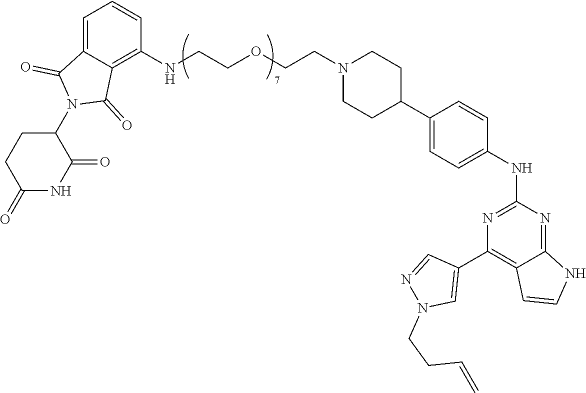 |
| C-13 | 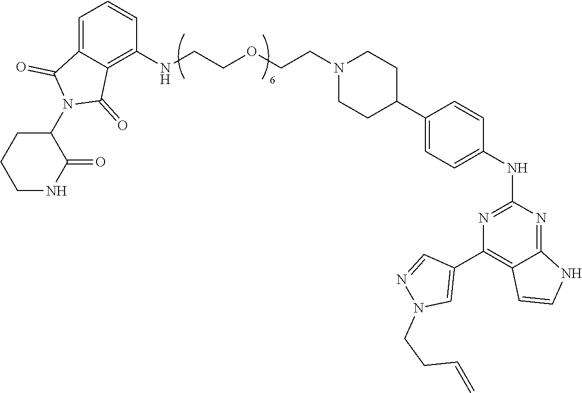 |
| C-14 | 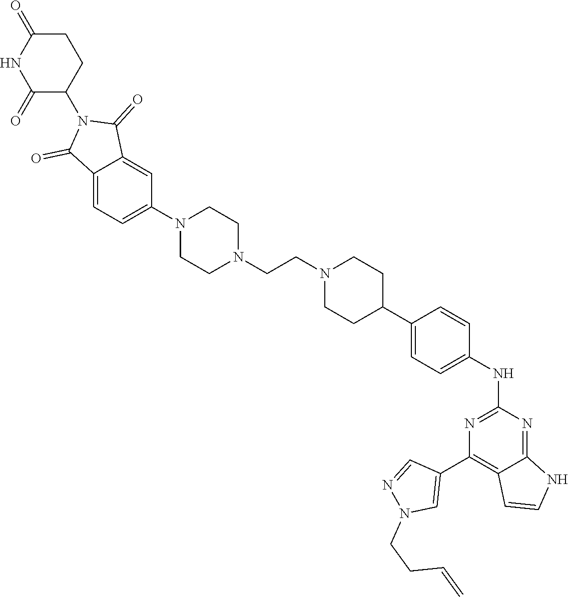 |
| C-15 | 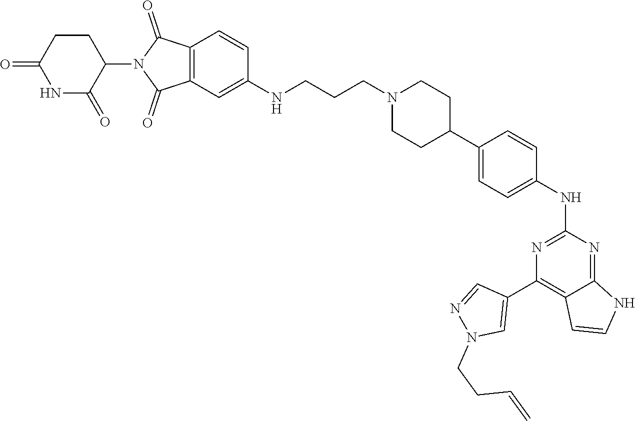 |
| C-16 | 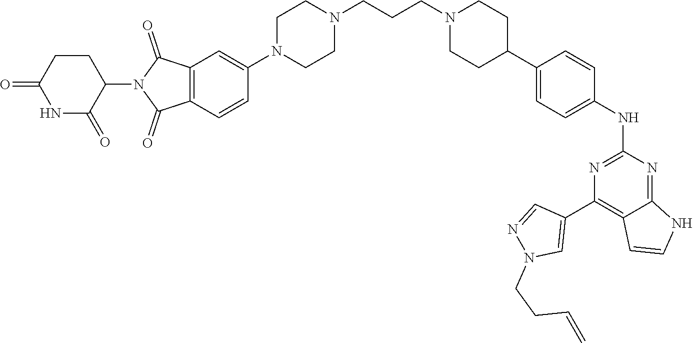 |
| C-17 | 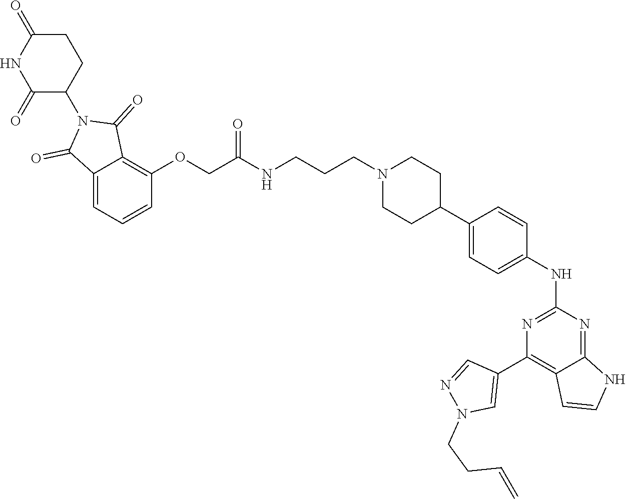 |
| C-18 | 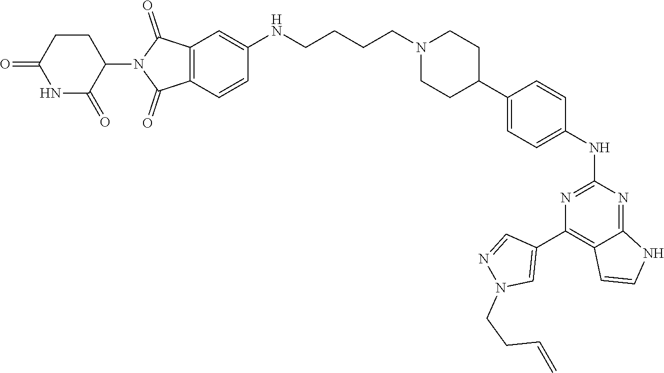 |

Representative examples of compounds of Formula II include, but are not limited to, the compounds found in Table D below:

TABLE D
Exemplary Compounds of Formula II

| Compound | |
|---|---|
| # | Structure |
| D-1 | 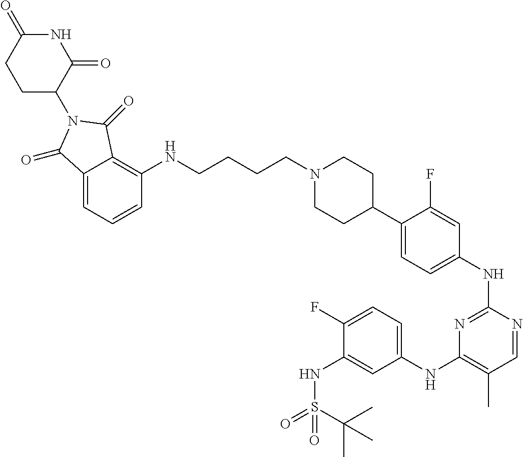 [SMILES: Cc1cnc(Nc2ccc(C3CCN(CCCCNc4cccc5c4C(=O)N(C4CCC(=O)NC4=O)C5=O)CC3)c(F)c2)nc1Nc1ccc(F)c(NS(=O)(=O)C(C)(C)C)c1] |
| D-2 | 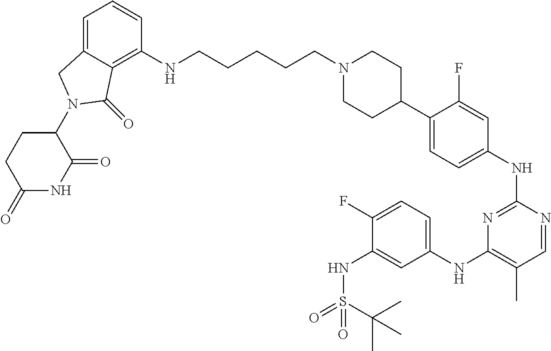 [SMILES: Cc1cnc(Nc2ccc(C3CCN(CCCCCNc4cccc5c4C(=O)N(C4CCC(=O)NC4=O)C5)CC3)c(F)c2)nc1Nc1ccc(F)c(NS(=O)(=O)C(C)(C)C)c1] |
| D-3 | 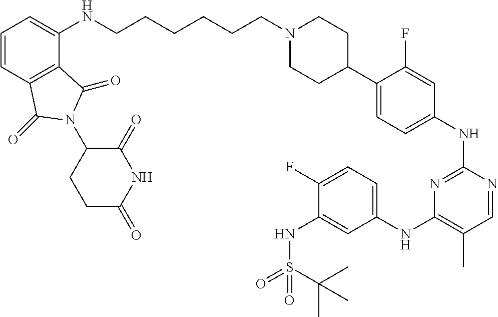 [SMILES: Cc1cnc(Nc2ccc(C3CCN(CCCCCCNc4cccc5c4C(=O)N(C4CCC(=O)NC4=O)C5=O)CC3)c(F)c2)nc1Nc1ccc(F)c(NS(=O)(=O)C(C)(C)C)c1] |
| D-4 | 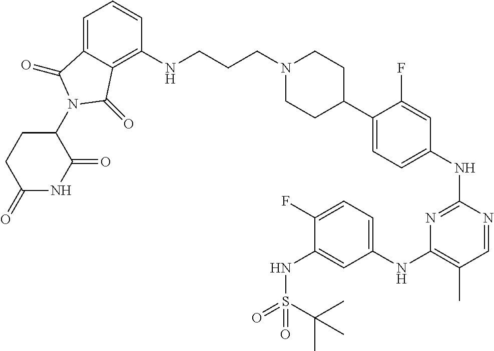 [SMILES: Cc1cnc(Nc2ccc(C3CCN(CCCNc4cccc5c4C(=O)N(C4CCC(=O)NC4=O)C5=O)CC3)c(F)c2)nc1Nc1ccc(F)c(NS(=O)(=O)C(C)(C)C)c1] |
| D-5 | 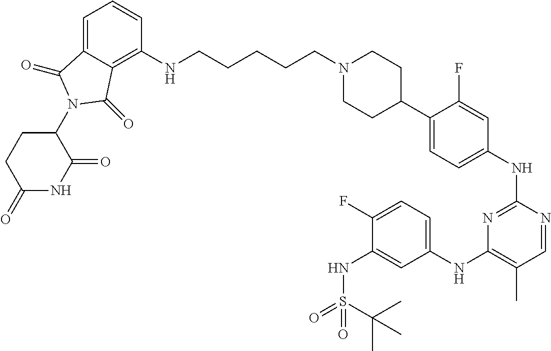 [SMILES: Cc1cnc(Nc2ccc(C3CCN(CCCCCNc4cccc5c4C(=O)N(C4CCC(=O)NC4=O)C5=O)CC3)c(F)c2)nc1Nc1ccc(F)c(NS(=O)(=O)C(C)(C)C)c1] |
| D-6 | 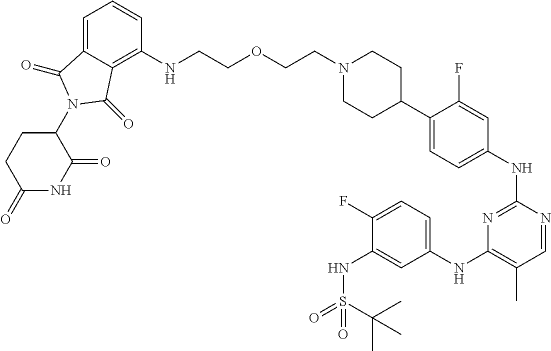 [SMILES: Cc1cnc(Nc2ccc(C3CCN(CCOCCNc4cccc5c4C(=O)N(C4CCC(=O)NC4=O)C5=O)CC3)c(F)c2)nc1Nc1ccc(F)c(NS(=O)(=O)C(C)(C)C)c1] |
| D-7 | 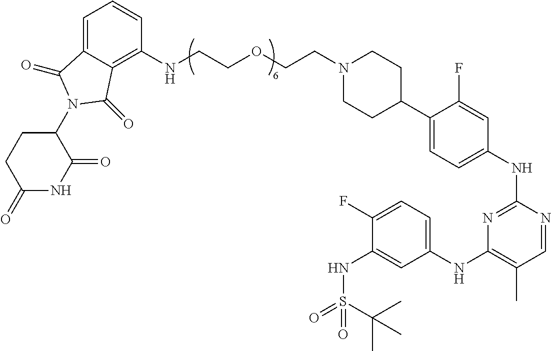 |
| D-8 | 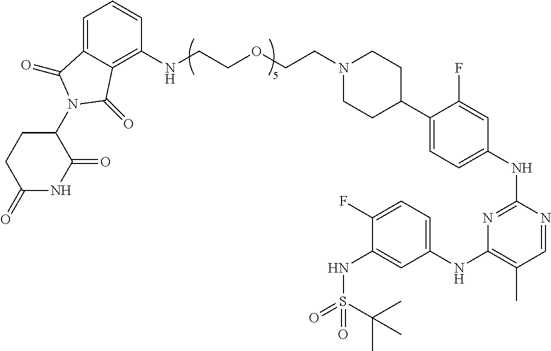 |
| D-9 | 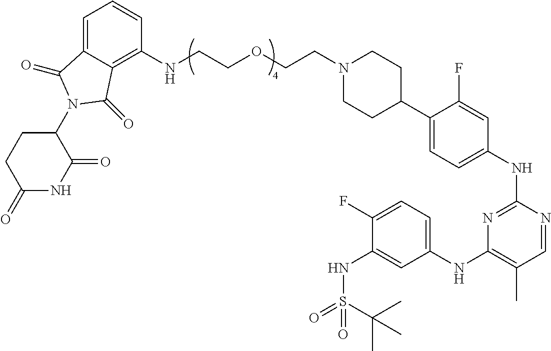 |
| D-10 | 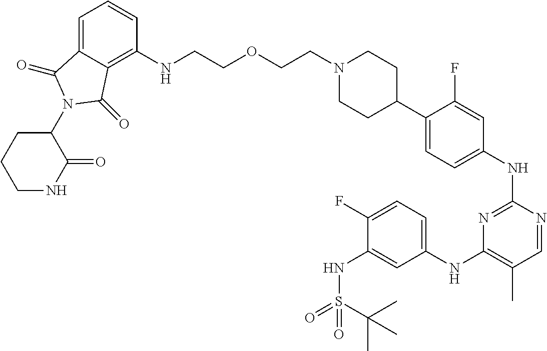 [SMILES: Cc1cnc(Nc2ccc(C3CCN(CCOCCNc4cccc5c4C(=O)N(C4CCCNC4=O)C5=O)CC3)c(F)c2)nc1Nc1ccc(F)c(NS(=O)(=O)C(C)(C)C)c1] |
| D-11 | 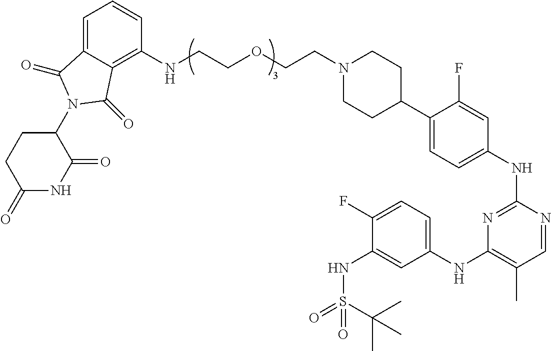 |
| D-12 | 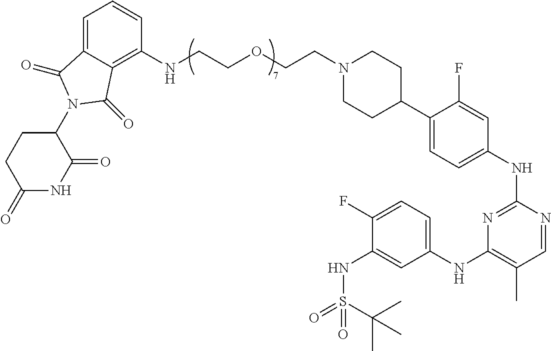 |
| D-13 | 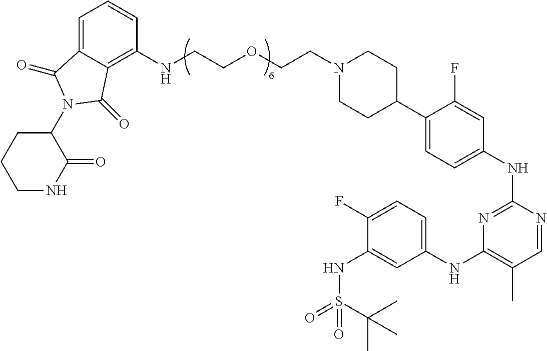 |
| D-14 | 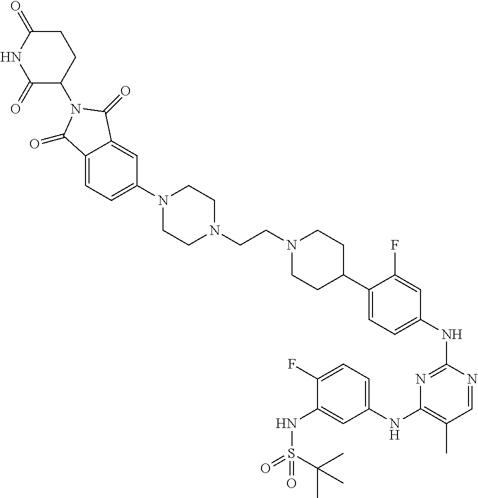 [SMILES: Cc1cnc(Nc2ccc(C3CCN(CCN4CCN(c5ccc6c(c5)C(=O)N(C5CCC(=O)NC5=O)C6=O)CC4)CC3)c(F)c2)nc1Nc1ccc(F)c(NS(=O)(=O)C(C)(C)C)c1] |
| D-15 | 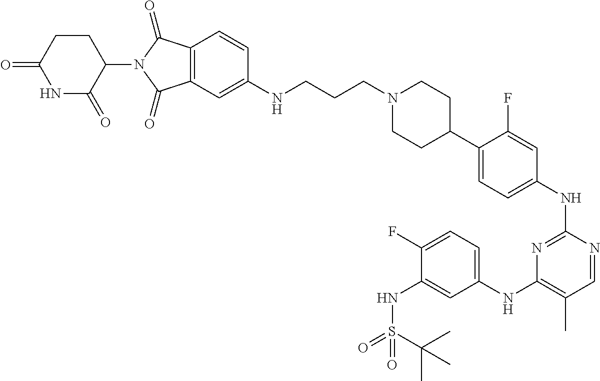 [SMILES: Cc1cnc(Nc2ccc(C3CCN(CCCNc4ccc5c(c4)C(=O)N(C4CCC(=O)NC4=O)C5=O)CC3)c(F)c2)nc1Nc1ccc(F)c(NS(=O)(=O)C(C)(C)C)c1] |
| D-16 | 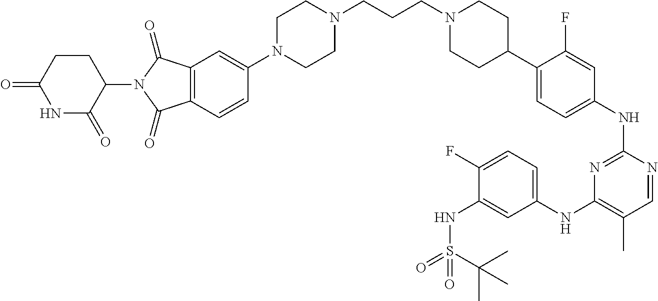 [SMILES: Cc1cnc(Nc2ccc(C3CCN(CCCN4CCN(c5ccc6c(c5)C(=O)N(C5CCC(=O)NC5=O)C6=O)CC4)CC3)c(F)c2)nc1Nc1ccc(F)c(NS(=O)(=O)C(C)(C)C)c1] |
| D-17 | 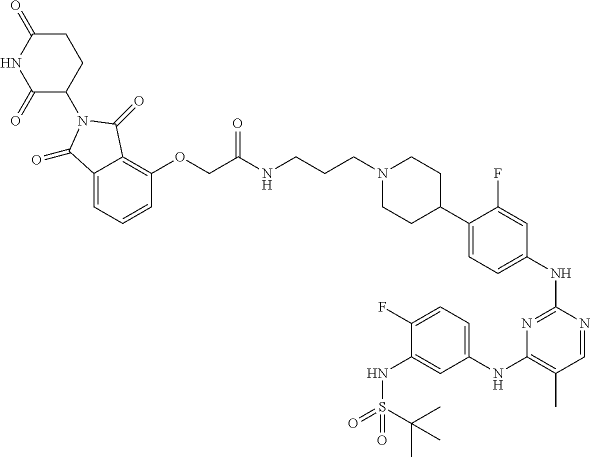 [SMILES: Cc1cnc(Nc2ccc(C3CCN(CCCNC(=O)COc4cccc5c4C(=O)N(C4CCC(=O)NC4=O)C5=O)CC3)c(F)c2)nc1Nc1ccc(F)c(NS(=O)(=O)C(C)(C)C)c1] |
| D-18 | 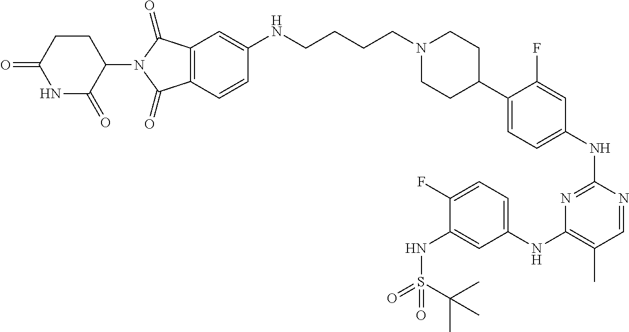 [SMILES: Cc1cnc(Nc2ccc(C3CCN(CCCCNc4ccc5c(c4)C(=O)N(C4CCC(=O)NC4=O)C5=O)CC3)c(F)c2)nc1Nc1ccc(F)c(NS(=O)(=O)C(C)(C)C)c1] |

Further representative examples of compounds of Formula II include, but are not limited to, the compounds found in Table E below:

TABLE E
Further Exemplary Compounds of Formula II

| Compound | |
|---|---|
| # | Structure |
| E-1 | 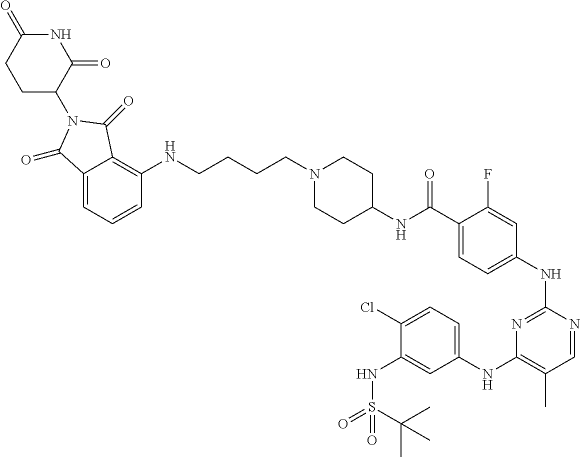 [SMILES: Cc1cnc(Nc2ccc(C(=O)NC3CCN(CCCCNc4cccc5c4C(=O)N(C4CCC(=O)NC4=O)C5=O)CC3)c(F)c2)nc1Nc1ccc(Cl)c(NS(=O)(=O)C(C)(C)C)c1] |
| E-2 | 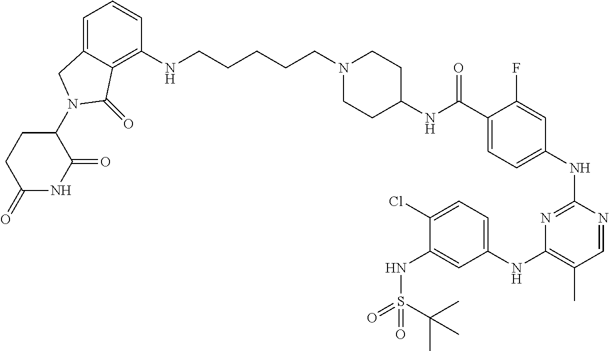 [SMILES: Cc1cnc(Nc2ccc(C(=O)NC3CCN(CCCCCNc4cccc5c4C(=O)N(C4CCC(=O)NC4=O)C5)CC3)c(F)c2)nc1Nc1ccc(Cl)c(NS(=O)(=O)C(C)(C)C)c1] |
| E-3 | 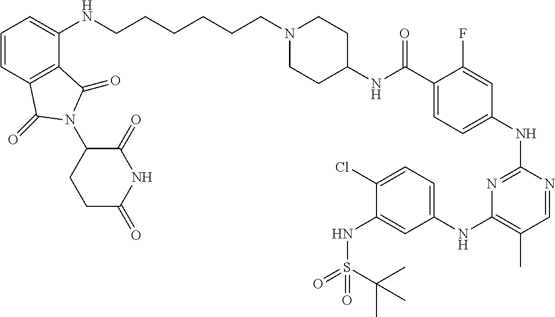 [SMILES: Cc1cnc(Nc2ccc(C(=O)NC3CCN(CCCCCCNc4cccc5c4C(=O)N(C4CCC(=O)NC4=O)C5=O)CC3)c(F)c2)nc1Nc1ccc(Cl)c(NS(=O)(=O)C(C)(C)C)c1] |
| E-4 | 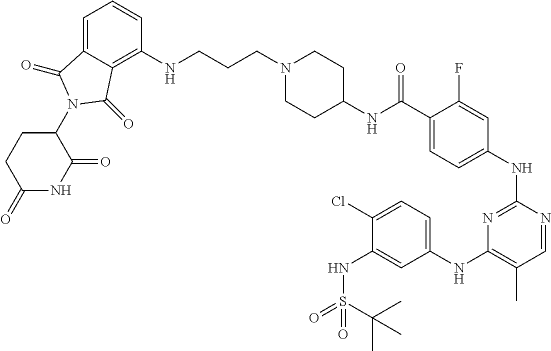 [SMILES: Cc1cnc(Nc2ccc(C(=O)NC3CCN(CCCNc4cccc5c4C(=O)N(C4CCC(=O)NC4=O)C5=O)CC3)c(F)c2)nc1Nc1ccc(Cl)c(NS(=O)(=O)C(C)(C)C)c1] |
| E-5 | 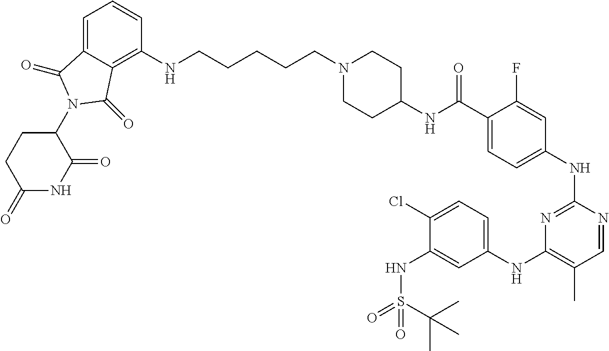 [SMILES: Cc1cnc(Nc2ccc(C(=O)NC3CCN(CCCCCNc4cccc5c4C(=O)N(C4CCC(=O)NC4=O)C5=O)CC3)c(F)c2)nc1Nc1ccc(Cl)c(NS(=O)(=O)C(C)(C)C)c1] |
| E-6 | 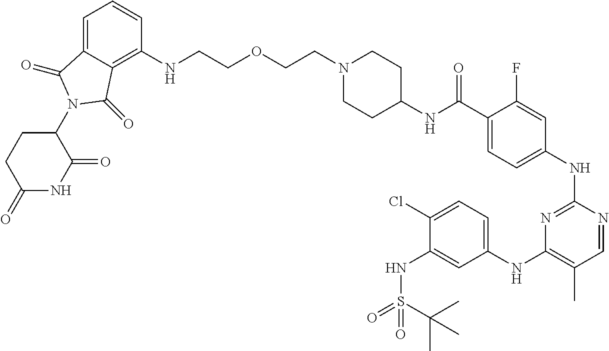 [SMILES: Cc1cnc(Nc2ccc(C(=O)NC3CCN(CCOCCNc4cccc5c4C(=O)N(C4CCC(=O)NC4=O)C5=O)CC3)c(F)c2)nc1Nc1ccc(Cl)c(NS(=O)(=O)C(C)(C)C)c1] |
| E-7 | 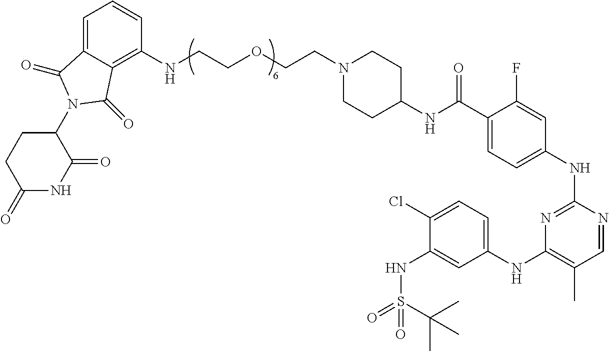 |
| E-8 | 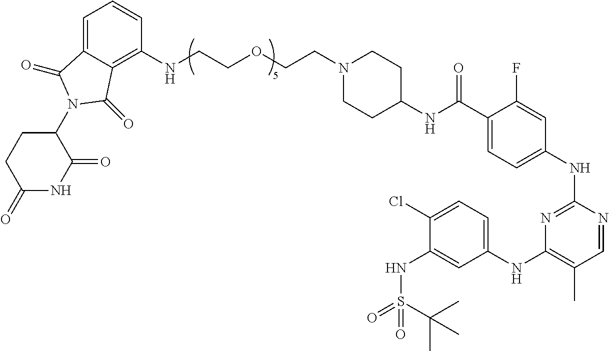 |
| E-9 | 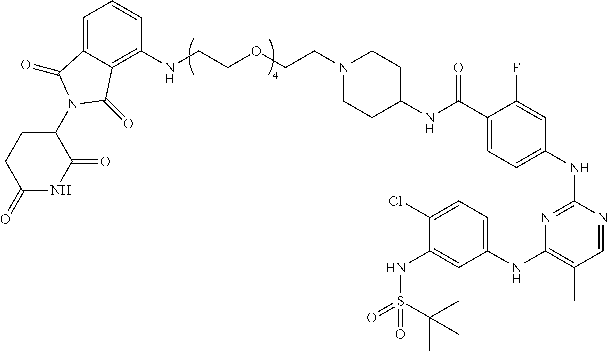 |
| E-10 | 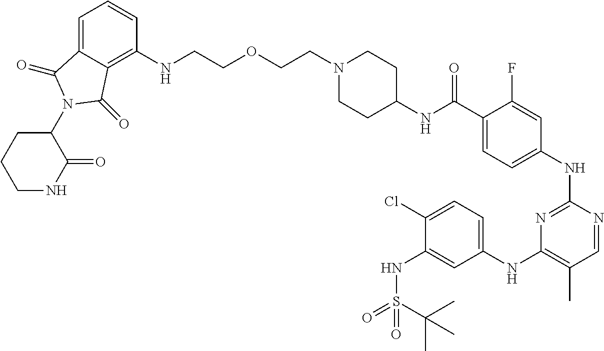 [SMILES: Cc1cnc(Nc2ccc(C(=O)NC3CCN(CCOCCNc4cccc5c4C(=O)N(C4CCCNC4=O)C5=O)CC3)c(F)c2)nc1Nc1ccc(Cl)c(NS(=O)(=O)C(C)(C)C)c1] |
| E-11 | 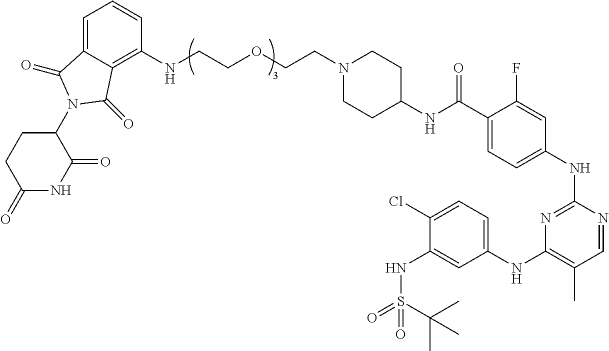 |
| E-12 | 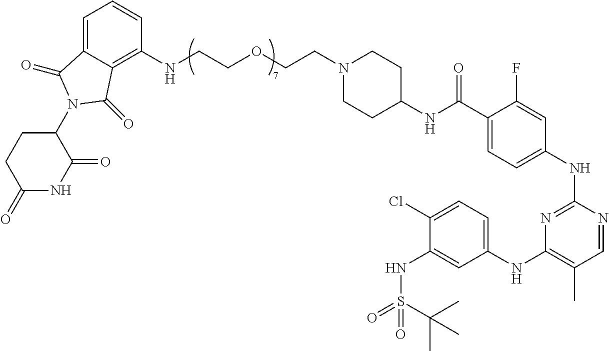 |
| E-13 | 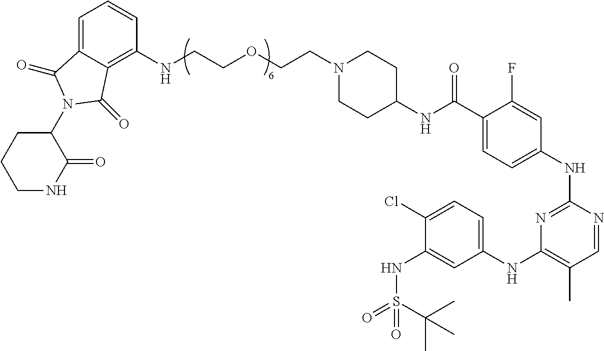 |
| E-14 | 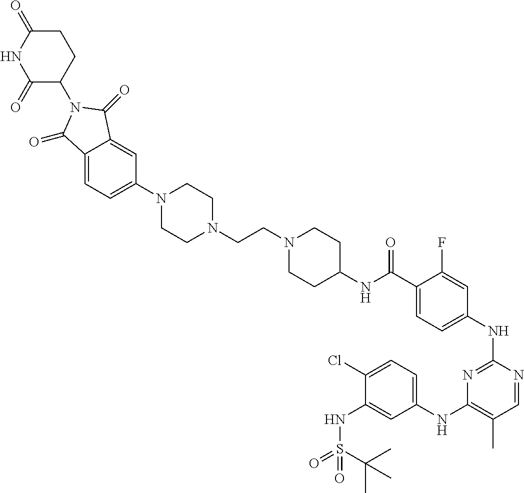 [SMILES: Cc1cnc(Nc2ccc(C(=O)NC3CCN(CCN4CCN(c5ccc6c(c5)C(=O)N(C5CCC(=O)NC5=O)C6=O)CC4)CC3)c(F)c2)nc1Nc1ccc(Cl)c(NS(=O)(=O)C(C)(C)C)c1] |
| E-15 | 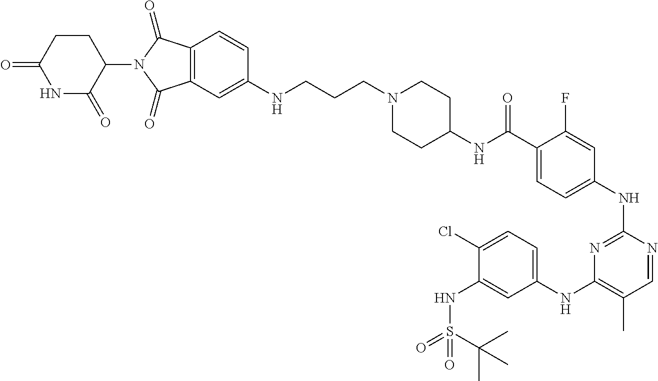 [SMILES: Cc1cnc(Nc2ccc(C(=O)NC3CCN(CCCNc4ccc5c(c4)C(=O)N(C4CCC(=O)NC4=O)C5=O)CC3)c(F)c2)nc1Nc1ccc(Cl)c(NS(=O)(=O)C(C)(C)C)c1] |
| E-16 | 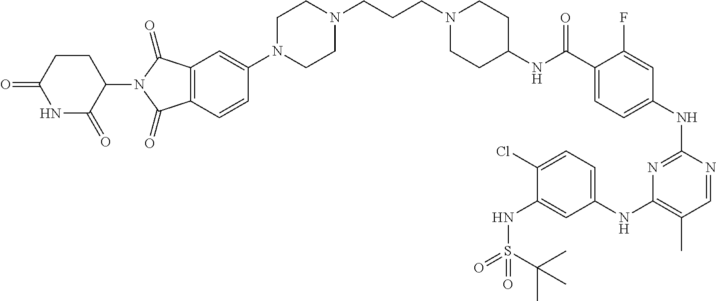 [SMILES: Cc1cnc(Nc2ccc(C(=O)NC3CCN(CCCN4CCN(c5ccc6c(c5)C(=O)N(C5CCC(=O)NC5=O)C6=O)CC4)CC3)c(F)c2)nc1Nc1ccc(Cl)c(NS(=O)(=O)C(C)(C)C)c1] |
| E-17 | 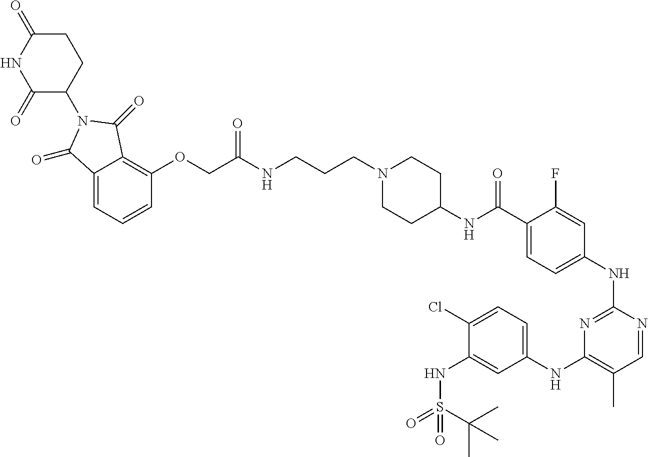 [SMILES: Cc1cnc(Nc2ccc(C(=O)NC3CCN(CCCNC(=O)COc4cccc5c4C(=O)N(C4CCC(=O)NC4=O)C5=O)CC3)c(F)c2)nc1Nc1ccc(Cl)c(NS(=O)(=O)C(C)(C)C)c1] |
| E-18 | 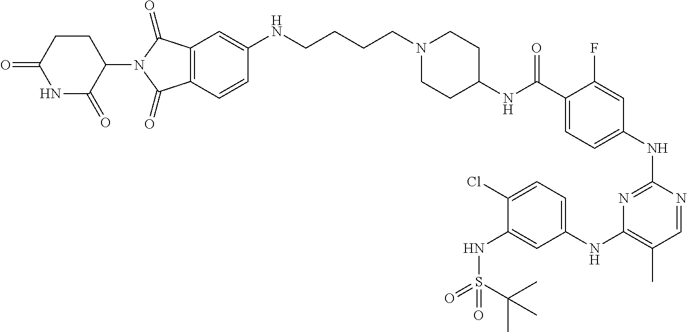 [SMILES: Cc1cnc(Nc2ccc(C(=O)NC3CCN(CCCCNc4ccc5c(c4)C(=O)N(C4CCC(=O)NC4=O)C5=O)CC3)c(F)c2)nc1Nc1ccc(Cl)c(NS(=O)(=O)C(C)(C)C)c1] |

Further representative examples of compounds of Formula II include, but are not limited to, the compounds found in Table F below:

TABLE F
Further Exemplary Compounds of Formula II

| Compound | |
|---|---|
| # | Structure |
| F-1 | 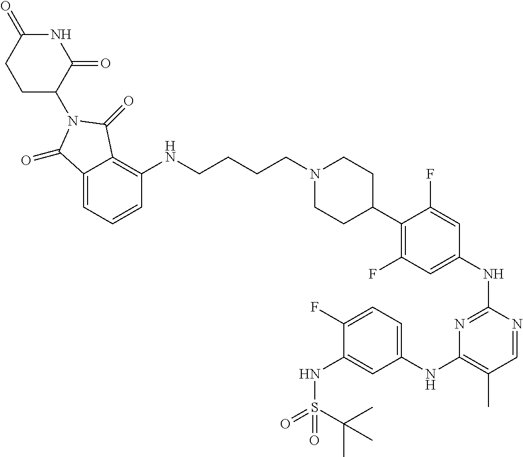 [SMILES: Cc1cnc(Nc2cc(F)c(C3CCN(CCCCNc4cccc5c4C(=O)N(C4CCC(=O)NC4=O)C5=O)CC3)c(F)c2)nc1Nc1ccc(F)c(NS(=O)(=O)C(C)(C)C)c1] |
| F-2 | 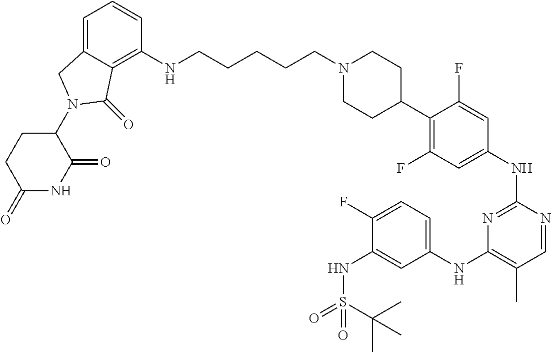 [SMILES: Cc1cnc(Nc2cc(F)c(C3CCN(CCCCCNc4cccc5c4C(=O)N(C4CCC(=O)NC4=O)C5)CC3)c(F)c2)nc1Nc1ccc(F)c(NS(=O)(=O)C(C)(C)C)c1] |
| F-3 | 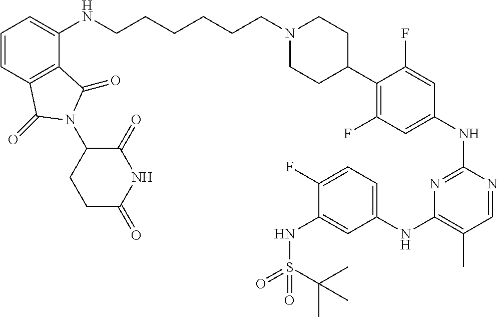 [SMILES: Cc1cnc(Nc2cc(F)c(C3CCN(CCCCCCNc4cccc5c4C(=O)N(C4CCC(=O)NC4=O)C5=O)CC3)c(F)c2)nc1Nc1ccc(F)c(NS(=O)(=O)C(C)(C)C)c1] |
| F-4 | 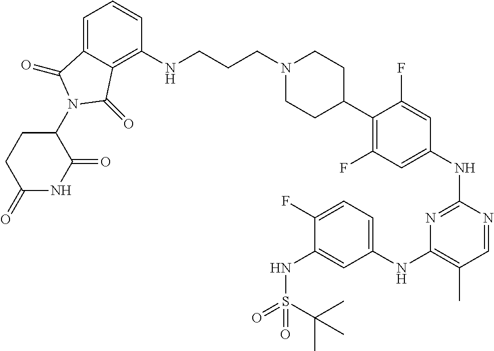 [SMILES: Cc1cnc(Nc2cc(F)c(C3CCN(CCCNc4cccc5c4C(=O)N(C4CCC(=O)NC4=O)C5=O)CC3)c(F)c2)nc1Nc1ccc(F)c(NS(=O)(=O)C(C)(C)C)c1] |
| F-5 | 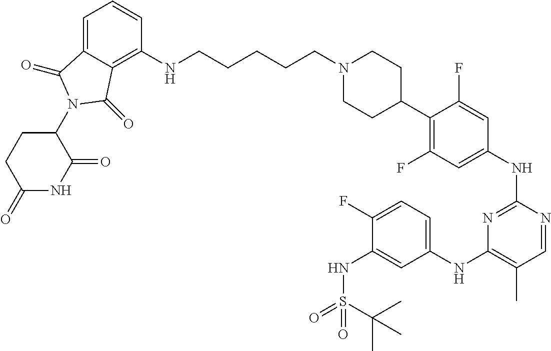 [SMILES: Cc1cnc(Nc2cc(F)c(C3CCN(CCCCCNc4cccc5c4C(=O)N(C4CCC(=O)NC4=O)C5=O)CC3)c(F)c2)nc1Nc1ccc(F)c(NS(=O)(=O)C(C)(C)C)c1] |
| F-6 | 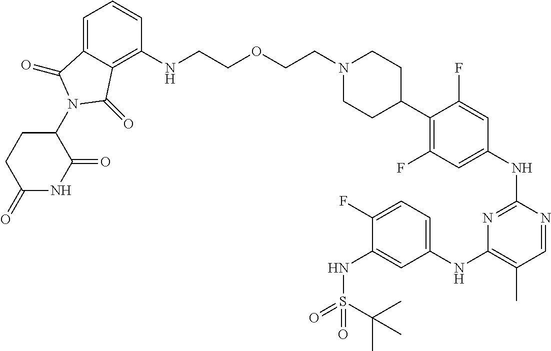 [SMILES: Cc1cnc(Nc2cc(F)c(C3CCN(CCOCCNc4cccc5c4C(=O)N(C4CCC(=O)NC4=O)C5=O)CC3)c(F)c2)nc1Nc1ccc(F)c(NS(=O)(=O)C(C)(C)C)c1] |
| F-7 | 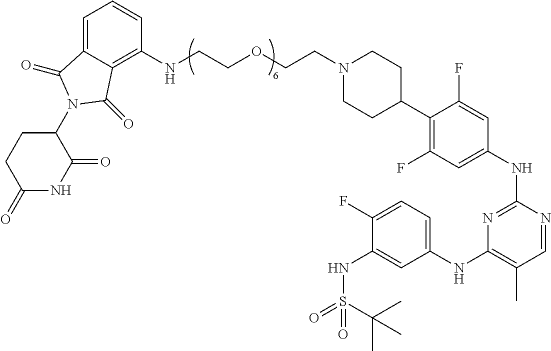 |
| F-8 | 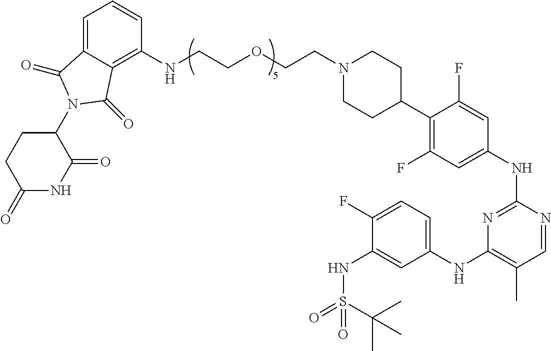 |
| F-9 | 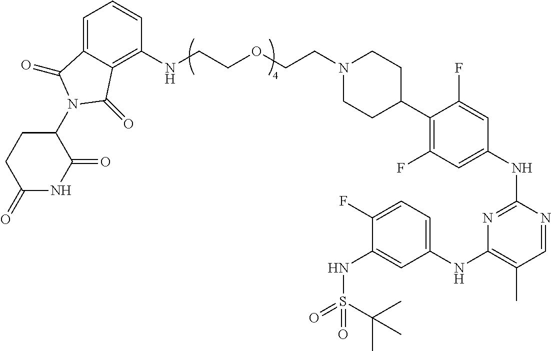 |
| F-10 | 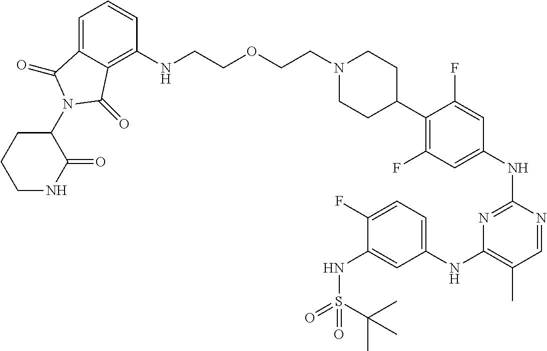 [SMILES: Cc1cnc(Nc2cc(F)c(C3CCN(CCOCCNc4cccc5c4C(=O)N(C4CCCNC4=O)C5=O)CC3)c(F)c2)nc1Nc1ccc(F)c(NS(=O)(=O)C(C)(C)C)c1] |
| F-11 | 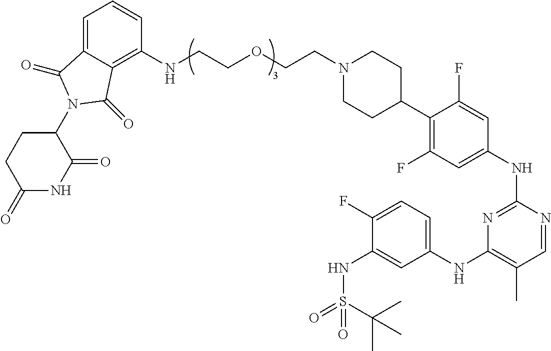 |
| F-12 | 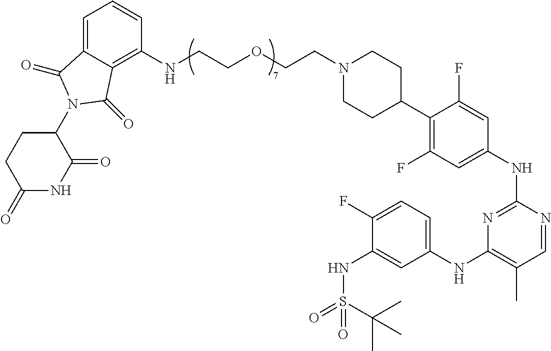 |
| F-13 | 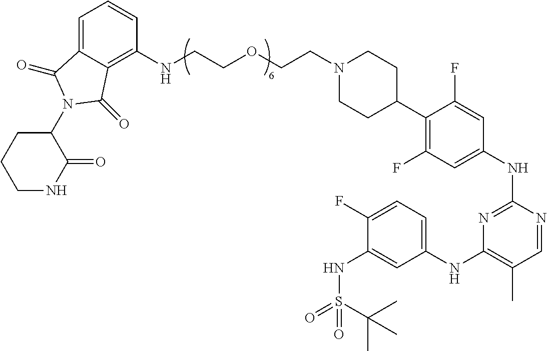 |
| F-14 | 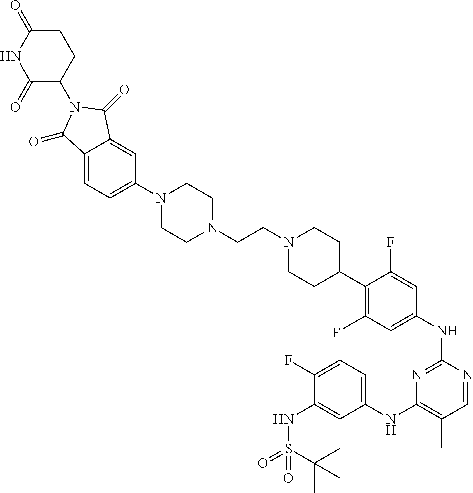 [SMILES: Cc1cnc(Nc2cc(F)c(C3CCN(CCN4CCN(c5ccc6c(c5)C(=O)N(C5CCC(=O)NC5=O)C6=O)CC4)CC3)c(F)c2)nc1Nc1ccc(F)c(NS(=O)(=O)C(C)(C)C)c1] |
| F-15 | 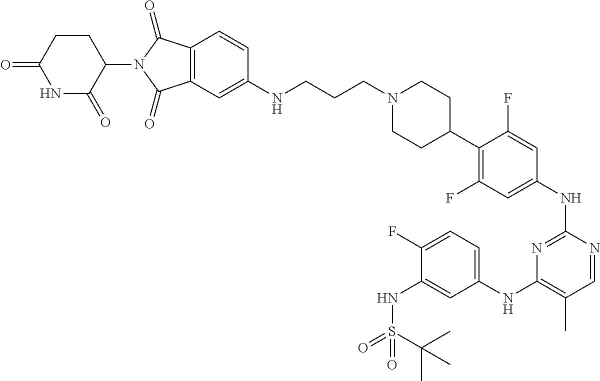 [SMILES: Cc1cnc(Nc2cc(F)c(C3CCN(CCCNc4ccc5c(c4)C(=O)N(C4CCC(=O)NC4=O)C5=O)CC3)c(F)c2)nc1Nc1ccc(F)c(NS(=O)(=O)C(C)(C)C)c1] |
| F-16 | 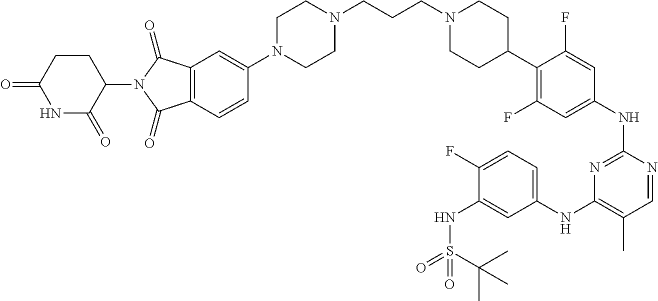 [SMILES: Cc1cnc(Nc2cc(F)c(C3CCN(CCCN4CCN(c5ccc6c(c5)C(=O)N(C5CCC(=O)NC5=O)C6=O)CC4)CC3)c(F)c2)nc1Nc1ccc(F)c(NS(=O)(=O)C(C)(C)C)c1] |
| F-17 | 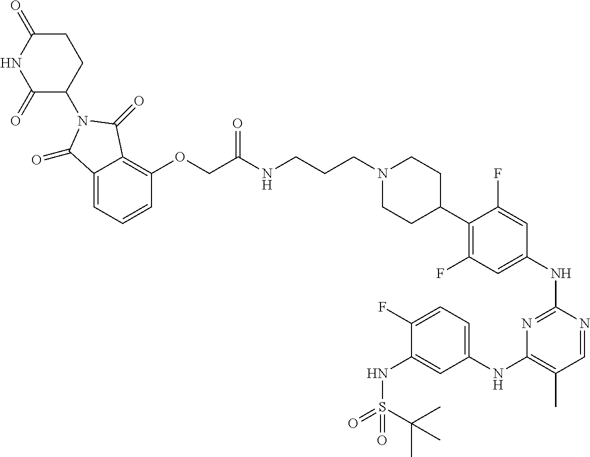 [SMILES: Cc1cnc(Nc2cc(F)c(C3CCN(CCCNC(=O)COc4cccc5c4C(=O)N(C4CCC(=O)NC4=O)C5=O)CC3)c(F)c2)nc1Nc1ccc(F)c(NS(=O)(=O)C(C)(C)C)c1] |
| F-18 | 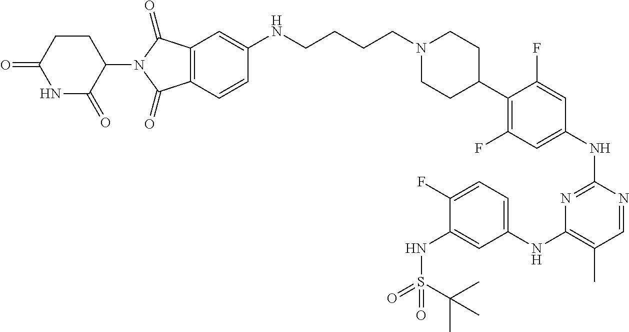 [SMILES: Cc1cnc(Nc2cc(F)c(C3CCN(CCCCNc4ccc5c(c4)C(=O)N(C4CCC(=O)NC4=O)C5=O)CC3)c(F)c2)nc1Nc1ccc(F)c(NS(=O)(=O)C(C)(C)C)c1] |

Further representative examples of compounds of Formula II include, but are not limited to, the compounds found in Table G below:

TABLE G
Further Exemplary Compounds of Formula II

| Compound | |
|---|---|
| # | Structure |
| G-1 | 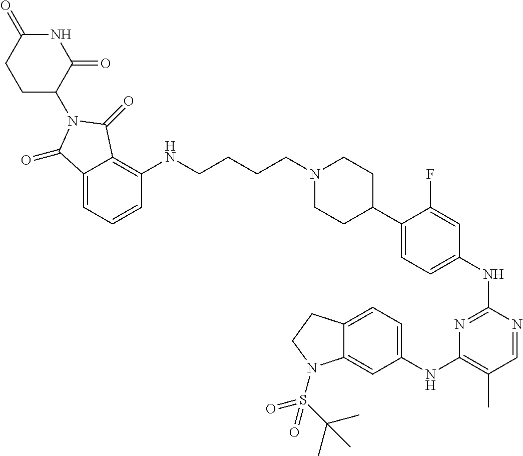 [SMILES: Cc1cnc(Nc2ccc(C3CCN(CCCCNc4cccc5c4C(=O)N(C4CCC(=O)NC4=O)C5=O)CC3)c(F)c2)nc1Nc1ccc2c(c1)N(S(=O)(=O)C(C)(C)C)CC2] |
| G-2 | 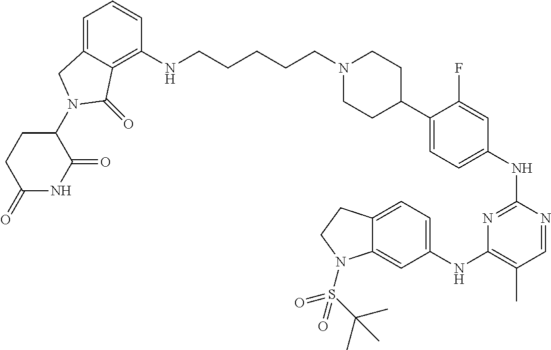 [SMILES: Cc1cnc(Nc2ccc(C3CCN(CCCCCNc4cccc5c4C(=O)N(C4CCC(=O)NC4=O)C5)CC3)c(F)c2)nc1Nc1ccc2c(c1)N(S(=O)(=O)C(C)(C)C)CC2] |
| G-3 | 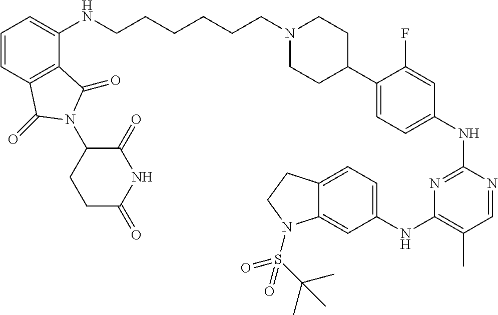 [SMILES: Cc1cnc(Nc2ccc(C3CCN(CCCCCCNc4cccc5c4C(=O)N(C4CCC(=O)NC4=O)C5=O)CC3)c(F)c2)nc1Nc1ccc2c(c1)N(S(=O)(=O)C(C)(C)C)CC2] |
| G-4 | 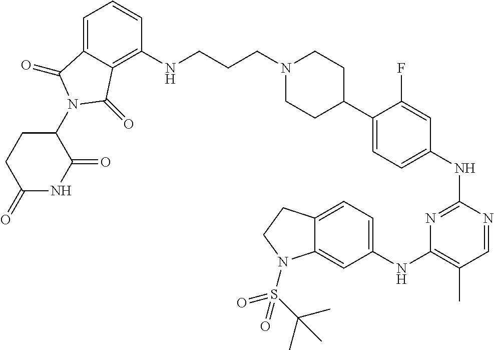 [SMILES: Cc1cnc(Nc2ccc(C3CCN(CCCNc4cccc5c4C(=O)N(C4CCC(=O)NC4=O)C5=O)CC3)c(F)c2)nc1Nc1ccc2c(c1)N(S(=O)(=O)C(C)(C)C)CC2] |
| G-5 | 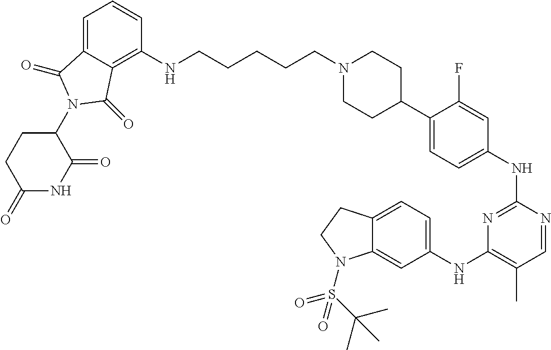 [SMILES: Cc1cnc(Nc2ccc(C3CCN(CCCCCNc4cccc5c4C(=O)N(C4CCC(=O)NC4=O)C5=O)CC3)c(F)c2)nc1Nc1ccc2c(c1)N(S(=O)(=O)C(C)(C)C)CC2] |
| G-6 | 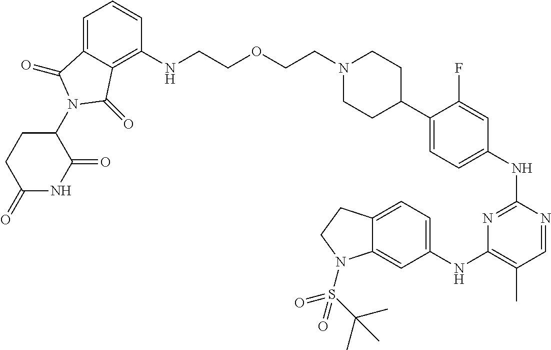 [SMILES: Cc1cnc(Nc2ccc(C3CCN(CCOCCNc4cccc5c4C(=O)N(C4CCC(=O)NC4=O)C5=O)CC3)c(F)c2)nc1Nc1ccc2c(c1)N(S(=O)(=O)C(C)(C)C)CC2] |
| G-7 | 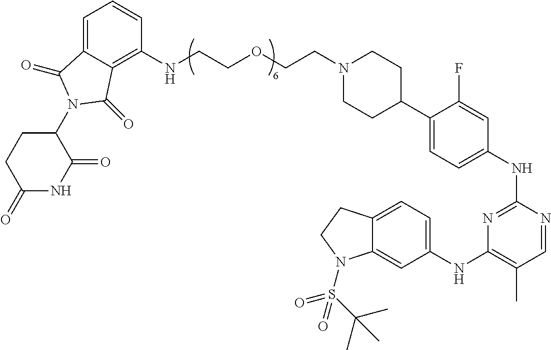 |
| G-8 | 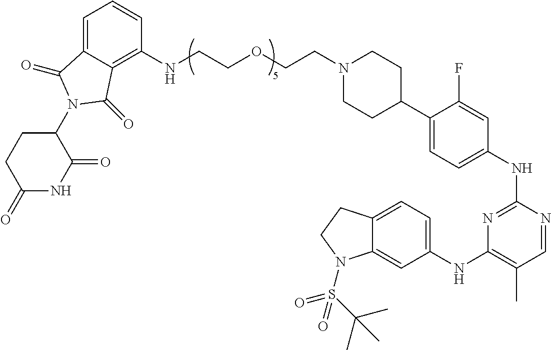 |
| G-9 | 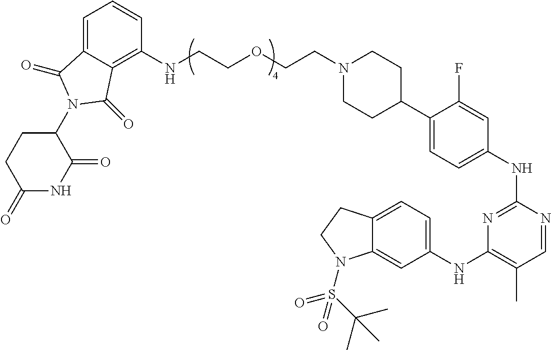 |
| G-10 | 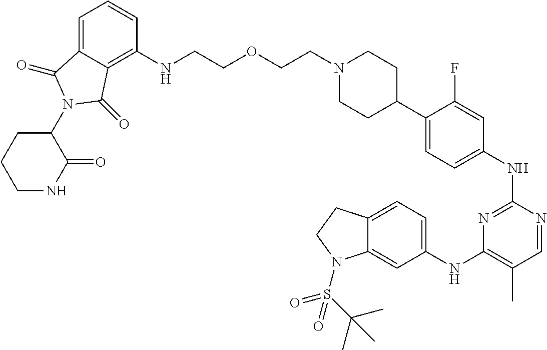 [SMILES: Cc1cnc(Nc2ccc(C3CCN(CCOCCNc4cccc5c4C(=O)N(C4CCCNC4=O)C5=O)CC3)c(F)c2)nc1Nc1ccc2c(c1)N(S(=O)(=O)C(C)(C)C)CC2] |
| G-11 | 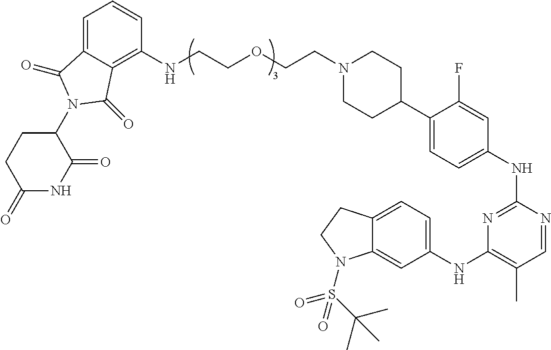 |
| G-12 | 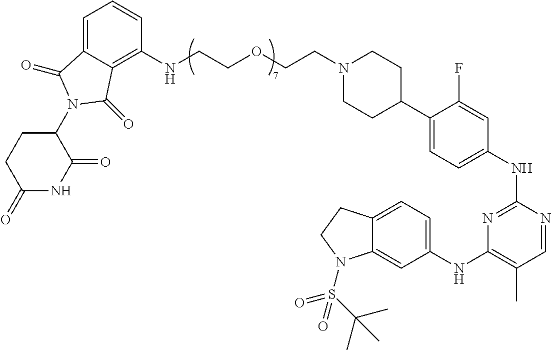 |
| G-13 | 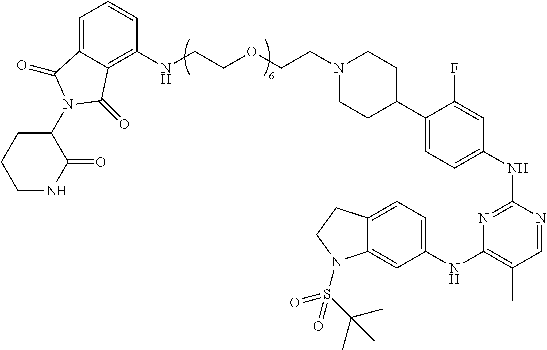 |
| G-14 | 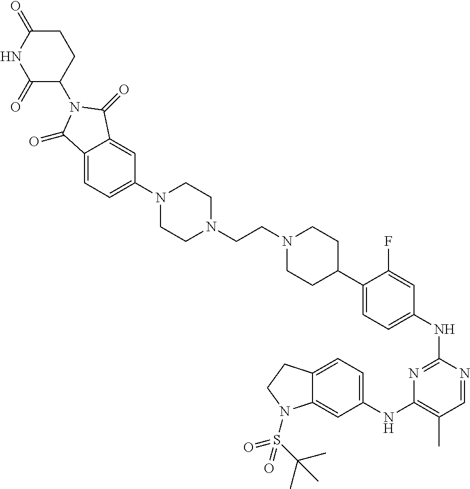 [SMILES: Cc1cnc(Nc2ccc(C3CCN(CCN4CCN(c5ccc6c(c5)C(=O)N(C5CCC(=O)NC5=O)C6=O)CC4)CC3)c(F)c2)nc1Nc1ccc2c(c1)N(S(=O)(=O)C(C)(C)C)CC2] |
| G-15 | 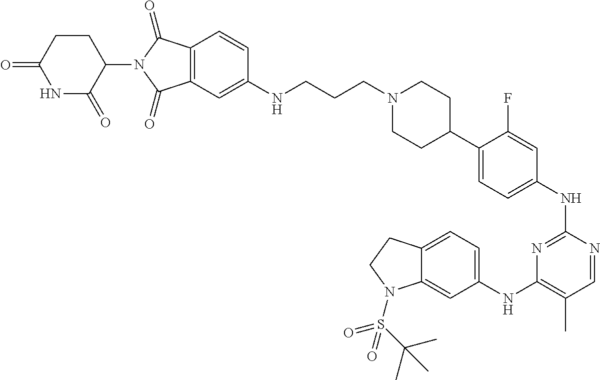 [SMILES: Cc1cnc(Nc2ccc(C3CCN(CCCNc4ccc5c(c4)C(=O)N(C4CCC(=O)NC4=O)C5=O)CC3)c(F)c2)nc1Nc1ccc2c(c1)N(S(=O)(=O)C(C)(C)C)CC2] |
| G-16 | 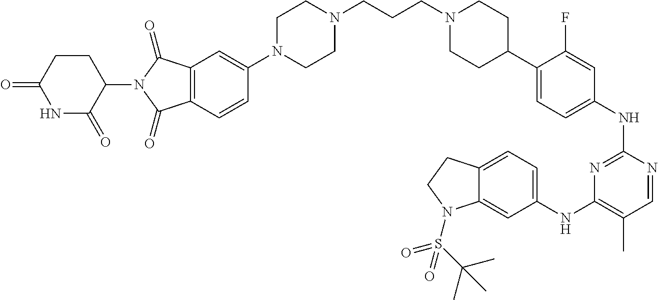 [SMILES: Cc1cnc(Nc2ccc(C3CCN(CCCN4CCN(c5ccc6c(c5)C(=O)N(C5CCC(=O)NC5=O)C6=O)CC4)CC3)c(F)c2)nc1Nc1ccc2c(c1)N(S(=O)(=O)C(C)(C)C)CC2] |
| G-17 | 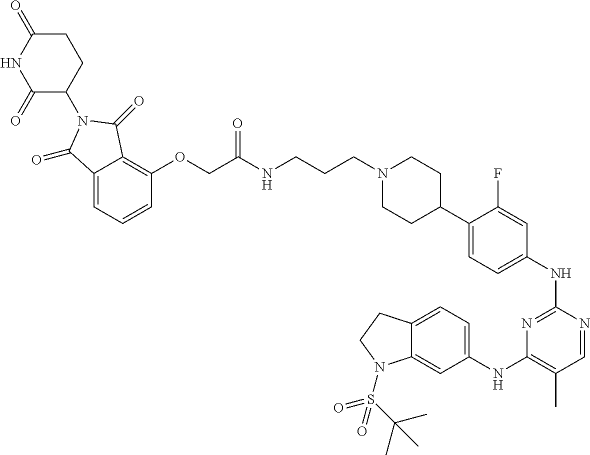 [SMILES: Cc1cnc(Nc2ccc(C3CCN(CCCNC(=O)COc4cccc5c4C(=O)N(C4CCC(=O)NC4=O)C5=O)CC3)c(F)c2)nc1Nc1ccc2c(c1)N(S(=O)(=O)C(C)(C)C)CC2] |
| G-18 | 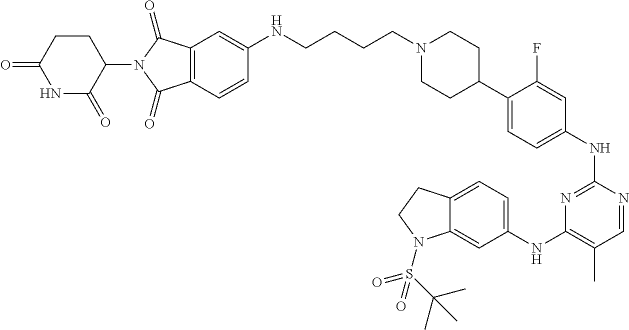 [SMILES: Cc1cnc(Nc2ccc(C3CCN(CCCCNc4ccc5c(c4)C(=O)N(C4CCC(=O)NC4=O)C5=O)CC3)c(F)c2)nc1Nc1ccc2c(c1)N(S(=O)(=O)C(C)(C)C)CC2] |

In another aspect, a compound is provided of Formula III or Formula IV:

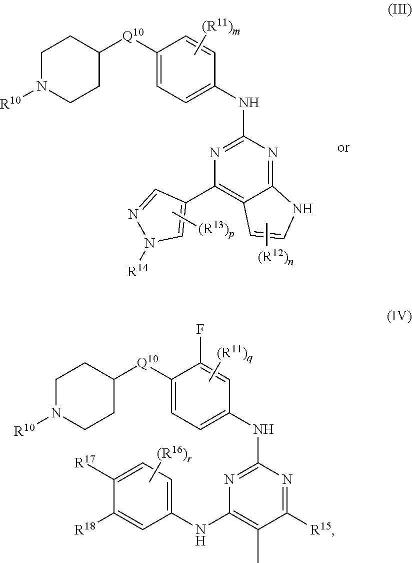

or a pharmaceutically acceptable salt thereof;
wherein:
R¹⁰ is selected from hydrogen or C₁-C₄ alkyl;
Q¹⁰ is a bond, —NH(C═O)—, or —C(═O)NH—;
R¹¹ is independently selected at each occurrence from hydrogen, alkyl, haloalkyl, alkoxy, alkenyl, alkynyl, aryl, heteroaryl, cycloalkyl, heterocycle, aldehyde amino, carboxylic acid, ester, ether, halo, hydroxy, keto, nitro, cyano, azido, silyl, sulfo-oxo, sulfonyl, sulfone, sulfoxide, sulfonylamino, and thiol;
m is selected from 0, 1, 2, 3, or 4;
q is 0, 1, 2, or 3;
R¹² is independently selected at each occurrence from hydrogen, alkyl, haloalkyl, alkoxy, alkenyl, alkynyl, aryl, heteroaryl, cycloalkyl, heterocycle, aldehyde amino, carboxylic acid, ester, ether, halo, hydroxy, keto, nitro, cyano, azido, silyl, sulfo-oxo, sulfonyl, sulfone, sulfoxide, sulfonylamino, and thiol;
n is 0, 1, or 2;
R¹³ is independently selected at each occurrence from hydrogen, alkyl, haloalkyl, alkoxy, alkenyl, alkynyl, aryl, heteroaryl, cycloalkyl, heterocycle, aldehyde amino, carboxylic acid, ester, ether, halo, hydroxy, keto, nitro, cyano, azido, silyl, sulfo-oxo, sulfonyl, sulfone, sulfoxide, sulfonylamino, and thiol;
p is 0, 1, or 2;
R¹⁴ is selected from

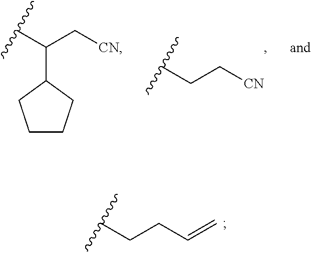

R¹⁵ is selected from hydrogen, alkyl, haloalkyl, alkoxy, alkenyl, alkynyl, aryl, heteroaryl, cycloalkyl, heterocycle, aldehyde amino, carboxylic acid, ester, ether, halo, hydroxy, keto, nitro, cyano, azido, silyl, sulfo-oxo, sulfonyl, sulfone, sulfoxide, sulfonylamino, and thiol;
R¹⁶ is independently selected at each occurrence from hydrogen, alkyl, haloalkyl, alkoxy, alkenyl, alkynyl, aryl, heteroaryl, cycloalkyl, heterocycle, aldehyde amino, carboxylic acid, ester, ether, halo, hydroxy, keto, nitro, cyano, azido, silyl, sulfo-oxo, sulfonyl, sulfone, sulfoxide, sulfonylamino, and thiol;
r is 0, 1, 2, or 3;
R¹⁷ is hydrogen or halogen;
R¹⁸ is —NHSO₂(C₁-C₄ alkyl); or
R¹⁷ and R¹⁸ are brought together with the carbon to which they are attached to form

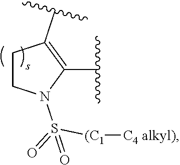

wherein s is 1 or 2.

In some embodiments of Formula III or Formula IV, R¹⁰ is hydrogen. In some embodiments of Formula III or Formula IV, R¹⁰ is methyl.

In some embodiments of Formula III or Formula IV, Q¹⁰ is a bond. In some embodiments of Formula III or Formula IV, Q¹⁰ is —NH(C═O)—. In some embodiments of Formula III or Formula IV, Q¹⁰ is —C(═O)NH—.

In some embodiments of Formula III, R¹⁴ is

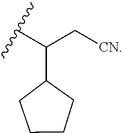 [SMILES: *C(CC#N)C1CCCC1]

In some embodiments of Formula III, R¹⁴ is

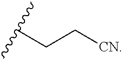 [SMILES: *CCC#N]

In some embodiments of Formula III, R¹⁴ is

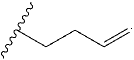

In some embodiments of Formula IV, R¹⁷ is fluoro. In some embodiments of Formula IV, R¹⁷ is chloro.

In some embodiments of Formula IV, R¹⁸ is —NHSO₂(tert-butyl).

In some embodiments of Formula IV, R¹⁷ and R¹⁸ are brought together with the carbons to which they are attached to form

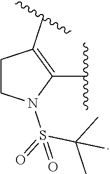

In yet another aspect, a compound is provided selected from:

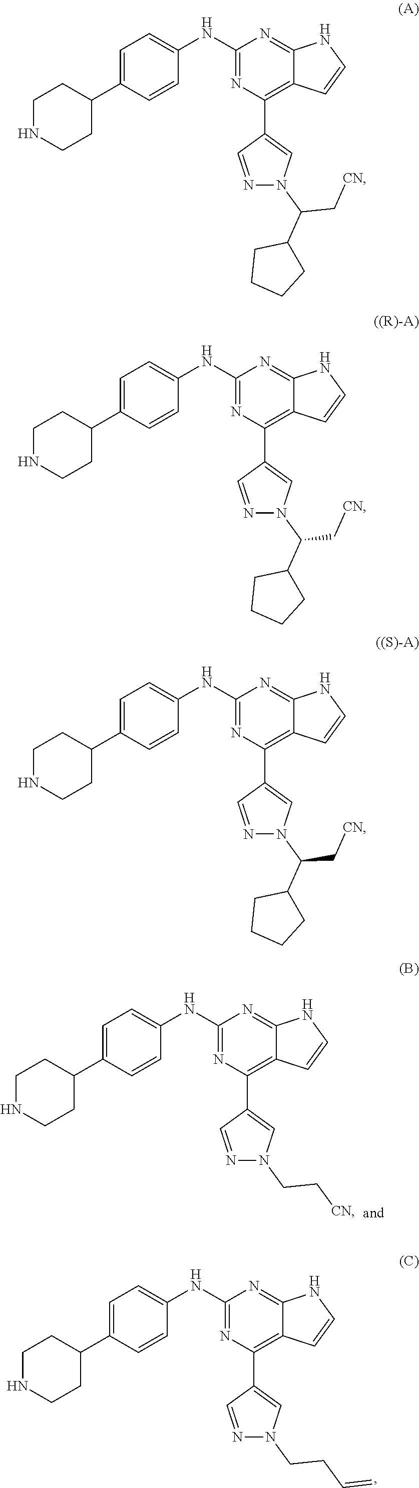

or a pharmaceutically acceptable salt thereof.

In yet another aspect, a compound is provided selected from:

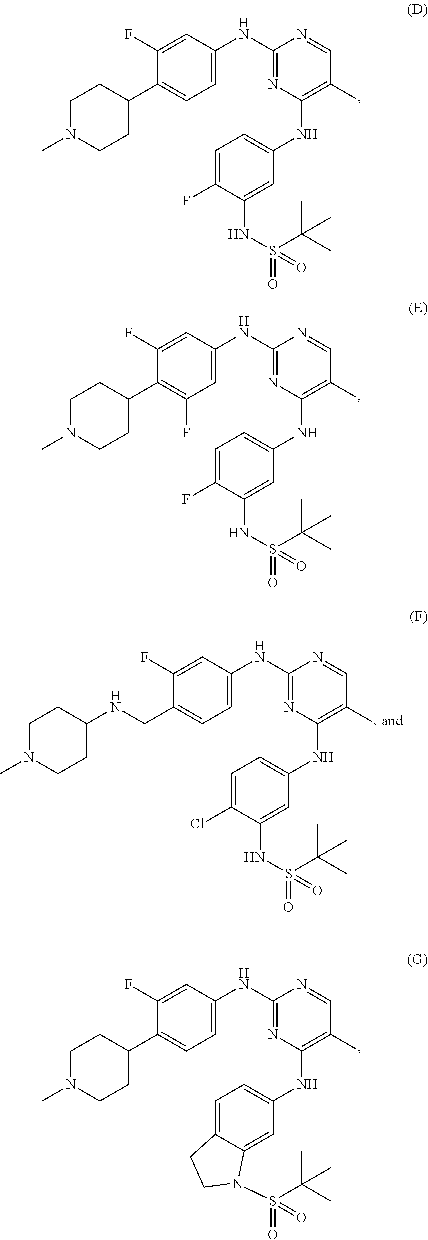

or a pharmaceutically acceptable salt thereof.

The present disclosure also includes compounds of the above formulae with at least one desired isotopic substitution of an atom, at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P, ³²P, ³⁵S, ³⁶C₁, and ¹²⁵I, respectively. In one embodiment, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed herein by substituting a readily available isotopically labeled reagent for a non-isotopically labeled reagent.

By way of general example and without limitation, isotopes of hydrogen, for example deuterium (2H) and tritium (³H) may optionally be used anywhere in described structures that achieves the desired result. Alternatively or in addition, isotopes of carbon, e.g., ¹³C and ¹⁴C, may be used. In one embodiment, the isotopic substitution is replacing hydrogen with a deuterium at one or more locations on the molecule to improve the performance of the molecule as a drug, for example, the pharmacodynamics, pharmacokinetics, biodistribution, half-life, stability, AUC, Tmax, Cmax, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example deuterium substitutions, can be partial or complete. Partial deuterium substitution means that at least one hydrogen is substituted with deuterium. In certain embodiments, the isotope is 80, 85, 90, 95, or 99% or more enriched in an isotope at any location of interest. In some embodiments, deuterium is 80, 85, 90, 95, or 99% enriched at a desired location. Unless otherwise stated, the enrichment at any point is above natural abundance, and in an embodiment is enough to alter a detectable property of the compounds as a drug in a human.

The compounds of the present disclosure may form a solvate with solvents (including water). Therefore, in one embodiment, the invention includes a solvated form of the active compound. The term “solvate” refers to a molecular complex of a compound of the present invention (including a salt thereof) with one or more solvent molecules. Non-limiting examples of solvents are water, ethanol, dimethyl sulfoxide, acetone and other common organic solvents. The term “hydrate” refers to a molecular complex comprising a disclosed compound and water. Pharmaceutically acceptable solvates in accordance with the invention include those wherein the solvent of crystallization may be isotopically substituted, e.g., D₂O, d₆-acetone, or d₆-DMSO. A solvate can be in a liquid or solid form.

A “prodrug” as used herein means a compound which when administered to a host in vivo is converted into a parent drug. As used herein, the term “parent drug” means any of the presently described compounds herein. Prodrugs can be used to achieve any desired effect, including to enhance properties of the parent drug or to improve the pharmaceutic or pharmacokinetic properties of the parent, including to increase the half-life of the drug in vivo. Prodrug strategies provide choices in modulating the conditions for in vivo generation of the parent drug. Non-limiting examples of prodrug strategies include covalent attachment of removable groups, or removable portions of groups, for example, but not limited to, acylating, phosphorylation, phosphonylation, phosphoramidate derivatives, amidation, reduction, oxidation, esterification, alkylation, other carboxy derivatives, sulfoxy or sulfone derivatives, carbonylation, or anhydrides, among others. In certain embodiments, the prodrug renders the parent compound more lipophilic. In certain embodiments, a prodrug can be provided that has several prodrug moieties in a linear, branched, or cyclic manner. For example, non-limiting embodiments include the use of a divalent linker moiety such as a dicarboxylic acid, amino acid, diamine, hydroxycarboxylic acid, hydroxyamine, di-hydroxy compound, or other compound that has at least two functional groups that can link the parent compound with another prodrug moiety, and is typically biodegradable in vivo. In some embodiments, 2, 3, 4, or 5 prodrug biodegradable moieties are covalently bound in a sequence, branched, or cyclic fashion to the parent compound. Non-limiting examples of prodrugs according to the present disclosure are formed with: a carboxylic acid on the parent drug and a hydroxylated prodrug moiety to form an ester; a carboxylic acid on the parent drug and an amine prodrug to form an amide; an amino on the parent drug and a carboxylic acid prodrug moiety to form an amide; an amino on the parent drug and a sulfonic acid to form a sulfonamide; a sulfonic acid on the parent drug and an amino on the prodrug moiety to form a sulfonamide; a hydroxyl group on the parent drug and a carboxylic acid on the prodrug moiety to form an ester; a hydroxyl on the parent drug and a hydroxylated prodrug moiety to form an ester; a phosphonate on the parent drug and a hydroxylated prodrug moiety to form a phosphonate ester; a phosphoric acid on the parent drug and a hydroxylated prodrug moiety to form a phosphate ester; a hydroxyl on the parent drug and a phosphonate on the prodrug to form a phosphonate ester; a hydroxyl on the parent drug and a phosphoric acid prodrug moiety to form a phosphate ester; a carboxylic acid on the parent drug and a prodrug of the structure HO—(CH₂)₂—O—(C₂₋₂₄ alkyl) to form an ester; a carboxylic acid on the parent drug and a prodrug of the structure HO—(CH₂)₂—S—(C₂₋₂₄alkyl) to form a thioester; a hydroxyl on the parent drug and a prodrug of the structure HO—(CH₂)₂—O—(C₂₋₂₄ alkyl) to form an ether; a hydroxyl on the parent drug and a prodrug of the structure HO—(CH₂)₂—O—(C₂₋₂₄ alkyl) to form an thioether; and a carboxylic acid, oxime, hydrazide, hydrazine, amine or hydroxyl on the parent compound and a prodrug moiety that is a biodegradable polymer or oligomer including but not limited to polylactic acid, polylactide-co-glycolide, polyglycolide, polyethylene glycol, polyanhydride, polyester, polyamide, or a peptide.

In some embodiments, a prodrug is provided by attaching a natural or non-natural amino acid to an appropriate functional moiety on the parent compound, for example, oxygen, nitrogen, or sulfur, and typically oxygen or nitrogen, usually in a manner such that the amino acid is cleaved in vivo to provide the parent drug. The amino acid can be used alone or covalently linked (straight, branched or cyclic) to one or more other prodrug moieties to modify the parent drug to achieve the desired performance, such as increased half-life, lipophilicity, or other drug delivery or pharmacokinetic properties. The amino acid can be any compound with an amino group and a carboxylic acid, which includes an aliphatic amino acid, alkyl amino acid, aromatic amino acid, heteroaliphatic amino acid, heteroalkyl amino acid, heterocyclic amino acid, or heteroaryl amino acid.

### Pharmaceutical Compositions

The compounds as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and a pharmaceutically acceptable carrier or excipient of some sort may be useful in a variety of medical and non-medical applications. For example, pharmaceutical compositions comprising an active compound and an excipient may be useful for the treatment or prevention of a cancer in a subject in need thereof.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the medical disorder, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications. Dosage can vary, and can be administered in one or more dose administrations daily, for one or several days.

### Methods of Use

The present disclosure also provides methods for treating or preventing cancer in a subject, comprising administering to the subject a therapeutically effective amount of a compound or composition disclosed herein. The methods can further comprise administering one or more additional therapeutic agents, for example anti-cancer agents or anti-inflammatory agents. Additionally, the method can further comprise administering a therapeutically effective amount of ionizing radiation to the subject.

Methods of killing a cancer or tumor cell are also provided comprising contacting the cancer or tumor cell with an effective amount of a compound or composition as described herein. In some embodiments, the compounds can inhibit JAK2. The methods can further include administering one or more additional therapeutic agents or administering an effective amount of ionizing radiation.

The disclosed methods can optionally include identifying a patient who is or can be in need of treatment of an oncological disorder. The patient can be a human or other mammal, such as a primate (monkey, chimpanzee, ape, etc.), dog, cat, cow pig, or horse, or other animals having an oncological disorder. In some aspects, the subject can receive the therapeutic compositions prior to, during, or after surgical intervention to remove part or all of a tumor.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can also be systemically administered, such as intravenously or orally, optionally in combination with a pharmaceutically acceptable carrier such as an inert diluent, or an assimilable edible carrier for oral delivery. In addition, the active compound can be incorporated into sustained release preparations and/or devices.

For the treatment of an oncological disorder, compounds, agents, and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds, agents, and compositions disclosed herein can be used in methods of treating cancer wherein the patient is to be treated or is or has been treated with mitotic inhibitors such as taxol or vinblastine, alkylating agents such as cyclophosphamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, imatinid or trastuzumab. These other substances or radiation treatments can be given at the same time as or at different times from the compounds disclosed herein. Examples of other suitable chemotherapeutic agents include, but are not limited to, altretamine, bleomycin, bortezomib, busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib, gemcitabine, hydroxyurea, idarubicin, ifosfamide, imatinib, irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uraxil, temozolomide, thiotepa, tioguanine/thioguanine, topotexan, treosulfan, vinblastine, vincristine, vindesine, and vinorelbine. Examples of suitable immunotherapeutic agents include, but are not limited to, alemtuzumab, cetuximab, gemtuzumab, iodine 131 tositumomab, rituximab, and trastuzumab. Cytotoxic agents include, for example, radioactive isotopes and toxins of bacterial, fungal, plant, or animal origin. Also disclosed are methods of treating an oncological disorder comprising administering an effective amount of a compound described herein prior to, subsequent to, and/or in combination with administration of a chemotherapeutic agent, an immunotherapeutic agent, a radiotherapeutic agent, or radiotherapy.

The term “neoplasia” or “cancer” is used throughout this disclosure to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue and most invade surrounding tissues, can metastasize to several sites, are likely to recur after attempted removal and may cause the death of the patient unless adequately treated. As used herein, the term neoplasia is used to describe all cancerous disease states and embraces or encompasses the pathological process associated with malignant, hematogenous, ascitic and solid tumors. The cancers which may be treated by the compositions disclosed herein may comprise carcinomas, sarcomas, lymphomas, leukemias, germ cell tumors, or blastomas.

Carcinomas which may be treated by the compositions of the present disclosure include, but are not limited to, acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocellular carcinoma, Hurthle cell carcinoma,hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lentivular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scirrhous carcinoma, carcinoma scrota, signet-ring cell carcinoma, carcinoma simplex, small cell carcinoma, solandoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, verrucous carcinoma, and carcinoma vilosum.

Representative sarcomas which may be treated by the compositions of the present disclosure include, but are not limited to, liposarcomas (including myxoid liposarcomas and pleomorphic liposarcomas), leiomyosarcomas, rhabdomyosarcomas, neurofibrosarcomas, malignant peripheral nerve sheath tumors, Ewing's tumors (including Ewing's sarcoma of bone, extraskeletal or non-bone) and primitive neuroectodermal tumors (PNET), synovial sarcoma, hemangioendothelioma, fibrosarcoma, desmoids tumors, dermatofibrosarcoma protuberance (DFSP), malignant fibrous histiocytoma(MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) and osteosarcoma (also known as osteogenic sarcoma) skeletal and extra-skeletal, and chondrosarcoma.

The compositions of the present disclosure may be used in the treatment of a lymphoma. Lymphomas which may be treated include mature B cell neoplasms, mature T cell and natural killer (NK) cell neoplasms, precursor lymphoid neoplasms, Hodgkin lymphomas, and immunodeficiency-associated lymphoproliferative disorders. Representative mature B cell neoplasms include, but are not limited to, B-cell chronic lymphocytic leukemia/small cell lymphoma, B-cell prolymphocytic leukemia, lymphoplasmacytic lymphoma (such as Waldenstram macroglobulinemia), splenic marginal zone lymphoma, hairy cell leukemia, plasma cell neoplasms (such as plasma cell myeloma/multiple myeloma, plasmacytoma, monoclonal immunoglobulin deposition diseases, and heavy chain diseases), extranodal marginal zone B cell lymphoma (MALT lymphoma), nodal marginal zone B cell lymphoma, follicular lymphoma, primary cutaneous follicular center lymphoma, mantle cell lymphoma, diffuse large B cell lymphoma, diffuse large B-cell lymphoma associated with chronic inflammation, Epstein-Barr virus-positive DLBCL of the elderly, lyphomatoid granulomatosis, primary mediastinal (thymic) large B-cell lymphoma, intravascular large B-cell lymphoma, ALK+ large B-cell lymphoma, plasmablastic lymphoma, primary effusion lymphoma, large B-cell lymphoma arising in HHV8-associated multicentric Castleman's disease, and Burkitt lymphoma/leukemia. Representative mature T cell and NK cell neoplasms include, but are not limited to, T-cell prolymphocytic leukemia, T-cell large granular lymphocyte leukemia, aggressive NK cell leukemia, adult T-cell leukemia/lymphoma, extranodal NK/T-cell lymphoma, nasal type, enteropathy-associated T-cell lymphoma, hepatosplenic T-cell lymphoma, blastic NK cell lymphoma, lycosis fungoides/Sezary syndrome, primary cutaneous CD30-positive T cell lymphoproliferative disorders (such as primary cutaneous anaplastic large cell lymphoma and lymphomatoid papulosis), peripheral T-cell lymphoma not otherwise specified, angioimmunoblastic T cell lymphoma, and anaplastic large cell lymphoma. Representative precursor lymphoid neoplasms include B-lymphoblastic leukemia/lymphoma not otherwise specified, B-lymphoblastic leukemia/lymphoma with recurrent genetic abnormalities, or T-lymphoblastic leukemia/lymphoma. Representative Hodgkin lymphomas include classical Hodgkin lymphomas, mixed cellularity Hodgkin lymphoma, lymphocyte-rich Hodgkin lymphoma, and nodular lymphocyte-predominant Hodgkin lymphoma.

The compositions of the present disclosure may be used in the treatment of a Leukemia. Representative examples of leukemias include, but are not limited to, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia, adult T-cell leukemia, clonal eosinophilias, and transient myeloproliferative disease.

The compositions of the present disclosure may be used in the treatment of a germ cell tumor, for example germinomatous (such as germinoma, dysgerminoma, and seminoma), non germinomatous (such as embryonal carcinoma, endodermal sinus tumor, choriocarcinoma, teratoma, polyembryoma, and gonadoblastoma) and mixed tumors.

The compositions of the present disclosure may be used in the treatment of blastomas, for example hepatoblastoma, medulloblastoma, nephroblastoma, neuroblastoma, pancreatoblastoma, pleuropulmonary blastoma, retinoblastoma, and glioblastoma multiforme.

Representative cancers which may be treated include, but are not limited to: bone and muscle sarcomas such as chondrosarcoma, Ewing's sarcoma, malignant fibrous histiocytoma of bone/osteosarcoma, osteosarcoma, rhabdomyosarcoma, and heart cancer; brain and nervous system cancers such as astrocytoma, brainstem glioma, pilocytic astrocytoma, ependymoma, primitive neuroectodermal tumor, cerebellar astrocytoma, cerebral astrocytoma, glioma, medulloblastoma, neuroblastoma, oligodendroglioma, pineal astrocytoma, pituitary adenoma, and visual pathway and hypothalamic glioma; breast cancers including invasive lobular carcinoma, tubular carcinoma, invasive cribriform carcinoma, medullary carcinoma, male breast cancer, Phyllodes tumor, and inflammatory breast cancer; endocrine system cancers such as adrenocortical carcinoma, islet cell carcinoma, multiple endocrine neoplasia syndrome, parathyroid cancer, phemochromocytoma, thyroid cancer, and Merkel cell carcinoma; eye cancers including uveal melanoma and retinoblastoma; gastrointestinal cancers such as anal cancer, appendix cancer, cholangiocarcinoma, gastrointestinal carcinoid tumors, colon cancer, extrahepatic bile duct cancer, gallbladder cancer, gastric cancer, gastrointestinal stromal tumor, hepatocellular cancer, pancreatic cancer, and rectal cancer; genitourinary and gynecologic cancers such as bladder cancer, cervical cancer, endometrial cancer, extragonadal germ cell tumor, ovarian cancer, ovarian epithelial cancer, ovarian germ cell tumor, penile cancer, renal cell carcinoma, renal pelvis and ureter transitional cell cancer, prostate cancer, testicular cancer, gestational trophoblastic tumor, urethral cancer, uterine sarcoma, vaginal cancer, vulvar cancer, and Wilms tumor; head and neck cancers such as esophageal cancer, head and neck cancer, nasopharyngeal carcinoma, oral cancer, oropharyngeal cancer, paranasal sinus and nasal cavity cancer, pharyngeal cancer, salivary gland cancer, and hypopharyngeal cancer; hematopoietic cancers such as acute biphenotypic leukemia, acute eosinophilic leukemia, acute lymphoblastic leukemia, acute myeloid leukemia, acute myeloid dendritic cell leukemia, AIDS-related lymphoma, anaplastic large cell lymphoma, angioimmunoblastic T-cell lymphoma, B-cell prolymphocytic leukemia, Burkitt's lymphoma, chronic lymphocytic leukemia, chronic myelogenous leukemia, cutaneous T-cell lymphoma, diffuse large B-cell lymphoma, follicular lymphoma, hairy cell leukemia, hepatosplenic T-cell lymphoma, Hodgkin's lymphoma, hairy cell leukemia, intravascular large B-cell lymphoma, large granular lymphocytic leukemia, lymphoplasmacytic lymphoma, lymphomatoid granulomatosis, mantle cell lymphoma, marginal zone B-cell lymphoma, Mast cell leukemia, mediastinal large B cell lymphoma, multiple myeloma/plasma cell neoplasm, myelodysplastic syndroms, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sezary syndrome, splenic marginal zone lymphoma, and T-cell prolymphocytic leukemia; skin cancers such as basal cell carcinoma, squamous cell carcinoma, skin adnexal tumors (such as sebaceous carcinoma), melanoma, Merkel cell carcinoma, sarcomas of primary cutaneous origin (such as dermatofibrosarcoma protuberans), and lymphomas of primary cutaneous origin (such as mycosis fungoides); thoracic and respiratory cancers such as bronchial adenomas/carcinoids, small cell lung cancer, mesothelioma, non-small cell lung cancer, pleuropulmonary blastoma, laryngeal cancer, and thymoma or thymic carcinoma; HIV/AIDs-related cancers such as Kaposi sarcoma; epithelioid hemangioendothelioma; desmoplastic small round cell tumor; and liposarcoma.

In another aspect, a method is provided for treating a JAK2-associated disease or disorder in a subject in need thereof comprising administering to the subject a therapeutically effective amount of a compound or composition described herein. The JAK2-associated disease can include any disease, disorder or condition that is directly or indirectly linked to expression or activity of JAK2, including overexpression and/or abnormal activity levels. A JAK2-associated disease can also include any disease, disorder or condition that can be prevented, ameliorated or cured by modulating JAK2 activity.

JAK2-associated diseases include disease involving the immune system such as, for example, organ transplant rejection (e.g., allograft rejection and graft-versus-host disease).

JAK2-associated diseases may also include autoimmune diseases such as multiple sclerosis, rheumatoid arthritis, psoriatic arthritis, type I diabetes, lupus, psoriasis, inflammatory bowl disease, ulcerative colitis, Crohn's disease, myasthenia gravis, immunoglobin nephropathies, autoimmune thyroid disorders, and the like. In some embodiments, the autoimmune disease is an autoimmune bullous skin disorder such as pemphigus vulgaris (PV) or bullous pemphigoid (BP).

In another example, JAK2-associated diseases include allergic conditions such as asthma, food allergies, atopic dermatitis and rhinitis. Further examples of JAK2-associated disease include viral disease such as Epstein Barr Virus (EBV), Hepatitis B, Hepatitis C, HIV, HTLV 1, Varicella-Zoster Virus (VZV) and Human Papilloma Virus (HPV).

### Kits

Kits for practicing the methods described herein are further provided. By “kit” is intended any manufacture (e.g., a package or a container) comprising at least one reagent, e.g., any one of the compounds described herein. The kit can be promoted, distributed, or sold as a unit for performing the methods described herein. Additionally, the kits can contain a package insert describing the kit and methods for its use. Any or all of the kit reagents can be provided within containers that protect them from the external environment, such as in sealed containers or pouches.

To provide for the administration of such dosages for the desired therapeutic treatment, in some embodiments, pharmaceutical compositions disclosed herein can comprise between 0.1% and 45%, and especially, 1 and 15%, by weight of the total of one or more of the compounds based on the weight of the total composition including carriers and/or diluents. Illustratively, dosage levels of the administered active ingredients can be: intravenous 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasally, 0.01 to about 20 mg/kg; and aerosol, 0.01 to about 20 mg/kg of animal (body) weight.

Also disclosed are kits that comprise a composition comprising a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more therapeutic agents, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are put forth so as to provide those of ordinary skill in the art with a complete disclosure and description of how the compounds, compositions, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric pressure.

Structural Insights into JAK2 Inhibition by Ruxolitinib, Fedratinib, and Derivatives Thereof

The discovery that aberrant activity of Janus kinase 2 (JAK2) is a driver of myeloproliferative neoplasms (MPNs) has led to significant efforts to develop small molecule inhibitors for this patient population. Ruxolitinib and fedratinib have been approved for use in MPN patients, while baricitinib, an achiral analogue of ruxolitinib, has been approved for rheumatoid arthritis. However, structural information on the interaction of these therapeutics with JAK2 remained unknown. Here, we introduce a new methodology for the large-scale production of JAK2 from mammalian cells, which enabled the first crystal structures of JAK2 bound to these drugs and derivatives thereof. Along with biochemical and cellular data, the results provide a comprehensive view of the shape complementarity required for chiral and achiral inhibitors to achieve highest activity, which may facilitate the development of more effective JAK2 inhibitors as therapeutics.

To date, no crystal structure of ruxolitinib has been reported with any of the JAK family proteins, making ruxolitinib the oldest FDA-approved kinase inhibitor without a co-crystal structure with its target protein. (see Zhou, T.; Georgeon, S.; Moser, R.; Moore, D. J.; Caflisch, A.; Hantschel, O. Specificity and mechanism-of-action of the JAK2 tyrosine kinase inhibitors ruxolitinib and SAR302503 (TG101348). Leukemia 2014, 28, 404-407; and Lamontanara, A. J.; Gencer, E. B.; Kuzyk, O.; Hantschel, O. Mechanisms of resistance to BCR-ABL and other kinase inhibitors. Biochim Biophys Acta 2013, 1834, 1449-1459) Likewise, structural information on the interaction of JAK2 with fedratinib and baricitinib remained unknown. Knowledge of these structures, however, is required for the development of more efficacious drugs to specifically target aberrant JAK2. Here we report the first crystal structures of ruxolitinib, fedratinib and baricitinib along with derivatives bound to the JAK2 kinase domain (KD). We introduce a new methodology to efficiently produce crystallization-grade JAK2 KD using mammalian cells, which enabled efficient structure-activity relationship (SAR) studies with inhibitors of different chemical scaffolds. A total of 14 high-resolution crystal structures were determined of JAK2 liganded with known and novel inhibitors. The data sets detail the requirements of small molecule inhibitors for shape complementarity with the ATP site of JAK2 and provide a structural basis for the stereoselective discrimination of enantiomers of ruxolitinib and derivatives thereof.

### Experimental

Compounds and reagents: Reagents for biochemical and crystallographic experiments were purchased from Fisher Scientific and Hampton Research unless otherwise indicated. Ruxolitinib (phosphate) was from LC Laboratories (R-6688, >99%), Tofacitinib (citrate) from MedChemExpress (HY-40354A, 99.1%), Baricitinib (free base) from Combi-blocks (QJ-1094, 98%), Fedratinib from MedChemExpress (HY-10409, 99.9%). The following antibodies were used for immunoblotting: His-HRP (ProteinTech, HRP-66005, 1:5,000), pJAK2 (Y1007/1008) (Cell Signaling, 3771, 1:1,000), actin (Sigma, A5441, 1:1,000), GAPDH-HRP (ProteinTech, HRP-60004, 1:10,000), anti-mouse-HRP IgG (Jackson Immuno Research, 115-035-003, 1:2,000).
General Synthetic Methods: All reagents were purchased from commercial suppliers and used without further purification. ¹H NMR spectra were recorded on Bruker 500 MHz spectrometer with CDCl₃, CD₃OD or DMSO-d₆ as the solvent. ¹³C NMR spectra were recorded at 125 MHz. All coupling constants are measured in Hertz (Hz) and the chemical shifts (δ_{H} and δ_{C}) are quoted in parts per million (ppm) relative to TMS (δ 0), which was used as the internal standard. High resolution mass spectroscopy was carried out on an Agilent 6210 LC/MS (ESI-TOF). HPLC-MS analysis was performed using Agilent 6120 single quadrupole 1220 LCMS equipped with Zorbax SB-C18 column (4.6×50 mm, 1.8 micron). The purity of final compounds that underwent biological assessment were >95% as measured by HPLC-MS. Thin layer chromatography was performed using silica gel 60 F254 plates (Fisher), with observation under UV when necessary. Anhydrous solvents (acetonitrile, dimethylformamide, ethanol, isopropanol, methanol and tetrahydrofuran) were used as purchased from Aldrich. Burdick and Jackson HPLC grade solvents (methanol, acetonitrile and water) were purchased from VWR for HPLC and high-resolution mass analysis. HPLC grade TFA was purchased from Fisher. Compound synthesis and characterization are detailed in the Supporting Information.

### Compound Synthesis and Characterization

Pyrrolopyrimidine inhibitors of JAK2. (S)-Ruxolitinib was prepared by a novel resolution of pyrazole-containing carboxylic acid 9 [from conjugate addition of 4-bromo-1H-pyrazole, and ester hydrolysis of 8′] as shown in Scheme 1.

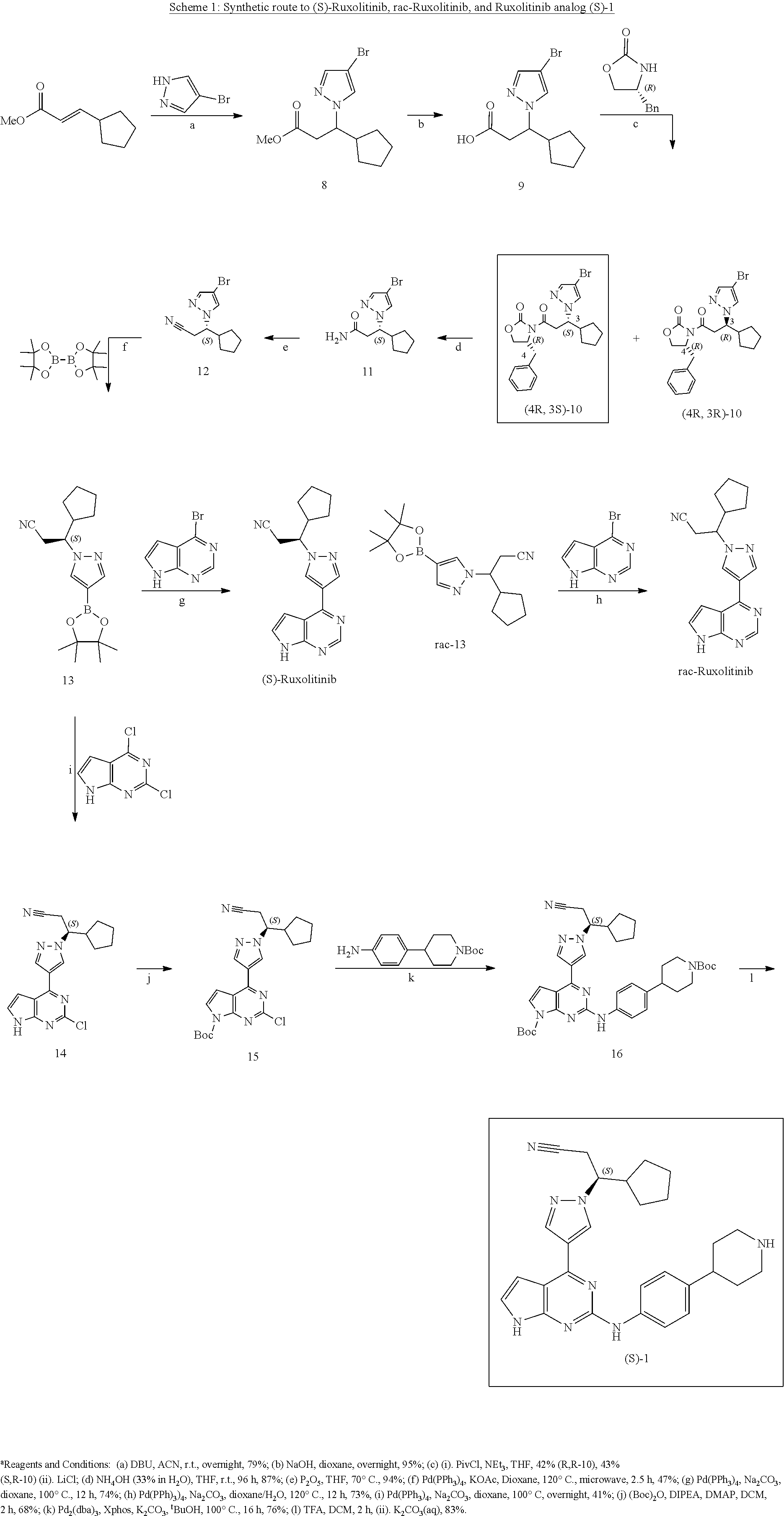

Reaction of 9 with the Evans oxazolidinone provided the diastereoisomeric oxazolidinones (4R,3R)-10 and (4R,3S)-10 which were easily separated to homogeneity by flash chromatography on silica gel. The faster-running fraction was identified as (4R,3S)-10 by correlation to the enantiomer of clinically used (R)-ruxolitinib using the synthetic route described in Cao, B.; Zhu, H.; Wu, J.; Tian, G. Intermediate of JAK inhibitor and preparation method thereof. China patent 2018, CN107759623A. Treatment of (4R,3S)-10 with ammonium hydroxide (see Steele, A. D.; Keohane, C. E.; Knouse, K. W.; Rossiter, S. E.; Williams, S. J.; Wuest, W. M. Diverted total synthesis of promysalin analogs demonstrates that an iron-binding motif is responsible for its narrow-spectrum antibacterial activity. Journal of the American Chemical Society 2016, 138, 5833-5836) gave the primary amide 11, which upon dehydration with phosphorus pentoxide gave the nitrile 12. Formation of the boronate ester 13 followed by Suzuki reaction with 4-bromo-7H-pyrrolo[2,3-d]pyrimidine provided (S)-ruxolitinib. The enantiomeric excess of the (S)-ruxolitinib was determined to be >99.99% by chiral HPLC. A sample of rac-ruxolitinib was prepared from rac-13 in the same way. The synthesis of the ruxolitinib analogs (R) and (S)-1 is shown in schemes 1 and 2. 2-Chloro-ruxolitinib (14) was prepared by Suzuki coupling of 13 with 2,4-dichloropyrrolopyrimidine (Scheme 1). Substitution of the chlorine atom with the anilinopyrimidine subsituent required protection of the pyrrole NH group. Thereby, Buchwald-Hartwig amination of the boc-protected pyrrolopyrimidine 15 gave 16 bearing the anilinopiperidine at the 2-position of the pyrimidine ring. Finally, removal of the protecting groups gave the desired (S)-1 bearing the required piperidine group oriented towards the solvent accessible region. Again, chiral HPLC analysis indicated that the enantiomeric excess of (S)-1 was >99.99% (as determined by the e.e. of its bis-boc protected precursor (S)-16). The enantiomer (R)-1 was prepared in the same way using the diasteroisomer (4R,3R)-10 (Scheme 2).

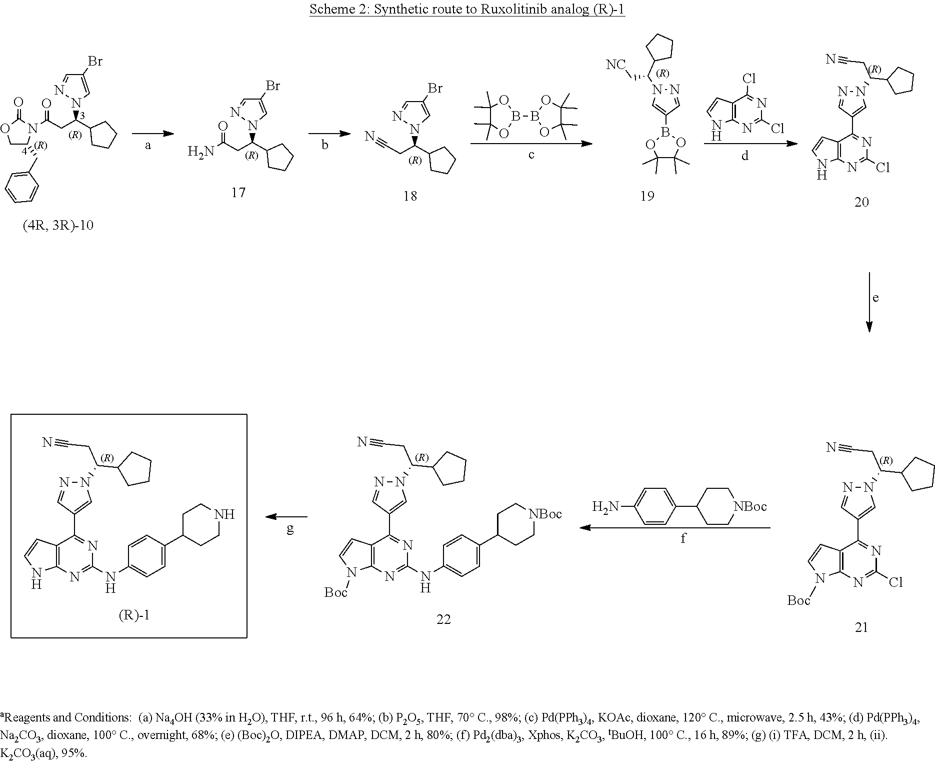

In this case the enantiomeric excess of (R)-1 was 98.6% (as determined by the e.e. of its bis-boc protected precursor (R)-22). Ruxolitinib derivatives lacking the cyclopentyl group have been shown to retain potent JAK2 inhibitory activity. (see Yao, L.; Mustafa, N.; Tan, E. C.; Poulsen, A.; Singh, P.; Duong-Thi, M. D.; Lee, J. X. T.; Ramanujulu, P. M.; Chng, W. J.; Yen, J. J. Y.; Ohlson, S.; Dymock, B. W. Design and synthesis of ligand efficient dual inhibitors of Janus kinase (JAK) and histone deacetylase (HDAC) based on ruxolitinib and vorinostat. J Med Chem 2017, 60, 8336-8357; and Yao, L.; Ramanujulu, P. M.; Poulsen, A.; Ohlson, S.; Dymock, B. W. Merging of ruxolitinib and vorinostat leads to highly potent inhibitors of JAK2 and histone deacetylase 6 (HDAC6). Bioorg Med Chem Lett 2018, 28, 2636-2640) Thus, the piperidine 2, incorporating the propionitrile substituted pyrazole, was prepared in a similar way by Suzuki reaction of addition of 2,4-dichloropyrrolopyrimidine and the boronic ester 27 [from acrylonitrile and 4-pyrazoleboronic pinacolate] to give the nitrile 28 (Scheme 3).

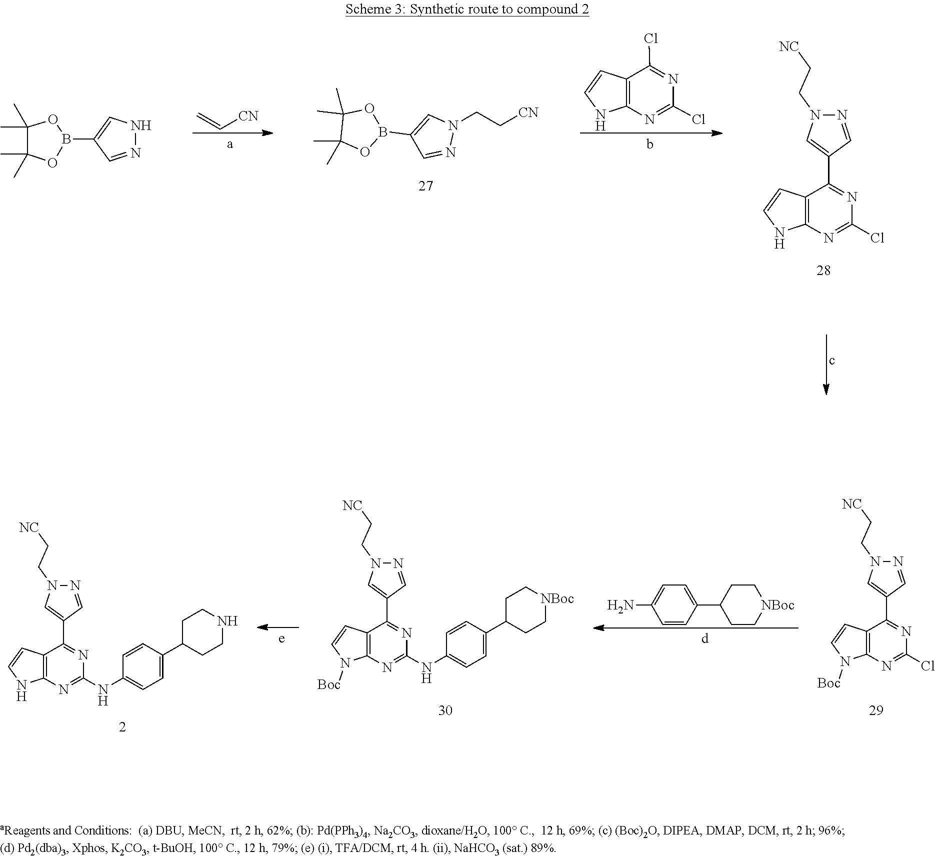

A similar sequence of Buchwald-Hartwig amination of the boc-protected 2-chloropyrrolopyrimidine 29 followed by deprotection of 30 provided the piperidine 2. The piperidine 3, bearing a butenyl substituted pyrazole group was prepared by the same methods used to make the piperidine 2 (Scheme 4).

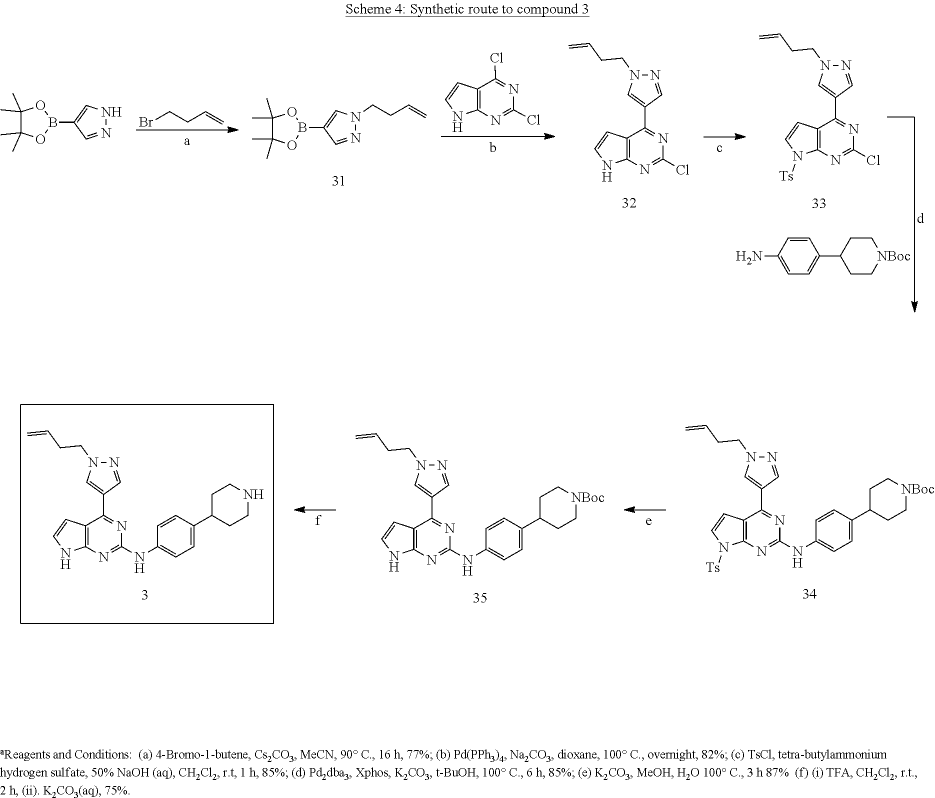

Methyl 3-(4-bromo-1H-pyrazol-1-yl)-3-cyclopentylpropanoate (8): To a solution of 3-cyclopentyl methacrylate (4.56 g, 29.58 mol, 1.0 eq.), in anhydrous acetonitrile (100 mL) added 4-bromopyrazole (4.77 g, 32.49 mol, 1.1 eq), 1,8-diazabicyclo[5.4.0]undec-7-ene (DBU) (6.63 mL, 44.37 mol, 1.5 eq). The reaction mixture was stirred overnight at room temperature. The mixture was concentrated under reduced pressure and the residue was dissolved in ethyl acetate (500 mL), added 1N HCl to adjust pH value to 3 or 4, and then washed with water (100 mL) and brine (100 mL). The organic layer was dried (Na₂SO₄), filtered and concentrated under reduced pressure to obtain the title compound as a colorless oil (7.02 g, 80%). This compound was used in the next step without purification. HPLC 98% (t_{R}=13.1 min, CH₃OH in 0.1% TFA water 5%-95% in 20 min); ¹H NMR (500 MHz, DMSO-d₆) δ 8.04 (s, 1H), 7.51 (s, 1H), 4.39 (m, 1H), 3.50 (s, 1H), 2.97 (m, 2H), 2.24 (m, 1H), 1.74 (m, 1H), 1.59-1.34 (m, 4H), 1.26-1.05 (m, 3H); HPLC-MS (ESI+): m/z 301.1 (M+H)⁺.
3-(4-Bromo-1H-pyrazol-1-yl)-3-cyclopentylpropanoic acid (9): To a solution of 8 (7.02 g, 23.40 mol, 1.0 eq.), in dioxane (140 mL), added sodium hydroxide (2.81 g, 70.20 mol, 3.0 eq) in water (70 mL). The reaction mixture was stirred overnight at room temperature.

The mixture was concentrated under reduced pressure and added water (100 mL) followed by 1N HCl to adjust pH to 3 or 4. The aqueous layer extracted with ethyl acetate (3×150 mL), dried (Na₂SO₄) and filtered, and concentrated under reduced pressure to obtain title compound as a white solid (6.20 g, 95%). This compound was used in the next step without further purification. HPLC 99% (t_{R}=12.3 min, CH₃OH in 0.1% TFA water 5%-95% in 20 min); ¹H NMR (500 MHz, DMSO-d₆) δ 12.21 (s, 1H), 8.02 (s, 1H), 7.51 (s, 1H), 4.37 (m, 1H), 2.86 (m, 2H), 2.23 (m, 1H), 1.75 (m, 1H), 1.60-1.35 (m, 4H), 1.26-1.06 (m, 3H); HPLC-MS (ESI+): m/z 287.2 (M+H)+.

(4R)-4-Benzyl-3-((3R)-3-(4-bromo-1H-pyrazol-1-yl)-3-cyclopentylpropanoyl)oxazolidin-2-one (4R,3R)-10: To a solution of 9 (5.73 g, 20.0 mol, 1.0 eq.) in tetrahydrofuran (172 mL), added pivaloyl chloride (4.93 mL, 40.0 mol, 2.0 eq) and triethylamine (8.34 mL, 60.0 mol, 3.0 eq.) at 0° C. The reaction mixture was stirred for 1 h at room temperature. To the reaction mixture, lithium chloride (1.70 g, 40.0 mol, 2.0 eq) and oxazolidinone (3.58 g, 20.0 mol, 1.0 eq) were added, and then the mixture was stirred overnight at room temperature. The reaction mixture was diluted with water (100 mL), evaporated under reduced pressure to remove tetrahydrofuran and extracted with ethyl acetate (3×150 mL). The organic layer evaporated under reduced pressure and the residue was purified by SiO₂ chromatography using ethyl acetate/hexane (20%) as eluent to give the title compound as a white solid (3.9 g, 42%, TLC: Rf=0.51 developed using ethyl acetate/hexane (2:3)). HPLC 97% (t_{R}=13.40 min, CH₃OH in 0.1% TFA water 5%-95% in 20 min); ¹H NMR (500 MHz, DMSO-d₆) δ 8.10 (d, J=0.7 Hz, 1H), 7.61 (s, 1H), 7.27-7.21 (m, 3H), 6.94-6.91 (m, 2H), 4.63-4.52 (m, 3H), 4.32 (t, J=8.7 Hz, 1H), 4.17 (dd, J=9.0, 3.1 Hz, 1H), 3.79 (dd, J=17.6, 10.6 Hz, 1H), 3.20 (dd, J=17.6, 2.9 Hz, 1H), 2.83-2.74 (m, 2H), 2.37-2.30 (m, 1H), 1.79-1.74 (m, 1H), 1.60-1.50 (m, 3H), 1.47-1.39 (m, 1H), 1.29-1.12 (m, 3H); HPLC-MS (ESI+): m/z 468.2 (M+Na)+.

(4R)-4-Benzyl-3-((3S)-3-(4-bromo-1H-pyrazol-1-yl)-3-cyclopentylpropanoyl)oxazolidin-2-one (4R,3S-10): The (4R,3S)-10 was purified using the procedure described for (4R,3R)-10 to obtain the title product as a white solid (4.0 g, 43%, TLC: Rf=0.62 developed using ethyl acetate/hexane (2:3). HPLC 98% (t_{R}=13.60 min, CH₃OH in 0.1% TFA water 5%-95% in 20 min); ¹H NMR (500 MHz, DMSO-d₆) δ 8.10 (d, J=0.8 Hz, 1H), 7.52 (d, J=0.6 Hz, 1H), 7.34-7.30 (m, 2H), 7.28-7.24 (m, 1H), 7.22-7.19 (m, 2H), 4.61-4.49 (m, 2H), 4.28 (t, J=8.5 Hz, 1H), 4.15 (dd, J=8.8, 2.8 Hz, 1H), 3.53 (dd, J=17.1, 10.4 Hz, 1H), 3.37 (dd, J=17.1, 3.0 Hz, 1H), 2.97 (dd, J=13.6, 3.2 Hz, 1H), 2.84 (dd, J=13.5, 8.3 Hz, 1H), 2.34-3.31 (m, 1H), 1.82-1.73 (m, 1H), 1.64-1.50 (m, 3H), 1.47-1.41 (m, 1H), 1.36-1.12 (m, 1H); HPLC-MS (ESI+): m/z 446.2 (M+H)⁺.

(S)-3-(4-Bromo-1H-pyrazol-1-yl)-3-cyclopentylpropanamide (11): The amide 11 was synthesized using the same procedure described for 17 from (4R,3S)-10 (7.6 mmol, 3.4 g) as a white solid (1.9 g, 87%). HPLC 97.2% (t_{R}=11.4 min, CH₃OH in 0.1% TFA water 5%-95% in 20 min); ¹H NMR (500 MHz, DMSO-d₆) δ 7.92 (s, 1H), 7.51 (s, 1H), 7.30 (s, 1H), 6.76 (s, 1H), 4.41 (td, J=9.7, 4.1 Hz, 1H), 2.76 (dd, J=15.3, 10.0 Hz, 1H), 2.57 (dd, J=15.3, 4.0 Hz, 1H), 2.24 (h, J=8.6 Hz, 1H), 1.79-1.70 (m, 1H), 1.59-1.35 (m, 4H), 1.24-1.06 (m, 3H); HPLC-MS (ESI+): m/z 286.2 (M+H)⁺.

(S)-3-(4-Bromo-1H-pyrazol-1-yl)-3-cyclopentylpropanenitrile (12): The nitrile 12 was synthesized using the same procedure described for 18, from amide 11 (6.6 mmol, 1.9 g) as a white solid (1.6 g, 94%). HPLC 97.2% (t_{R}=12.5 min, CH₃OH in 0.1% TFA water 5%-95% in 20 min); ¹H NMR (500 MHz, DMSO-d₆) δ 8.13 (d, J=0.7 Hz, 1H), 7.63 (s, 1H), 4.38 (td, J=9.4, 4.7 Hz, 1H), 3.15-3.05 (m, 2H), 2.36-2.26 (m, 1H), 1.80-1.69 (m, 2H), 1.62-1.37 (m, 4H), 1.30-1.19 (m, 2H), 1.08 (dq, J=12.7, 8.3 Hz, 1H); HPLC-MS (ESI+): m/z 268.0 (M+H)⁺.

(S)-3-Cyclopentyl-3-(4-(4,4,5,5-tetramethyl-1,3,2-dioxaborolan-2-yl)-1H-pyrazol-1-yl)propanenitrile (13): The boronic ester 13 was synthesized using the same procedure described for 13 from (S)-bromopyrazole 19 (1.1 mmol, 0.3 g), as a white solid (165 mg, 47%). ¹H NMR (500 MHz, DMSO-d₆) δ 8.08 (s, 1H), 7.95 (s, 1H), 4.43 (td, J=9.7, 4.3 Hz, 1H), 3.15-3.09 (m, 2H), 2.37-2.27 (m, 1H), 1.80-1.71 (m, 1H), 1.58-1.37 (m, 4H), 1.25 (s, 12H), 1.20-1.15 (m, 3H).

(S)-3-(4-(7H-Pyrrolo[2,3-d]pyrimidin-4-yl)-1H-pyrazol-1-yl)-3-cyclopentylpropanenitrile [(S)-Ruxolitinib]: (S)-Ruxolitinib was synthesized using the same procedure described for 14 from (S)-13 (0.15 mmol, 49 mg), as a white solid (35 mg, 74%). HPLC 99.17% (t_{R}=12.57 min, CH₃OH 75% in 0.1% TFA water, 20 min); ¹H NMR (500 MHz, DMSO) δ 12.10 (s, 1H), 8.79 (s, 1H), 8.67 (s, 1H), 8.37 (s, 1H), 7.59 (dd, J=3.5, 2.4 Hz, 1H), 6.98 (dd, J=3.6, 1.7 Hz, 1H), 4.54 (td, J=9.8, 4.0 Hz, 1H), 3.26 (dd, J=17.2, 9.8 Hz, 2H), 2.42 (dd, J=17.1, 8.3 Hz, 1H), 1.86-1.78 (m, 1H), 1.65-1.40 (m, 4H), 1.35-1.17 (m, 3H); HPLC-MS (ESI+): m/z 307.2 (M+H)⁺. HRMS (ESI+) m/z calculated for C₁₇H₁₉N₆(M+H)⁺307.1666, found 307.1667.

(3RS)-3-(4-(7H-Pyrrolo[2,3-d]pyrimidin-4-yl)-1H-pyrazol-1-yl)-3-cyclopentylpropanenitrile (rac-Ruxolitinib): To a solution of 4-bromo-7H-pyrrolo[2,3-d]pyrimidine (198 mg, 1.0 mmol, 1.0 equiv.), 3-cyclopentyl-3-(4-(4,4,5,5-tetramethyl-1,3,2-dioxaborolan-2-yl)-1H-pyrazol-1-yl)propanenitrile (378 mg, 1.2 mmol, 1.2 equiv.)[prepared from commercially available 4-(4,4,5,5-Tetramethyl-1,3,2-dioxaborolan-2-yl)-1H-pyrazole and 3-cyclopentylprop-2-enenitrile according to a reported procedure³²], and Na₂CO₃ (340 mg, 3.2 mmol, 3.2 equiv.) in dioxane (4 mL) and H₂O (1 mL) was added Pd(PPh₃)₄(40 mg, 0.033 mmol, 0.033 equiv.). The pressure tube was purged with argon for 15 min, sealed and then placed in pre-heated oil bath 120° C. The mixture was stirred at this temperature for 12 h and then cooled to room temperature and partitioned between saturated NH₄Cl (20 mL) and EtOAc (10 mL). The layers were separated, and the aqueous layer was extracted twice more with EtOAc (10 mL). The combined organic extracts were washed with brine, dried (MgSO₄) and concentrated under reduced pressure to give a yellow oil. Purification by flash chromatography (SiO₂, 75% EtOAc in hexane) provided the title compound rac-Ruxolitinib as a brown powder (0.224 g, 73%). ¹H NMR (500 MHz, DMSO-d₆) δ: 12.11 (s, 1H), 8.80 (d, J=0.7 Hz, 1H), 8.68 (s, 1H), 8.37 (s, 1H), 7.60 (dd, J=3.6, 2.4 Hz, 1H), 6.99 (dd, J=3.6, 1.8 Hz, 1H), 4.54 (td, J=9.8, 4.1 Hz, 1H), 3.20 (m, 3H), 2.43 (m, 1H), 1.82 (m, 1H), 1.61 (m, 3H), 1.46 (m, 1H), 1.39 (m, 2H), 1.18 (m, 1H). HPLC-MS (ESI+): m/z 307.4 [100% (M+H)⁺], HRMS (ESI+) m/z calculated for C₁₇H₁₉N₆(M+H)⁺307.1666, found 307.1662.

(S)-3-(4-(2-Chloro-7H-pyrrolo[2,3-d]pyrimidin-4-yl)-1H-pyrazol-1-yl)-3-cyclopentylpropanenitrile (14): The pyrrolopyrimidine 14 was synthesized using the same procedure described for 20 from (S)-boronic ester 13 (1.1 mmol, 0.3 g), as a white oil (151 mg, 41%). HPLC 78.0% (t_{R}=11.99 min, CH₃OH in 0.1% TFA water 5%-95% in 20 min); ¹H NMR (500 MHz, DMSO-d₆) δ 12.32 (s, 1H), 8.84 (d, J=0.7 Hz, 1H), 8.39 (s, 1H), 7.64 (dd, J=3.6, 2.3 Hz, 1H), 7.05 (dd, J=3.6, 1.8 Hz, 1H), 4.56 (td, J=9.8, 4.1 Hz, 1H), 3.29-3.21 (m, 2H), 2.43 (q, J=8.6 Hz, 1H), 1.82 (td, J=11.9, 7.2 Hz, 1H), 1.66-1.41 (m, 4H), 1.33-1.23 (m, 3H); HPLC-MS (ESI+): m/z 341.2 (M+H)⁺.

tert-Butyl (S)-2-chloro-4-(1-(2-cyano-1-cyclopentylethyl)-1H-pyrazol-4-yl)-7H-pyrrolo[2,3-d]pyrimidine-7-carboxylate (15): The boc-protected pyrrolopyrimidine 15 was synthesized by the same procedure described for 21 from (S)-pyrrolopyrimidine 14 (0.27 mmol, 92 mg) as a white solid (70 mg, 68%). HPLC 95.8% (t_{R}=13.6 min, CH₃OH in 0.1% TFA water 5%-95% in 20 min); ¹H NMR (500 MHz, DMSO-d₆) δ 8.91 (d, J=0.7 Hz, 1H), 8.44 (s, 1H), 7.92 (d, J=4.1 Hz, 1H), 7.27 (d, J=4.2 Hz, 1H), 4.56 (td, J=9.7, 4.1 Hz, 1H), 3.29-3.21 (m, 2H), 2.43 (q, J=8.6 Hz, 1H), 1.82 (td, J=12.2, 7.4 Hz, 1H), 1.64 (s, 8H), 1.58-1.41 (m, 4H), 1.35-1.23 (m, 3H); HPLC-MS (ESI+): m/z 441.2 (M+H)⁺.

tert-Butyl (S)-2-((4-(1-(tert-butoxycarbonyl)piperidin-4-yl)phenyl)amino)-4-(1-(2-cyano-1-cyclopentylethyl)-1H-pyrazol-4-yl)-7H-pyrrolo[2,3-d]pyrimidine-7-carboxylate (16): The bis-boc-protected pyrrolopyrimidine 16 was synthesized using the same procedure described for 22, from the (S)-pyrrolopyrimidine derivative 15 (0.09 mmol, 60 mg) as a yellow solid (47 mg, 76%). HPLC 97.0% (t_{R}=15.04 min, CH₃OH in 0.1% TFA water 5%-95% in 20 min); ¹H NMR (500 MHz, DMSO-d₆) δ 10.85 (s, 1H), 9.43 (s, 2H), 8.75 (d, J=0.8 Hz, 1H), 8.33 (s, 1H), 7.92 (d, J=8.6 Hz, 2H), 7.56 (d, J=4.1 Hz, 1H), 7.15 (d, J=8.5 Hz, 2H), 7.04 (d, J=4.2 Hz, 1H), 4.56 (td, J=9.6, 4.4 Hz, 2H), 3.26-3.21 (m, 2H), 2.65-2.62 (m, 1H), 2.45-2.40 (m, 1H), 1.85-1.80 (m, 1H), 1.76 (d, J=13.1 Hz, 2zH), 1.66-1.52 (m, 15H), 1.43 (s, 9H), 1.36-1.26 (m, 5H); HPLC-MS (ESI+): m/z 681.4 (M+H)⁺.

(S)-3-Cyclopentyl-3-(4-(2-((4-(piperidin-4-yl)phenyl)amino)-7H-pyrrolo[2,3-d]pyrimidin-4-yl)-1H-pyrazol-1-yl)propanenitrile [(S)-1]: The anilinopiperidine (S)-1 was synthesized using the same procedure described for (R)-1 from (S)-pyrrolopyrimidine 16 (0.06 mmol, 42 mg) as a yellow solid (24 mg, 83%). HPLC 97.6% (t_{R}=7.76 min, CH₃OH 50% and water 50% in 20 min); ¹H NMR (500 MHz, DMSO-d₆) δ 11.49 (s, 1H), 9.04 (s, 1H), 8.69 (s, 1H), 8.30 (s, 1H), 7.82-7.78 (m, 2H), 7.22 (dd, J=3.7, 2.1 Hz, 1H), 7.15-7.11 (m, 2H), 6.79 (dd, J=3.6, 1.6 Hz, 1H), 4.57 (td, J=9.7, 4.2 Hz, 1H), 3.26-3.20 (m, 2H), 3.08 (d, J=11.9 Hz, 2H), 2.68-2.61 (m, 2H), 1.87-1.80 (m, 1H), 1.73 (d, J=12.6 Hz, 2H), 1.65-1.42 (m, 7H), 1.38-1.20 (m, 6H); HPLC-MS (ESI+): m/z 481.5 (M+H)⁺. HRMS (ESI+) m/z calculated for C₂₈H₃₃N₈(M+H)⁺481.2823, found 481.2830.

(R)-3-(4-Bromo-1H-pyrazol-1-yl)-3-cyclopentylpropanamide (17): To a solution of (4R,3R)-10 (3.0 g, 6.7 mol, 1.0 eq.), in tetrahydrofuran (180 mL) was added ammonium hydroxide (30%, 120 mL). The reaction mixture was stirred for 2 days at room temperature. The mixture was diluted with methanol (250 mL), concentrated under reduced pressure and this process was repeated another 2 times. The residue obtained was purified by reverse phase chromatography over C-18 silica gel using methanol/dichloromethane (gradient elution 0 to 5%) as eluent to give the title compound as a white solid (1.24 g, 64%). HPLC 95% (tR=11.44 min, CH₃OH in 0.1% TFA water 5%-95% in 20 min); ¹H NMR (500 MHz, DMSO-d₆) δ 7.92 (d, J=0.7 Hz, 1H), 7.51 (d, J=0.7 Hz, 1H), 7.29 (s, 1H), 6.75 (s, 1H), 4.41 (td, J=9.7, 4.0 Hz, 1H), 2.79-2.57 (m, 1H), 2.27-2.20 (m, 1H), 1.78-1.72 (m, 1H), 1.60-1.36 (m, 4H), 1.24-1.04 (m, 3H); HPLC-MS (ESI+): m/z 286.1 (M+H)⁺.

(R)-3-(4-Bromo-1H-pyrazol-1-yl)-3-cyclopentylpropanenitrile (18): To a solution of 17 (1.24 g, 4.3 mol, 1.0 eq.), in tetrahydrofuran (50 mL), added phosphorus pentoxide (1.84 g, 13.0 mol, 3 eq.) under argon. The reaction mixture was stirred for 2 hours at 70° C., diluted with ethyl acetate (200 mL), and quenched by adding saturated sodium bicarbonate (200 mL). The aqueous layer was back-extracted with ethyl acetate (2×100 mL). The organic phases were combined, washed with water, brine, dried over anhydrous Na₂SO₄ and filtered. The filtrate was concentrated under reduced pressure and purified by SiO₂ chromatography using methanol/dichloromethane (5%) as eluent to provide the title compound as a white solid (1.13 g, 98%). HPLC 100% (t_{R}=12.56 min, CH₃OH in 0.1% TFA water 5%-95% in 20 min); ¹H NMR (500 MHz, DMSO-d₆) δ 8.12 (d, J=0.5 Hz, 1H), 7.63 (d, J=0.5 Hz, 1H), 4.38 (td, J=9.4, 4.7 Hz, 1H), 3.15-3.08 (m, 2H), 2.33-2.28 (m, 1H), 1.79-1.72 (m, 1H), 1.61-1.38 (m, 4H), 1.30-1.05 (m, 3H); HPLC-MS (ESI+): m/z 268.0 (M+H)⁺.

(R)-3-Cyclopentyl-3-(4-(4,4,5,5-tetramethyl-1,3,2-dioxaborolan-2-yl)-1H-pyrazol-1-yl)propanenitrile (19): To a 20 mL microwave vial, added 18 (0.30 g, 1.12 mmol, 1.0 eq.), 4,4,5,5-tetramethyl-2-(4,4,5,5-tetramethyl-1,3,2-dioxaborolan-2-yl)-1,3,2-dioxaborolane (0.30 g, 1.19 mmol, 1.1 eq.), potassium acetate (329 mg, 3.36 mmol, 3.0 eq.), and 1,4-dioxane (4.0 mL). The resulting reaction mixture was degassed by bubbling argon for 5 minutes before being treated with tetrakis(triphenylphosphine)palladium(0) (65 mg, 0.06 mmol, 0.05 eq.). The resulting reaction mixture was heated to 120° C. in a microwave reactor for 2 hours. The reaction mixture was filtered through a celite bed and the celite bed was washed with dichloromethane and the organic layer was diluted with water (10 mL). The aqueous layer was extracted with dichloromethane (2×10 mL). The combined organic layers were concentrated under reduced pressure, and the crude product was purified using SiO₂ chromatography (ethyl acetate 0-30% in hexanes) to yield the title compound as a yellow oil (153 mg, 43%). ¹H NMR (500 MHz, DMSO-d₆) δ 8.09 (d, J=0.7 Hz, 1H), 7.66 (d, J=0.7 Hz, 1H), 4.43 (td, J=9.6, 4.3 Hz, 1H), 3.19-3.05 (m, 2H), 2.33-2.31 (mz, 1H), 1.80-1.73 (m, 1H), 1.61-1.39 (m, 4H), 1.27 (s, 12H), 1.20-1.15 (m, 3H).

(R)-3-(4-(2-Chloro-7H-pyrrolo[2,3-d]pyrimidin-4-yl)-1H-pyrazol-1-yl)-3-cyclopentylpropanenitrile (20): To a 50 mL round bottom flask added 2,4-dichloro-7H-pyrrolo[2,3-d]pyrimidine (87.0 mg, 0.46 mmol, 0.95 eq.), 19 (153.0 mg, 0.49 mmol, 1 eq.), sodium carbonate (191 mg, 1.38 mmol, 3 eq.), water (0.5 mL), 1,4-dioxane (2.0 mL), and the resulting reaction mixture was degassed by bubbling argon for 5 minutes before adding Pd(PPh₃)₄(17 mg, 0.01 mmol, 0.03 eq.). The resulting reaction mixture was heated to 100° C. under argon overnight. The reaction mixture was gradually cooled down to ambient temperature and filtered through a celite bed. The celite bed was washed with dichloromethane (10 mL) and the organic layer was diluted with water (10 mL). The aqueous layer was extracted with dichloromethane (2×10 mL). The combined organic layers were concentrated under reduced pressure to remove solvents, and the crude product was purified by SiO₂ chromatography (ethyl acetate 0-60% in hexanes) to yield the title compound as a yellow solid (94 mg, 68%). HPLC 74.7% (tR=11.93 min, CH₃OH in 0.1% TFA water 5%-95% in 20 min); ¹H NMR (600 MHz, DMSO-d₆) δ 12.32 (s, 1H), 8.84 (d, J=0.8 Hz, 1H), 8.39 (s, 1H), 7.64 (d, J=3.6, 1H), 7.05 (d, J=3.6, 1H), 4.56 (td, J=9.8, 4.0 Hz, 1H), 3.27-3.18 (m, 2H), 2.45-2.38 (m, 1H), 1.86-1.78 (m, 1H), 1.64-1.44 (m, 4H), 1.35-1.24 (m, 3H); HPLC-MS (ESI+): m/z 341.2 (M+H)⁺.

tert-Butyl (R)-2-chloro-4-(1-(2-cyano-1-cyclopentylethyl)-1H-pyrazol-4-yl)-7H-pyrrolo[2,3-d]pyrimidine-7-carboxylate (21): To a solution of pyrrolopyrimidine 20 (94.0 mg, 0.28 mmol, 1.0 eq.), in dichloromethane (2.0 mL), added N,N-diisopropylethylamine (0.058 mL, 0.33 mmol, 1.2 eq) and then added di-tert-butyl dicarbonate (90.3 mg, 0.41 mmol, 1.5 eq) and 4-dimethylaminopyridine (7.0 mg, 0.06 mmol, 0.2 eq). The reaction mixture was stirred for 2 hours at room temperature. The reaction mixture was diluted with water (10 mL) and extracted with dichloromethane (3×10 mL). The organic phase was evaporated under reduced pressure and the residue was purified by SiO₂ chromatography using ethyl acetate/hexane (50%) as eluent to give the title compound as a white solid (98 mg, 80%). HPLC 95.2% (t_{R}=13.63 min, CH₃OH in 0.1% TFA water 5%-95% in 20 min); ¹H NMR (600 MHz, DMSO-d₆) δ 8.90 (d, J=0.8 Hz, 1H), 8.43 (s, 1H), 7.91 (d, J=4.1 Hz, 1H), 7.26 (d, J=4.2 Hz, 1H), 4.55 (td, J=9.8, 4.0 Hz, 1H), 3.24-3.16 (m, 2H), 1.85-1.78 (m, 1H), 1.58-1.40 (m, 4H), 1.35-1.22 (m, 12H); HPLC-MS (ESI+): m/z 441.2 (M+H)⁺.

tert-Butyl (R)-2-((4-(1-(tert-butoxycarbonyl)piperidin-4-yl)phenyl)amino)-4-(1-(2-cyano-1-cyclopentylethyl)-1H-pyrazol-4-yl)-7H-pyrrolo[2,3-d]pyrimidine-7-carboxylate (22): A mixture of the boc-pyrrolopyrimidine 21 (47.0 mg, 0.11 mmol, 1.0 eq.), tert-butyl 4-(4-aminophenyl)piperidine-1-carboxylate (30.0 mg, 0.11 mmol, 1.0 eq.) and potassium carbonate (30.0 mg, 2 mmol, 2.0 eq.) in anhydrous tert-butyl alcohol (2 mL) was degassed by bubbling argon. To this mixture, added 2-dicyclohexylphosphino-2′,4′,6′-triisopropylbiphenyl (10.0 mg, 0.021 mmol, 0.2 eq.) and tris(dibenzylideneacetone)dipalladium (10.0 mg, 0.01 mmol, 0.1 eq.). The reaction was refluxed overnight, then allowed to cool to room temperature, water was added (10 mL) and the mixture was extracted with dichloromethane (3×10 mL). The combined organic extracts were washed with brine, dried (Na₂SO₄), filtered and the solvent was removed under reduced pressure. The residue was purified by SiO₂ chromatography using ethyl acetate/hexane (50%) as eluent to give the title compound as a yellow solid (54 mg, 89%).

HPLC 97.1% (t_{R}=15.13 min, CH₃OH in 0.1% TFA water 5%-95% in 20 min); ¹H NMR (500 MHz, DMSO-d₆) δ 9.43 (s, 1H), 8.75 (d, J=0.7 Hz, 1H), 8.33 (s, 1H), 7.93-7.90 (m, 2H), 7.56 (d, J=4.2 Hz, 1H), 7.15 (d, J=8.6 Hz, 2H), 7.04 (d, J=4.2 Hz, 1H), 4.56 (td, J=9.6, 4.3 Hz, 1H), 3.27-3.20 (m, 2H), 2.46-2.43 (m, 1H), 1.87-1.79 (m, 1H), 1.76 (d, J=13.0 Hz, 2H), 1.66 (s, 9H), 1.57-1.45 (m, 6H), 1.43 (s, 9H), 1.37-1.16 (m, 8H); HPLC-MS (ESI+): m/z 681.5 (M+H)⁺.

(R)-3-Cyclopentyl-3-(4-(2-((4-(piperidin-4-yl)phenyl)amino)-7H-pyrrolo[2,3-d]pyrimidin-4-yl)-1H-pyrazol-1-yl)propanenitrile [(R)-1)]: The bis-boc-protected derivative 16 (54.0 mg, 0.08 mmol, 1.0 eq.) was suspended in dichloromethane (2 mL) and trifluoroacetic acid (0.12 mL, 1.59 mmol, 20.0 eq.) was added. The mixture was stirred at room temperature for 2 hours and concentrated under reduced pressure. The residue was dissolved in chloroform and washed with 10% potassium carbonate (aq.). The aqueous layer was extracted with chloroform (3×10 mL) and combined organic phase was dried (Na₂SO₄) and filtered. The filtrate was concentrated under reduced pressure and the product was dried under vacuum to afford the title compound as a white solid (47 mg, 95%). HPLC 98.7% (t_{R}=11.00 min, CH₃OH in 0.1% TFA water 5%-95% in 20 min); ¹H NMR (500 MHz, DMSO-d₆) δ 11.49 (s, 1H), 9.01 (s, 1H), 8.68 (s, 1H), 8.30 (s, 1H), 7.80-7.76 (m, 2H), 7.21 (d, J=3.6 Hz, 1H), 7.14-7.10 (m, 2H), 6.78 (d, J=3.6 Hz, 1H), 4.56 (td, J=9.6, 4.1 Hz, 1H), 3.22-3.17 (m, 2H), 3.02 (d, J=11.9 Hz, 1H), 2.44-2.42 (m, 1H), 1.81-1.85 (m, 1H), 1.68 (d, J=13.0 Hz, 2H), 1.61-1.44 (m, 6H), 1.36-1.22 (m, 8H); HPLC-MS (ESI+): m/z 681.5 (M+H)⁺. HRMS (ESI+) m/z calculated for C₂₈H₃₃N₈(M+H)⁺481.2823, found 481.2830.

3-(4-(4,4,5,5-Tetramethyl-1,3,2-dioxaborolan-2-yl)-1H-pyrazol-1-yl)propanenitrile (27): A 100 mL round bottom flask was charged with 4-(4,4,5,5-tetramethyl-1,3,2-dioxaborolan-2-yl)-1H-pyrazole (5.0 g, 25.8 mmol, 1.0 equiv.), acrylonitrile (1.04 g, 52.0 mmol, 2.0 equiv.), DBU (1.97 g, 12.9 mmol, 0.5 equiv.), and acetonitrile (50 mL). The mixture was stirred at room temperature overnight. After cooling, the mixture was diluted with ethyl acetate, washed with water twice and brine once, and dried over Na₂SO₄. After filtration the filtrate was concentrated under reduced pressure to yield the nitrile 27 as yellow oil (3.9 g, 62%). ¹H NMR (500 MHz, DMSO-d₆) δ 8.04 (d, J=0.6 Hz, 1H), 7.65 (d, J=0.7 Hz, 1H), 4.41 (t, J=6.4 Hz, 2H), 3.07 (t, J=6.4 Hz, 2H), 1.26 (s, 9H).

3-(4-(2-Chloro-7H-pyrrolo[2,3-d]pyrimidin-4-yl)-1H-pyrazol-1-yl)propanenitrile (28): To a 50 mL round bottom flask was added 2,4-dichloro-7H-pyrrolo[2,3-d]pyrimidine (3.0 g, 12.0 mmol, 1.2 equiv.), 3-(4-(4,4,5,5-tetramethyl-1,3,2-dioxaborolan-2-yl)-1H-pyrazol-1-yl)propanenitrile 27 (0.51 g, 10.0 mmol, 1.0 equiv.), sodium carbonate (3.4 g, 31.6 mmol, 3.16 equiv.), water (H₂O, 13 mL) and 1,4-dioxane (53 mL), the resulting reaction mixture was degassed by bubbling argon for 5 min before being treated with Pd(PPh₃)₄(384 mg, 0.333 mmol). The resulting reaction mixture was heated to 100° C. under argon overnight. The reaction mixture was gradually cooled to ambient temperature before being filtered through a Celite bed. The Celite bed was washed with dichloromethane before the filtrate and washes were combined. The two layers were separated, and the aqueous layer was extracted with dichloromethane. The combined organic layers were concentrated under reduced pressure to remove solvents, and the residue was purified by column chromatography (EtOAc 0 to 50% in hexanes) to yield the title compound 28 as a brown solid (1.8 g, 69%). ¹H NMR (500 MHz, DMSO-d₆) δ:12.33 (s, 1H), 8.81 (d, J=0.7 Hz, 1H), 8.37 (d, J=0.7 Hz, 1H), 7.64 (dd, J=3.6, 2.3 Hz, 1H), 7.03 (dd, J=3.6, 1.7 Hz, 1H), 4.53 (t, J=6.4 Hz, 2H), 3.19 (t, J=6.4 Hz, 2H). HPLC-MS (ESI+): m/z 273.4 (M+1)⁺, 567.3 (2M+Na)⁺.

tert-Butyl-2-chloro-4-(1-(2-cyanoethyl)-1H-pyrazol-4-yl)-7H-pyrrolo[2,3-d]pyrimidine-7-carboxylate (29): To a solution of the chloropyrrolopyrimidine 28 (850 mg, 3.12 mmol, 1.2 equiv.) in DCM (15 mL) was added (Boc)₂O (1.0 g, 4.68 mmol, 1.5 equiv.), DIPEA (0.49 g, 3.75 mmol, 1.2 equiv.) and DMAP (76 mg, 0.624 mmol, 0.2 equiv.). The reaction mixture was stirred at room temperature for 12 h, quenched with water and extracted with EtOAc (2×20 mL). The organic layer was dried (Na₂SO₄) and concentrated in vacuo to yield the title compound 29 as a brown solid (1.12 g, 96%). ¹H NMR (500 MHz, DMSO-d₆) δ: 8.87 (d, J=0.9 Hz, 1H), 8.40 (d, J=0.7 Hz, 1H), 7.91 (d, J=4.1 Hz, 1H), 7.25 (d, J=4.2 Hz, 1H), 4.53 (t, J=6.4 Hz, 2H), 3.19 (t, J=6.4 Hz, 2H), 1.64 (s, 9H). HPLC-MS (ESI+): m/z 373.1 [60%, (M+H)⁺], 769.2 [100% (2M+Na)⁺].

tert-Butyl-2-((4-(1-(tert-butoxycarbonyl)piperidin-4-yl)phenyl)amino)-4-(1-(2-cyanoethyl)-1H-pyrazol-4-yl)-7H-pyrrolo[2,3-d]pyrimidine-7-carboxylate (30): A mixture of tert-butyl-2-chloro-4-(1-(2-cyanoethyl)-1H-pyrazol-4-yl)-7H-pyrrolo[2,3-d]pyrimidine-7-carboxylate (29) (0.37 g, 1.0 mmol, 1.2 equiv.), tert-butyl-4-(4-aminophenyl)piperidine-1-carboxylate (0.33 g, 1.2 mmol, 1.2 equiv.) and K₂CO₃ (0.21 g, 1.5 mmol) in anhydrous ^{t}BuOH (10 mL) was degassed under argon. To this mixture was added XPhos (48 mg, 0.1 mmol) and Pd₂(dba)₃ (46 mg, 0.05) and the reaction was heated under for 6 h, then allowed to cool. To this mixture was added water (40 mL) and the mixture was extracted with CH₂Cl₂. The combined organic extracts were washed with brine, dried, filtered and the solvent removed under reduced pressure. The residue was purified by flash chromatography (50% EtOAc/hexanes) to yield the title compound 30 as a brown solid (0.40 g, 79%). ¹H NMR (500 MHz, DMSO-d₆) δ 9.43 (s, 1H), 8.72 (d, J=1.6 Hz, 1H), 8.31 (d, J=1.5 Hz, 1H), 7.90 (d, J=8.6 Hz, 1H), 7.55 (d, J=4.2 Hz, 1H), 7.14 (d, J=8.7 Hz, 2H), 7.01 (d, J=4.2 Hz, 1H), 4.53 (m, 2H), 4.02 (m, 2H), 3.16 (m, 2H), 2.80 (m, 2H), 2.6 (m, 1H), 1.75 (m, 2H), 1.65 (s, 9H), 1.47 (m, 2H), 1.42 (s, 9H). HPLC-MS (ESI+): m/z 613.1 [100%, (M+H)⁺].

3-(4-(2-((4-(Piperidin-4-yl)phenyl)amino)-7H-pyrrolo[2,3-d]pyrimidin-4-yl)-1H-pyrazol-1-yl)propanenitrile (2): To a solution of the bis-boc-protected pyrrolopyrimidine 30 (0.306 g, 0.5 mmol) in DCM (3 mL) was added TFA (3 mL) under argon atmosphere and the reaction mixture was stirred at room temperature for 4 h. After this time there was no starting material present (as measured by HPLC), and the reaction was quenched with sat. NaHCO₃ solution and extracted with DCM (2×10 mL). The combined organic layers were dried over (Na₂SO₄) and evaporated to dryness in vacuo to yield the title compound 2 as a brown solid (0.121 g, 81%). ¹H NMR (500 MHz, DMSO-d₆) δ: 11.49 (s, 1H), 9.06 (s, 1H), 8.67 (s, 1H), 8.29 (s, 1H), 7.80 (d, J=8.3 Hz, 2H), 7.22 (d, J=3.6 Hz, 1H), 7.13 (d, J=8.3 Hz, 2H), 6.77 (d, J=3.6 Hz, 1H), 4.53 (t, J=6.4 Hz, 2H), 3.13 (m, 4H), 2.69 (m, 2H), 2.60 (m, 1H), 1.77 (dd, J=13.6, 3.4 Hz, 2H), 1.58 (m, 2H). HPLC-MS (ESI+): m/z 207.2 [100% (½M+H)²+], 413.3 [40% (M+H)⁺]. m/z calculated for C₂₃H₂₅N₈(M+H)⁺413.2197, found 413.2191.

1-(But-3-en-1-yl)-4-(4,4,5,5-tetramethyl-1,3,2-dioxaborolan-2-yl)-1H-pyrazole (31): A 100 mL round bottom flask was charged with 4-(4,4,5,5-tetramethyl-1,3,2-dioxaborolan-2-yl)-1H-pyrazole (0.5 g, 2.58 mmol, 1.0 eq.), 4-bromobutene (0.48 g, 3.6 mmol, 1.4 eq.), cesium carbonate (1.67 g, 5.16 mmol, 2 eq.), and acetonitrile (10 mL). The reaction mixture was stirred at 90° C. overnight. After cooling, the mixture was quenched with water (30 mL) and extracted with ethyl acetate (3×30 mL). The organic layers were combined, washed with brine (50 mL), dried (Na₂SO₄) and filtered. The solvent was evaporated under reduced pressure to yield the title product as yellow oil. ¹H NMR (500 MHz, chloroform-d) δ 7.78 (s, 1H), 7.67 (s, 1H), 5.78 (m, 1H), 5.09 (m, 2H), 4.20 (t, J=7.5 Hz, 2H), 2.63 (m, 2H), 1.31 (s, 12H).

4-(1-(But-3-en-1-yl)-1H-pyrazol-4-yl)-2-chloro-7H-pyrrolo[2,3-d]pyrimidine (32): A 50 mL round bottom flask was charged with 2,4-dichloro-7H-pyrrolo[2,3-d]pyrimidine (0.3 g, 1.6 mmol, 1.0 eq.), 1-(but-3-en-1-yl)-4-(4,4,5,5-tetramethyl-1,3,2-dioxaborolan-2-yl)-1H-pyrazole 31 (0.51 g, 2.08 mmol, 1.5 eq.), sodium carbonate (0.33 g, 3.2 mmol, 2.0 eq.), water (H₂O, 5 mL) and 1,4-dioxane (15 mL). The reaction mixture was degassed by bubbling argon for 5 min before treating with Pd(PPh₃)₄(199 mg, 0.172 mmol, 0.107 eq.). The mixture was heated to 100° C. under argon overnight. The reaction mixture was gradually cooled down to ambient temperature, quenched with water (20 mL) and the mixture was extracted with ethyl acetate (3×30 mL). The organic layers were combined, washed with brine (50 mL), dried (Na₂SO₄) and filtered. The solvent was evaporated under reduced pressure and the residue was triturated with Et₂O and hexanes to give 32 as a yellow solid (0.32 g, 82%). ¹H NMR (500 MHz, DMSO-d₆) δ 12.28 (s, 1H), 8.69 (s, 1H), 8.28 (s, 1H), 7.60 (dd, J=3.0, 1.5 Hz, 1H), 7.02 (d, J=3.0 Hz, 1H), 5.84 (m, 1H), 5.08 (m, 2H), 4.31 (t, J=7.5 Hz, 2H), 2.64 (m, 2H). HPLC-MS (ESI+): m/z 274.2 (M+1)⁺, 569.2 (2M+Na)⁺.

4-(1-(But-3-en-1-yl)-1H-pyrazol-4-yl)-2-chloro-7-tosyl-7H-pyrrolo[2,3-d]pyrimidine (33): To a solution of 4-(1-(but-3-en-1-yl)-1H-pyrazol-4-yl)-2-chloro-7H-pyrrolo[2,3-d]pyrimidine 32 (1.0 g, 5.32 mmol. 1.0 eq.), p-toluenesulfonyl chloride (1.1 g, 5.85 mmol, 1.1 eq.) and tetra-butylammonium hydrogen sulfate (0.090 g, 0.27 mmol, 0.05 eq.) in dichloromethane (20 mL) was added NaOH (50% aq, 0.2 mL). The reaction mixture was stirred at room temperature for 30 minutes. After completion of the reaction, (as indicated by TLC) the reaction mixture was diluted with H₂O (20 mL). The organic layer was separated, washed with brine (50 mL), dried (Na₂SO₄) and filtered. The organic layer was evaporated under reduced pressure to obtain a light yellow solid, which was purified by SiO₂ chromatography using hexanes/ethyl acetate (5:1) as eluent to give the title product (1.3 g, 85%). ¹H NMR (500 MHz, DMSO-d₆) δ 8.75 (s, 1H), 8.29 (s, 1H), 8.04 (d, J=4.5 Hz, 1H), 8.03 (d, J=8.5 Hz, 2H), 7.50 (d, J=8.5 Hz, 2H), 7.37 (d, J=4.5 Hz, 1H), 5.81 (m, 1H), 5.05 (m, 2H), 4.29 (t, J=7.5 Hz, 2H), 2.61 (m, 2H). 2.37 (s, 3H). HPLC-MS (ESI+): m/z 428.1 (M+1)⁺, 877.1 (2M+Na)⁺.

tert-Butyl 4-(4-((4-(1-(But-3-en-1-yl)-1H-pyrazol-4-yl)-7-tosyl-7H-pyrrolo[2,3-d]pyrimidin-2-yl)amino)phenyl)piperidine-1-carboxylate (34): A mixture of 4-(1-(but-3-en-1-yl)-1H-pyrazol-4-yl)-2-chloro-7-tosyl-7H-pyrrolo[2,3-d]pyrimidine 33 (300 mg, 0.7 mmol, 1.0 eq.), tert-butyl 4-(4-aminophenyl)piperidine-1-carboxylate (200 mg, 0.74 mmol, 1.06 eq.) and K₂CO₃ (200 mg, 1.4 mmol, 2.0 eq.) in anhydrous ^{t}BuOH (15 mL) was degassed by bubbling argon for 5 min. XPhos (33 mg, 0.07 mmol, 0.1 eq.) and Pd₂(dba)₃ (32 mg, 0.035, 0.05 eq.) were added and the mixture was refluxed overnight. The mixture was allowed to cool to room temperature and quenched with water (40 mL). The mixture was extracted with CH₂Cl₂ (3×30 mL). The combined organic extracts were washed with brine, dried (Na₂SO₄), filtered and the solvent was removed under reduced pressure. The residue was purified by SiO₂ chromatography (0 to 60% EtOAc in Hexanes) to afford the title compound as yellow solid (330 mg, 85%). ¹H NMR (500 MHz, DMSO-d₆) δ 9.52 (s, 1H), 8.60 (s, 1H), 8.19 (s, 1H), 8.02 (d, J=8.0 Hz, 2H), 7.87 (d, J=8.0 Hz, 2H), 7.70 (d, J=4.0 Hz, 1H), 7.40 (d, J=8.0 Hz, 2H), 7.24 (d, J=8.0 Hz, 2H), 5.79 (m, 1H), 5.07 (m, 2H), 4.30 (t, J=7.5 Hz, 2H), 4.09 (br s, 2H), 2.82 (br s, 2H), 2.69-2.58 (m, 4H), 2.32 (s, 3H), 1.80 (m, 2H), 1.53 (m, 2H), 1.48 (s, 9H). HPLC-MS (ESI+): m/z 668.3 (M+1)⁺.

tert-Butyl 4-(4-((4-(1-(but-3-en-1-yl)-1H-pyrazol-4-yl)-7H-pyrrolo[2,3-d]pyrimidin-2-yl)amino)phenyl)piperidine-1-carboxylate (35): To a solution of tert-butyl 4-(4-((4-(1-(but-3-en-1-yl)-1H-pyrazol-4-yl)-7-tosyl-7H-pyrrolo[2,3-d]pyrimidin-2-yl)amino)phenyl)piperidine-1-carboxylate 34 (165 mg, 0.29 mmol, 1.0 eq.) in MeOH (5 mL) and H₂O (3 mL) was added K₂CO₃ (165 mg, 1.20 mmol, 4.0 eq.) and the mixture was refluxed for 3 h. The mixture was diluted with H₂O (30 mL) and extracted with EtOAc (3×30 mL). The combined organic extracts were washed with brine, dried (Na₂SO₄), filtered and the solvent was removed under reduced pressure to give a yellow residue. The residue was purified by SiO₂ flash chromatography (0 to 70% EtOAc in hexanes) to give the title compound (105 mg, 87%) as a white solid. ¹H NMR (500 MHz, DMSO-d₆) δ 11.47 (s, 1H), 9.03 (s, 1H), 8.55 (s, 1H), 8.20 (s, 1H), 7.81 (d, J=9.0 Hz, 2H), 7.19 (m, 1H), 7.14 (d, J=9.0 Hz, 2H), 6.76 (m, 1H), 5.86 (m, 1H), 5.10 (m, 2H), 4.32 (t, J=7.0 Hz, 2H), 4.10 (br s, 2H), 2.79 (br s, 2H), 2.65-2.58 (m, 3H), 1.76 (m, 2H), 1.49 (m, 2H), 1.42 (s, 9H). HPLC-MS (ESI+): m/z 514.4 (M+1)⁺.

4-(1-(But-3-en-1-yl)-1H-pyrazol-4-yl)-N-(4-(piperidin-4-yl)phenyl)-7H-pyrrolo[2,3-d]pyrimidin-2-amine (3): tert-Butyl-4-(4-((4-(1-(but-3-en-1-yl)-1H-pyrazol-4-yl)-7H-pyrrolo[2,3-d]pyrimidin-2-yl)amino)phenyl)piperidine-1-carboxylate 35 (0.22 g, 0.43 mmol, 1.0 eq.) was suspended in CH₂Cl₂ (2 mL) and TFA (0.65 mL, 8.5 mmol, 20 eq.) was added. The mixture was stirred at r.t. for 2 h and concentrated under reduced pressure. The residue was dissolved in CHCl₃ (20 mL) and washed with 10% K₂CO₃ (aq). The aqueous layer was extracted with CHCl₃ (3×20 mL). The organic layers were combined, dried (Na₂SO₄) and filtered. The filtrate was concentered under reduced pressure and the residue was dried under vacuum to afford the titled compound as an off-white solid (0.13 g, 75%). HPLC 99.8% (t_{R}=8.90 min, CH₃OH 45% in H₂O (0.1% TFA), 1 m/min, 20 min); ¹H NMR (500 MHz, DMSO-d₆) δ 11.47 (s, 1H), 9.00 (s, 1H), 8.55 (s, 1H), 8.20 (s, 1H), 7.79 (d, J=7.5 Hz, 2H), 7.19 (d, J=4 Hz, 1H), 7.12 (d, J=7.5 Hz, 2H), 6.76 (d, J=4 Hz, 1H), 5.86 (m, 1H), 5.10 (m, 2H), 4.32 (t, J=7.0 Hz, 2H), 3.02 (br d, J=12.0 Hz, 2H), 2.65 (q, J=7 Hz, 2H), 2.59 (t, J=12.5 Hz, 2H), 1.68 (d, J=12.0 Hz, 2H), 1.53 (m, 2H); ¹³C NMR (125 MHz, DMSO-d₆) δ 156.4, 154.2, 151.3, 140.0, 139.3, 139.1, 135.3, 131.3, 126.9, 123.6, 121.2, 118.7, 117.7, 107.6, 100.5, 51.3, 47.2, 42.4, 35.0, 34.3; HPLC-MS (ESI+): m/z 414.3 (M+1)⁺; HRMS (ESI+) m/z calculated for C₂₄H₂₈N₇(M+H)⁺414.2401, found 414.2402.

### Dianilinopyrimidine Inhibitors of JAK2

N-(2-Fluoro-5-((2-((3-fluoro-4-(1-methylpiperidin-4-yl)phenyl)amino)-5-methylpyrimidin-4-yl)amino)phenyl)-2-methylpropane-2-sulfonamide (4). This was prepared according the method reported in WO2020/051572.

N-(5-((2-((3,5-Difluoro-4-(1-methylpiperidin-4-yl)phenyl)amino)-5-methylpyrimidin-4-yl)amino)-2-fluorophenyl)-2-methylpropane-2-sulfonamide (5). This was prepared according the method reported in WO2020/051572.

4-((4-((4-Chloro-3-((1,1-dimethylethyl)sulfonamido)phenyl)amino)-5-methylpyrimidin-2-yl)amino)-2-fluoro-N-(1-methylpiperidin-4-yl)benzamide (6). This was prepared according the method reported in U.S. Pat. No. 10,106,507.

N⁴-(1-(tert-Butylsulfonyl)indolin-6-yl)-N²-(3-fluoro-4-(1-methylpiperidin-4-yl)phenyl)-5-methylpyrimidine-2,4-diamine (7). This was prepared according the method reported in WO2020/051572.

Cloning and expression of JAK2 kinase domain: The DNA of human JAK2 KD (JH1), encoding amino acids 840-1132, was synthesized and cloned into pcDNA 3.3 (GeneArt). The construct was expressed using a CMV promoter and contained a Hiss N-terminal affinity tag followed by a TEV cleavage site. Expi293F cells (Invitrogen) were grown, maintained, and treated in shaking culture at 37° C. with 8% CO₂ in Expi293 medium (Invitrogen). Recombinant JAK2 was expressed in Expi293F cells using Transporter 5 transfection reagent as described by the manufacturer (PolySciences). One hour after adding the plasmid:transfection complex, varying concentrations of JAK2 inhibitor or transcription enhancer were added. The transfection proceeded for 24 hours before harvest. Cells used for the preparation of crystallization-grade JAK2 were incubated with 1 μM Ruxolitinib and 4 mM butyric acid for 23 hours.

Purification of JAK2 kinase domain: Purification of JAK2 KD was performed at 4° C. Expi293F cells were centrifuged at 1,000×g for 30 minutes at 4° C. Cells were resuspended in lysis buffer (50 mM HEPES pH 7.5, 250 mM NaCl, 10% glycerol, 5 mM β-mercaptoethanol, 5 mM MgCl₂, 0.1 mM ATP, 10 mM imidazole, 1 mM PMSF, 0.5% Triton X100). The cells were sonicated at 40% power for 2 minutes on ice and then centrifuged at 40,000×g for 1 hour at 4° C. to clarify the lysate. The soluble lysate was loaded onto two 5 mL HisTrap FF columns in tandem (GE Healthcare). The columns were washed with 10 mM imidazole, 40 mM imidazole, 100 mM imidazole, and 200 mM imidazole before applying a gradient up to 600 mM imidazole. The protein was concentrated to 10 mL using a 10,000 MWCO filter and loaded onto an S75 gel filtration column (GE Healthcare) that was pre-equilibrated with 40 mM bicine pH 8.6, 100 mM NaCl, 10% glycerol. The single JAK2 peak, as determined by SDS-PAGE and western blot analysis (>99% purity), was collected, concentrated to 8.1 mg/mL using a 3,000 MWCO spin concentrator, flash-frozen in liquid N₂ and stored at −80° C.

Cellular Thermal Shift Assay: Cellular thermal shift assay (CETSA) was used to compare melting curves from ligand-based thermal stabilization of JAK2 KD. Expi293F cells were transfected with His-JAK2 KD from pcDNA 3.3 as described above. Ruxolitinib (20 μM), or the equivalent amount of DMSO, was added to the cells and incubated for one hour at 37° C. After one hour, the cells were centrifuged at 300×g for three minutes at room temperature. The cells were washed in 15 mL PBS and centrifuged again at 300×g for three minutes. The cells were resuspended in 1 mL PBS and approximately 3×10⁶ cells were added to PCR tubes that were incubated at 40, 43, 46, 49, 52, 55, 58, 61, 64, or 67° C. for three minutes. The tubes were then flash-frozen in liquid N₂ and thawed twice at 25° C. The cells were vortexed at 20,000×g for 20 minutes at 4° C. The soluble lysate was transferred to a new tube, 5× SDS-PAGE loading buffer was added, and 13 μL (equivalent of 3.3×10⁵ cells) was added to an SDS-PAGE gel. The gel was transferred using the eBlot transfer system (GenScript), blocked with 5% BSA in TBS-T, and blotted for anti-His-HRP, anti-actin or anti-GAPDH-HRP for 2 hours at room temperature. The anti-actin blot was washed then incubated with anti-mouse-HRP conjugated secondary antibody at 1:2,000 for one hour at room temperature. The blots were incubated with SignalFire ECL (Cell Signaling, 6883) and imaged on a GE Healthcare AmerSham Imager 600.

Isothermal Dose-Response: Isothermal dose-response (ITDR) measures protein stabilization as a function of increasing inhibitor concentration. After performing the CETSA assay described above, the data were graphed and a temperature at the IC₅₀ value (47° C.) was used for the isothermal dose-response experiment. Expi293F cells were transfected with His-JAK2 KD. The cells were centrifuged at 300×g for three minutes at room temperature. Cells were resuspended at a density of 4×10⁷ cells/mL. Serial dilutions were performed yielding three-fold dilutions ranging from 10 nM to 20 μM of inhibitor with constant amounts of DMSO. Cells (approximately 1.2×10⁶ cells) were added to the compounds and were incubated for 30 minutes at 37° C. with shaking every 10 minutes. The tubes were heated at 47° C. for three minutes. The cells were then vitrified and thawed twice at 25° C. before being vortexed at 20,000×g for 20 minutes at 4° C. The soluble supernatant was transferred to a new tube, mixed with 5× SDS-loading dye, and resolved on SDS-PAGE. The western blot transfer and incubation is the same as described above. Data were normalized to 0% and 100%.

Cell Viability Assays: UKE-1 cells were cultured in RPMI-1640 with 10% FBS at 37° C. with 5% CO2. Suspension cells were seeded at 20,000 cells per well. Cells were incubated with increasing concentrations of compound ranging from 1 nM to 10 μM (as denoted in the figure legends) in the presence of vehicle (0.2% DMSO) with six replicates per concentration for 72 hours at 37° C. After drug treatment, 15 μL of CellTiter Blue (Promega) was added to each well, mixed for one minute using an orbital shaker, and incubated at 37° C. for three hours. Fluorescence was measured using a Wallac EnVision 2130 plate reader (PerkinElmer). Excitation and emission filters of 570 nm and 615 nm were used, respectively. Cell viability data were analyzed using GraphPad Prism6.

Protein Crystallography: Purified His-JAK2 KD at 8.1 mg/mL was incubated with 1 mM Ruxolitinib and then added to 0.2M NaCl, 0.1M Bis-Tris pH 5.5, and 25% PEG 3350 in a 1:1 v/v ratio on a hanging drop coverslip with the crystallization solution at 293K. Rod-like crystals formed within ten hours and grew to a maximum size over five days. Ligand exchange was performed by moving JAK2-ruxolitinib crystals into the crystallization solution containing 1 mM inhibitor of interest for 3 days. The crystals were preserved in a cryoprotectant containing the crystallization solution and 30% glycerol. All data was collected on the 23-ID beamline, GM/CA at the Advanced Photon Source (Argonne, IL). Data were processed and scaled with XDS. Molecular replacement (using PDB 2XA4 as the search model) and refinements were carried out using PHENIX, and model building was performed using Coot. Figures were prepared using PyMOL (Schradinger, LLC). All structures were validated and deposited in the PDB.

### Results

### Ruxolitinib Significantly Increases the Stability of JAK2 KD Overexpressed in Expi293F Cells

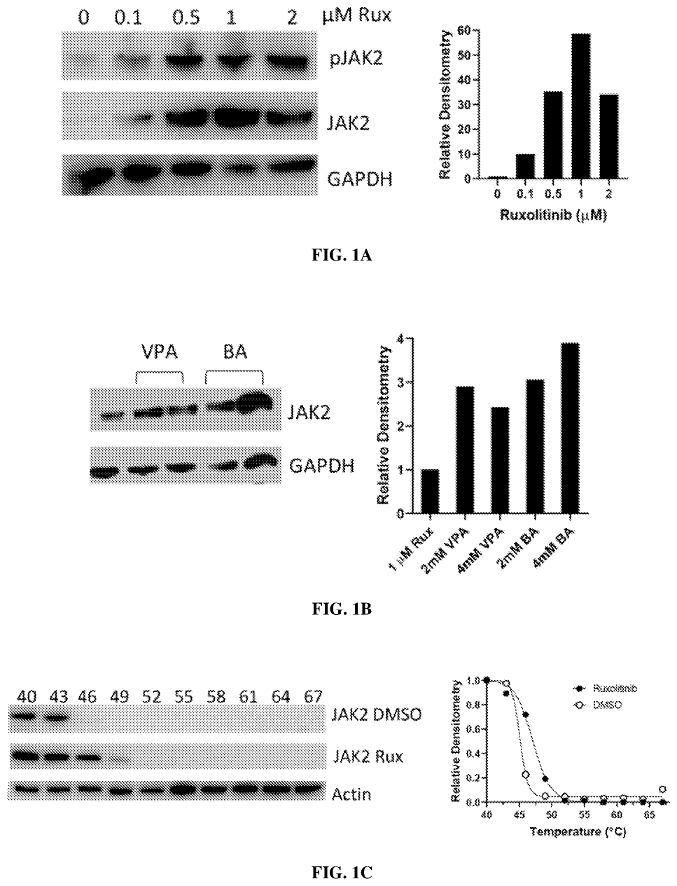

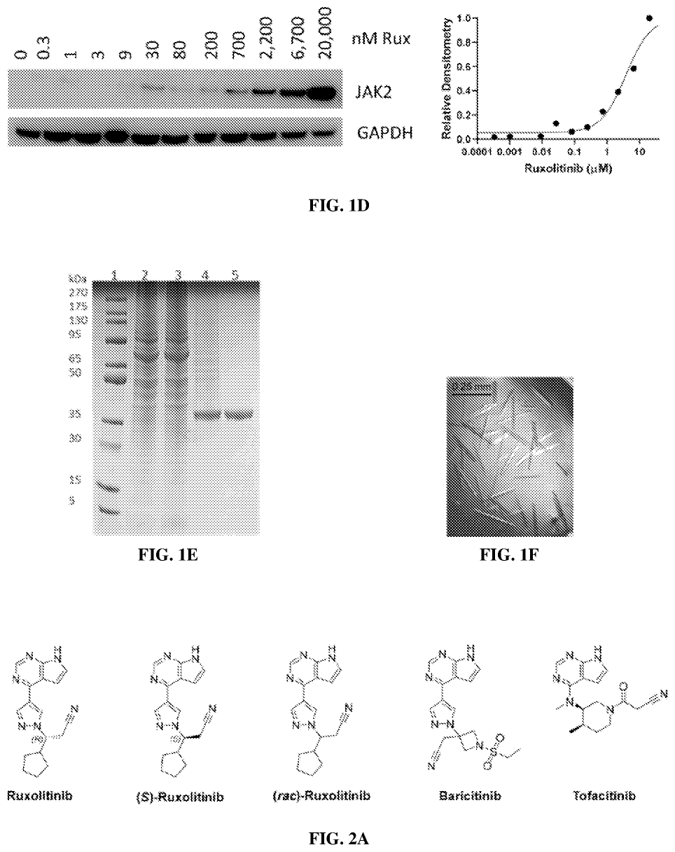

Previously, JAK2 KD has been predominantly purified from insect cells; but this process takes several weeks to generate and amplify the baculovirus to produce large enough quantities of purified protein. Upon transient expression of JAK2 KD in Expi293F cells, the presence of ruxolitinib during the transfection increased the levels of phosphorylated JAK2 (pY1007), as previously reported, as well the overall amount of JAK2 KD in a concentration dependent manner (FIG. 1A). Protein levels of recombinant JAK2 KD from Expi293F cells were approximately 60-fold higher with 1 μM ruxolitinib than in untreated cells. Additionally, by including histone deacetylase (HDAC) inhibitors, valproic acid or butyric acid, during expression alongside ruxolitinib, JAK2 KD expression increased four-fold over that of ruxolitinib alone (FIG. 1B).

Next, we investigated the thermostability of JAK2 KD upon transient expression in the presence of ruxolitinib using a cellular thermal shift assay (CETSA) (see Jafari, R.; Almqvist, H.; Axelsson, H.; Ignatushchenko, M.; Lundback, T.; Nordlund, P.; Martinez Molina, D. The cellular thermal shift assay for evaluating drug target interactions in cells. Nat Protoc 2014, 9, 2100-2122) and isothermal dose-response (ITDR). At 1 μM concentration, ruxolitinib elevated the cellular thermostability of JAK2 KD by 2° C., from 45-47° C. (FIG. 1C). At 47° C., the amount of expressed JAK2 KD increased in dose-response with ruxolitinib (EC₅₀=3.8 μM) (FIG. 1D). Expression in the absence of ruxolitinib yielded very little soluble protein and purity could not be achieved above 50% (data not shown). Adding ruxolitinib to cells during transfection resulted in significantly more protein, which could be purified to homogeneity using Ni-affinity and size exclusion chromatography with yields of 3 mg/L culture (FIG. 1E). Purified JAK2 KD yielded X-ray grade crystals within 24 hours (FIG. 1F).

### Structure-Activity Relationship (SAR) Studies of Diverse Small Molecule JAK2 Inhibitors

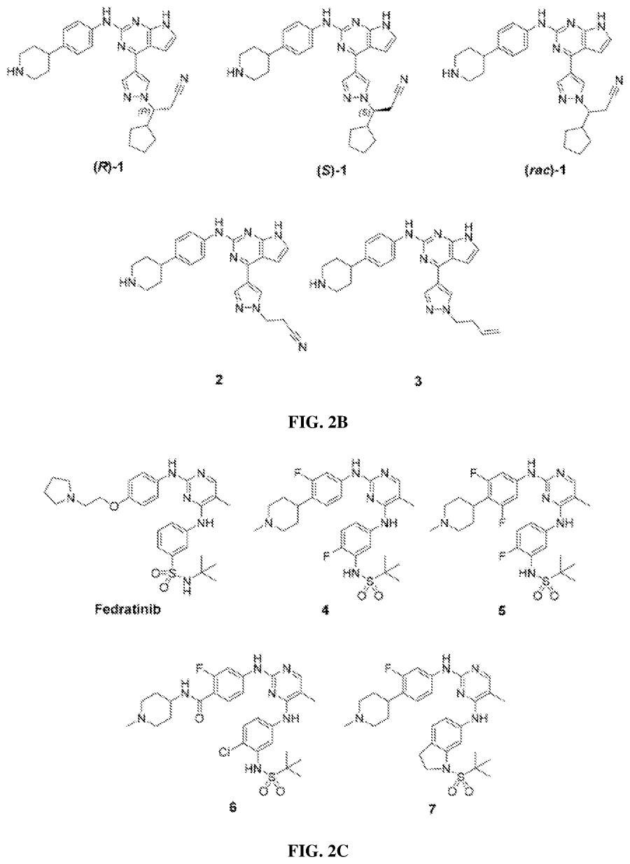

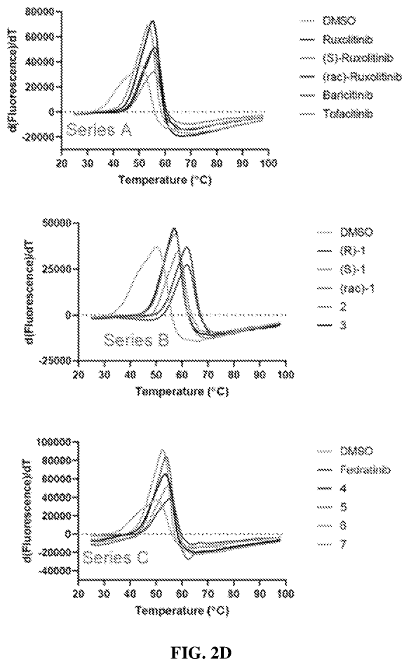

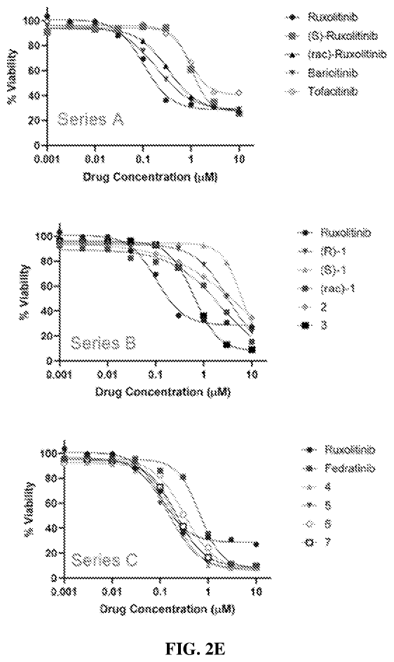

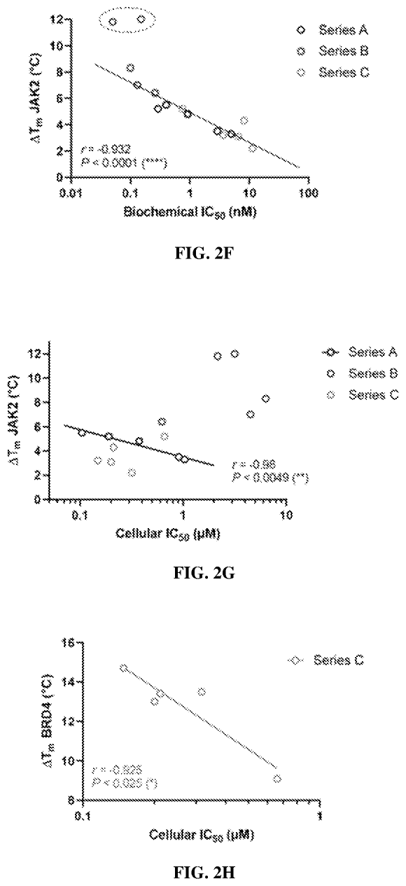

To assess the suitability of purified JAK2 KD for SAR studies for drug development campaigns, three series of JAK2 inhibitors (FIG. 2A-C) were subjected to binding studies by differential scanning fluorimetry (DSF). Series A consists of ruxolitinib enantiomers and FDA-approved derivatives baricitinib and tofacitinib, while series B consists of piperidine-aniline analogues of ruxolitinib. Series C comprises the FDA-approved JAK2 inhibitor fedratinib and other diaminopyrimidines, which were further developed as dual JAK2-BRD4 inhibitors from early lead compounds. (see Ember, S. W.; Lambert, Q. T.; Berndt, N.; Gunawan, S.; Ayaz, M.; Tauro, M.; Zhu, J. Y.; Cranfill, P. J.; Greninger, P.; Lynch, C.

C.; Benes, C. H.; Lawrence, H. R.; Reuther, G. W.; Lawrence, N. J.; Schonbrunn, E. Potent dual BET bromodomain-kinase inhibitors as value-added multitargeted chemical probes and cancer therapeutics. Mol Cancer Ther 2017, 16, 1054-1067) The melting temperature of JAK2 in the absence of inhibitor was 50° C., and thermal shifts in the presence of 100 μM inhibitor ranged from 3.3 to 5.5° C. for series A, 6.4 to 12° C. for series B and 2.2 to 5.2° C. for series C (FIG. 2D and Table 1).

TABLE 1
Binding and inhibitory potential of investigated JAK2 inhibitors

| | | | | UKE-1 |
|---|---|---|---|---|
| | | Binding | Biochemical | cell growth |
| | | potential¹ | inhibition² | inhibition³ |
| Series | Compound ID | ΔTₘ (° C.) | IC₅₀ (nM) | IC₅₀ (μM) |
| A | Ruxolitinib | 5.5 ± 0.06 | 0.40 ± 0.005 | 0.1 ± 0.02 |
| | (S)-Ruxolitinib | 3.3 ± 0.04 | 5.0 ± 0.1 | 1.04 ± 0.09 |
| | (rac)-Ruxolitinib | 4.8 ± 0.05 | 0.92 ± 0.06 | 0.38 ± 0.08 |
| | Baricitinib | 5.2 ± 0.08 | 0.29 ± 0.13 | 0.19 ± 0.03 |
| | Tofacitinib | 3.5 ± 0.08 | 2.9 ± 0.39 | 0.91 ± 0.1 |
| B | (R)-1 | 12 ± 0.02 | 0.15 ± 0.03 | 3.2 ± 0.76 |
| | (S)-1 | 8.3 ± 0.08 | 0.099 ± 0.02 | 6.3 ± 5.4 |
| | (rac)-1 | 11.8 ± 0.05 | <0.05 | 2.2 ± 1.9 |
| | 2 | 7 ± 0.03 | 0.13 ± 0.048 | 4.5 ± 5.9 |
| | 3 | 6.4 ± 0.04 | 0.26 ± 0.04 | 0.62 ± 0.04 |
| C | Fedratinib | 5.2 ± 0.07 | 0.75 ± 0.39 | 0.66 ± 0.06 |
| | 4 | 3.1 ± 0.09 | 6.5 ± 3.5 | 0.2 ± 0.02 |
| | 5 | 3.2 ± 0.04 | 8.2 ± 5.7 | 0.15 ± 0.01 |
| | 6 | 2.2 ± 0.06 | 11.4 ± 0.87 | 0.32 ± 0.02 |
| | 7 | 4.3 ± 0.05 | 3.6 ± 1.1 | 0.21 ± 0.02 |

¹Standard deviation (SD) from two DSF data sets, each performed in quadruplicate.
²SD from two data sets of radiometric assay by Reaction Biology.
³Standard error of the mean (SEM) for data from three experiments, each performed in hexaplicate.

To evaluate if the thermal shifts reflect inhibitory activity against JAK2 in cells, compounds were characterized for growth inhibitory activity of UKE-1 cells, which are driven by V617F mutant JAK2 (FIG. 2E). (see Quentmeier, H.; MacLeod, R. A.; Zaborski, M.; Drexler, H. G. JAK2 V617F tyrosine kinase mutation in cell lines derived from myeloproliferative disorders. Leukemia 2006, 20, 471-476) The resulting IC₅₀ values ranged from 0.1 to 1.0 μM for series A, 0.6 to 6.3 μM for series B and 0.15 to 0.66 μM for series C. Additionally, compounds were characterized for inhibition of enzymatic JAK2 activity in a radiometric assay by Reaction Biology Corp., IC₅₀ values ranging from 0.29 to 5.0 nM for series A, 0.05 to 0.26 nM for series B, and 0.75 to 11.4 nM for series C (Table 1). Statistical significance of the data sets was evaluated by Pearson's correlation analyses (FIG. 2F-H). Thermal shifts showed excellent correlation with enzymatic inhibition values across all compounds, except for (R)-1 and rac-1 of series B, the high inhibitory activity of which likely exceeded the sensitivity of the radiometric assay applied (IC₅₀<0.15 nM) (FIG. 2F). By contrast, only series A significantly correlated with cellular inhibition data (FIG. 2G). Series B probably suffers from poor cell permeability, while series C showed higher cell growth inhibitory activity than expected from JAK2 inhibition alone. The increased cellular activity of series C is likely caused by their dual activity against JAK2 and BRD4, as UKE-1 cells are highly sensitive to inhibition of BRD4. The thermal shifts exerted by these compounds towards BRD4 correlated significantly with inhibitory activity against UKE-1 cell growth (FIG. 2H).

### Structural Basis of JAK2 Inhibition by Ruxolitinib and Baricitinib.

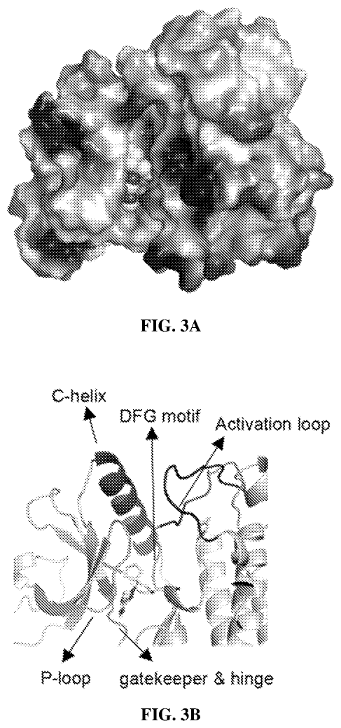

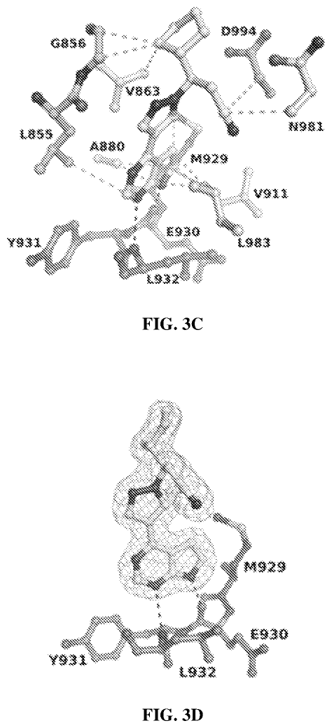

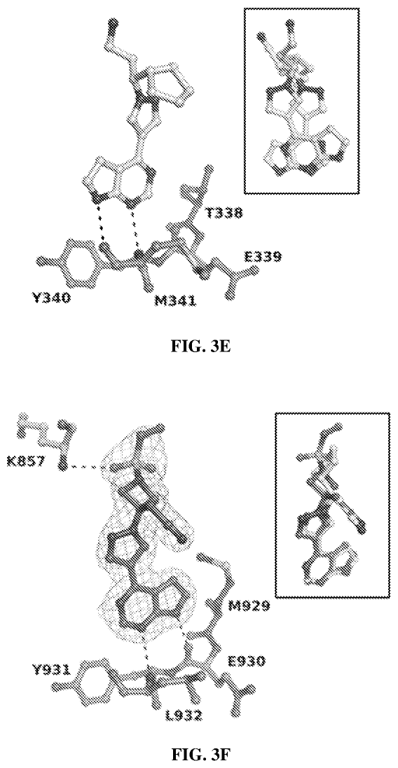

Structural information on the JAK2-ruxolitinib complex was previously limited to molecular dynamics simulations or by prediction based on the known structures of c-SRC with ruxolitinib and of JAK2 with tofacitinib. (see Duan, Y.; Chen, L.; Chen, Y.; Fan, X. G. c-Src binds to the cancer drug Ruxolitinib with an active conformation. PLoS One 2014, 9, e106225; and Williams, N. K.; Bamert, R. S.; Patel, O.; Wang, C.; Walden, P. M.; Wilks, A. F.; Fantino, E.; Rossjohn, J.; Lucet, I. S. Dissecting specificity in the Janus kinases: the structures of JAK-specific inhibitors complexed to the JAK1 and JAK2 protein tyrosine kinase domains. J Mol Biol 2009, 387, 219-232) Crystals of JAK2 KD grown in the absence of added inhibitor (FIG. 1F) showed that ruxolitinib was still bound to protein despite ˜100,000-fold dilution throughout the purification process, reflecting the high binding affinity of ruxolitinib for JAK2 KD. However, bound ruxolitinib could be readily displaced by other inhibitors through in-diffusion of crystals with 1 mM inhibitor for 72 hours prior to data collection. Ruxolitinib is housed deep inside the ATP site, anchored through H-bonding interactions between the pyrrolopyrimidine moiety and main chain atoms of Glu930 and Leu932 of the hinge region (FIG. 3A-D).

The inhibitor is held in place through several van-der-Waals (VDW) hydrophobic interactions with surrounding residues, including the P-loop (Leu855, Gly856) and the DFG motif (Asp994). The binding pose of ruxolitinib in JAK2 agrees with molecular dynamics predictions, but significantly differs from that observed in the crystal structure of c-SRC (FIG. 3E). In SRC, ruxolitinib is rotated ˜180° relative to the hinge region, likely caused by repulsion and/or steric hindrance with the gatekeeper residue Thr338. In JAK2, the hydrophobic and flexible gatekeeper residue Met929 accommodates ruxolitinib through multiple VDW interactions. Accordingly, ruxolitinib inhibitory activity is three orders of magnitudes higher for JAK2 over SRC. To confirm the pyrrolopyrimidine binding mode of ruxolitinib in JAK2, a co-crystal structure was determined with baricitinib showing almost identical positioning of the two inhibitors in the ATP site (FIG. 3F).

### Stereoselective Discrimination of Ruxolitinib and Aniline Derivatives by JAK2

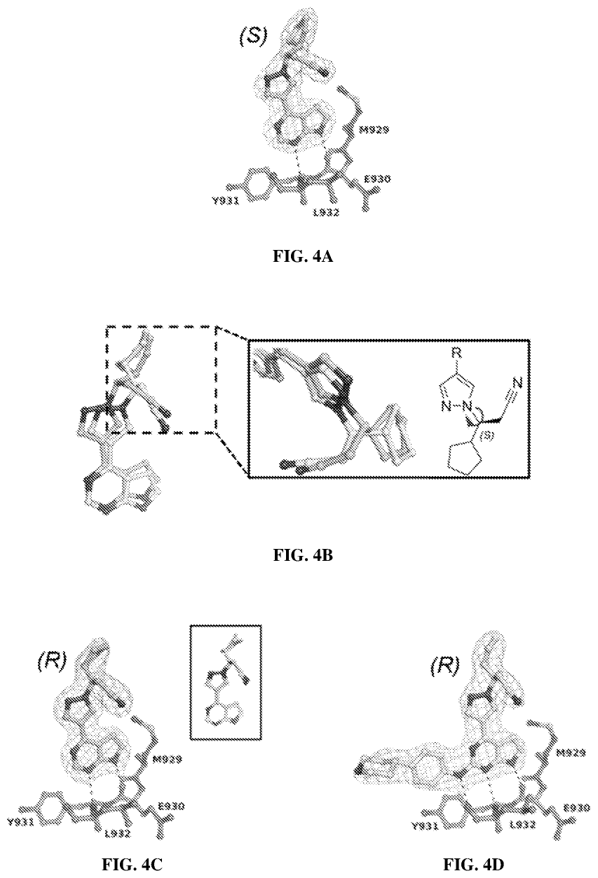

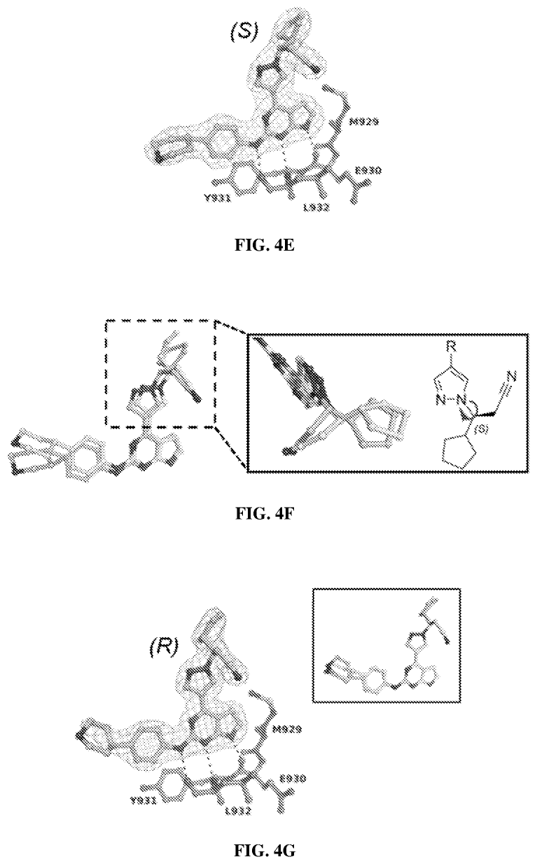

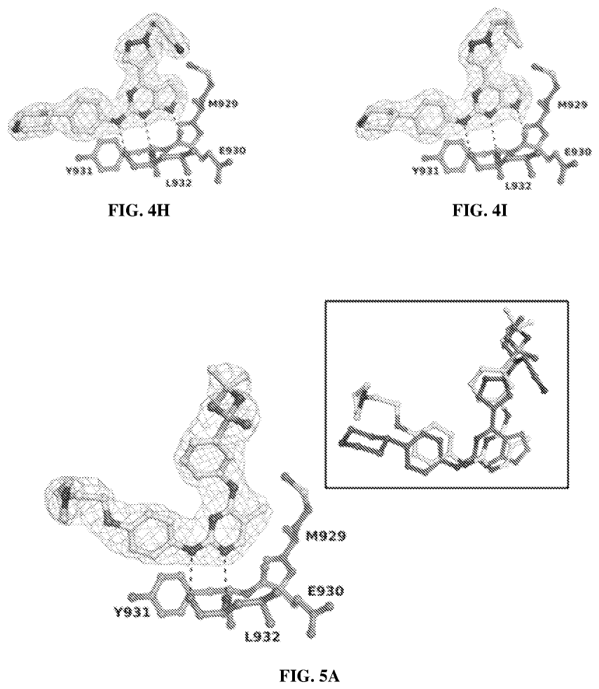

The high diffraction power of the JAK2 KD crystals prompted us to evaluate the structure-activity relationship of enantiomers of ruxolitinib and analogues thereof. The S-isomer of ruxolitinib is ˜10-fold less active against JAK2 than the R-isomer (Table 1). The co-crystal structure revealed that (S)-ruxolitinib adopts a binding pose similar to (R)-ruxolitinib (FIG. 4A) and achieves shape complementarity with the ATP site through −180° rotation about the chiral center (FIG. 4B). Using the racemic mixture of ruxolitinib, (rac)-ruxolitinib, the resulting co-crystal structure revealed exclusively the R-isomer bound (FIG. 4C). Likewise, the R- and S-enantiomers of aniline derivatives of ruxolitinib, (R)-1 and (S)-1, assumed the same orientation for the cyclopentyl and propionitrile moieties in the ATP site (FIG. 4D-F). Furthermore, the racemic mixture (rac-1) showed only the R-isomer bound (FIG. 4G). The data demonstrate that JAK2 discriminates between the R and S stereoisomers of ruxolitinib and derivatives by preferentially interacting with the R-isomer. Ruxolitinib derivatives devoid of a chiral center and the cyclopentyl group (2 and 3) exhibited the same preference of the respective propionitrile and butenyl moieties for positioning in the sub-pocket that accommodates the propionitrile of ruxolitinib (FIG. 4H, I). Notably, the aniline derivatives interact in the ATP site through an additional H-bond with the hinge region (FIG. 4D-I), which explains the substantial increase in JAK2 inhibitory activity of series B over ruxolitinib and series A (Table 1).

### Structural Basis of JAK2 Inhibition by Diaminopyrimidines

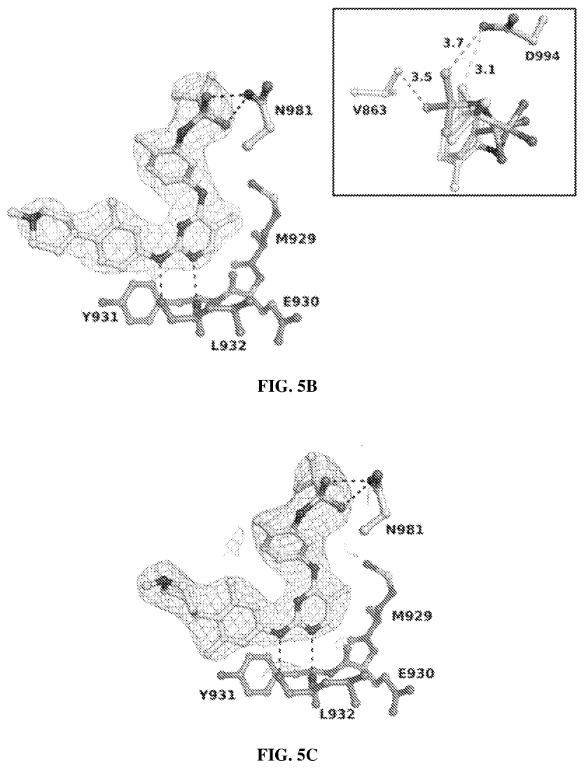

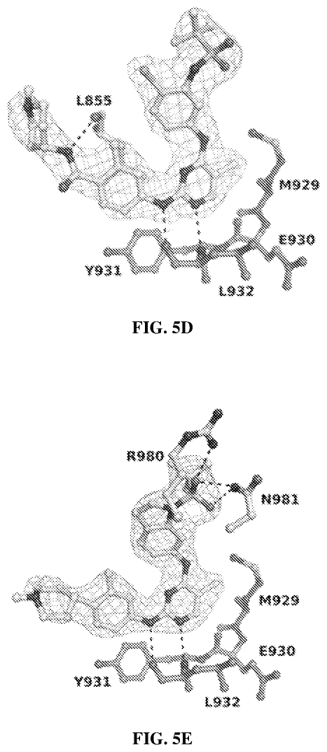

To further probe the ATP site, co-crystal structures were determined with dianilinopyrimidine-containing inhibitors of series C. All inhibitors interacted with the hinge region similarly to the aniline derivatives of ruxolitinib (FIG. 5). However, the lack of a pyrrolo moiety allows only for the establishment of H-bonds with the main chain atoms of Leu932, but not with Glu930. This is reflected in a 10-fold reduced biochemical potency of inhibitors of series C against JAK2 (Table 1). Reversal of the sulfonamide moiety and addition of fluorine (4), which is beneficial for binding to the acetyl-lysine binding site of BRD4, slightly tilts the aniline ring such that the tert-butyl moiety moves away from Va1863 and positions close to the carboxyl group of Asp994 of the DFG motif (FIG. 5B). Although the reversed sulfonamide enables potential H-bonds with the side chain of Asn981, the unfavorably close distance between the hydrophobic tert-butyl and Asp994 appears to cause slightly reduced inhibitory activity for 4 and related compounds 5 and 6 (FIG. 5C, D). Constraining the sulfonamide moiety using an indoline moiety (7) was well tolerated (FIG. 5E) and showed highest inhibitory activity against JAK2 across series C (Table 1).

### DISCUSSION AND CONCLUSIONS

JAK2 presents challenges for structural studies as it is not stable upon recombinant overexpression, and typical protein purifications require over 10 L insect cultures from bioreactors to obtain a few milligrams of crystallization-grade protein. (see Hall, T.; Emmons, T. L.; Chrencik, J. E.; Gormley, J. A.; Weinberg, R. A.; Leone, J. W.; Hirsch, J. L.; Saabye, M. J.; Schindler, J. F.; Day, J. E.; Williams, J. M.; Kiefer, J. R.; Lightle, S. A.; Harris, M. S.; Guru, S.; Fischer, H. D.; Tomasselli, A. G. Expression, purification, characterization and crystallization of non- and phosphorylated states of JAK2 and JAK3 kinase domain. Protein Expr Purif 2010, 69, 54-63; Zimmermann, K.; Sang, X.; Mastalerz, H. A.; Johnson, W. L.; Zhang, G.; Liu, Q.; Batt, D.; Lombardo, L. J.; Vyas, D.; Trainor, G. L.; Tokarski, J. S.; Lorenzi, M. V.; You, D.; Gottardis, M. M.; Lippy, J.; Khan, J.; Sack, J. S.; Purandare, A. V. 9H-Carbazole-1-carboxamides as potent and selective JAK2 inhibitors. Bioorg Med Chem Lett 2015, 25, 2809-2812; Vazquez, M. L.; Kaila, N.; Strohbach, J. W.; Trzupek, J. D.; Brown, M. F.; Flanagan, M. E.; Mitton-Fry, M. J.; Johnson, T. A.; TenBrink, R. E.; Arnold, E. P.; Basak, A.; Heasley, S. E.; Kwon, S.; Langille, J.; Parikh, M. D.; Griffin, S. H.; Casavant, J. M.; Duclos, B. A.; Fenwick, A. E.; Harris, T. M.; Han, S.; Caspers, N.; Dowty, M. E.; Yang, X.; Banker, M. E.; Hegen, M.; Symanowicz, P. T.; Li, L.; Wang, L.; Lin, T. H.; Jussif, J.; Clark, J. D.; Telliez, J. B.; Robinson, R. P.; Unwalla, R. Identification of N-{cis-3-[Methyl(7H-pyrrolo [2,3-d]pyrimidin-4-yl)amino]cyclobutyl}propane-1-sulfo namide (PF-04965842): A selective JAK1 clinical candidate for the treatment of autoimmune diseases. J Med Chem 2018, 61, 1130-1152 and Zak, M.; Mendonca, R.; Balazs, M.; Barrett, K.; Bergeron, P.; Blair, W. S.; Chang, C.; Deshmukh, G.; Devoss, J.; Dragovich, P. S.; Eigenbrot, C.; Ghilardi, N.; Gibbons, P.; Gradl, S.; Hamman, C.; Hanan, E. J.; Harstad, E.; Hewitt, P. R.; Hurley, C. A.; Jin, T.; Johnson, A.; Johnson, T.; Kenny, J. R.; Koehler, M. F.; Bir Kohli, P.; Kulagowski, J. J.; Labadie, S.; Liao, J.; Liimatta, M.; Lin, Z.; Lupardus, P. J.; Maxey, R. J.; Murray, J. M.; Pulk, R.; Rodriguez, M.; Savage, S.; Shia, S.; Steffek, M.; Ubhayakar, S.; Ultsch, M.; van Abbema, A.; Ward, S. I.; Xiao, L.; Xiao, Y. Discovery and optimization of C-2 methyl imidazopyrrolopyridines as potent and orally bioavailable JAK1 inhibitors with selectivity over JAK2. J Med Chem 2012, 55, 6176-6193) Recombinant overexpression of JAK2 KD using mammalian Expi293F cells requires only 24 hours growth post transfection and 1 liter of culture is sufficient to purify milligram quantities of crystallization-grade protein for biochemical and structural studies. This was achieved using ruxolitinib during expression, which significantly enhanced protein stability and purification yields and enabled the first crystal structures of JAK2 expressed in mammalian cells. The crystal structure data suggest that ruxolitinib improves the thermostability of JAK2 by decreasing flexible regions and causing a more rigid structure with well-defined activation and P-loops. Notably, ruxolitinib did not affect the proliferation of Expi293F or HEK293 cells even at 10 μM after 72 hours. This strategy provided faster, more efficient, and cost-effective production of recombinant JAK2, and may be applicable to other difficult to overexpress kinases provided that the inhibitor is specific, potent, and non-lethal over the timeframe of expression.

Robust crystallization conditions and ease of in-diffusion of inhibitors resulted in 14 novel co-crystal structures of JAK2 liganded with various inhibitors of three chemical scaffolds. Along with binding and inhibition data, this information provides a comprehensive view of the ATP site and the shape complementarity required for small molecules to achieve highest inhibitory activity. The (R) and (S) stereoisomers of ruxolitinib and aniline derivatives thereof were thoroughly characterized for binding potential and inhibitory activity against cancer cells driven by constitutively active V617F JAK2. While (S)-ruxolitinib is a formidable inhibitor of JAK2 (IC₅₀=5 nM), the (R)-isomer exerted >10-fold higher activity with respect to binding and cell kill potential. Co-crystal structures obtained with the isolated isomers along with racemic mixtures demonstrate that JAK2 discriminates against the S-configuration. The (S)-isomer mimics the conformation of the preferred R-isomer through rotation about the chiral center, but the resulting binding pose is less compatible with the ATP site. Notably, the achiral analogue baricitinib adopts a conformation almost identical to that of ruxolitinib, which is reflected in the similar values obtained for binding, enzymatic and cellular activities of these FDA-approved inhibitors.

Piperidine aniline derivatives of both (R)- and (S)-ruxolitinib showed greatly enhanced binding and inhibitory potential towards JAK2; the picomolar activities could not be resolved by the steady-state assays employed here. This substantial increase in activity is attributable to an additional H-bond established with the hinge region. Although these compounds suffer from poor cell penetration as indicated by a significant loss of cell inhibitory activity, solubilizing groups other than piperidine may alleviate this drawback.

Diaminopyrimidine inhibitors of JAK2, including fedratinib, mimic the binding pose of aniline derivatives of ruxolitinib. However, they lack the potential to establish an H-bond with Glu930 of the hinge region, which is reflected in reduced but still formidable inhibitory activities (IC₅₀ between 0.75 and 11.4 nM). These compounds also inhibit BET bromodomains to varying degrees and appear to exert strong cell growth inhibitory activity by predominantly targeting BRD4. Previously, structural modeling, binding and enzymatic assays using full-length JAK2 expressed in insect cells showed high affinity of fedratinib for the substrate-binding site.³ The co-crystal structure of JAK2 kinase domain with fedratinib (or any other compound studied herein) showed inhibitor bound exclusively to the ATP site. It is conceivable that fedratinib interaction with the substrate binding site occurs in the full-length enzyme, but not in the isolated kinase domain. Combined, our findings may serve the iterative drug design process in developing JAK2 inhibitors with increased efficacy to combat cancers caused by this enzyme.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A compound of Formula I or Formula II 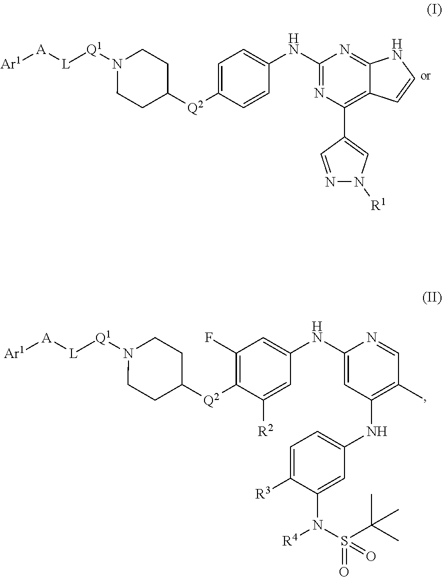
or a pharmaceutically acceptable salt thereof;
wherein:
Ar¹ is selected from 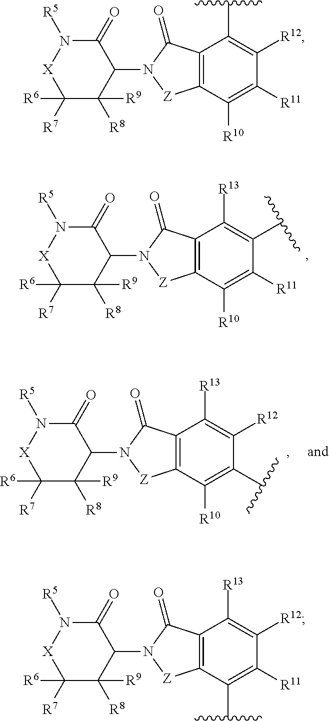
A is selected from —O—, —S—, —NH—, —CH₂—, —O—(C₁-C₄ alkyl)-C(O)NH—, and 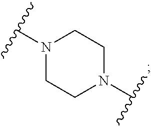
L is selected from C₂-C₁₅ alkyl and —(CH₂CH₂O)ₙ(C₁-C₄ alkyl)-, wherein n is selected from 1, 2, 3, 4, 5, 6, 7, and 8;
Q¹ is a bond or —C(═O)—;
Q² is a bond, —NHC(═O)—, or —C(═O)NH—;
R¹ is selected from 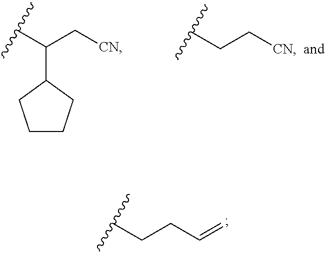
R² is selected from hydrogen or F;
R³ is selected from Cl or F;
R⁴ is hydrogen; or
R³ and R⁴ are brought together with the atoms to which they are attached to form a pyrrolidine ring;
X is selected from —C(═O)— and —CH₂;
Z is selected from —CH₂— and —C(═O)—;
R⁵ is selected from hydrogen and C₁-C₈ alkyl;
R⁶, R⁷, R⁸, and R⁹ are each independently selected from hydrogen, halogen, —NH₂, —OH, —NO₂, —CN, C₁-C₄ alkyl, C₂-C₄ alkenyl, C₁-C₄ haloalkyl, C₁-C₄ cyanoalkyl, C₁-C₄ hydroxyalkyl, C₁-C₄ haloalkoxy, C₁-C₄ alkoxy, C₁-C₄ alkylamino, (independently C₁-C₄ dialkylamino, and C₁-C₄ aminoalkyl; and
R¹⁰, R¹¹, R¹², and R¹³ are each independently selected from hydrogen, halogen, —NH₂, —OH, —NO₂, —CN, C₁-C₄ alkyl, C₂-C₄ alkenyl, C₁-C₄ haloalkyl, C₁-C₄ cyanoalkyl, C₁-C₄ hydroxyalkyl, C₁-C₄ haloalkoxy, C₁-C₄ alkoxy, C₁-C₄ alkylamino, independently C₁-C₄ dialkylamino, and C₁-C₄ aminoalkyl.

2-8. (canceled)

9. A compound of claim 1, wherein L is selected from ethyl, n-propyl, and isopropyl, n-pentyl and neopentyl.

10. (canceled)

11. A compound of claim 1, wherein L is selected from —(CH₂CH₂O)ₙ(CH₂)—, —(CH₂CH₂O)ₙ(CH₂CH₂)—, —(CH₂CH₂O)ₙ(CH₂CH₂CH₂)—, and —(CH₂CH₂O)ₙ(CH(CH₃)CH₂)—.

12-15. (canceled)

16. A compound of claim 1, wherein Ar¹ is selected from

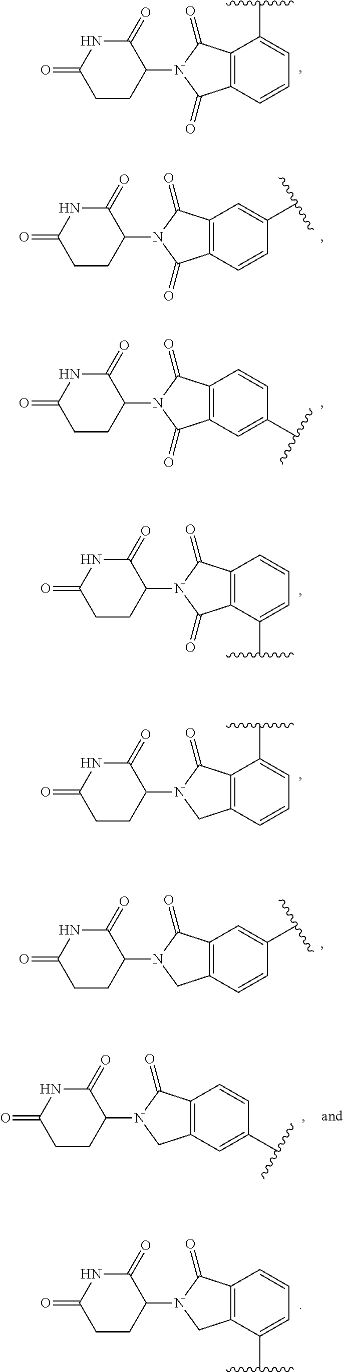

17. (canceled)

18. A compound of claim 1, wherein 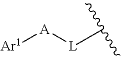
is selected from:

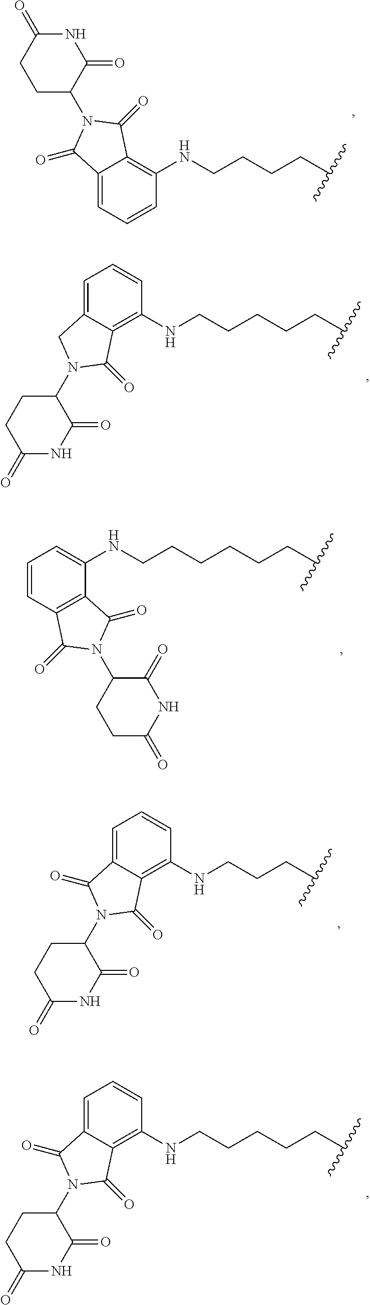

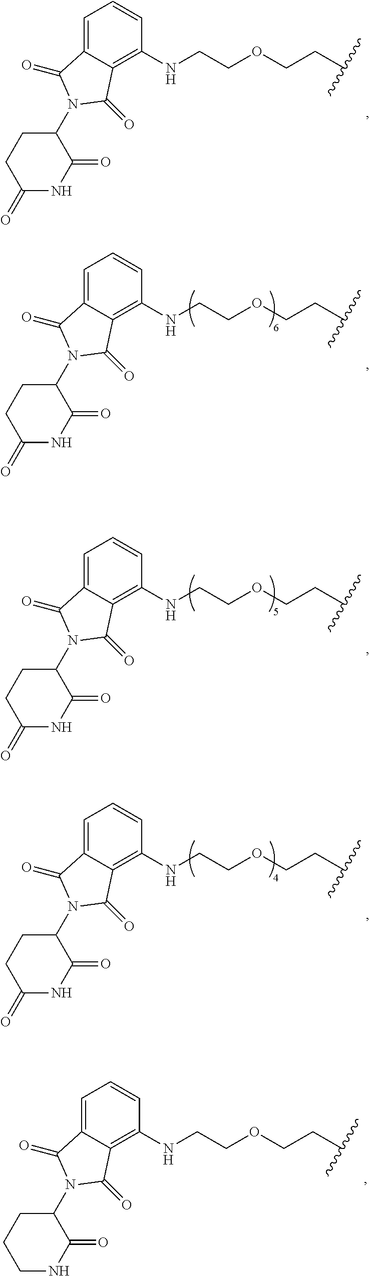

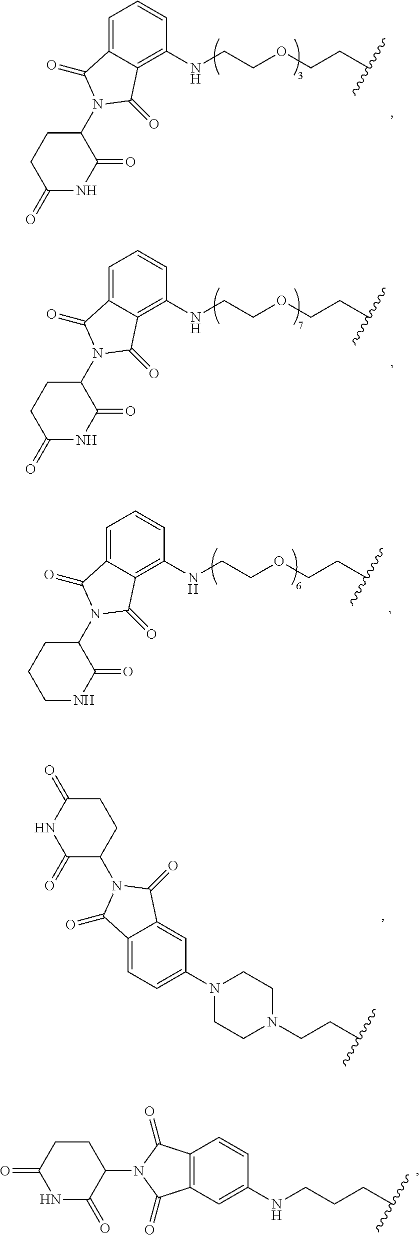

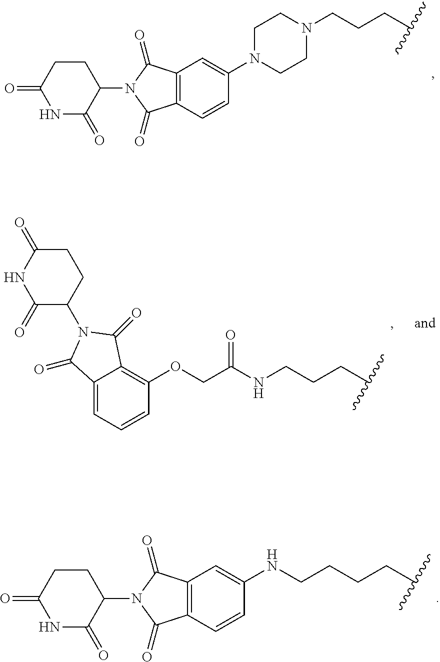 [SMILES: *CCCN1CCN(c2ccc3c(c2)C(=O)N(C2CCC(=O)NC2=O)C3=O)CC1 ; *CCCNC(=O)COc1cccc2c1C(=O)N(C1CCC(=O)NC1=O)C2=O ; *CCCCNc1ccc2c(c1)C(=O)N(C1CCC(=O)NC1=O)C2=O]

19-27. (canceled)

28. A compound of claim 1, selected from: 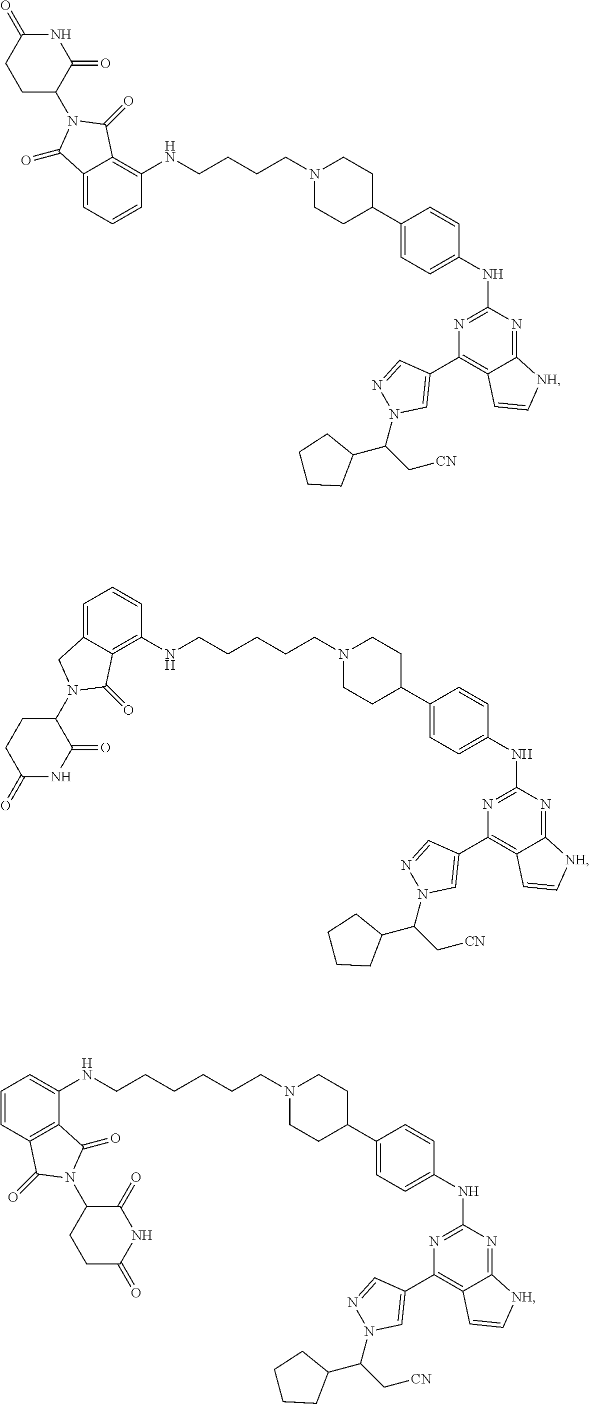 [SMILES: N#CCC(C1CCCC1)n1cc(-c2nc(Nc3ccc(C4CCN(CCCCNc5cccc6c5C(=O)N(C5CCC(=O)NC5=O)C6=O)CC4)cc3)nc3[nH]ccc23)cn1 ; N#CCC(C1CCCC1)n1cc(-c2nc(Nc3ccc(C4CCN(CCCCCNc5cccc6c5C(=O)N(C5CCC(=O)NC5=O)C6)CC4)cc3)nc3[nH]ccc23)cn1 ; N#CCC(C1CCCC1)n1cc(-c2nc(Nc3ccc(C4CCN(CCCCCCNc5cccc6c5C(=O)N(C5CCC(=O)NC5=O)C6=O)CC4)cc3)nc3[nH]ccc23)cn1] 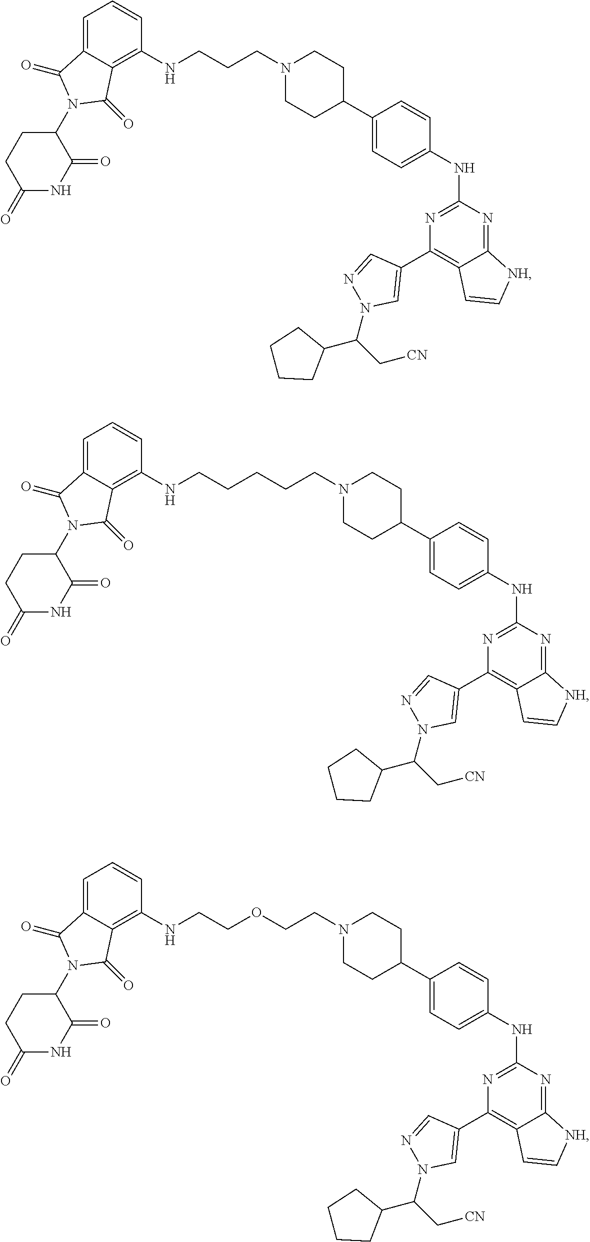 [SMILES: N#CCC(C1CCCC1)n1cc(-c2nc(Nc3ccc(C4CCN(CCCNc5cccc6c5C(=O)N(C5CCC(=O)NC5=O)C6=O)CC4)cc3)nc3[nH]ccc23)cn1 ; N#CCC(C1CCCC1)n1cc(-c2nc(Nc3ccc(C4CCN(CCCCCNc5cccc6c5C(=O)N(C5CCC(=O)NC5=O)C6=O)CC4)cc3)nc3[nH]ccc23)cn1 ; N#CCC(C1CCCC1)n1cc(-c2nc(Nc3ccc(C4CCN(CCOCCNc5cccc6c5C(=O)N(C5CCC(=O)NC5=O)C6=O)CC4)cc3)nc3[nH]ccc23)cn1] 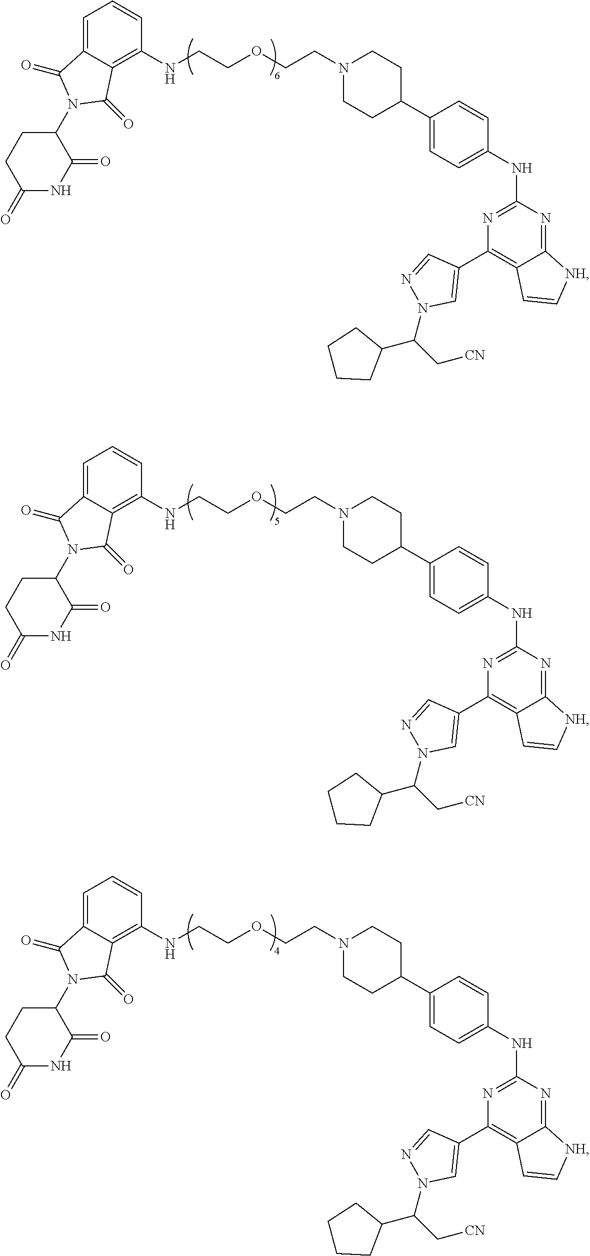 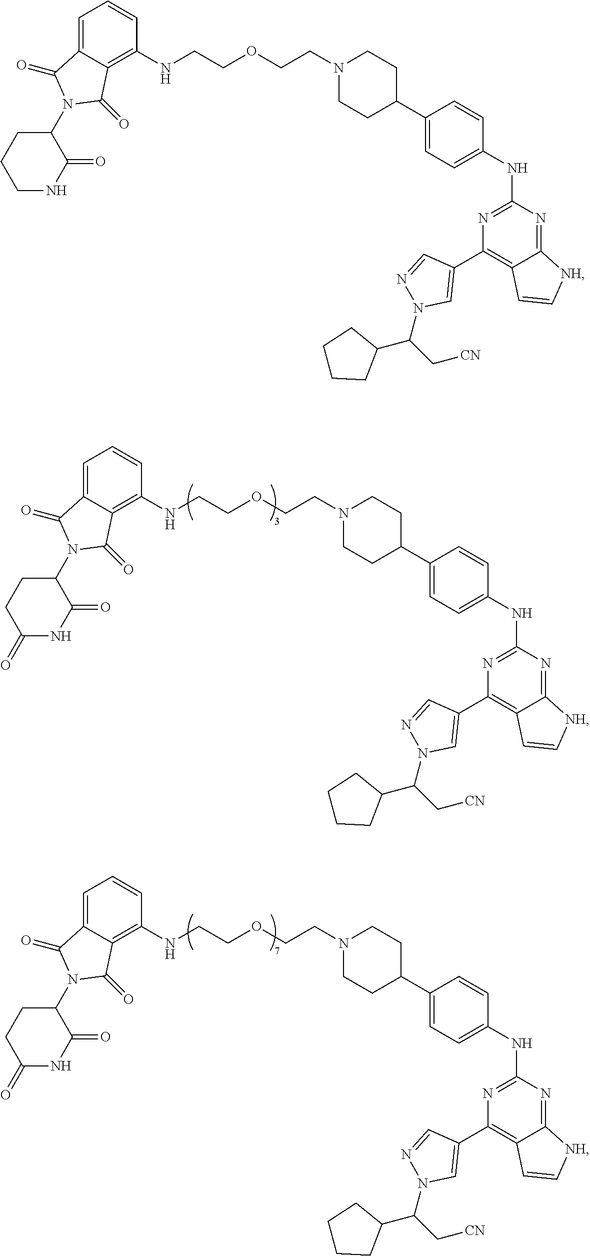 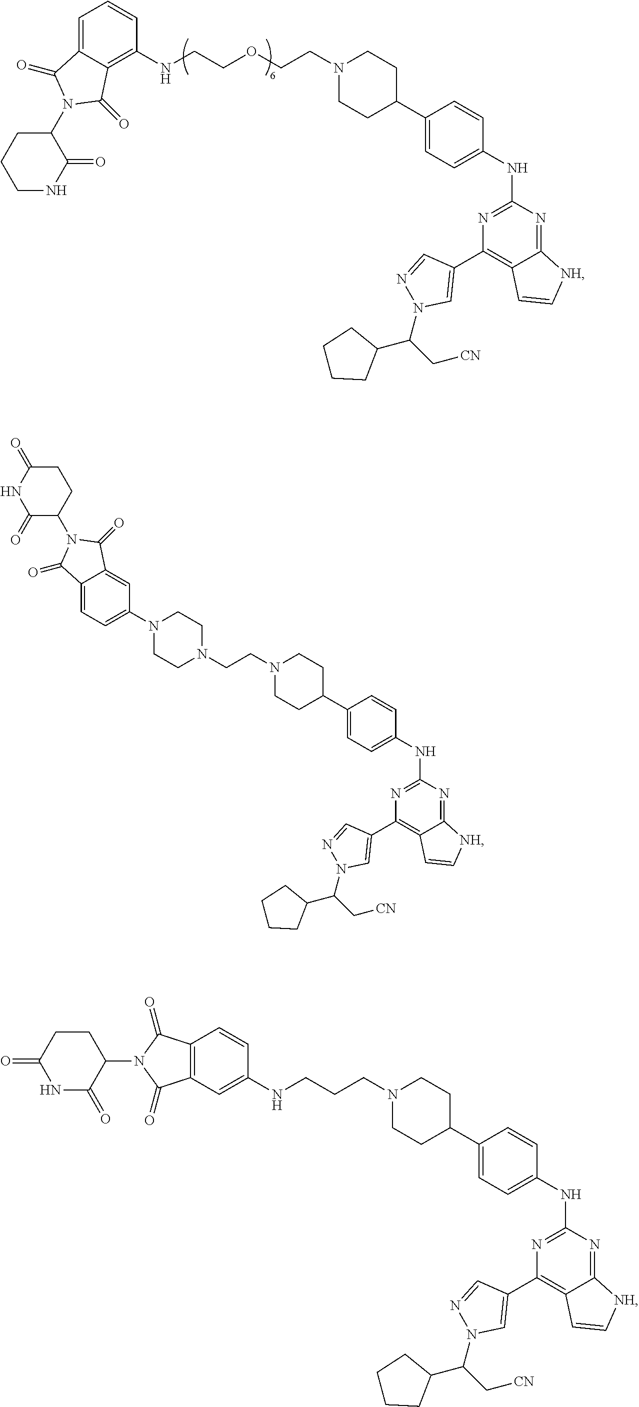 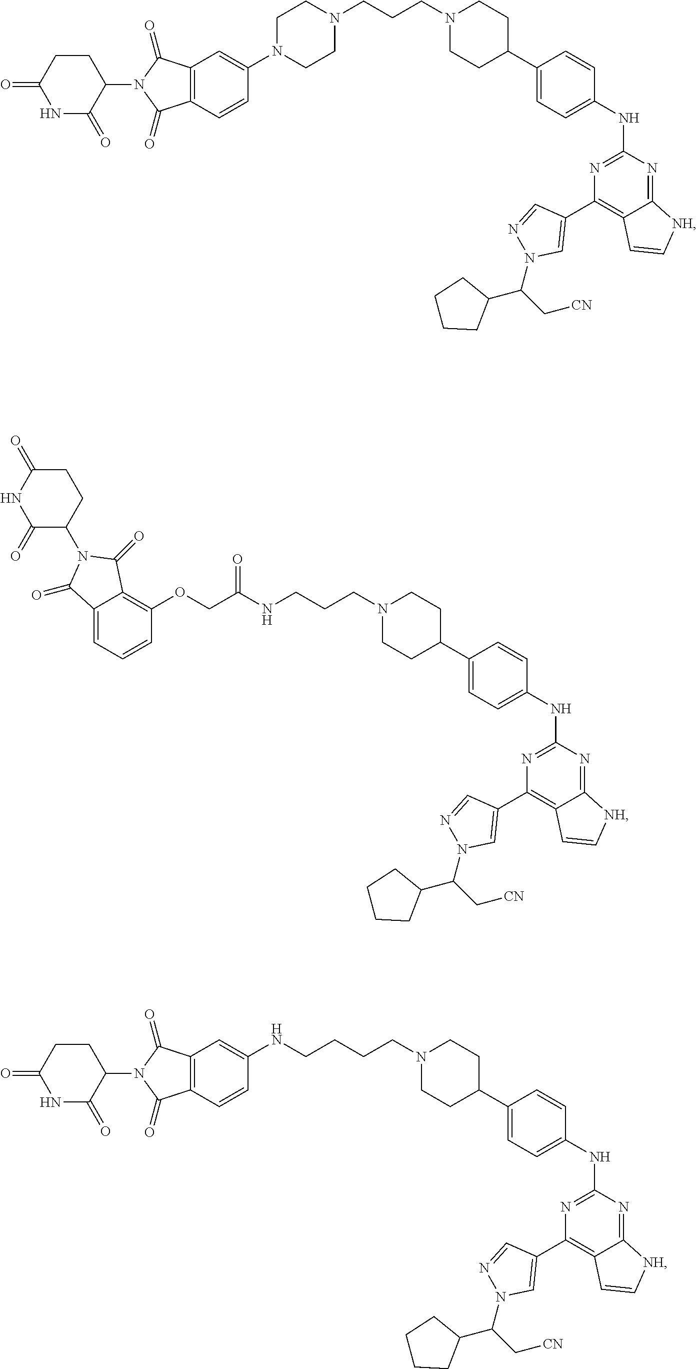 [SMILES: N#CCC(C1CCCC1)n1cc(-c2nc(Nc3ccc(C4CCN(CCCN5CCN(c6ccc7c(c6)C(=O)N(C6CCC(=O)NC6=O)C7=O)CC5)CC4)cc3)nc3[nH]ccc23)cn1 ; N#CCC(C1CCCC1)n1cc(-c2nc(Nc3ccc(C4CCN(CCCNC(=O)COc5cccc6c5C(=O)N(C5CCC(=O)NC5=O)C6=O)CC4)cc3)nc3[nH]ccc23)cn1 ; N#CCC(C1CCCC1)n1cc(-c2nc(Nc3ccc(C4CCN(CCCCNc5ccc6c(c5)C(=O)N(C5CCC(=O)NC5=O)C6=O)CC4)cc3)nc3[nH]ccc23)cn1] 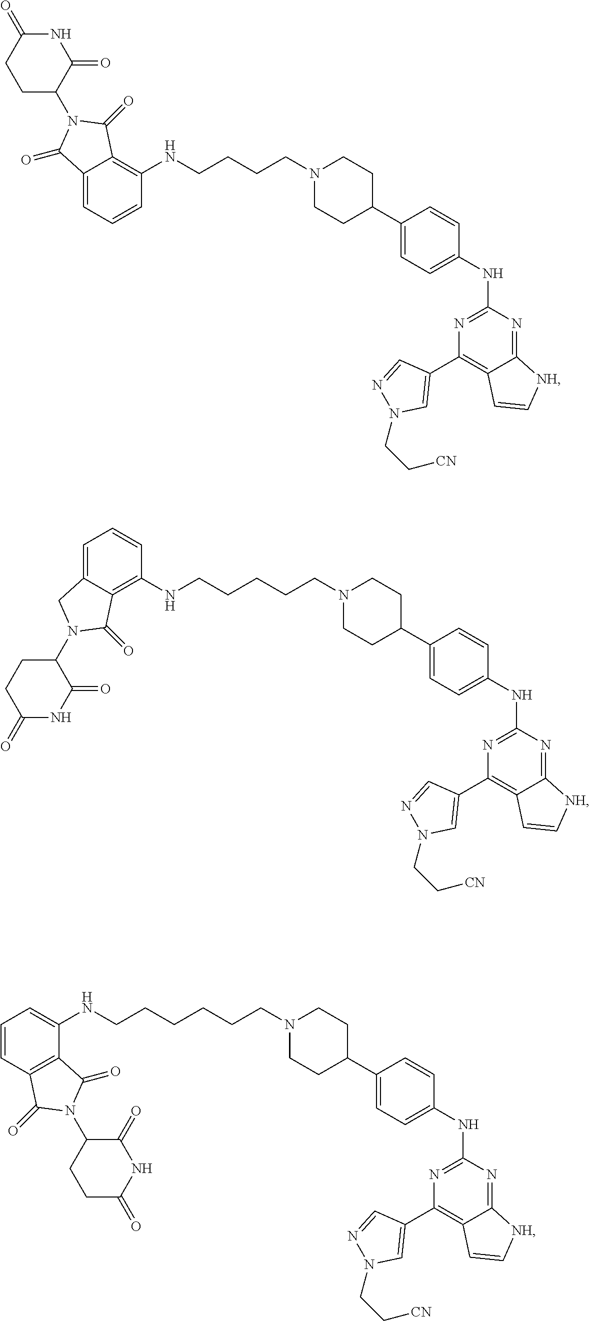 [SMILES: N#CCCn1cc(-c2nc(Nc3ccc(C4CCN(CCCCNc5cccc6c5C(=O)N(C5CCC(=O)NC5=O)C6=O)CC4)cc3)nc3[nH]ccc23)cn1 ; N#CCCn1cc(-c2nc(Nc3ccc(C4CCN(CCCCCNc5cccc6c5C(=O)N(C5CCC(=O)NC5=O)C6)CC4)cc3)nc3[nH]ccc23)cn1 ; N#CCCn1cc(-c2nc(Nc3ccc(C4CCN(CCCCCCNc5cccc6c5C(=O)N(C5CCC(=O)NC5=O)C6=O)CC4)cc3)nc3[nH]ccc23)cn1] 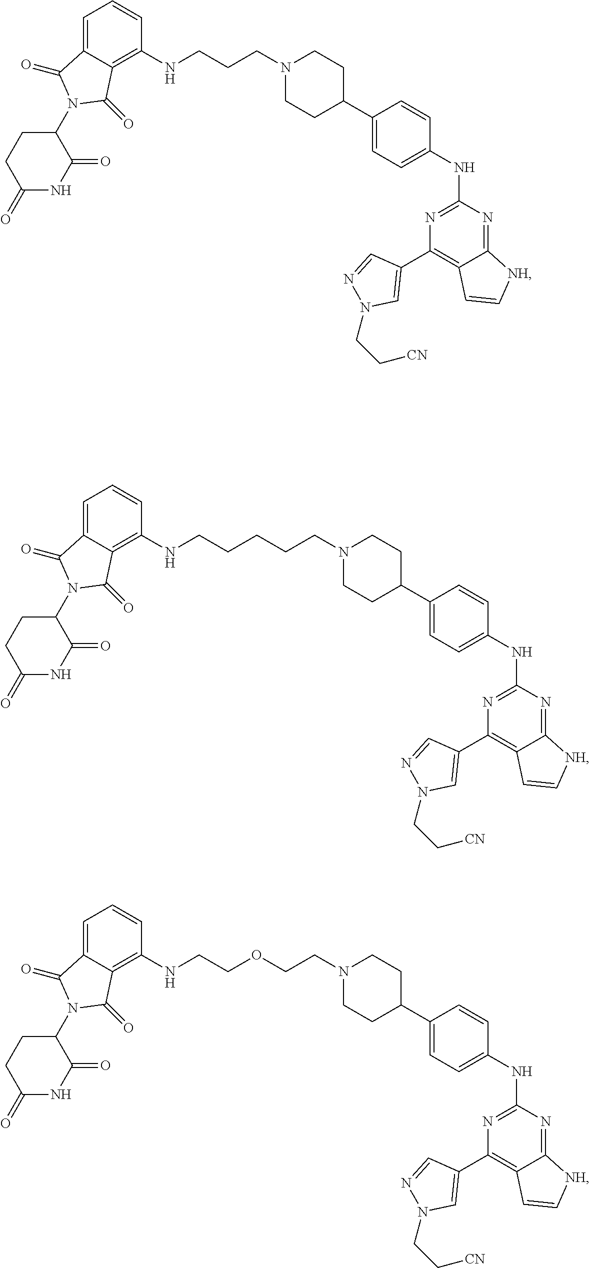 [SMILES: N#CCCn1cc(-c2nc(Nc3ccc(C4CCN(CCCNc5cccc6c5C(=O)N(C5CCC(=O)NC5=O)C6=O)CC4)cc3)nc3[nH]ccc23)cn1 ; N#CCCn1cc(-c2nc(Nc3ccc(C4CCN(CCCCCNc5cccc6c5C(=O)N(C5CCC(=O)NC5=O)C6=O)CC4)cc3)nc3[nH]ccc23)cn1 ; N#CCCn1cc(-c2nc(Nc3ccc(C4CCN(CCOCCNc5cccc6c5C(=O)N(C5CCC(=O)NC5=O)C6=O)CC4)cc3)nc3[nH]ccc23)cn1] 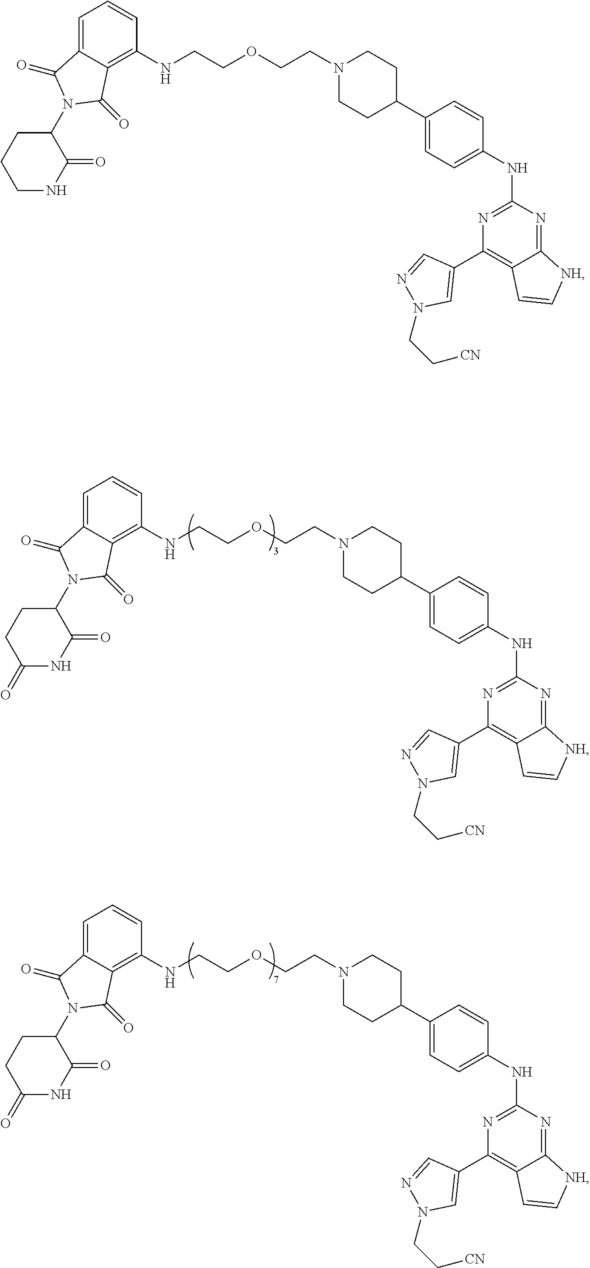 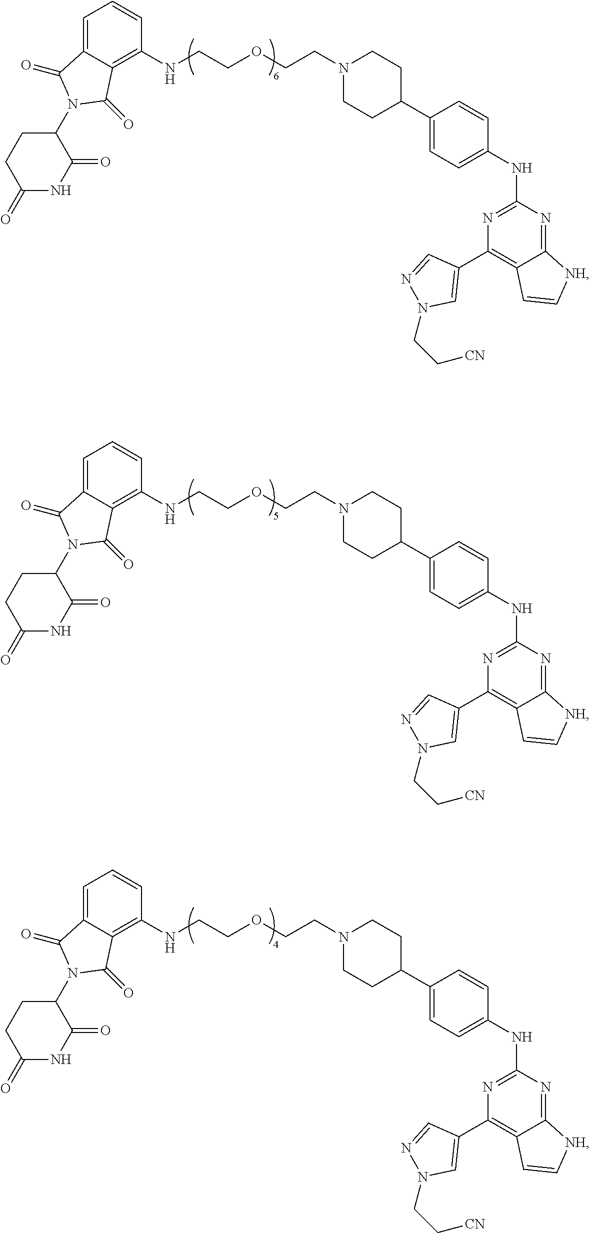 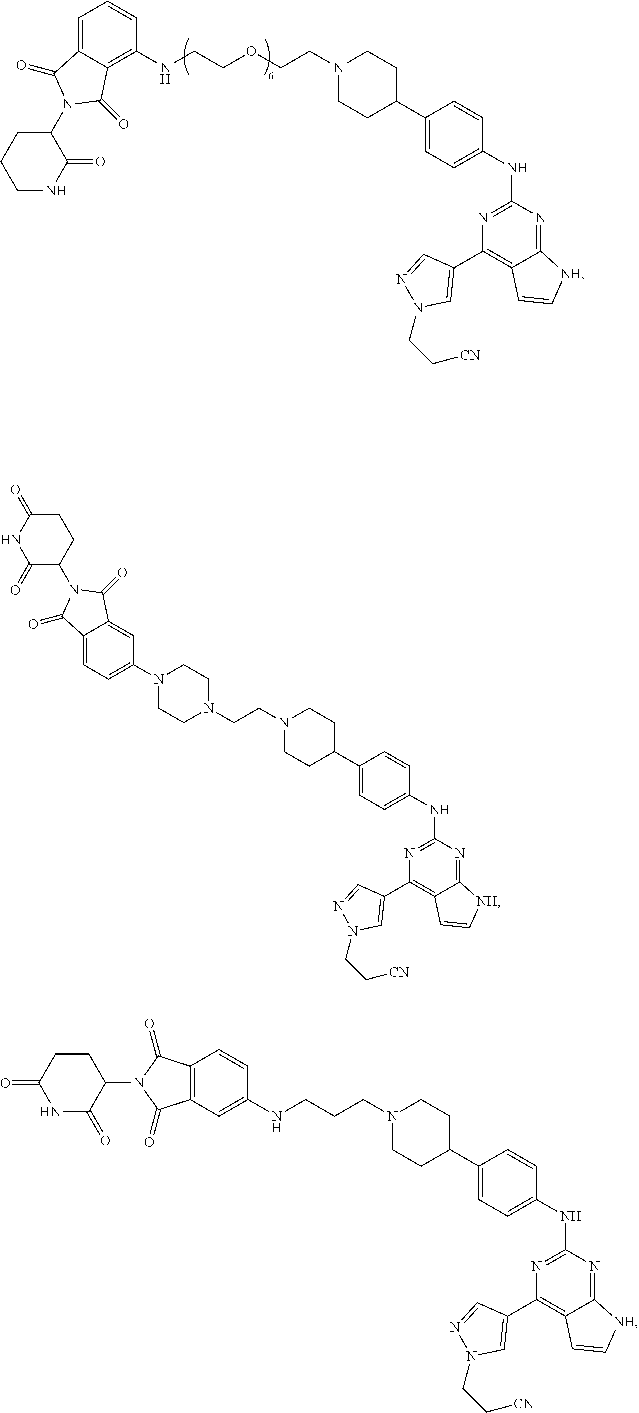 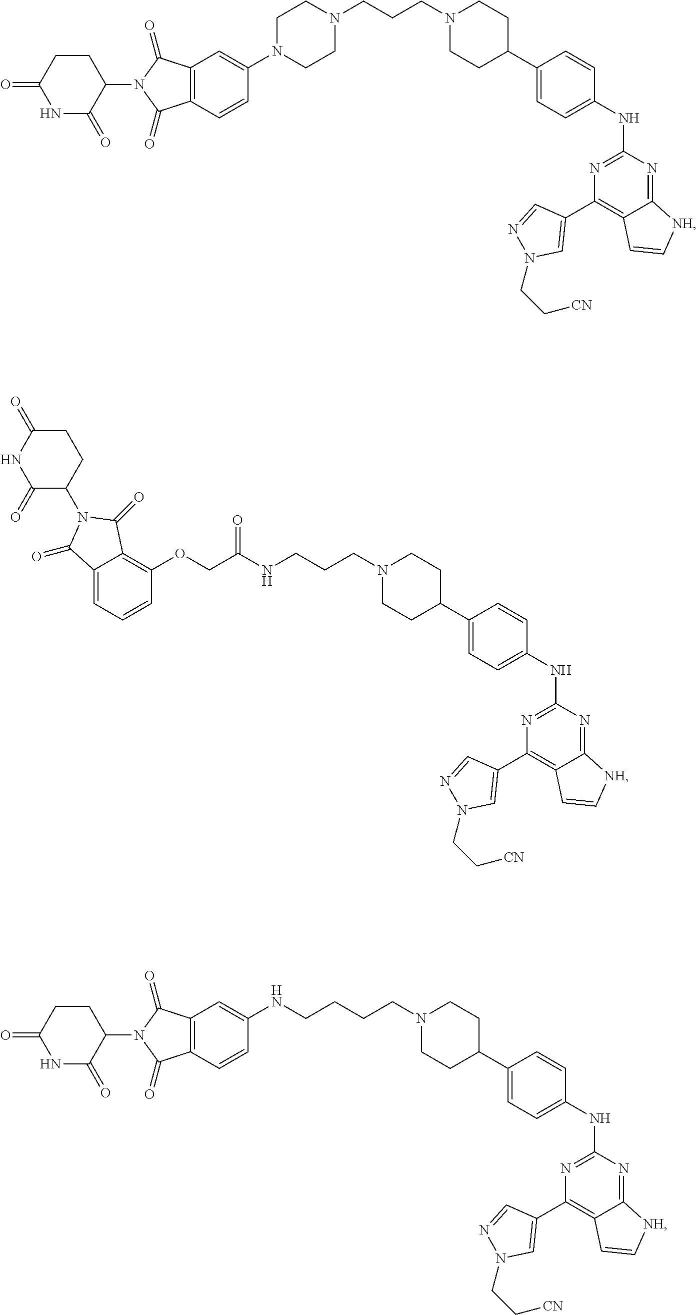 [SMILES: N#CCCn1cc(-c2nc(Nc3ccc(C4CCN(CCCN5CCN(c6ccc7c(c6)C(=O)N(C6CCC(=O)NC6=O)C7=O)CC5)CC4)cc3)nc3[nH]ccc23)cn1 ; N#CCCn1cc(-c2nc(Nc3ccc(C4CCN(CCCNC(=O)COc5cccc6c5C(=O)N(C5CCC(=O)NC5=O)C6=O)CC4)cc3)nc3[nH]ccc23)cn1 ; N#CCCn1cc(-c2nc(Nc3ccc(C4CCN(CCCCNc5ccc6c(c5)C(=O)N(C5CCC(=O)NC5=O)C6=O)CC4)cc3)nc3[nH]ccc23)cn1] 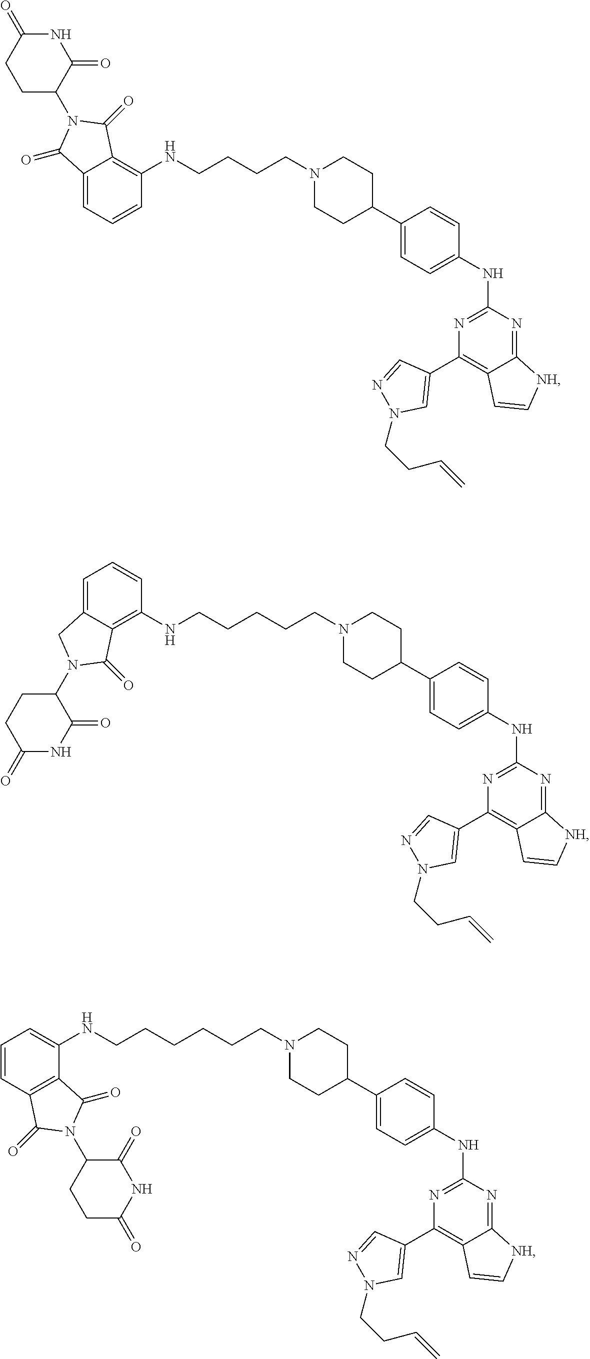 [SMILES: C=CCCn1cc(-c2nc(Nc3ccc(C4CCN(CCCCNc5cccc6c5C(=O)N(C5CCC(=O)NC5=O)C6=O)CC4)cc3)nc3[nH]ccc23)cn1 ; C=CCCn1cc(-c2nc(Nc3ccc(C4CCN(CCCCCNc5cccc6c5C(=O)N(C5CCC(=O)NC5=O)C6)CC4)cc3)nc3[nH]ccc23)cn1 ; C=CCCn1cc(-c2nc(Nc3ccc(C4CCN(CCCCCCNc5cccc6c5C(=O)N(C5CCC(=O)NC5=O)C6=O)CC4)cc3)nc3[nH]ccc23)cn1] 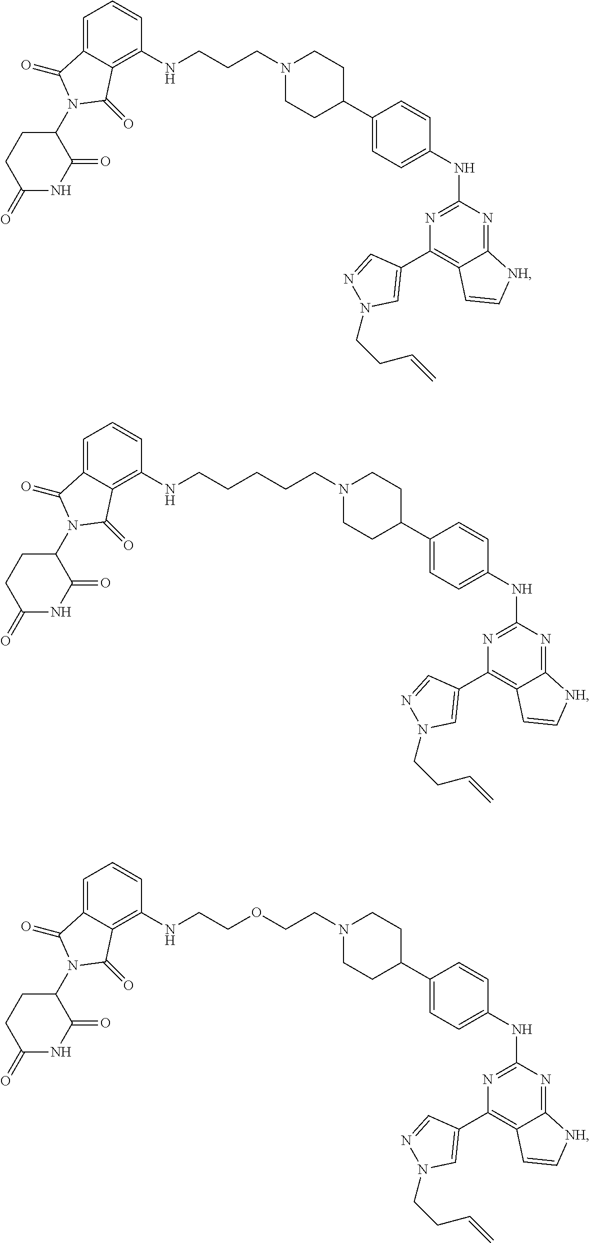 [SMILES: C=CCCn1cc(-c2nc(Nc3ccc(C4CCN(CCCNc5cccc6c5C(=O)N(C5CCC(=O)NC5=O)C6=O)CC4)cc3)nc3[nH]ccc23)cn1 ; C=CCCn1cc(-c2nc(Nc3ccc(C4CCN(CCCCCNc5cccc6c5C(=O)N(C5CCC(=O)NC5=O)C6=O)CC4)cc3)nc3[nH]ccc23)cn1 ; C=CCCn1cc(-c2nc(Nc3ccc(C4CCN(CCOCCNc5cccc6c5C(=O)N(C5CCC(=O)NC5=O)C6=O)CC4)cc3)nc3[nH]ccc23)cn1] 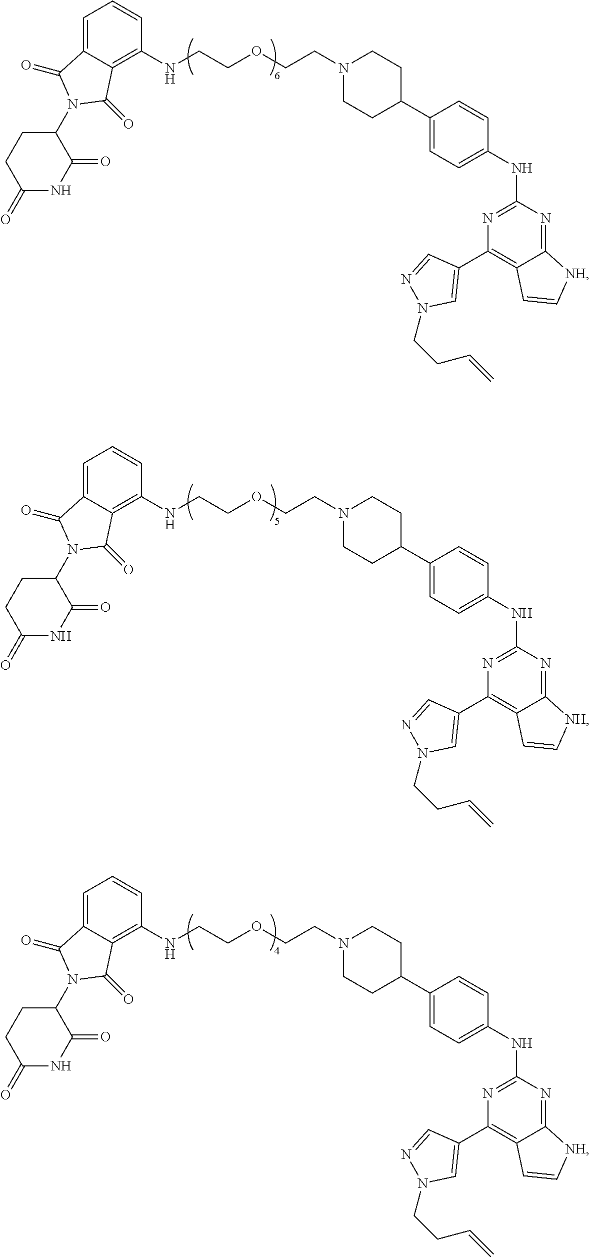 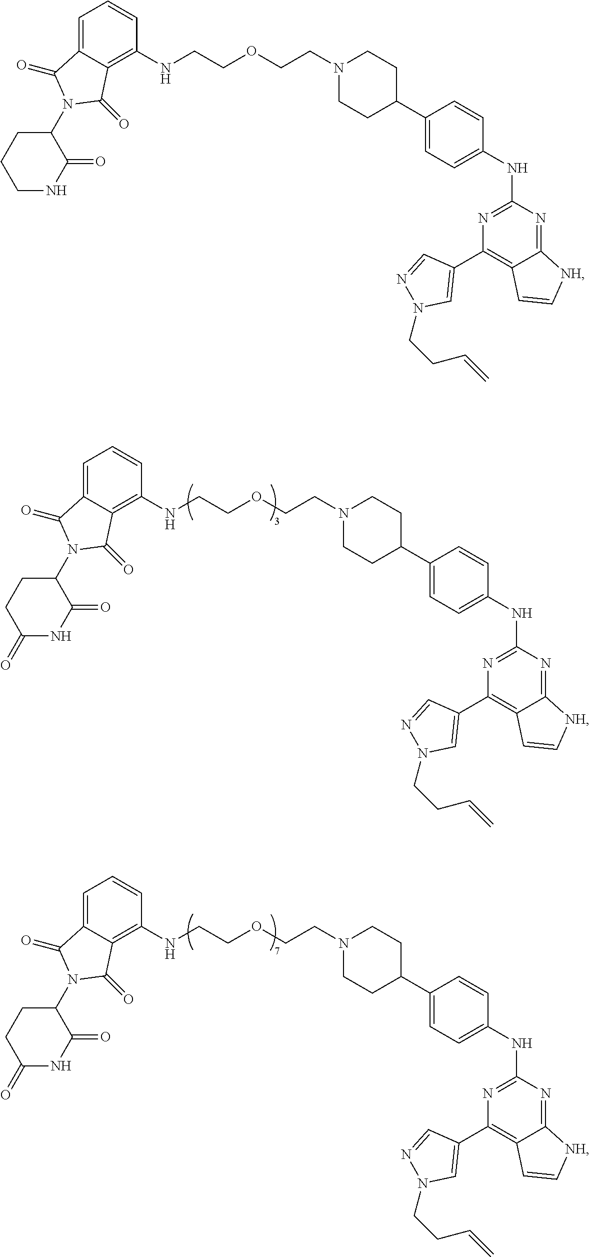 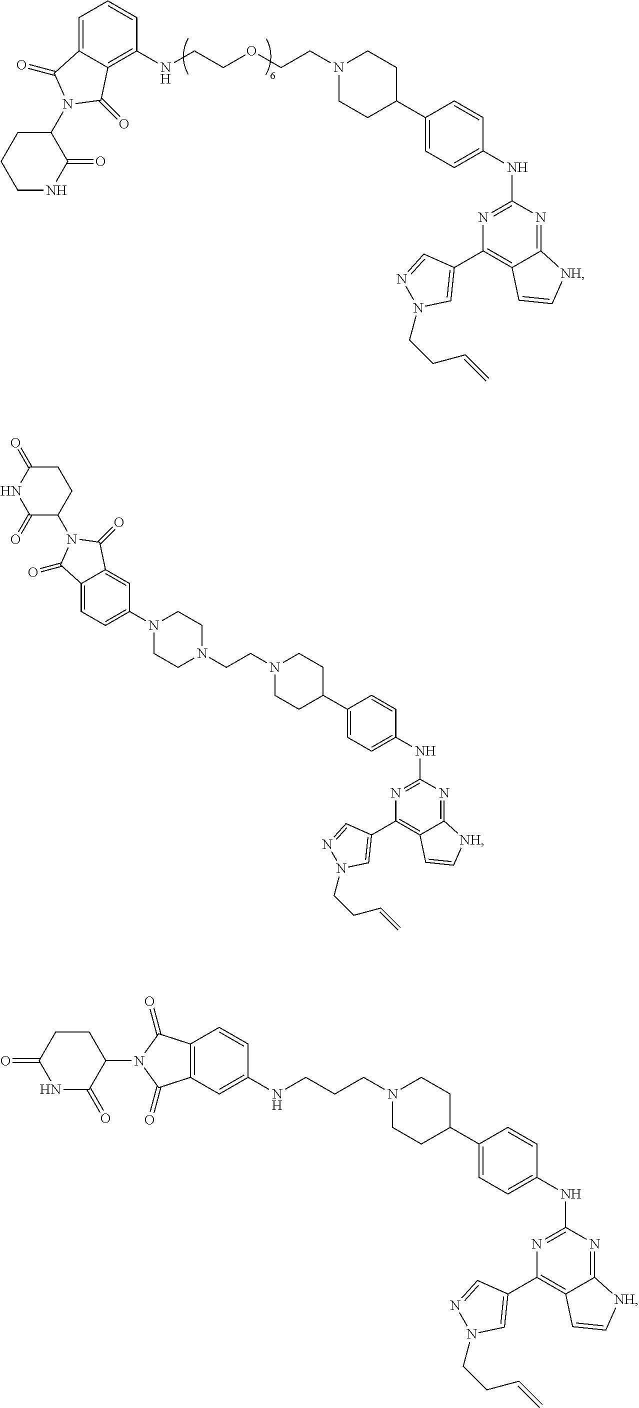 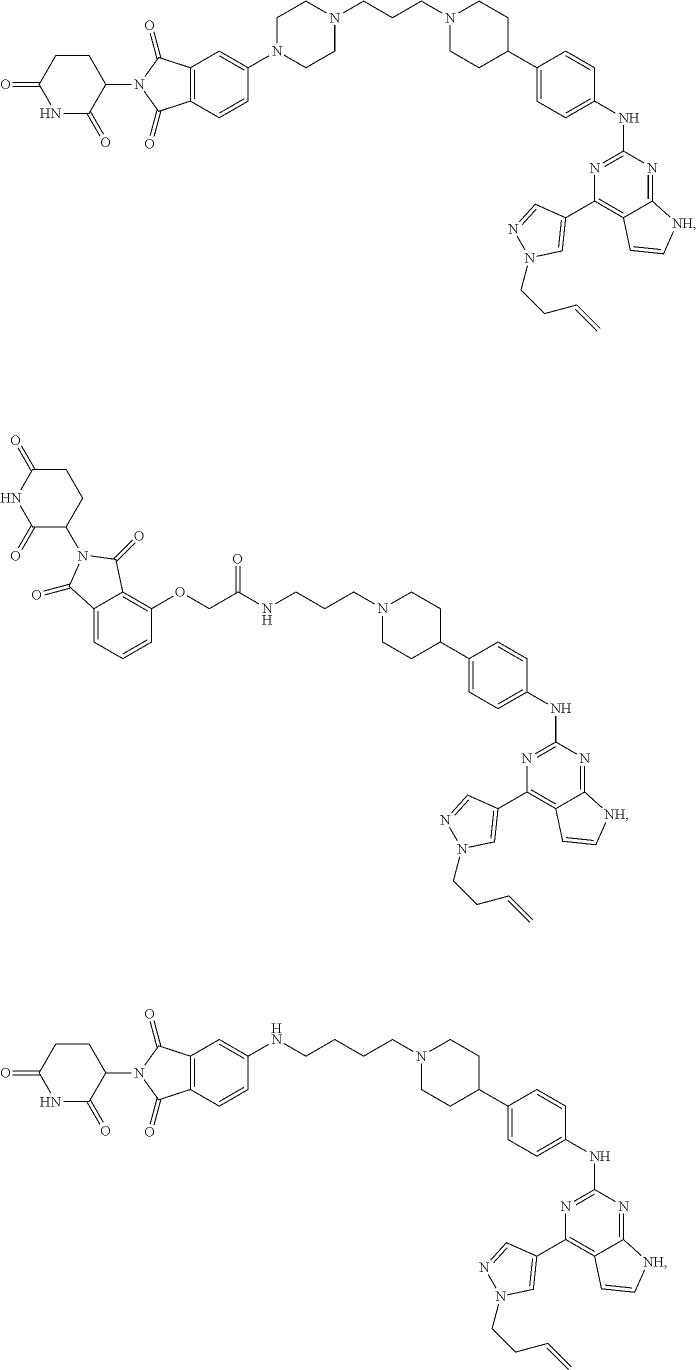 [SMILES: C=CCCn1cc(-c2nc(Nc3ccc(C4CCN(CCCN5CCN(c6ccc7c(c6)C(=O)N(C6CCC(=O)NC6=O)C7=O)CC5)CC4)cc3)nc3[nH]ccc23)cn1 ; C=CCCn1cc(-c2nc(Nc3ccc(C4CCN(CCCNC(=O)COc5cccc6c5C(=O)N(C5CCC(=O)NC5=O)C6=O)CC4)cc3)nc3[nH]ccc23)cn1 ; C=CCCn1cc(-c2nc(Nc3ccc(C4CCN(CCCCNc5ccc6c(c5)C(=O)N(C5CCC(=O)NC5=O)C6=O)CC4)cc3)nc3[nH]ccc23)cn1] 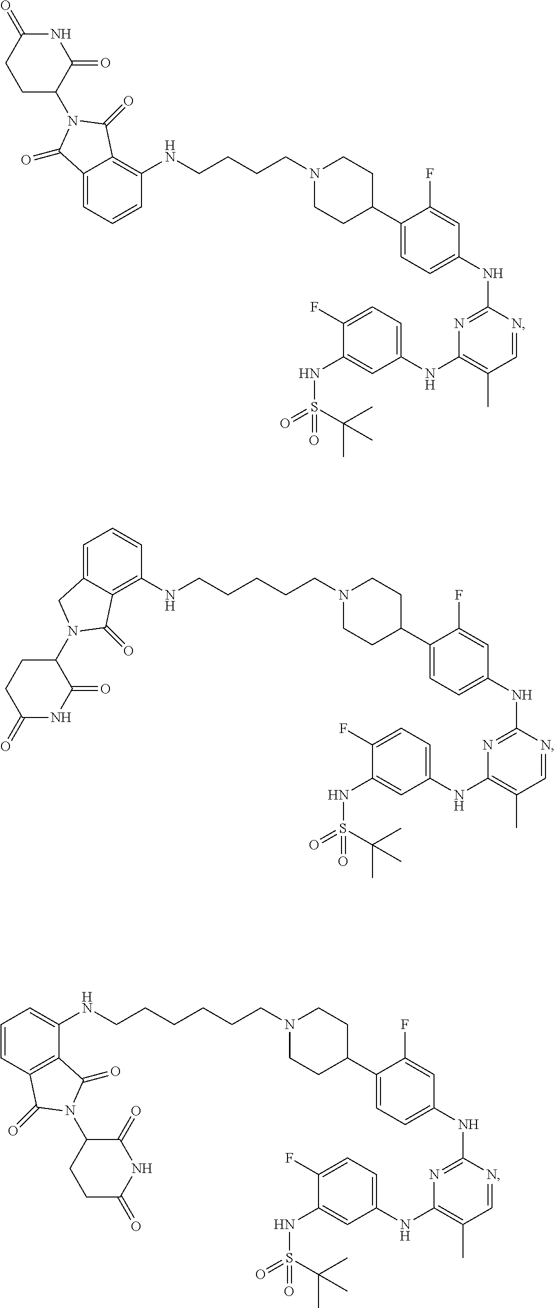 [SMILES: Cc1cnc(Nc2ccc(C3CCN(CCCCNc4cccc5c4C(=O)N(C4CCC(=O)NC4=O)C5=O)CC3)c(F)c2)nc1Nc1ccc(F)c(NS(=O)(=O)C(C)(C)C)c1 ; Cc1cnc(Nc2ccc(C3CCN(CCCCCNc4cccc5c4C(=O)N(C4CCC(=O)NC4=O)C5)CC3)c(F)c2)nc1Nc1ccc(F)c(NS(=O)(=O)C(C)(C)C)c1 ; Cc1cnc(Nc2ccc(C3CCN(CCCCCCNc4cccc5c4C(=O)N(C4CCC(=O)NC4=O)C5=O)CC3)c(F)c2)nc1Nc1ccc(F)c(NS(=O)(=O)C(C)(C)C)c1] 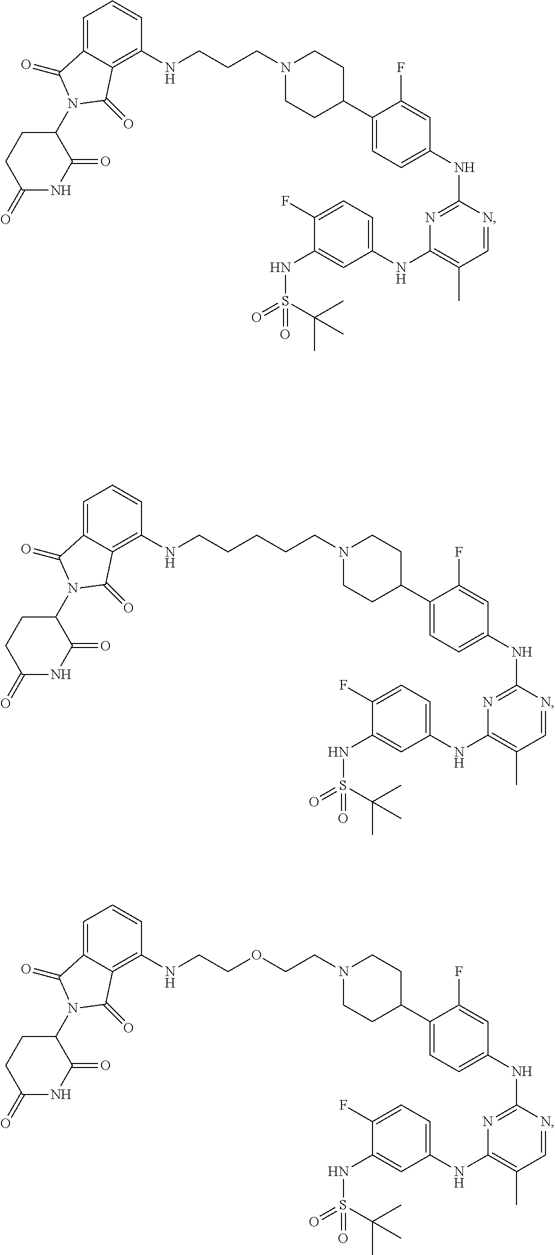 [SMILES: Cc1cnc(Nc2ccc(C3CCN(CCCNc4cccc5c4C(=O)N(C4CCC(=O)NC4=O)C5=O)CC3)c(F)c2)nc1Nc1ccc(F)c(NS(=O)(=O)C(C)(C)C)c1 ; Cc1cnc(Nc2ccc(C3CCN(CCCCCNc4cccc5c4C(=O)N(C4CCC(=O)NC4=O)C5=O)CC3)c(F)c2)nc1Nc1ccc(F)c(NS(=O)(=O)C(C)(C)C)c1 ; Cc1cnc(Nc2ccc(C3CCN(CCOCCNc4cccc5c4C(=O)N(C4CCC(=O)NC4=O)C5=O)CC3)c(F)c2)nc1Nc1ccc(F)c(NS(=O)(=O)C(C)(C)C)c1] 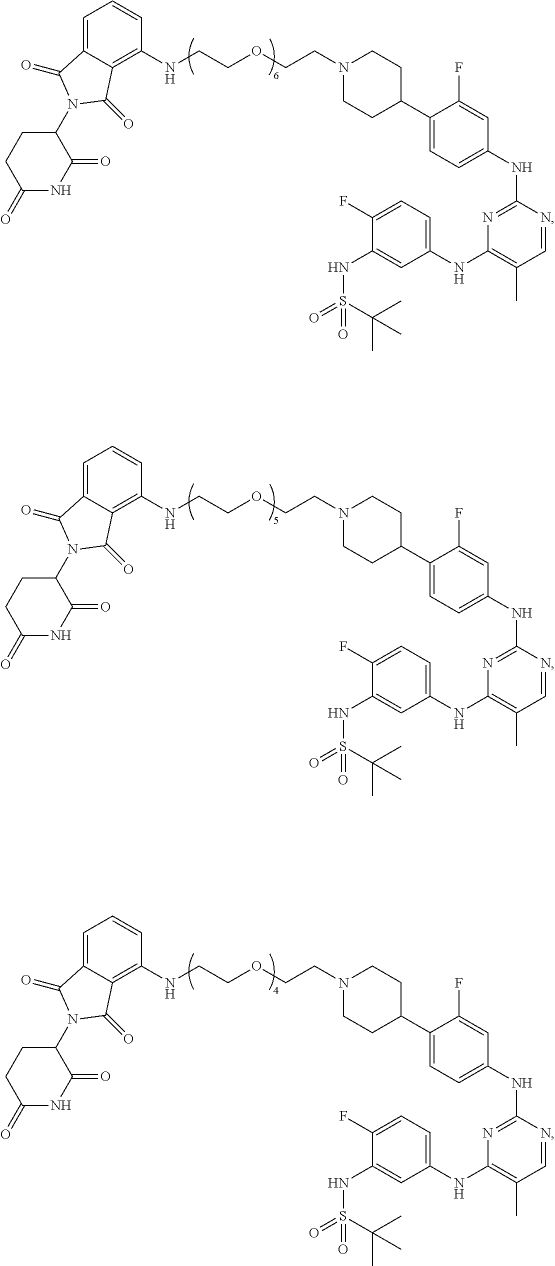 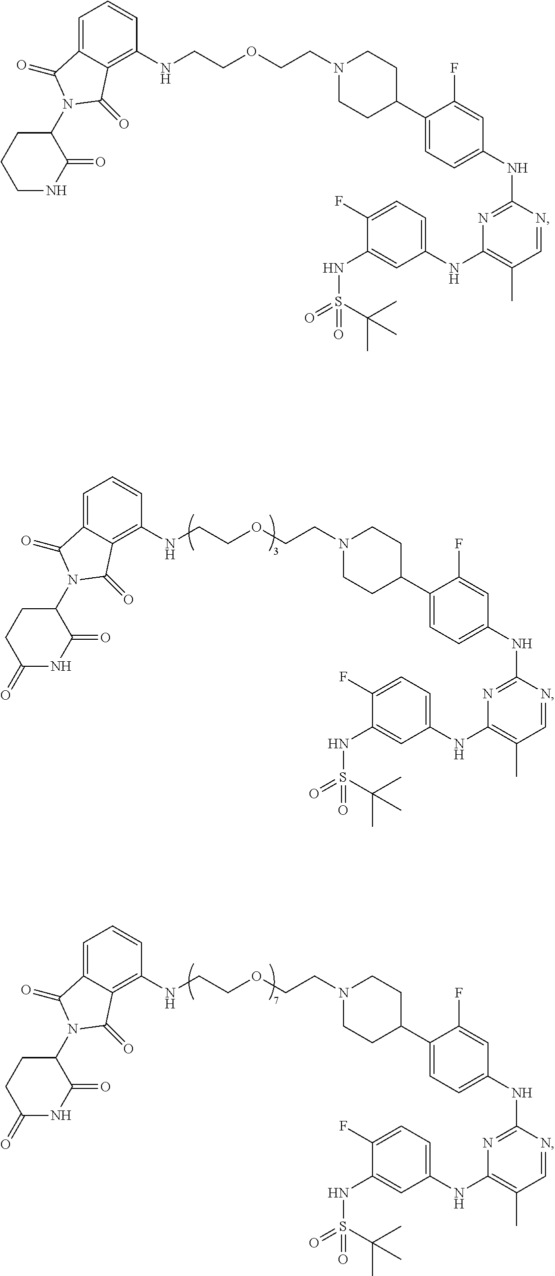 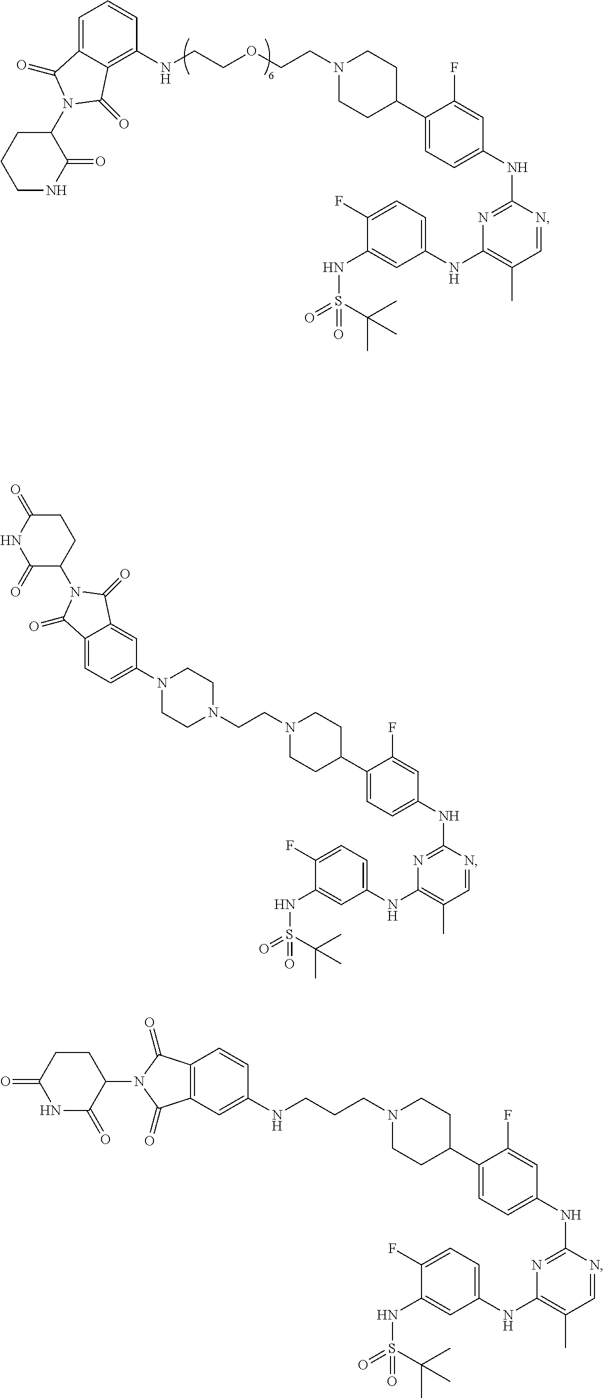 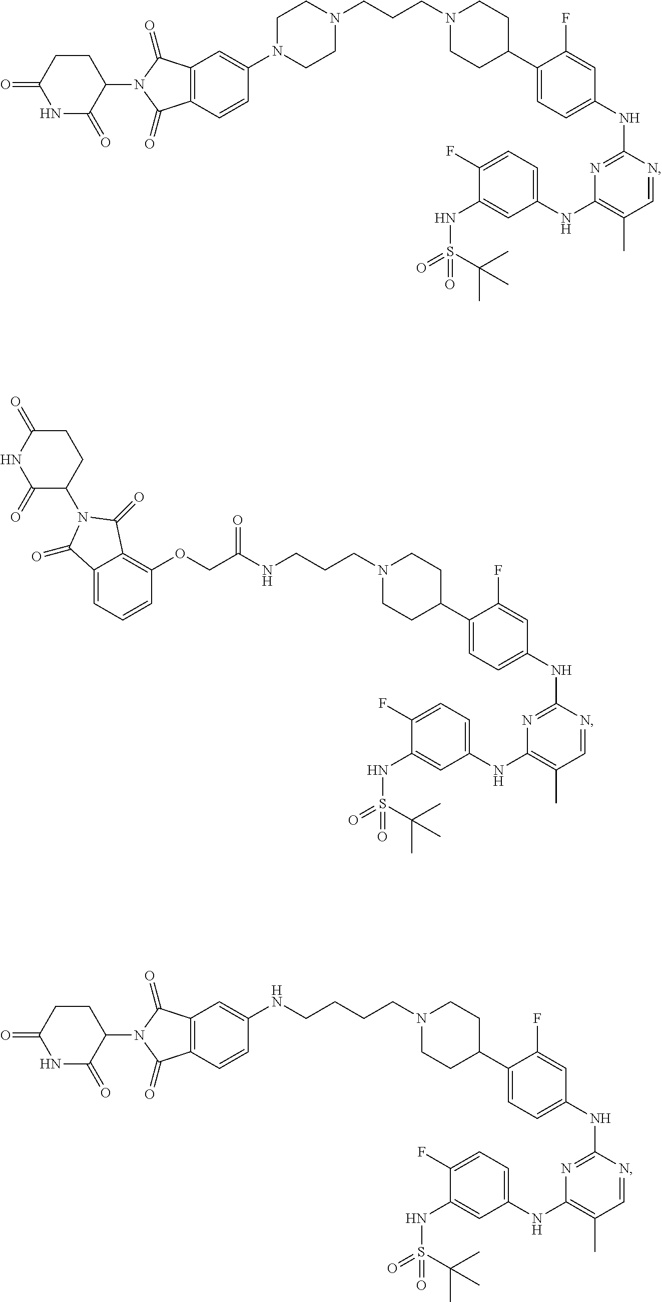 [SMILES: Cc1cnc(Nc2ccc(C3CCN(CCCN4CCN(c5ccc6c(c5)C(=O)N(C5CCC(=O)NC5=O)C6=O)CC4)CC3)c(F)c2)nc1Nc1ccc(F)c(NS(=O)(=O)C(C)(C)C)c1 ; Cc1cnc(Nc2ccc(C3CCN(CCCNC(=O)COc4cccc5c4C(=O)N(C4CCC(=O)NC4=O)C5=O)CC3)c(F)c2)nc1Nc1ccc(F)c(NS(=O)(=O)C(C)(C)C)c1 ; Cc1cnc(Nc2ccc(C3CCN(CCCCNc4ccc5c(c4)C(=O)N(C4CCC(=O)NC4=O)C5=O)CC3)c(F)c2)nc1Nc1ccc(F)c(NS(=O)(=O)C(C)(C)C)c1] 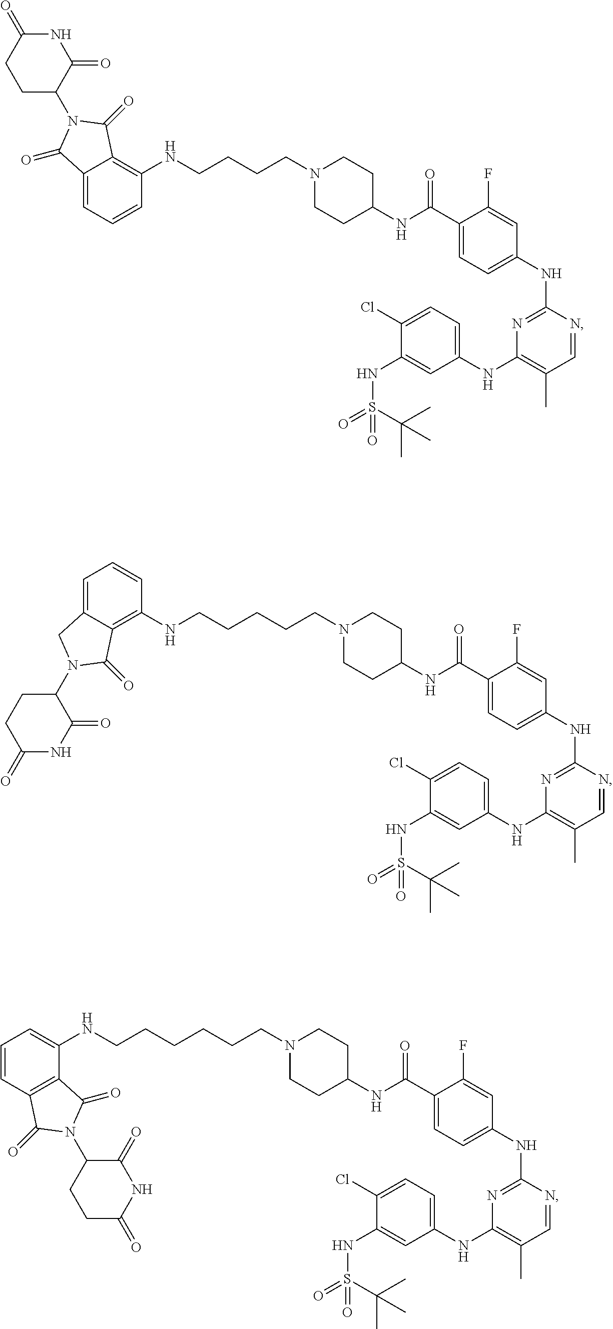 [SMILES: Cc1cnc(Nc2ccc(C(=O)NC3CCN(CCCCNc4cccc5c4C(=O)N(C4CCC(=O)NC4=O)C5=O)CC3)c(F)c2)nc1Nc1ccc(Cl)c(NS(=O)(=O)C(C)(C)C)c1 ; Cc1cnc(Nc2ccc(C(=O)NC3CCN(CCCCCNc4cccc5c4C(=O)N(C4CCC(=O)NC4=O)C5)CC3)c(F)c2)nc1Nc1ccc(Cl)c(NS(=O)(=O)C(C)(C)C)c1 ; Cc1cnc(Nc2ccc(C(=O)NC3CCN(CCCCCCNc4cccc5c4C(=O)N(C4CCC(=O)NC4=O)C5=O)CC3)c(F)c2)nc1Nc1ccc(Cl)c(NS(=O)(=O)C(C)(C)C)c1] 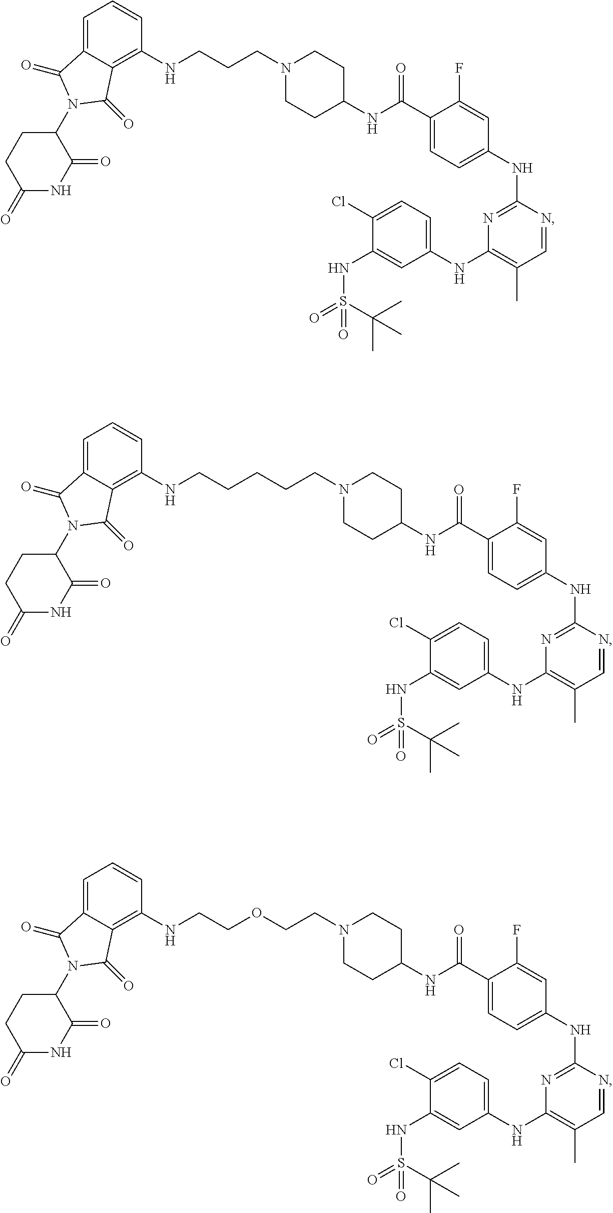 [SMILES: Cc1cnc(Nc2ccc(C(=O)NC3CCN(CCCNc4cccc5c4C(=O)N(C4CCC(=O)NC4=O)C5=O)CC3)c(F)c2)nc1Nc1ccc(Cl)c(NS(=O)(=O)C(C)(C)C)c1 ; Cc1cnc(Nc2ccc(C(=O)NC3CCN(CCCCCNc4cccc5c4C(=O)N(C4CCC(=O)NC4=O)C5=O)CC3)c(F)c2)nc1Nc1ccc(Cl)c(NS(=O)(=O)C(C)(C)C)c1 ; Cc1cnc(Nc2ccc(C(=O)NC3CCN(CCOCCNc4cccc5c4C(=O)N(C4CCC(=O)NC4=O)C5=O)CC3)c(F)c2)nc1Nc1ccc(Cl)c(NS(=O)(=O)C(C)(C)C)c1] 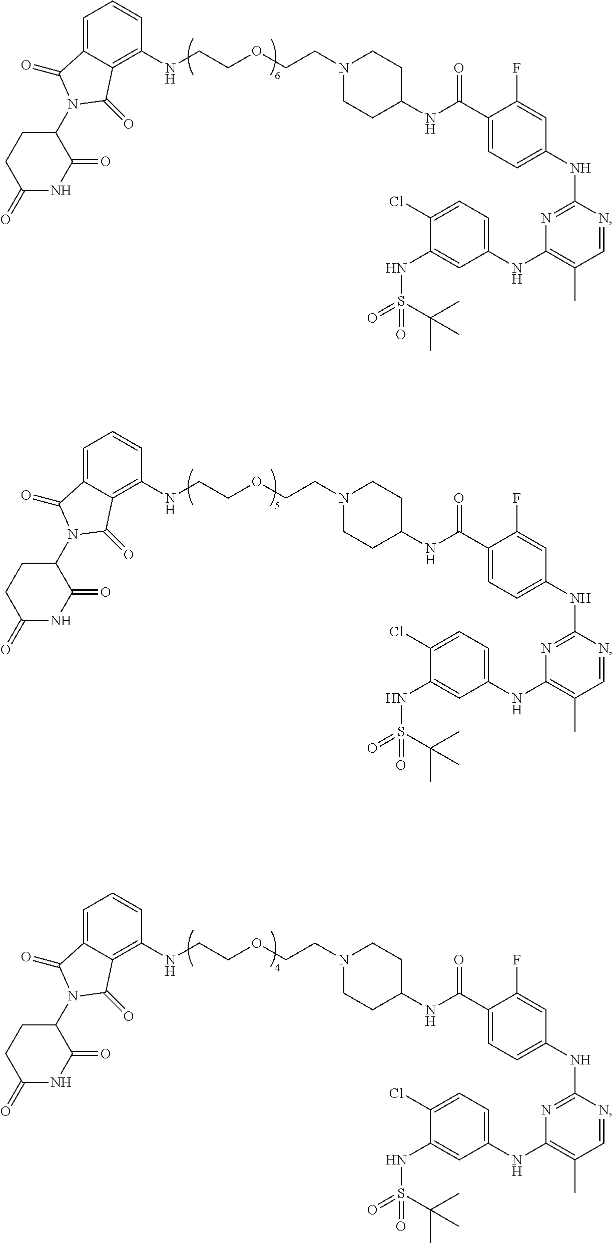 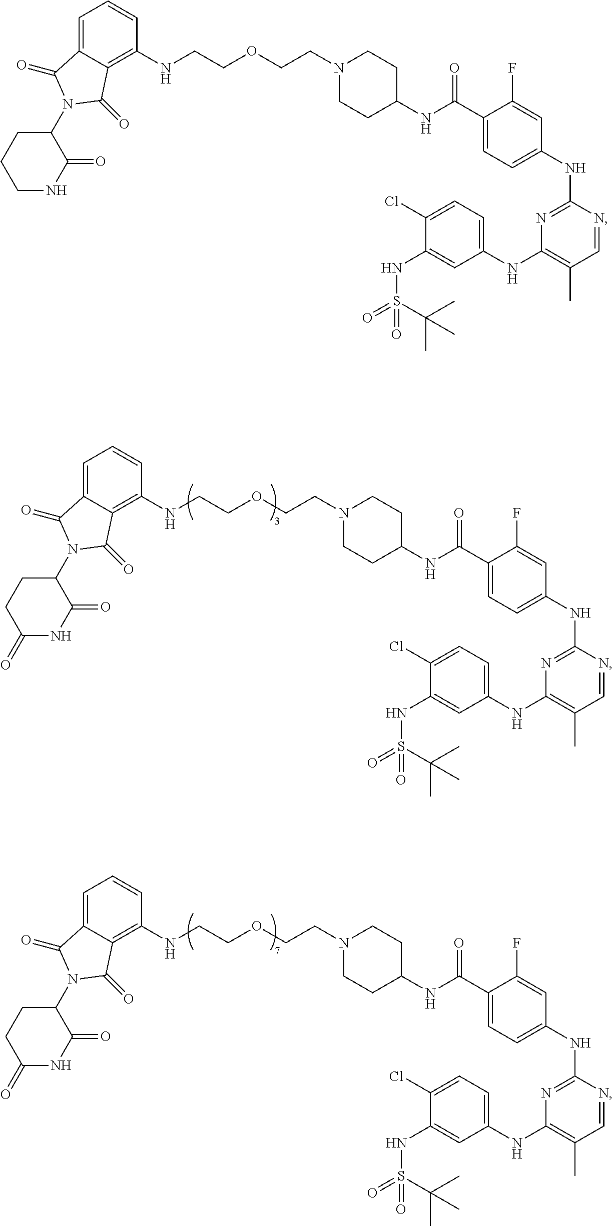 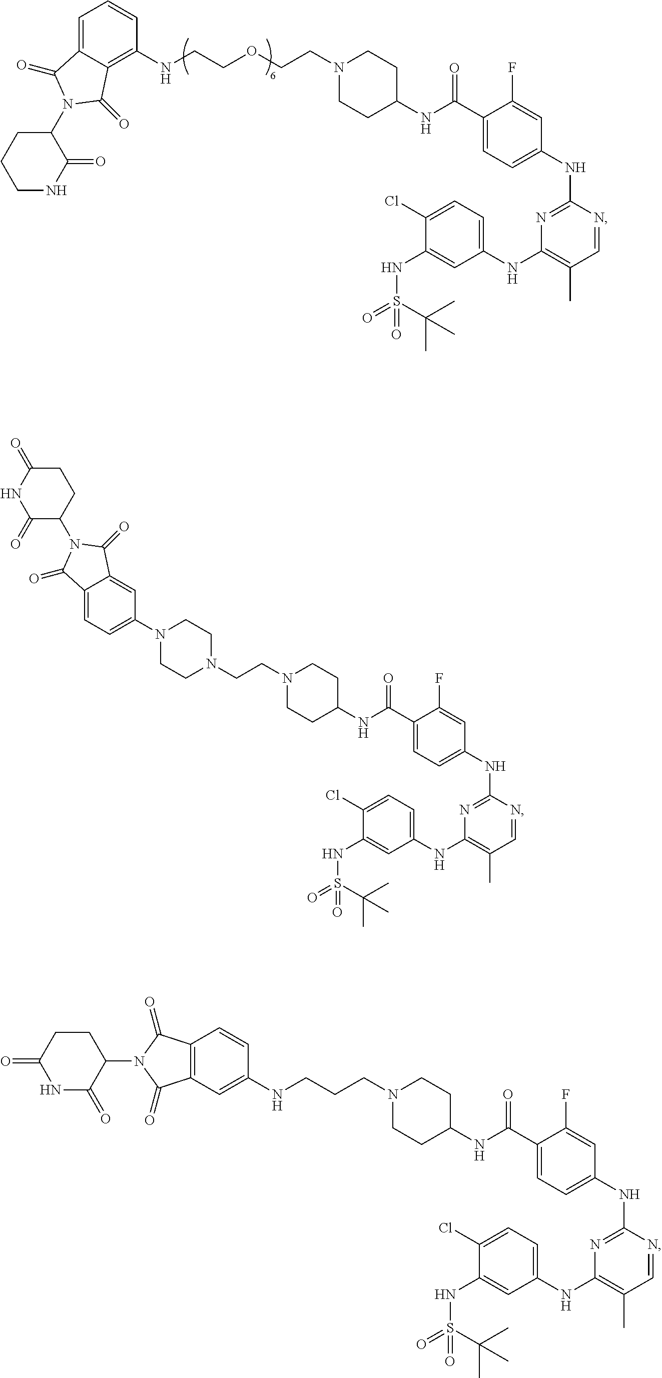 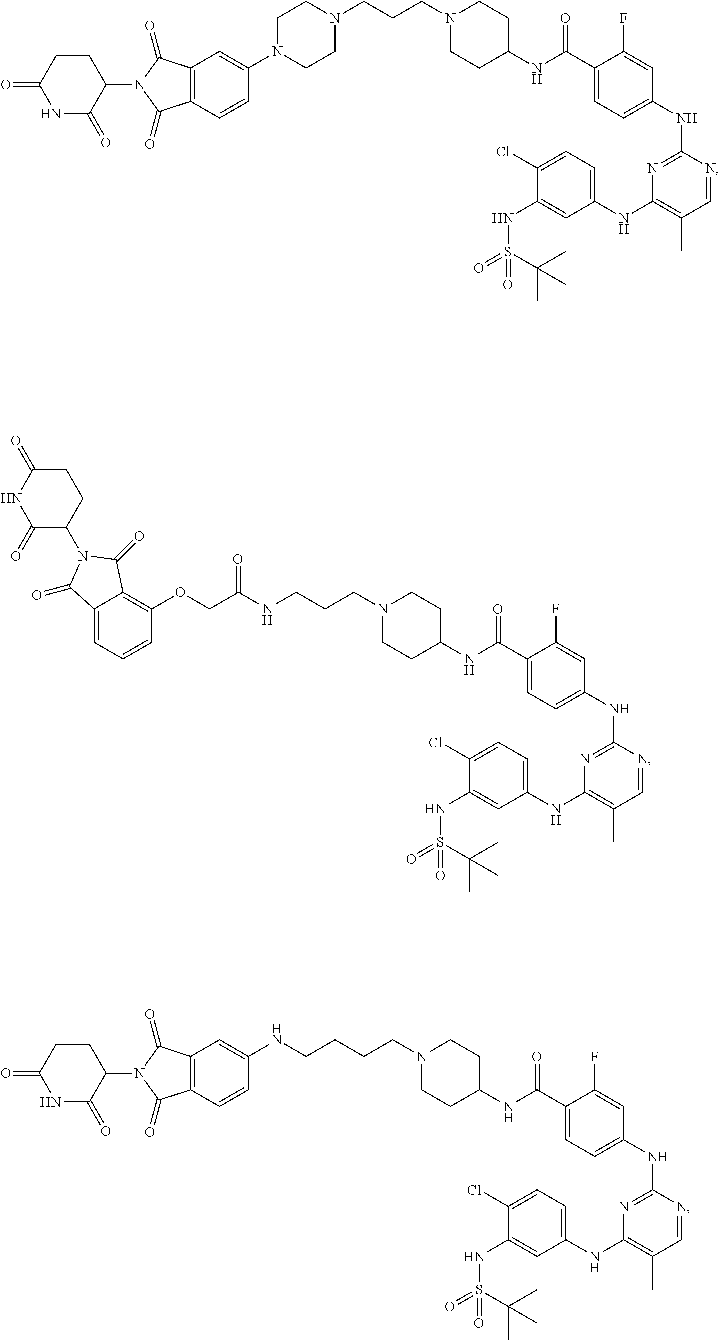 [SMILES: Cc1cnc(Nc2ccc(C(=O)NC3CCN(CCCN4CCN(c5ccc6c(c5)C(=O)N(C5CCC(=O)NC5=O)C6=O)CC4)CC3)c(F)c2)nc1Nc1ccc(Cl)c(NS(=O)(=O)C(C)(C)C)c1 ; Cc1cnc(Nc2ccc(C(=O)NC3CCN(CCCNC(=O)COc4cccc5c4C(=O)N(C4CCC(=O)NC4=O)C5=O)CC3)c(F)c2)nc1Nc1ccc(Cl)c(NS(=O)(=O)C(C)(C)C)c1 ; Cc1cnc(Nc2ccc(C(=O)NC3CCN(CCCCNc4ccc5c(c4)C(=O)N(C4CCC(=O)NC4=O)C5=O)CC3)c(F)c2)nc1Nc1ccc(Cl)c(NS(=O)(=O)C(C)(C)C)c1] 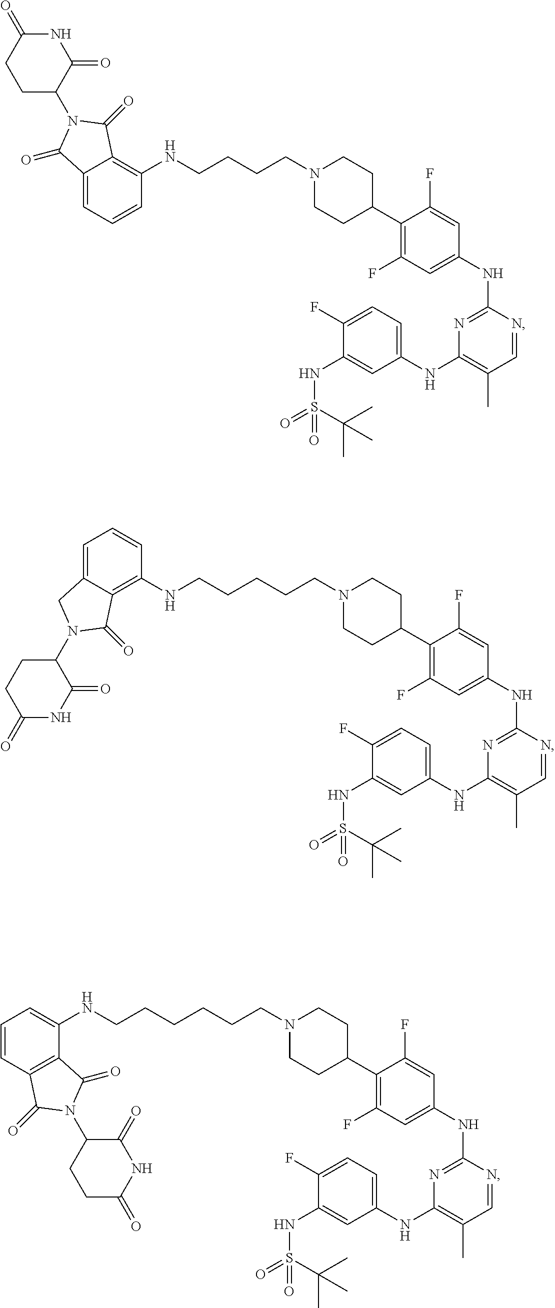 [SMILES: Cc1cnc(Nc2cc(F)c(C3CCN(CCCCNc4cccc5c4C(=O)N(C4CCC(=O)NC4=O)C5=O)CC3)c(F)c2)nc1Nc1ccc(F)c(NS(=O)(=O)C(C)(C)C)c1 ; Cc1cnc(Nc2cc(F)c(C3CCN(CCCCCNc4cccc5c4C(=O)N(C4CCC(=O)NC4=O)C5)CC3)c(F)c2)nc1Nc1ccc(F)c(NS(=O)(=O)C(C)(C)C)c1 ; Cc1cnc(Nc2cc(F)c(C3CCN(CCCCCCNc4cccc5c4C(=O)N(C4CCC(=O)NC4=O)C5=O)CC3)c(F)c2)nc1Nc1ccc(F)c(NS(=O)(=O)C(C)(C)C)c1] 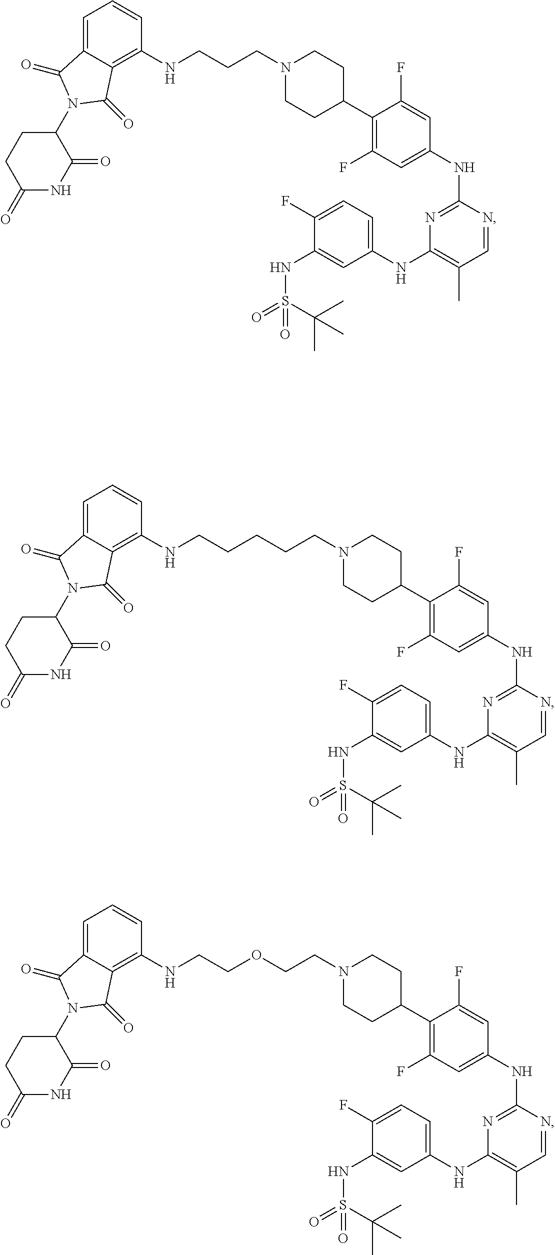 [SMILES: Cc1cnc(Nc2cc(F)c(C3CCN(CCCNc4cccc5c4C(=O)N(C4CCC(=O)NC4=O)C5=O)CC3)c(F)c2)nc1Nc1ccc(F)c(NS(=O)(=O)C(C)(C)C)c1 ; Cc1cnc(Nc2cc(F)c(C3CCN(CCCCCNc4cccc5c4C(=O)N(C4CCC(=O)NC4=O)C5=O)CC3)c(F)c2)nc1Nc1ccc(F)c(NS(=O)(=O)C(C)(C)C)c1 ; Cc1cnc(Nc2cc(F)c(C3CCN(CCOCCNc4cccc5c4C(=O)N(C4CCC(=O)NC4=O)C5=O)CC3)c(F)c2)nc1Nc1ccc(F)c(NS(=O)(=O)C(C)(C)C)c1] 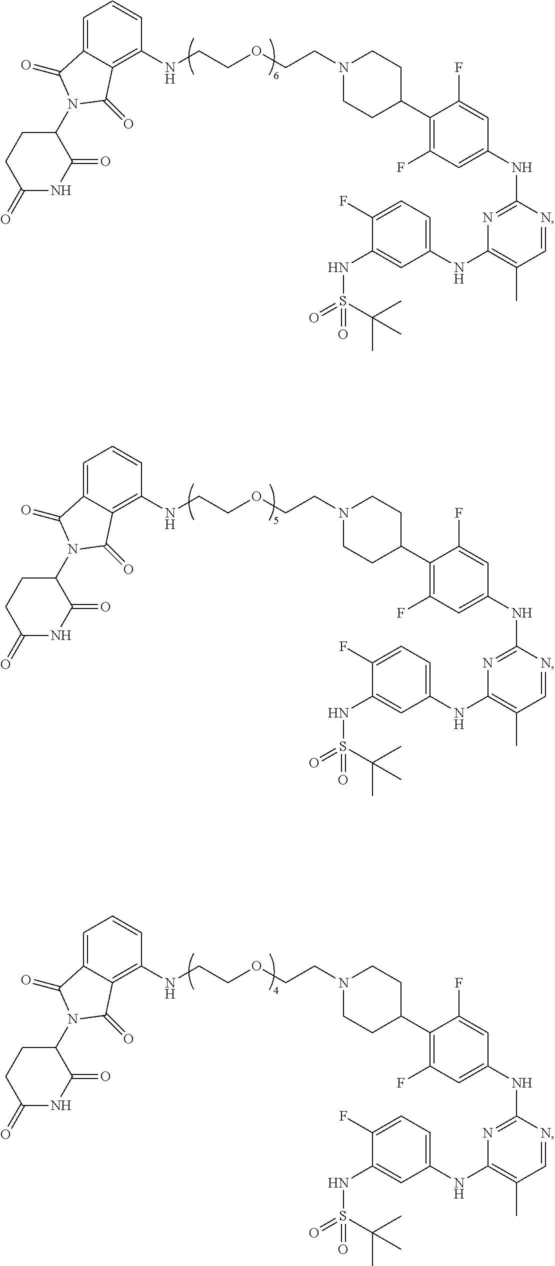 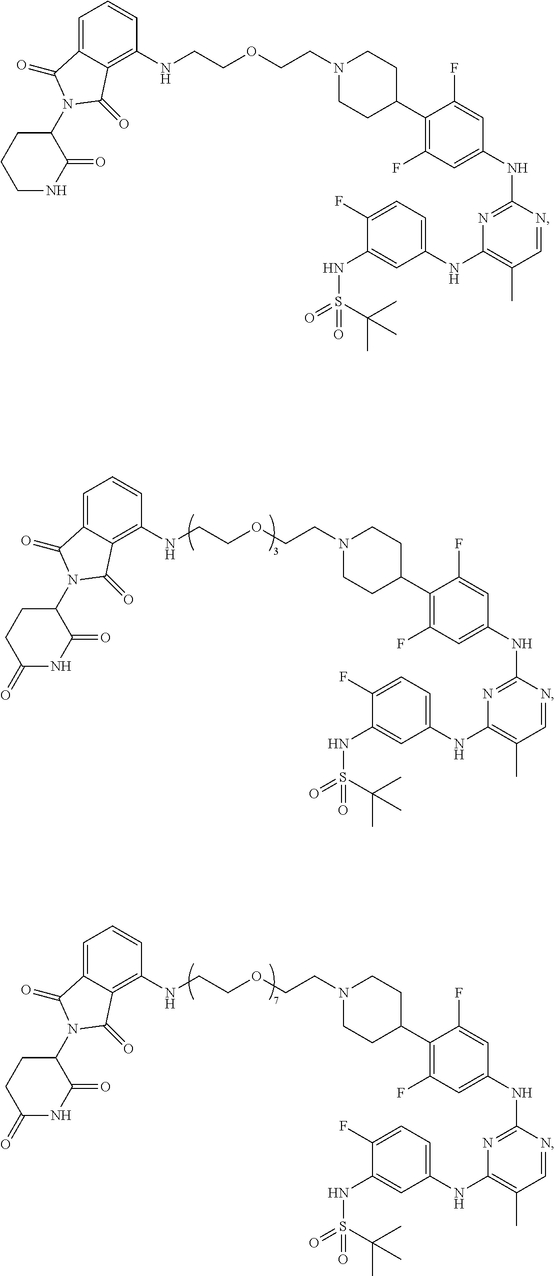 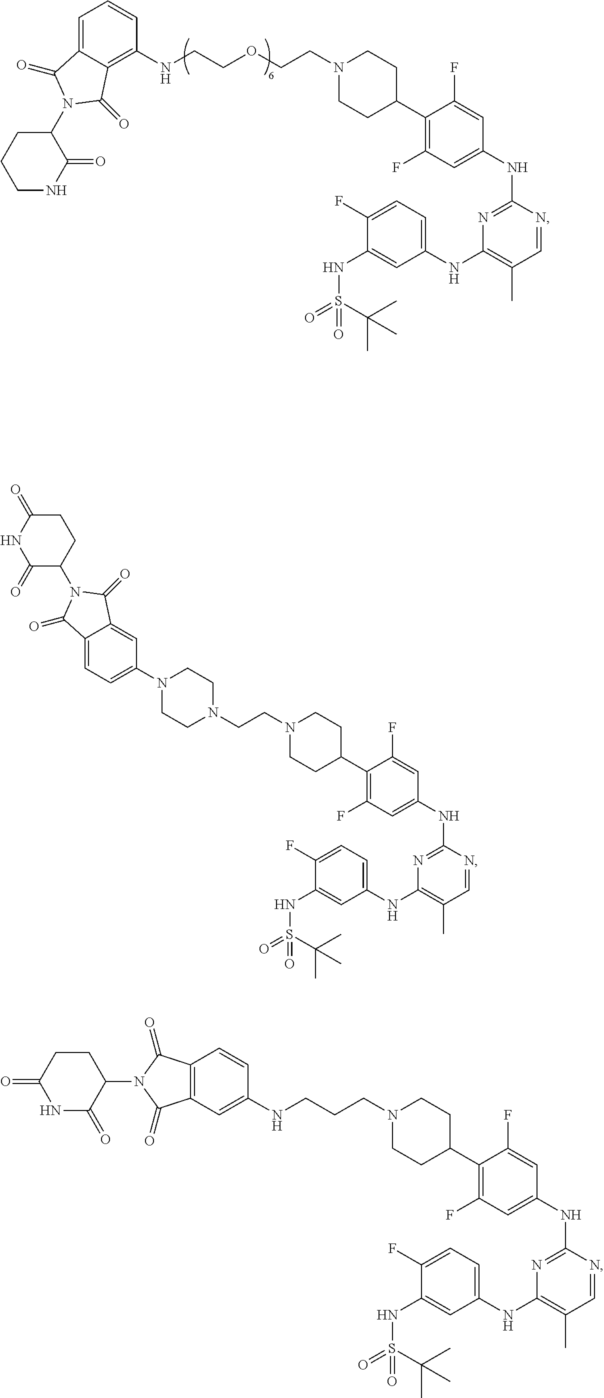 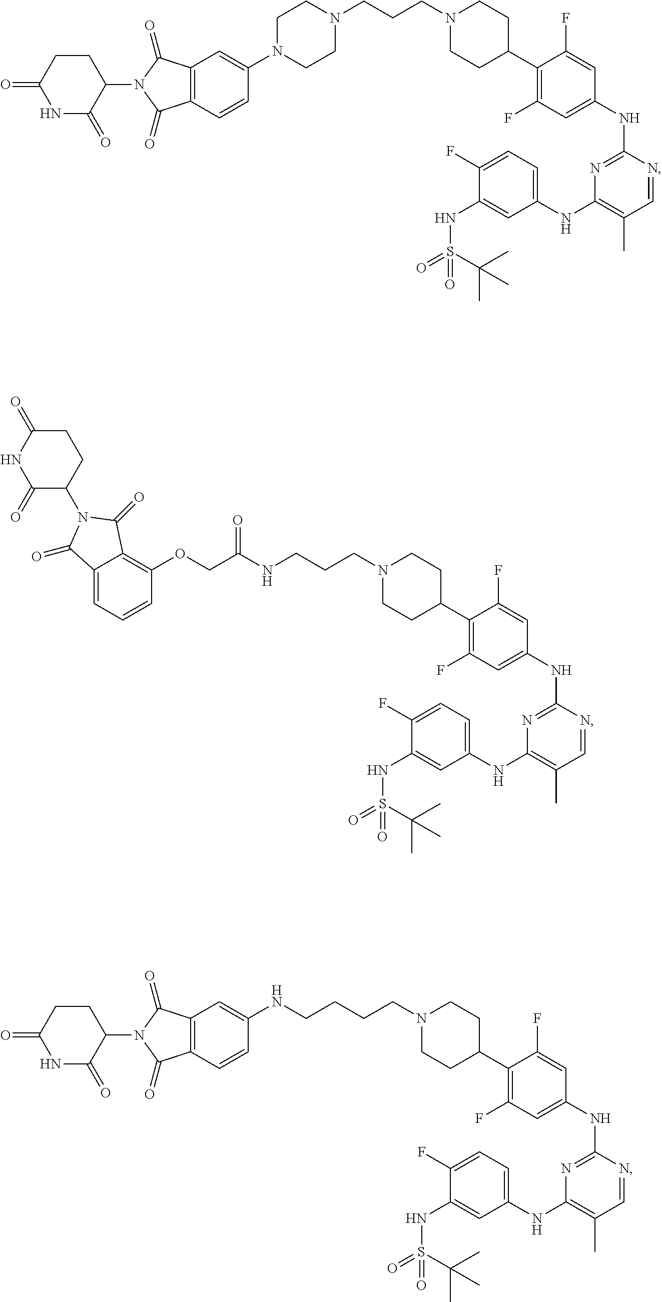 [SMILES: Cc1cnc(Nc2cc(F)c(C3CCN(CCCN4CCN(c5ccc6c(c5)C(=O)N(C5CCC(=O)NC5=O)C6=O)CC4)CC3)c(F)c2)nc1Nc1ccc(F)c(NS(=O)(=O)C(C)(C)C)c1 ; Cc1cnc(Nc2cc(F)c(C3CCN(CCCNC(=O)COc4cccc5c4C(=O)N(C4CCC(=O)NC4=O)C5=O)CC3)c(F)c2)nc1Nc1ccc(F)c(NS(=O)(=O)C(C)(C)C)c1 ; Cc1cnc(Nc2cc(F)c(C3CCN(CCCCNc4ccc5c(c4)C(=O)N(C4CCC(=O)NC4=O)C5=O)CC3)c(F)c2)nc1Nc1ccc(F)c(NS(=O)(=O)C(C)(C)C)c1] 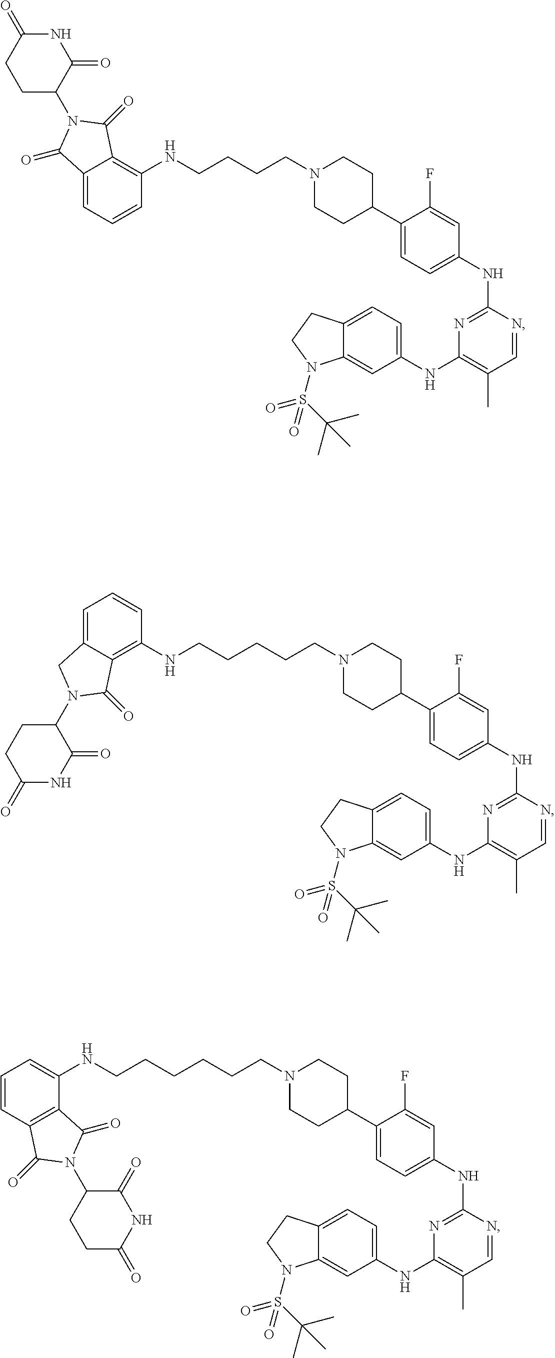 [SMILES: Cc1cnc(Nc2ccc(C3CCN(CCCCNc4cccc5c4C(=O)N(C4CCC(=O)NC4=O)C5=O)CC3)c(F)c2)nc1Nc1ccc2c(c1)N(S(=O)(=O)C(C)(C)C)CC2 ; Cc1cnc(Nc2ccc(C3CCN(CCCCCNc4cccc5c4C(=O)N(C4CCC(=O)NC4=O)C5)CC3)c(F)c2)nc1Nc1ccc2c(c1)N(S(=O)(=O)C(C)(C)C)CC2 ; Cc1cnc(Nc2ccc(C3CCN(CCCCCCNc4cccc5c4C(=O)N(C4CCC(=O)NC4=O)C5=O)CC3)c(F)c2)nc1Nc1ccc2c(c1)N(S(=O)(=O)C(C)(C)C)CC2] 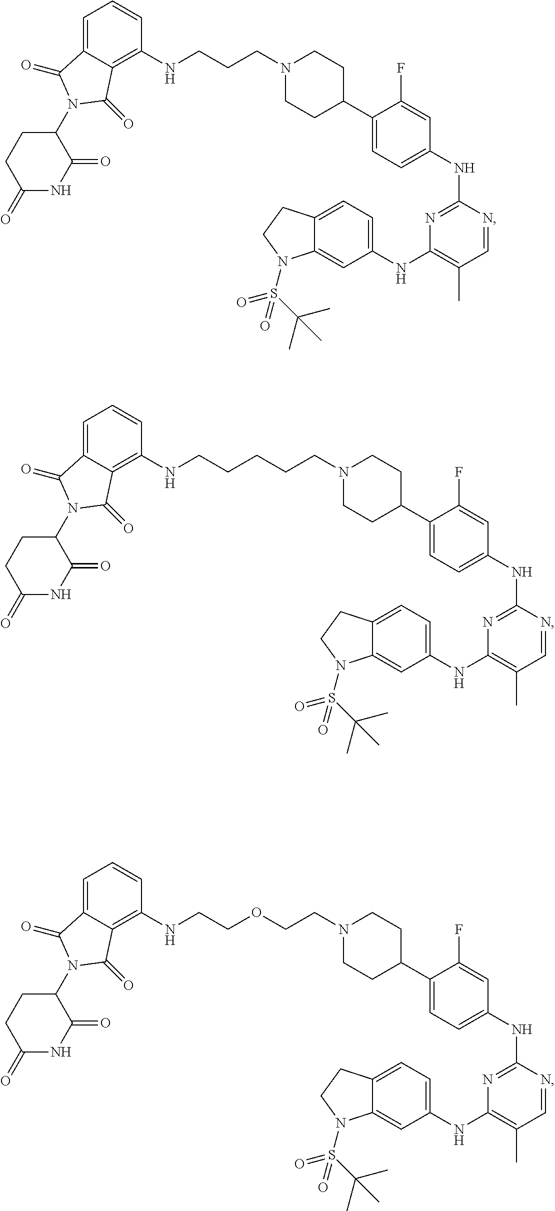 [SMILES: Cc1cnc(Nc2ccc(C3CCN(CCCNc4cccc5c4C(=O)N(C4CCC(=O)NC4=O)C5=O)CC3)c(F)c2)nc1Nc1ccc2c(c1)N(S(=O)(=O)C(C)(C)C)CC2 ; Cc1cnc(Nc2ccc(C3CCN(CCCCCNc4cccc5c4C(=O)N(C4CCC(=O)NC4=O)C5=O)CC3)c(F)c2)nc1Nc1ccc2c(c1)N(S(=O)(=O)C(C)(C)C)CC2 ; Cc1cnc(Nc2ccc(C3CCN(CCOCCNc4cccc5c4C(=O)N(C4CCC(=O)NC4=O)C5=O)CC3)c(F)c2)nc1Nc1ccc2c(c1)N(S(=O)(=O)C(C)(C)C)CC2] 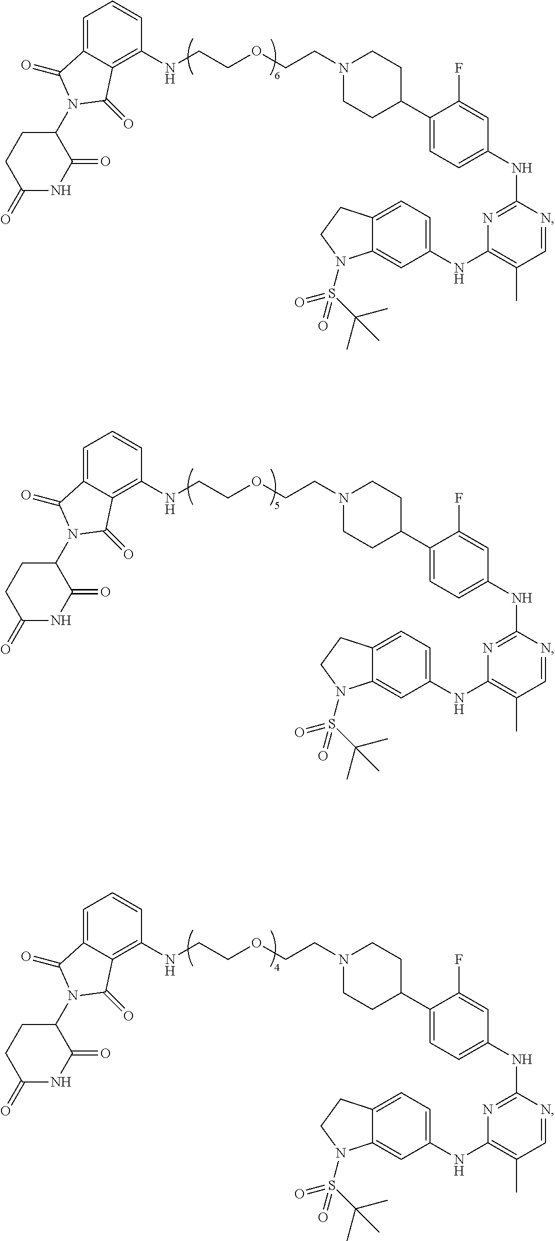 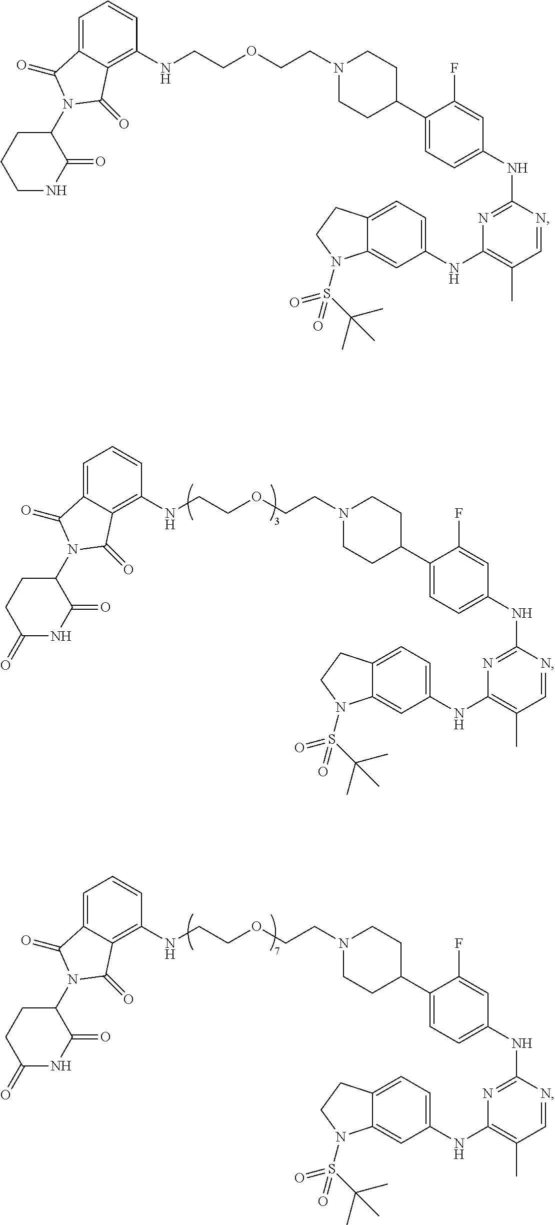 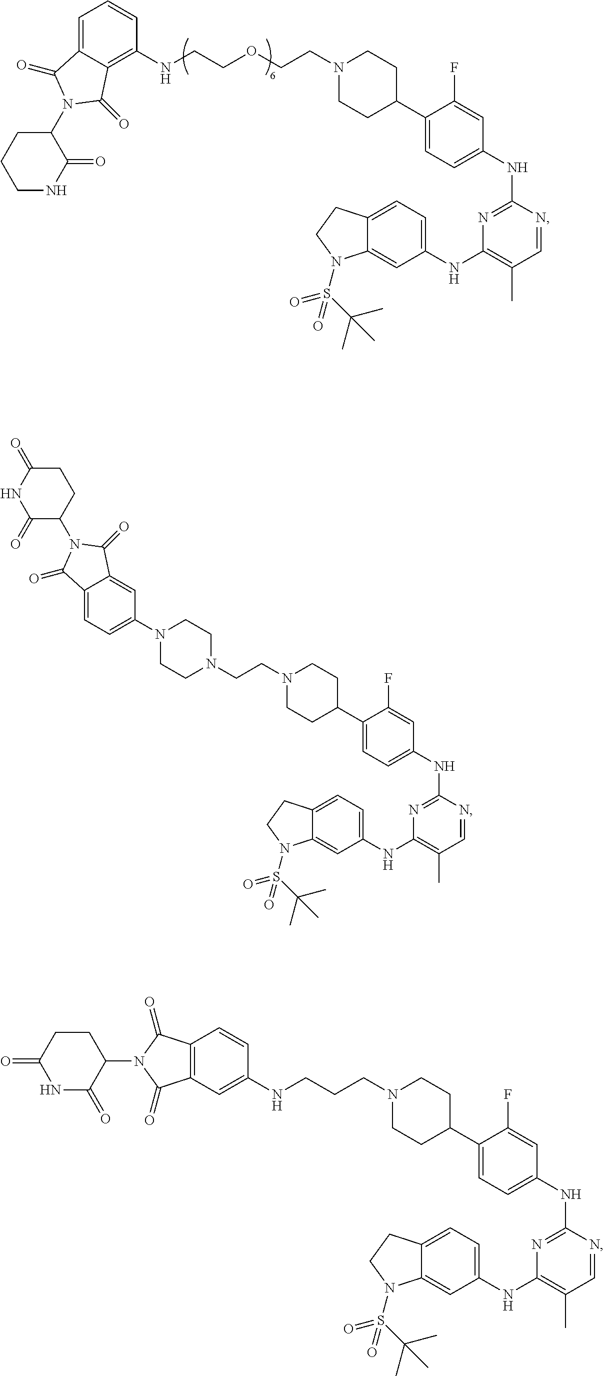 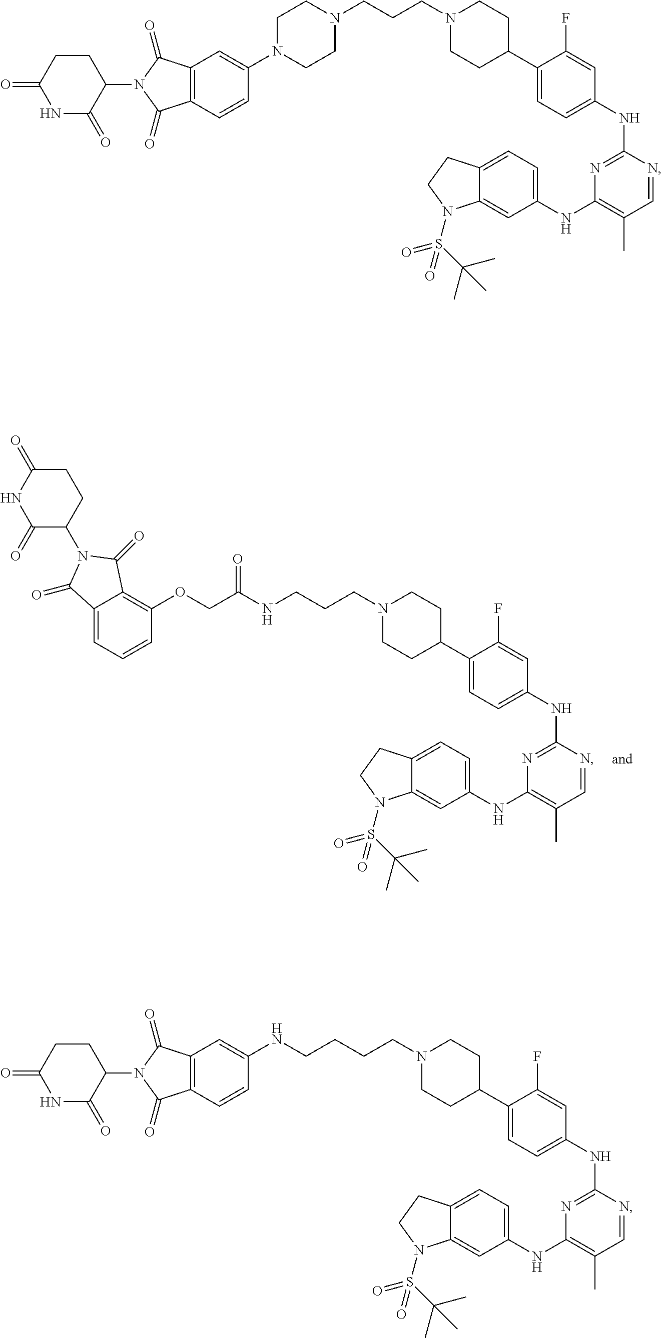 [SMILES: Cc1cnc(Nc2ccc(C3CCN(CCCN4CCN(c5ccc6c(c5)C(=O)N(C5CCC(=O)NC5=O)C6=O)CC4)CC3)c(F)c2)nc1Nc1ccc2c(c1)N(S(=O)(=O)C(C)(C)C)CC2 ; Cc1cnc(Nc2ccc(C3CCN(CCCNC(=O)COc4cccc5c4C(=O)N(C4CCC(=O)NC4=O)C5=O)CC3)c(F)c2)nc1Nc1ccc2c(c1)N(S(=O)(=O)C(C)(C)C)CC2 ; Cc1cnc(Nc2ccc(C3CCN(CCCCNc4ccc5c(c4)C(=O)N(C4CCC(=O)NC4=O)C5=O)CC3)c(F)c2)nc1Nc1ccc2c(c1)N(S(=O)(=O)C(C)(C)C)CC2]
or a pharmaceutically acceptable salt thereof.

29. A compound of Formula III or Formula IV 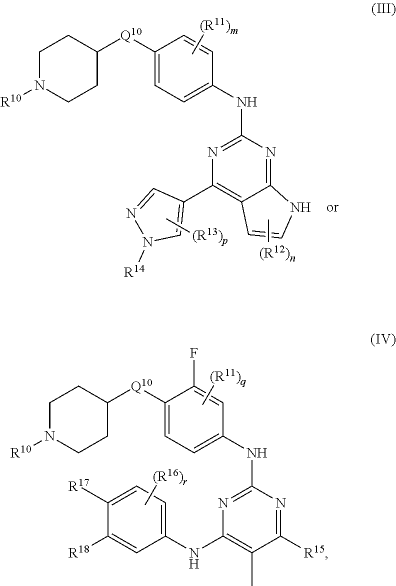
or a pharmaceutically acceptable salt thereof;
wherein:
R¹⁰ is selected from hydrogen or C₁-C₄ alkyl;
Q¹⁰ is a bond, —NH(C═O)—, or —C(═O)NH—;
R¹¹ is independently selected at each occurrence from hydrogen, alkyl, haloalkyl, alkoxy, alkenyl, alkynyl, aryl, heteroaryl, cycloalkyl, heterocycle, aldehyde amino, carboxylic acid, ester, ether, halo, hydroxy, keto, nitro, cyano, azido, silyl, sulfo-oxo, sulfonyl, sulfone, sulfoxide, sulfonylamino, and thiol;
m is selected from 0, 1, 2, 3, or 4;
q is 0, 1, 2, or 3;
R¹² is independently selected at each occurrence from hydrogen, alkyl, haloalkyl, alkoxy, alkenyl, alkynyl, aryl, heteroaryl, cycloalkyl, heterocycle, aldehyde amino, carboxylic acid, ester, ether, halo, hydroxy, keto, nitro, cyano, azido, silyl, sulfo-oxo, sulfonyl, sulfone, sulfoxide, sulfonylamino, and thiol;
n is 0, 1, or 2;
R¹³ is independently selected at each occurrence from hydrogen, alkyl, haloalkyl, alkoxy, alkenyl, alkynyl, aryl, heteroaryl, cycloalkyl, heterocycle, aldehyde amino, carboxylic acid, ester, ether, halo, hydroxy, keto, nitro, cyano, azido, silyl, sulfo-oxo, sulfonyl, sulfone, sulfoxide, sulfonylamino, and thiol;
p is 0, 1, or 2;
R¹⁴ is selected from 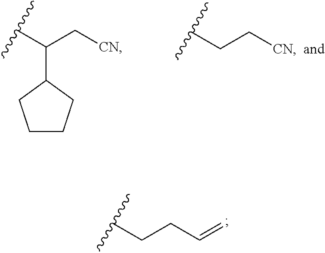
R¹⁵ is selected from hydrogen, alkyl, haloalkyl, alkoxy, alkenyl, alkynyl, aryl, heteroaryl, cycloalkyl, heterocycle, aldehyde amino, carboxylic acid, ester, ether, halo, hydroxy, keto, nitro, cyano, azido, silyl, sulfo-oxo, sulfonyl, sulfone, sulfoxide, sulfonylamino, and thiol;
R¹⁶ is independently selected at each occurrence from hydrogen, alkyl, haloalkyl, alkoxy, alkenyl, alkynyl, aryl, heteroaryl, cycloalkyl, heterocycle, aldehyde amino, carboxylic acid, ester, ether, halo, hydroxy, keto, nitro, cyano, azido, silyl, sulfo-oxo, sulfonyl, sulfone, sulfoxide, sulfonylamino, and thiol;
r is 0, 1, 2, or 3;
R¹⁷ is hydrogen or halogen;
R¹⁸ is —NHSO₂(C₁-C₄ alkyl); or
R¹⁷ and R¹⁸ are brought together with the carbon to which they are attached to form 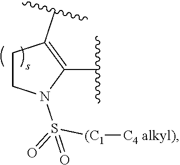
wherein s is 1 or 2.

30. A compound of claim 29, wherein R¹⁰ is hydrogen or methyl.

31. (canceled)

32. A compound of claim 29, wherein Q¹⁰ is a bond or —NH(C═O)—.

33-36. (canceled)

37. A compound of claim 29, wherein R¹⁷ is fluoro or chloro.

38. (canceled)

39. A compound of claim 29, wherein R¹⁸ is —NHSO₂(tert-butyl).

40. A compound of claim 29, wherein R¹⁷ and R¹⁸ are brought together with the carbons to which they are attached to form

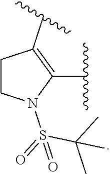

41. A compound selected from: 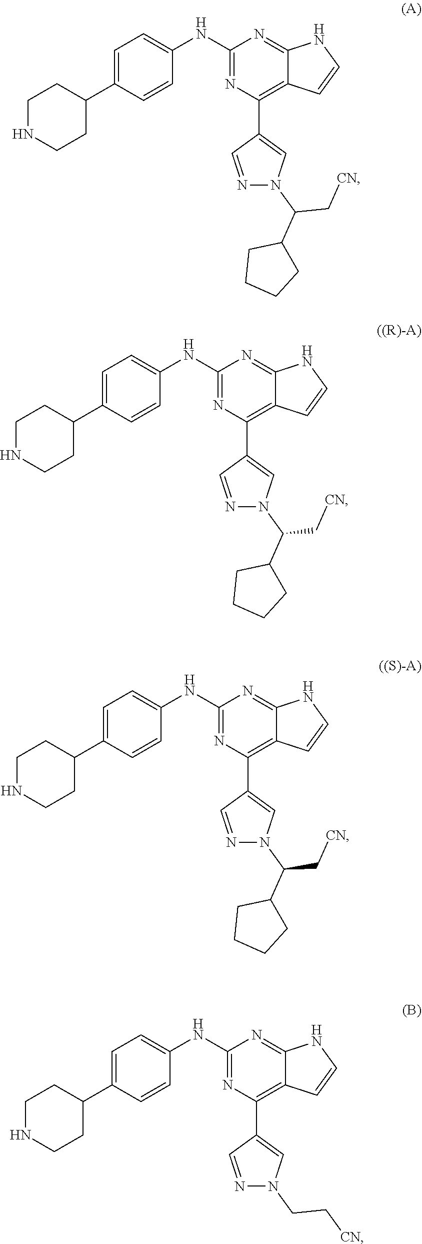 [SMILES: N#CCC(C1CCCC1)n1cc(-c2nc(Nc3ccc(C4CCNCC4)cc3)nc3[nH]ccc23)cn1 ; N#CC[C@H](C1CCCC1)n1cc(-c2nc(Nc3ccc(C4CCNCC4)cc3)nc3[nH]ccc23)cn1 ; N#CC[C@@H](C1CCCC1)n1cc(-c2nc(Nc3ccc(C4CCNCC4)cc3)nc3[nH]ccc23)cn1 ; N#CCCn1cc(-c2nc(Nc3ccc(C4CCNCC4)cc3)nc3[nH]ccc23)cn1] 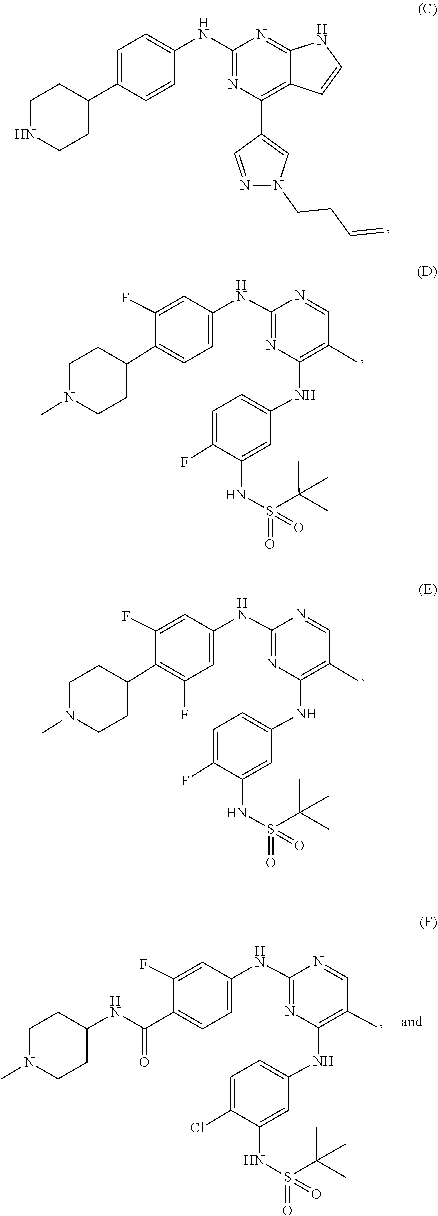 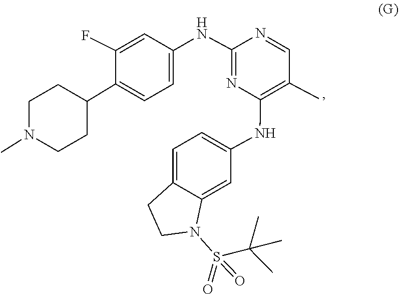
or a pharmaceutically acceptable salt thereof.

42. (canceled)

43. A pharmaceutical composition comprising a compound of of claim 1, or a pharmaceutically acceptable salt thereof, and a pharmaceutically acceptable carrier or excipient.

44. A method of treating an oncological disorder in a subject in need thereof comprising administering to the subject a therapeutically effective amount of a compound of claim 1, or a pharmaceutically acceptable salt thereof.

45. The method of claim 44, wherein the oncological disorder is a cancer.

46. The method of claim 45, wherein the cancer comprises a carcinoma, a sarcoma, a lymphoma, a leukemia, a germ cell tumor, or a blastoma.

47. The method of claim 45, wherein the cancer is selected from a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and myeloma.

48. The method of claim 45, wherein the cancer comprises acute lymphoblastic leukemia (ALL).

49-50. (canceled)
